(19) United States
(12) Patent Application Publication
(10) Pub. No.: US 2018/0246095 A1
(43) Pub. Date: Aug. 30, 2018

(54) COMPOSITIONS AND METHODS FOR DIAGNOSING AND TREATING LYME DISEASE

(71) Applicant: THE JOHNS HOPKINS UNIVERSITY, Baltimore, MD (US)

(72) Inventors: Ying ZHANG, Ellicott City, MD (US); Jie FENG, Lanzhou (CN)

(21) Appl. No.: 15/753,111

(22) PCT Filed: Aug. 17, 2016
(86) PCT No.: PCT/US2016/047366

§ 371 Date: Feb. 15, 2018

Related U.S. Application Data
(60) Provisional application No. 62/207,151, filed on Aug. 19, 2015.

(51) Int. Cl. G01N 33/569; A61K 31/65; A61K 31/43; A61P 31/04; C12Q 1/689

(52) U.S. Cl. CPC G01N 33/56911; A61K 31/65; A61K 31/43; G01N 2333/20; C12Q 1/689; C12Q 2600/158; A61P 31/04

(57) ABSTRACT

The present invention relates to compositions and methods of diagnosing and treating Lyme disease.

### RELATED APPLICATIONS

This application is an International Patent Application that claims the benefit of priority under 35 U.S.C. § 119(e) of U.S. Provisional Application No. 62/207,151, filed on Aug. 19, 2015, which is incorporated herein by reference in its entirety.

### FIELD OF THE INVENTION

The present invention relates to compositions and methods for treating and diagnosing Lyme disease. More particularly, the present invention relates to methods and compositions for treating Lyme disease based, in part, on genes whose expression is differentially modulated in antibiotic resistant strains of Borrelia burgdorferi.

### BACKGROUND OF THE INVENTION

Lyme disease caused by Borrelia burgdorferi is the most common vector borne disease in the United States and Europe. The current treatment for Lyme disease is a 2-4 week antibiotic monotherapy with doxycycline, amoxicillin or cefuroxime. While this treatment is effective for the majority of Lyme disease patients, about 10-20% of patients still have persisting symptoms such as fatigue, muscular pain, and neurological impairment—a collection of symptoms called Post Treatment Lyme Disease Syndrome (PTLDS)—that may last up to six months after the treatment. While the cause of PTLDS remains unclear, several hypotheses have been proposed to explain PTLDS, including: host response to continued presence of bacterial debris, autoimmunity, co-infections, and presence of bacterial persisters that were not killed by the current Lyme antibiotics. Consistent with the presence of bacterial persisters in various animal models such as mice, dogs and monkeys have shown B. burgdorferi could still be detected after treatment with different antibiotics, even though viable organisms could not be cultured. In vitro studies also demonstrated that B. burgdorferi could develop antibiotic tolerant persisters. Therefore, there is an urgent need to identify targets for more effective treatment of Lyme disease

### SUMMARY OF THE INVENTION

The present invention relates to compositions for treating and diagnosing Lyme disease. More particularly, the present invention relates to methods and compositions for treating Lyme disease based, in part, on genes whose expression is differentially modulated in antibiotic resistant strains of Borrelia burgdorferi.

### Definitions

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

As used herein, and unless otherwise indicated, the term “antisense oligonucleotide” refers to an oligonucleotide having a sequence complementary to a target DNA or RNA sequence.

As used herein, the term “antisense strand” of an siRNA or RNAi agent e.g., an antisense strand of an siRNA duplex or siRNA sequence, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific RNA interference (RNAi), e.g., complementarity sufficient to trigger the destruction of the desired target mRNA by the RNAi machinery or process. The term “sense strand” or “second strand” of a siRNA or RNAi agent e.g., an antisense strand of an siRNA duplex or siRNA sequence, refers to a strand that is complementary to the antisense strand or first strand. Antisense and sense strands can also be referred to as first or second strands, the first or second strand having complementarity to the target sequence and the respective second or first strand having complementarity to said first or second strand

As used herein, “comparing” in relation to “the proportion, level, or cellular localization, to a standard proportion, level, or cellular localization” refers to making an assessment of how the proportion, level, or cellular localization of a transcript or protein in a sample relates to the proportion, level, or cellular localization of a transcript or protein of the standard.

As used herein, and unless otherwise indicated, the term “complementary,” when used to describe a sequence in relation to a target sequence, means that the sequence is able to bind to the target sequence in a cellular environment in a manner sufficient to disrupt the function (e.g., replication, splicing, transcription or translation) of the gene comprising the target sequence. The binding may result from interactions such as, but not limited to, nucleotide base parings (e.g., A-T/G-C). In particular embodiments of the invention, a sequence is complementary when it hybridizes to its target sequence under high stringency, e.g., conditions for hybridization and washing under which nucleotide sequences, which are at least 60 percent (preferably greater than about 70, 80, or 90 percent) identical to each other, typically remain hybridized to each other. Such stringent conditions are known to those skilled in the art, and can be found, for example, in Current Protocols in Molecular Biology, John Wiley & Sons, N.Y. (1989), 6.3.1-6.3.6, which is incorporated herein by reference. Another example of stringent hybridization conditions is hybridization of the nucleotide sequences in 6× sodium chloride/sodium citrate (SSC) at about 45° C., followed by 0.2×SSC, 0.1% SDS at 50-65° C. Particularly preferred stringency conditions are hybridization in 6x sodium chloride/sodium citrate (SSC) at about 45° C., followed by one or more washes in 0.2.×SSC, 0.1% SDS at 50° C. Depending on the conditions under which binding sufficient to disrupt the functions of a gene occurs, a sequence complementary to a target sequence within the gene need not be 100 percent identical to the target sequence. For example, a sequence can be complementary to its target sequence when at least about 70%, 80%, 90%, or 95% of its nucleotides bind via matched base pairings with nucleotides of the target sequence.

As used herein, “correlating” in reference to a parameter, e.g., a modulated ratio, level, or localization in a sample or tissue from the subject, may be an indication that B. burgdorferi infection is absent, present, persisting, and/or getting worse or better. “Correlating” or “normalization” as used according to the present invention may be by any method of relating levels of expression or localization of markers to a standard valuable for the: the diagnosis of Lyme disease, prediction of Lyme disease presence, progression, assessment of efficacy of clinical treatment, selection of a subject for a particular treatment, monitoring of the progress of treatment with a B. burgdorferi directed therapy, and in the context of a screening assay, for the identification of a B. burgdorferi related therapeutic.

When used to describe the sequences of siRNAs, the term “corresponding to,” as used herein, means that a siRNA has a sequence that is identical or complementary to the portion of target mRNA that is transcribed from the denoted DNA sequence.

As used herein, and unless otherwise indicated, the term “inhibiting the synthesis or expression” of a gene means impeding, slowing or preventing one or more steps by which the end-product protein encoded by said gene is synthesized. Typically, the inhibition involves blocking of one or more steps in the gene's replication, transcription, splicing or translation through a mechanism that comprises recognition of a target site located within the gene or transcript sequence based on sequence complementation. In a specific embodiment, inhibition of B. burgdorferi reduces the amount of B. burgdorferi in the cell by greater than about 20%, 40%, 60%, 80%, 85%, 90%, 95%, or 100%. The amount of B. burgdorferi can be determined by well-known methods including, but are not limited to, densitometer, fluorometer, radiography, gram staining luminometer, antibody-based methods and activity measurements, PCR, and/or mass spectrometry.

As used herein, the term “isolated RNA” (e.g., “isolated ssRNA”, “isolated siRNA” or “isolated ss-siRNA”) refers to RNA molecules which are substantially free of other cellular material, or culture medium when produced by recombinant techniques, or substantially free of chemical precursors or other chemicals when chemically synthesized.

The term “measuring” means methods which include detecting the presence or absence of a biomarker(s) in a sample, quantifying the amount of biomarker(s) in the sample, and/or qualifying the type of biomarker(s). Measuring can be accomplished by methods known in the art and those further described herein including, but not limited to, immunoassay, PCR, qPCR, RT PCR, etc.

As used herein, the term “molecule” when used without other qualification, e.g., nucleic acid molecule, refers to both compounds of biological origin or character (e.g., proteins, DNA, RNA, antibodies, etc.) and compounds which are synthetic organic compounds (e.g., aspirin, ibuprofen, ampicillin, etc.).

The term “sample,” as used herein, refers to a biological sample obtained for the purpose of evaluation in vitro. In the methods of the present invention, the sample or patient sample may comprise anybody fluid including, but not limited to, blood, serum, plasma, urine, saliva, and synovial fluid. A sample may also comprise any cells, tissue samples or cell components (such as cellular membranes or cellular components) obtained from a patient. In a further embodiment, a sample may refer to a stool sample.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

The terms “subject” or “patient” are used interchangeably herein, and is meant a mammalian subject to be treated, with human subjects being preferred. In some cases, the terms may refer to treatment in experimental animals, in veterinary application, and in the development of animal models for disease, including, but not limited to, rodents including mice, rats, and hamsters; and primates.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, for example, as described herein. In one embodiment, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

A “target gene” is a gene whose expression is to be selectively inhibited or “silenced.” In certain embodiments, this silencing is achieved by cleaving the mRNA of the target gene by an siRNA that is created from an engineered RNA precursor by a cell's RNAi system. One portion or segment of a duplex stem of the RNA precursor is an anti-sense strand that is complementary, e.g., fully complementary, to a section of about 18 to about 40 or more nucleotides of the mRNA of the target gene.

### BRIEF DESCRIPTION OF THE DRAWINGS

FIG. 1A is a diagram depicting genes that are differentially expressed in doxycycline tolerant B. burgdorferi persisters. The green upward pentagons indicate upregulated genes, and the red downward pentagons indicate downregulated genes. Pathways in bold frame were differentially expressed in both doxycycline and amoxicillin tolerant persisters.

FIG. 1B is a diagram depicting genes that are differentially expressed in amoxicillin tolerant B. burgdorferi persisters. The green upward pentagons indicate upregulated genes, and the red downward pentagons indicate downregulated genes. Pathways in bold frame were differentially expressed in both doxycycline and amoxicillin tolerant persisters.

### DETAILED DESCRIPTION OF THE INVENTION

Lyme disease caused by Borrelia burgdorferi is the most common vector borne disease in the United States and Europe (Lyme Disease. Center for Disease Control; 2015; Available from the world wide web at (www)cec.gov/lyme/, and Rizzoli A, et al. Euro Surveill. 2011; 16(27)). The current treatment for Lyme disease is a 2-4 week antibiotic monotherapy with doxycycline, amoxicillin or cefuroxime (Wormser G P, et al. Clinical infectious diseases: an official publication of the Infectious Diseases Society of America. [Practice Guideline]. 2006 Nov. 1; 43(9):1089-134). While this treatment is effective for the majority of Lyme disease patients, about 10-20% of patients still have persisting symptoms such as fatigue, muscular pain, and neurological impairment (Lyme Disease. Center for Disease Control; 2015; Available from: the world wide web at (www)cec.gov/lyme)—a collection of symptoms called Post Treatment Lyme Disease Syndrome (PTLDS) (CDC. Post-Treatment Lyme Disease Syndrome)—that may last up to six months after the treatment. While the cause of PTLDS remains unclear and controversial, several hypotheses have been proposed to explain PTLDS, including host response to continued presence of bacterial debris (Bockenstedt L K, et al., J Clin Invest. 2012 Jul. 2; 122(7):2652-60), autoimmunity (Steere A C, et al., Journal of autoimmunity. 2001 May; 16(3):263-8), co-infections (Stricker R B. Clinical infectious diseases: an official publication of the Infectious Diseases Society of America. 2007 Jul. 15; 45(2):149-57), and presence of bacterial persisters not killed by the current Lyme antibiotics (Stricker R B. Clinical infectious diseases: an official publication of the Infectious Diseases Society of America. 2007 Jul. 15; 45(2):149-57). Consistent with the presence of bacterial persisters not killed by current antibiotics, experiments in various animal models such as mice, dogs and monkeys have shown that B. burgdorferi could still be detected after treatment with different Lyme antibiotics though viable organisms could not be cultured (Barthold S W, et al., Antimicrobial Agents and Chemotherapy. 2010 February; 54(2):643-51, Embers M E, et al. PLoS One. 2012; 7(1):e29914 and Straubinger R K, et al., J Clin Microbiol. 1997 January; 35(1):111-6). In vitro studies also demonstrated that B. burgdorferi could develop antibiotic tolerant persisters (Feng J, et al. Emerg Microb Infect. 2014; Jul. 2, 2014:3, e49). Although persister mechanisms have been reported in the model organism E. coli (Zhang Y. Emerg Microb Infect. 2014; 3, e3), the mechanisms that give rise to B. burgdorferi persisters remain unknown. Herein, RNA Sequencing (RNA-Seq) was performed to determine the gene expression profile of B. burgdorferi persisters that survived antibiotic treatment using next generation sequencing to shed light on the mechanisms of B. burgdorferi persistence.

Borrelia burgdorferi

Borrelia species known to cause Lyme disease are collectively known as Borrelia burgdorferi. Borrelia are microaerophilic and slow-growing, which is the primary reason for the long delays when diagnosing Lyme disease. Borrelia burgdorferi has been found to have great strain diversity wherein the strains differ in clinical symptoms and/or presentation as well as geographic distribution. Except for Borrelia recurrentis, which is transmitted by the body louse, all known species are believed to be transmitted by ticks.

### Prevalence

The rate of Lyme disease infection has increased to 7.9 cases for every 100,000 persons. Although Lyme disease has now been reported in 49 of 50 states in the U.S. (all states except Hawaii), about 99% of all reported cases are confined to just five geographic areas (New England, Mid-Atlantic, East-North Central, South Atlantic, and West North-Central). In Europe, cases of B. burgdorferi-infected ticks are found predominantly in Norway, the Netherlands, Germany, France, Italy, Slovenia, and Poland, but have also been isolated in almost every country on the continent. Borrelia burgdorferi-infested ticks are being found more frequently in Japan, as well as in northwest China and far eastern Russia. In western and sub-Saharan Africa, tick-borne relapsing fever has been recognized for over a century.

### Lifecycle

The lifecycle of B. burgdorferi is complex, requiring ticks, and species that are competent reservoirs for the organism, which often include small rodents. For example, mice are the primary reservoir for the bacteria.

B. burgdorferi B31 Strain

The genome of B. burgdorferi (B31 strain) was the third microbial genome ever to be sequenced, and its chromosome contains 910,725 base pairs and 853 genes. One of the most striking features of B. burgdorferi is its unusual genome, which is far more complex than that of its spirochetal cousin Treponema pallidum, the agent of syphilis. In addition to a linear chromosome, the genome of B. burgdorferi strain B31 includes 21 plasmids (12 linear and 9 circular), the largest number of plasmids found in any known bacterium. Genetic exchange, including plasmid transfers, contributes to the genetic heterogeneity and pathogenicity of the organism. Long-term culture of B. burgdorferi results in a loss of some plasmids and changes in expressed protein profiles. Associated with the loss of plasmids is a loss in the ability of the organism to infect laboratory animals, suggesting the plasmids encode key genes involved in virulence.

B. burgdorferi Structure and Growth

B. burgdorferi is a highly specialized, motile, two-membrane, flat-waved spirochete, ranging from about 9 to 32 micrometers in length. Because of its double-membrane envelope, it is often mistakenly described as being Gram negative, although it does stain weakly positive in Gram stain. The bacterial membranes in at least the B31, NL303 and N40 strains of B. burgdorferi do not contain lipopolysaccharide, which is extremely atypical for Gram negative bacteria; instead, the membranes contain glycolipids. However, the membranes in the B31 strain have been found to contain a lipopolysaccharide-like component. B. burgdorferi is a microaerophilic organism, requiring little oxygen to survive. Unlike most bacteria, B. burgdorferi does not use iron, hence avoiding the difficulty of acquiring iron during infection. It lives primarily as an extracellular pathogen, although in vitro it can also hide intracellularly

B. burgdorferi is very slow growing, with a doubling time of 12-18 hours. Since most antibiotics kill bacteria only when they are dividing, this longer doubling time necessitates the use of relatively longer treatment courses for Lyme disease.

### Outer Surface Proteins

The outer membrane of B. burgdorferi is composed of various unique outer surface proteins (Osp) that have been characterized (e.g., OspA through OspF). The Osp proteins are lipoproteins anchored by N-terminally attached fatty acid molecules to the membrane. OspA, OspB, and OspD are expressed by B. burgdorferi residing in the gut of unfed ticks, suggesting they promote the persistence of the spirochete in ticks between blood meals. During transmission to the mammalian host, when the nymphal tick begins to feed and the spirochetes in the midgut begin to multiply rapidly, most spirochetes cease expressing OspA on their surfaces. Simultaneous with the disappearance of OspA, the spirochete population in the midgut begins to express an OspC and migrates to the salivary gland. Upregulation of OspC begins during the first day of feeding and peaks 48 hours after attachment.

### Mechanisms of Persistence

While B. burgdorferi is susceptible to a number of antibiotics in vitro, reports are contradictory as to the efficacy of antibiotics in vivo. B. burgdorferi may persist in humans and animals for months or years. Some studies have suggested persistence of infection despite antibiotic therapy, although others have suggested that antibiotics rapidly end infections.

B. burgdorferi can invade a variety of cultured cells, including endothelium, fibroblasts, lymphocytes, macrophages, keratinocytes, synovium, and most recently neuronal and glial cells. By hiding inside these cells during human infection, B. burgdorferi may be able to evade the immune system and be protected to varying degrees against some antibiotics, sometimes allowing the infection to persist.

The formation of rounded forms of B. burgdorferi cells, sometimes called spheroplasts, which either lack a cell wall or have a damaged cell wall, has been observed in vitro, in vivo, and in an ex vivo model. The finding that energy is required for the spiral bacterium to convert to this form suggests that these altered forms have a survival function, and are not merely end-stage degeneration products.

Like the Borrelia that causes relapsing fever, B. burgdorferi has the ability to vary its surface proteins in response to immune attack. This ability is related to the genomic complexity of B. burgdorferi, and is another way B. burgdorferi evades the immune system to establish a chronic infection.

### Biomarkers

clpP

ClpP protease is an intracellular protease which may recognize and degrade misfolded proteins with the aid of ClpX, C or A subunits.

### Heat Shock Proteins

Heat shock proteins (HSPs) function as intra-cellular chaperones for other proteins. They play an important role in protein-protein interactions such as folding and assisting in the establishment of proper protein conformation and prevention of unwanted protein aggregation. HSPs aid in transporting proteins across membranes within a cell.

### Transporter Proteins

Transporter proteins (phosphotransferase system, maltose and glucose uptake transporter, Na⁺/Ca²⁺ exchanger, dicarboxylate/amino acid uptake transporter and Na/H antiporter) facilitate in uptek of nutrients and regulate intracellular ion concentration.

### Membrane Proteins

Membrane proteins (e.g., S1 antigen, S2, bmpD, BB_0807, BB_G28) interact with biological membranes and target over 50% of all modern medicinal drugs. Membrane proteins perform a variety of functions including transport of signals, transport proteins, enzymatic activities, and cell adhesion interactions (e.g., proteins involved in immune response).

### Detection of Biomarkers

### Detection by Mass Spectrometry

In another aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these. In a particular method, the mass spectrometer is a laser desorption/ionization mass spectrometer. In a specific embodiment, the mass spectrometric technique comprises surface enhanced laser desorption and ionization or “SELDI,” as described, for example, in U.S. Pat. No. 6,225,047 and U.S. Pat. No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

### Detection by Immunoassay

In another embodiment, the biomarkers of the present invention can be measured by immunoassay Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

The present invention contemplates traditional immunoassays including, for example, sandwich immunoassays including ELISA or fluorescence-based immunoassays, as well as other enzyme immunoassays. Nephelometry is an assay performed in liquid phase, in which antibodies are in solution. Binding of the antigen to the antibody results in changes in absorbance, which may be measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry. The Quantikine immunoassay developed by R&D Systems, Inc. (Minneapolis, Minn.) may also be used in the methods of the present invention.

### Detection by Electrochemicaluminescent Assay

In several embodiments, the B. burgdorferi biomarkers and other biomarkers may be detected by means of an electrochemicaluminescent assay developed by Meso Scale Discovery (Gaithersburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Application Publication No's: 2009/0170121; 2009/006339; 2009/0065357; 2006/0172340; 2006/0019319; 2005/0142033; 2005/0052646; 2004/0022677; 2003/0124572; 2003/0113713; 2003/0003460; 2002/0137234; 2002/0086335; and 2001/0021534.

### Detection by Other Methods

The B. burgdorferi biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### Sample Preparation

In several embodiments of the present invention, a blood sample is tested for the presence or absence of one or more biomarkers. The step of collecting a sample such as a blood sample from a subject can be carried out by phlebotomy or any other suitable technique. The blood sample may be further processed to provide a serum sample or other suitable blood fraction, such as plasma.

In alternative embodiments of the present invention, a tissue sample may be taken and tested for the presence or absence of one or more biomarkers. Tissue or cell samples can be removed from almost any part of the body. In particular, biopsy methods include needle (e.g. fine needle aspiration), endoscopic, and excisional. Variations of these methods and the necessary devices used in such methods are known to those of ordinary skill in the art.

### Biomarker Panels

The skilled artisan will appreciate there are many ways to use the measurements of two or more markers in order to improve the diagnostic question under investigation. In a quite simple, but nonetheless often effective approach, a positive result is assumed if a sample is positive for at least one of the markers investigated.

Frequently, however, the combination of markers is evaluated. Preferably the values measured for markers of a marker panel, e.g., for B. burgdorferi biomarkers, are mathematically combined and the combined value is correlated to the underlying diagnostic question. Biomarker values may be combined by any appropriate state of the art mathematical method. Well-known mathematical methods for correlating a marker combination to a disease employ methods like discriminant analysis (DA) (e.g., linear-, quadratic-, regularized-DA), Kernel Methods (e.g., SVM), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in correlating biomarker combination of the present invention e.g. to the absence or presence of Lyme disease is selected from DA (e.g., Linear-, Quadratic-, Regularized Discriminant Analysis), Kernel Methods (e.g., SVM), Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression). Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

### Determining Risk of Developing Disease

In a specific embodiment, the present invention provides methods for determining the risk of developing disease (e.g. Lyme disease) in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level.

### Determining Stage of Disease

In another embodiment, the present invention provides methods for determining the stage of disease (e.g. Lyme disease) in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

### Determining Course (Progression) of Disease

In one embodiment, the present invention provides methods for determining the course of disease (e.g. Lyme disease) in a subject. Disease course refers to changes in disease status over time, including disease progression (worsening) and disease regression (improvement). Over time, the amounts or relative amounts (e.g., the pattern) of the biomarker(s) changes. Accordingly, this method involves measuring one or more biomarkers in a subject at least two different time points, e.g., a first time and a second time, and comparing the change in amounts, if any. The course of disease is determined based on these comparisons.

### Subject Management

In certain embodiments of the methods of determining Lyme disease status, the methods further comprise managing subject treatment based on the status. Such management includes the actions of the physician or clinician subsequent to determining Lyme disease status. For example, if a physician makes a diagnosis of Lyme disease, based on the teachings herein, then a certain regime of treatment, such as prescription or administration a specific of therapeutic agent(s) might follow. Alternatively, a diagnosis of non-Lyme disease might be followed with further testing to confirm the absence of B. burgdorferi infection, or to determine a specific disease that the patient might be suffering from. Also, further tests may be called for if the diagnostic test gives an inconclusive result on Lyme disease status.

### Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of one or more of the biomarkers of the present invention may change toward a non-disease profile. Therefore, one can follow the course of the amounts of one or more biomarkers in the subject during the course of treatment. Accordingly, this method involves measuring one or more biomarkers in a subject receiving drug therapy, and correlating the amounts of the biomarkers with the disease status of the subject. One embodiment of this method involves determining the levels of one or more biomarkers at least two different time points during a course of drug therapy, e.g., a first time and a second time, and comparing the change in amounts of the biomarkers, if any. For example, the one or more biomarkers can be measured before and after drug administration or at two different time points during drug administration. The effect of therapy is determined based on these comparisons. If a treatment is effective, then one or more biomarkers will trend toward normal, while if treatment is ineffective, the one or more biomarkers will trend toward disease indications. If a treatment is effective, then the one or more biomarkers will trend toward normal, while if treatment is ineffective, the one or more biomarkers will trend toward disease indications.

### Kits for the Detection of Lyme Disease

In another aspect, the present invention provides kits for determining Lyme disease, which kits are used to detect the biomarker and other biomarkers. In a specific embodiment, the kit is provided as an ELISA kit comprising an antibody to biomarker. The ELISA kit may comprise a solid support, such as a chip, microtiter plate (e.g., a 96-well plate), bead, or resin having a biomarker capture agent attached thereon. The kit may further comprise a means for detecting the biomarker, such as an antibody, and a secondary antibody-signal complex such as horseradish peroxidase (HRP)-conjugated goat anti-rabbit IgG antibody and tetramethyl benzidine (TMB) as a substrate for HRP.

The kit for determining Lyme disease may be provided as an immuno-chromatography strip comprising a membrane on which an antibody is immobilized, and a means for detecting the biomarker, e.g., a gold particle bound biomarker antibody, where the membrane, includes NC membrane and PVDF membrane. The kit may comprise a plastic plate on which a sample application pad, a gold particle bound biomarker antibody temporally immobilized on a glass fiber filter, a nitrocellulose membrane on which a biomarker antibody band and a secondary antibody band are immobilized and an absorbent pad are positioned in a serial manner, so as to keep continuous capillary flow of blood serum.

A patient can be diagnosed by adding a sample from the patient to the kit and detecting said biomarker conjugated with a biomarker antibody, specifically, by a method which comprises the steps of: (i) collecting sample from the patient; (ii) centrifuging sample; (iii) adding the sample from patient to a diagnostic kit; and, (iv) detecting said biomarker conjugated with an antibody. In this method, the antibodies are brought into contact with the patient's sample. If said biomarker is present in the sample, the antibodies will bind to the sample, or a portion thereof. In other kit and diagnostic embodiments, sample need not be collected from the patient (i.e., it is already collected).

The kit can also comprise a washing solution or instructions for making a washing solution, in which the combination of the capture agent and the washing solution allows capture of the biomarker or biomarkers on the solid support for subsequent detection by, e.g., an antibody or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected. In yet another embodiment, the kit can comprise one or more containers with biomarker samples, to be used as standard(s) for calibration.

Treatment of Lyme Disease by Targeting B. burgdorferi

The present invention generally relates to the treatment and management of Lyme disease by targeting B. burgdorferi. In one aspect, the present invention relates to inhibiting the expression of B. burgdorferi. Inhibition may be achieved by impeding any steps in the replication, transcription, splicing or translation of the B. burgdorferi gene. In another aspect, the present invention relates to interfering, inhibiting, or otherwise preventing the functional aspects of the B. burgdorferi protein.

Antibodies to B. burgdorferi

The present invention contemplates the use of antibodies specific for B. burgdorferi in the treatment and prevention of Lyme disease. The phrases “binding specificity,” “binding specifically to, “specific binding” or otherwise any reference to an antibody to B. burgdorferi, refers to a binding reaction that is determinative of the presence of the corresponding B. burgdorferi antigen to the antibody in a heterogeneous population of antigens and other biologics. The parameters required to achieve such specificity can be determined routinely, using conventional methods in the art including, but not limited to, competitive binding studies. The binding affinity of an antibody can also be readily determined, for example, by Scatchard analysis (Scatchard, Ann. NY Acad. Sci. 51: 660-672, 1949). In some embodiments, the immunoglobulins of the present invention bind to B. burgdorferi at least about 5, at least about 10, at least about 100, at least about 10³, at least about 10⁴, at least 10⁵, and at least 10⁶ fold higher than to other proteins.

Various procedures known in the art may be used for the production of antibodies to B. burgdorferi family members or any subunit thereof, or B. burgdorferi, or a fragment, derivative, homolog or analog of the protein. Antibodies of the present invention include, but are not limited to, synthetic antibodies, polyclonal antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, e.g., molecules that contain an antigen binding site that immunospecifically binds to an antigen (e.g., one or more complementarity determining regions (CDRs) of an antibody).

Another embodiment for the preparation of antibodies according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics in rational design is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting antibodies disclosed herein, but with altered and even improved characteristics. More specifically, under this rational design approach, peptide mapping may be used to determine “active” antigen recognition residues, and along with molecular modeling and molecular dynamics trajectory analysis, peptide mimic of the antibodies containing antigen contact residues from multiple CDRs may be prepared.

In some embodiments, an antibody specifically binds an epitope of the B. burgdorferi protein. It is to be understood that the peptide regions may not necessarily precisely map one epitope, but may also contain B. burgdorferi sequence that is not immunogenic. Methods of predicting other potential epitopes to which an immunoglobulin of the invention can bind are well-known to those of skill in the art and include, without limitation, Kyte-Doolittle Analysis (Kyte, J. and Dolittle, R. F., 157 J. MOL. BIOL. 105-32 (1982)); Hopp and Woods Analysis (Hopp, T. P. and Woods, K. R., 78 PROC. NATL. ACAD. Sci. USA 3824-28 (1981); Hopp, T. J. and Woods, K. R., 20 MOL. IMMUNOL. 483-89 (1983); Hopp, T. J., 88 J. IMMUNOL. METHODS 1-18 (1986)); Jameson-Wolf Analysis (Jameson, B. A. and Wolf, H., 4 COMPUT. APPL. BIOSCI. 181-86 (1988)); and Emini Analysis (Emini et al., 140 VIROLOGY 13-20 (1985)).

Amino acid sequence variants of the antibodies of the present invention may be prepared by introducing appropriate nucleotide changes into the polynucleotide that encodes the antibody or by peptide synthesis. Such modifications include, for example, deletions from, and/or insertions into and/or substitutions of, residues within the amino acid sequences of the antibody. Any combination of deletions, insertions, and substitutions may be made to arrive at the final construct.

Amino acid sequence insertions include amino-terminal and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of a polypeptide that increases the serum half-life of the antibody.

Another type of antibody variant is an amino acid substitution variant. These variants have at least one amino acid residue in the antibody molecule replaced by a different residue. For example, the sites of greatest interest for substitutional mutagenesis of antibodies include the hypervariable regions, but framework region (FR) alterations are also contemplated.

A useful method for the identification of certain residues or regions of the B. burgdorferi antibodies that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed antibody variants screened for the desired activity.

Substantial modifications in the biological properties of the antibody can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability, particularly where the antibody is an immunoglobulin fragment such as an Fv fragment.

Another type of substitutional variant involves substituting one or more hypervariable region residues of a parent antibody. Generally, the resulting variant(s), i.e., functional equivalents as defined above, selected for further development will have improved biological properties relative to the parent antibody from which they are generated. A convenient way for generating such substitutional variants is by affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the antibody-antigen complex to identify contact points between the antibody and antigen. Such contact residues and neighboring residues are candidates for substitution according to the techniques elaborated herein. Once generated, the panel of variants is subjected to screening as described herein and antibodies with superior properties in one or more relevant assays may be selected for further development.

It may be desirable to modify the antibodies of the present invention, i.e., create functional equivalents, with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Caron et al., 176 J. EXP MED. 1191-95 (1992); Shopes, 148 J. IMMUNOL. 2918-22 (1992). Homodimeric antibodies with enhanced anti-tumor activity may also be prepared using heterobifunctional cross-linkers as described in Wolff et al., 53 CANCER RESEARCH 2560-65 (1993). Alternatively, an antibody can be engineered which has dual Fc regions and may thereby have enhanced complement lysis and ADCC capabilities. Stevenson et al., 3 ANTI-CANCER DRUG DESIGN 219-30 (1989).

To increase the serum half-life of an antibody, one may incorporate a salvage receptor binding epitope into the antibody (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Polynucleotide molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the anti-B. burgdorferi antibodies of the present invention.

### Small Molecule Inhibitors

In another aspect, the present invention provides methods and compositions for treating Lyme disease associated with B. burgdorferi. Any compound that inhibits the action of B. burgdorferi may be used in the present invention.

### Pharmaceutical Compositions for the Treatment of Lyme Disease

The present invention also provides pharmaceutical compositions. Such compositions comprise a therapeutically effective amount of a B. burgdorferi therapeutic agent and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the B. burgdorferi therapeutic agent is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water can be a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the B. burgdorferi therapeutic, preferably in purified form, together with a suitable amount of carrier so as to provide the form for proper administration to the patient. The formulation should suit the mode of administration.

In a specific embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The B. burgdorferi therapeutic agent(s) of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

Particular pharmaceutical compositions and dosage forms comprise a B. burgdorferi therapeutic agent of the invention, or a pharmaceutically acceptable prodrug, salt, solvate, or clathrate thereof, optionally in combination with one or more additional active agents.

### Routes of Administration

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means.

### Dosage Determinations

In general, the pharmaceutical compositions disclosed herein may be used alone or in concert with other therapeutic agents at appropriate dosages defined by routine testing in order to obtain optimal efficacy while minimizing any potential toxicity. The dosage regimen utilizing a composition of the present invention may be selected in accordance with a variety of factors including type, species, age, weight, sex, medical condition of the patient; the severity of the condition to be treated; the route of administration; the renal and hepatic function of the patient; and the particular composition employed. A physician of ordinary skill can readily determine and prescribe the effective amount of the drug required to prevent, counter, or arrest the progress of the condition.

Optimal precision in achieving concentrations of drug within the range that yields maximum efficacy with minimal toxicity may require a regimen based on the kinetics of the composition's availability to one or more target sites. Distribution, equilibrium, and elimination of a drug may be considered when determining the optimal concentration for a treatment regimen. The dosages of a composition disclosed herein may be adjusted when combined to achieve desired effects. On the other hand, dosages of these various therapeutic agents may be independently optimized and combined to achieve a synergistic result wherein the pathology is reduced more than it would be if either agent were used alone.

In particular, toxicity and therapeutic efficacy of a composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Compositions exhibiting large therapeutic indices are preferred except when cytotoxicity of the composition is the activity or therapeutic outcome that is desired. Although compositions that exhibit toxic side effects may be used, a delivery system can target such compositions to the site of affected tissue in order to minimize potential damage to uninfected cells and, thereby, reduce side effects. Generally, the compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

### Dosages

More specifically, the compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of humans or animals can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

### Combination Therapy

It would be readily apparent to one of ordinary skill in the art that the compositions of the present invention (e.g., siRNAs, antisense oligonucleotides, and other therapeutic agents, e.g., doxycyclindescribed herein) can be combined with one or more of other Lyme disease therapies. The determination of the identity and amount of second anti-Lyme disease agent(s) for use in a method of the present invention can be readily made by ordinarily skilled medical practitioners using standard techniques known in the art.

The compositions of the present invention and second anti-Lyme disease agents can be administered simultaneously or sequentially by the same or different routes of administration. In particular, the compositions of the present invention can be administered simultaneously or sequentially with certain antibiotics, including doxycycline, amoxicillin, or cefuroxime axetil. Additionally, ceftriaxone, cefotaxime, penicillin G, or bismacine may be administered simultaneously.

In another aspect, the B. burgdorferi therapeutic agents may be combined with other agents including, but not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

In various embodiments, the B. burgdorferi therapeutic agent in combination with a second therapeutic agent may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, one or more compounds of the present invention and one or more other therapies are cyclically administered. Cycling therapy involves the administration of a first therapy (e.g., a first B. burgdorferi therapeutic agent) for a period of time, followed by the administration of a second therapy (e.g. a second B. burgdorferi therapeutic agent, or another therapeutic agent) for a period of time, optionally, followed by the administration of a third therapy for a period of time and so forth, and repeating this sequential administration, e.g., the cycle in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

### Kits

The present invention also provides kits for use in treating and/or diagnosing Lyme disease. The kits of the present invention include one or more containers comprising B. burgdorferi therapeutic agents (siRNAs, antibodies, small molecules, etc.), and in some embodiments, further comprise instructions for use in accordance with any of the methods described herein. The kit may further comprise a description of selecting an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of the present invention are provided in suitable packaging. Suitable packaging include, but is not limited to, vials, bottles, jars, flexible packaging (e.g., sealed Mylar or plastic bags), and the like. Kits may optionally provide additional components such as buffers and interpretative information.

The instructions relating to the use of the therapeutic compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the composition as disclosed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compositions and instructions for use and packaged in quantities sufficient for storage and use in pharmacies, for example, hospital pharmacies and compounding pharmacies.

### EXAMPLES

### Example 1: RNA Sequencing (RNA-Seq) to Determine Gene Expression Profile of B. burgdorferi Persisters that Survived Antibiotic Treatment

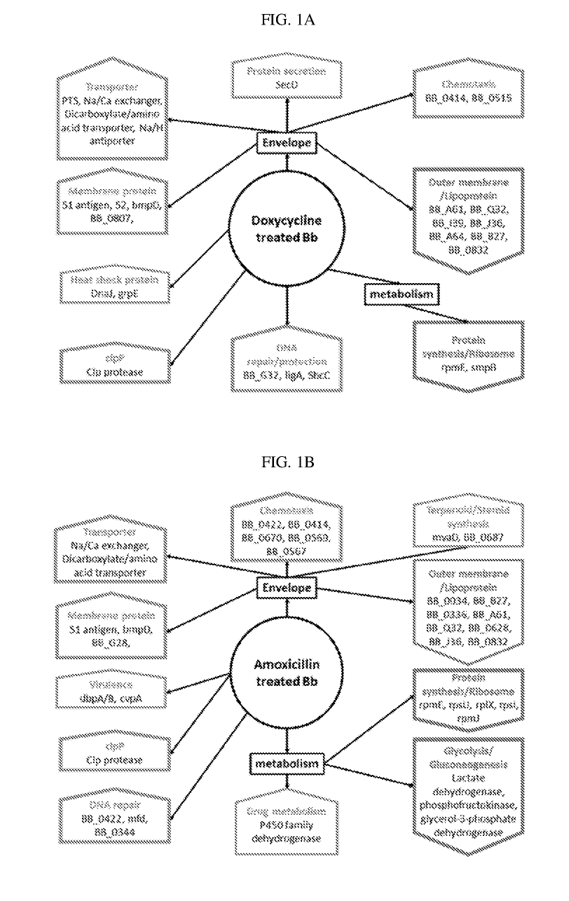

RNA Sequencing (RNA-Seq) was performed to determine the gene expression profile of B. burgdorferi persisters that survived antibiotic treatment using next generation sequencing to shed light on the mechanisms of B. burgdorferi persistence. B. burgdorferi B31 cultures were grown for 5 days to 1×10⁷ spirochetes/ml in BSK-H medium followed by treatment with 50 μg/ml doxycycline or 50 μg/ml amoxicillin for 6 days. Triplicate biological samples were used for each group. The total RNA was extracted and ribosomal RNA was removed prior to preparation of cDNA libraries for Illumina sequencing. The up-regulated and down-regulated genes in the amoxicillin and doxycycline treated B. burgdorferi persisters were analyzed and compared with drug free control (FIG. 1A and FIG. 1B). The RNA-Seq data was analyzed by BLAST analysis and mapped to KEGG pathways.

### Example 2: RT-PCR on Upregulated and Downregulated Candidate Genes of Doxycycline Treated B. burgdorferi

Real-time quantitative reverse transcription PCR (RT-PCR) was performed for candidate genes that were upregulated (bmpD and clpP) or down-regulated (gene of membrane protein BB_0428 and rpmE) to validate the RNA-Seq data.

For doxycycline treated B. burgdorferi persisters, a total of 675 genes were differentially expressed between doxycycline tolerant persisters and drug free group, with 335 genes upregulated and 340 genes down-regulated in doxycycline treated group (Table 1). A total of 35 genes were identified by more than two-fold changes among up-regulated genes (FIG. 1A), including five transporter genes (BB_0164, BB_0116, BB_0637, BB_0729 and BB_B29), four bacterial envelope protein coding genes (BB_0158, BB_A05, BB_0385 and BB_0201), three DNA repair related genes (BB_G32, BB_0552, BB_0830), two bacterial chemotaxis genes (BB_0114 and BB_0515), one bacterial secretion gene secD (BB_0652), and the clpP (BB_0757) gene encoding Clp protease. On the other hand, the majority of down-regulated genes (33 genes) were associated with genes encoding outer membrane proteins and ribosome proteins.

clpP encoding ATP-dependent Clp protease proteolytic subunit was the most highly upregulated (30 fold) in doxycycline treated B. burgdorferi persisters. ClpP protease is an intracellular protease which could recognize and degrade misfolded proteins with the aid of ClpX, C or A subunits. Doxycycline could disturb bacterial protein synthesis by binding to the 30S ribosomal subunit that might lead to produce misfolded proteins. The up-regulation of clpP could be a response to this situation. Meanwhile genes encoding heat shock proteins (HSP) were found to be upregulated. The HSP molecular chaperones could stabilize new proteins to ensure correct folding and help to refold damaged proteins under stress, which could be important for persister survival.

Five up-regulated transporter genes (encoding phosphotransferase system, maltose and glucose uptake transporter, Na⁺/Ca²⁺ exchanger, dicarboxylate/amino acid uptake transporter and Na/H antiporter) could facilitate uptake of nutrients (carbohydrates and amino acid) and regulate intracellular ion concentration under the pressure of doxycycline. Some up-regulated genes were associated with DNA repair, which might help to maintain stability of DNA under the doxycycline stress.

Cell structure proteins (envelope proteins) were biggest part of the total protein content, and inhibition of protein synthesis might cause cell envelope defect. Most lipoproteins and outer membrane proteins were down-regulated, while the S1 and S2 antigens and some inner membrane proteins were up-regulated. The down-regulation of outer membrane lipoproteins could reduce a large demand for protein synthesis that would help B. burgdorferi persist under doxycycline stress. On the other hand, defect of outer membrane may allow strengthening of inner membrane structure by up-regulating membrane proteins. Ribosome is the target of doxycycline which inhibits protein synthesis. One persistence strategy of B. burgdorferi might be reducing the availability of drug targets. Five ribosomal genes were found to be down-regulated in the doxycycline treated B. burgdorferi persisters. Down-regulation of ribosome proteins may also reduce the metabolism and the demand for nutrients and energy, allowing cells to transition to persistence.

TABLE 1
Genes upregulated or downregulated in order of fold change in doxycycline tolerant B. burgdorferi persisters

| | | | Expression | | | | |
|---|---|---|---|---|---|---|---|
| Name | Synonym | Product | Ctrl | Dox | Amo | foldDox | foldAmo |
| — | BB_0557 | phosphocarrier protein HPr | 0 | 56 | 8 | — | — |
| fliQ | BB_0274 | flagellar biosynthesis protein FliQ | 0 | 42 | 50 | — | — |
| — | BB_0726 | ATP-binding protein | 0 | 35 | 10 | — | — |
| blyA | BB_R23 | pore-forming hemolysin | 0 | 29 | 18 | — | — |
| — | BB_G31 | hypothetical protein | 0 | 27 | 15 | — | — |
| — | BB_r01 | 5S ribosomal RNA | 0 | 26 | 3 | — | — |
| — | BB_Q19 | hypothetical protein | 0 | 24 | 54 | — | — |
| fliR | BB_0273 | flagellar biosynthetic protein FliR | 0 | 18 | 28 | — | — |
| — | BB_A49 | hypothetical protein | 0 | 18 | 23 | — | — |
| — | BB_0282 | flagellar protein FlbD | 0 | 17 | 18 | — | — |
| secE | BB_0395 | protein translocase subunit SecE | 0 | 16 | 17 | — | — |
| — | BB_0403 | hypothetical protein | 0 | 16 | 11 | — | — |
| tRNA- | BB_t18 | Gln tRNA | 0 | 16 | 0 | — | — |
| Gln-1 | | | | | | | |
| — | BB_A45 | hypothetical protein | 0 | 16 | 13 | — | — |
| flgD | BB_0284 | flagellar hook capping protein | 0 | 15 | 8 | — | — |
| tRNA- | BB_t01 | Cys tRNA | 0 | 14 | 8 | — | — |
| Cys-1 | | | | | | | |
| ruvA | BB_0023 | Holliday junction DNA helicase | 0 | 12 | 15 | — | — |
| | | RuvA | | | | | |
| rplN | BB_0488 | 50S ribosomal protein L14 | 0 | 12 | 12 | — | — |
| bppB | BB_M36 | BppB | 0 | 11 | 17 | — | — |
| tRNA- | BB_t10 | Met tRNA | 0 | 11 | 9 | — | — |
| Met-3 | | | | | | | |
| — | BB_P40 | hypothetical protein | 0 | 10 | 2 | — | — |
| — | BB_N07 | hypothetical protein | 0 | 10 | 27 | — | — |
| — | BB_0234 | integral membrane protein | 0 | 9 | 2 | — | — |
| — | BB_0391 | 50S ribosomal protein L10 | 0 | 9 | 10 | — | — |
| — | BB_Q26 | hypothetical protein | 0 | 8 | 9 | — | — |
| — | BB_0124 | hypothetical protein | 0 | 8 | 5 | — | — |
| tRNA- | BB_t02 | Phe tRNA | 0 | 8 | 8 | — | — |
| Phe-2 | | | | | | | |
| — | BB_0212 | hypothetical protein | 0 | 8 | 2 | — | — |
| — | BB_0701 | hypothetical protein | 0 | 8 | 2 | — | — |
| — | BB_Q25 | hypothetical protein | 0 | 7 | 8 | — | — |
| — | BB_0095 | hypothetical protein | 0 | 7 | 2 | — | — |
| tRNA- | BB_t15 | Pro tRNA | 0 | 7 | 15 | — | — |
| Pro-1 | | | | | | | |
| — | BB_J24 | hypothetical protein | 0 | 7 | 0 | — | — |
| — | BB_A32 | hypothetical protein | 0 | 7 | 6 | — | — |
| — | BB_0309 | hypothetical protein | 0 | 6 | 8 | — | — |
| tRNA- | BB_t09 | Gly tRNA | 0 | 6 | 6 | — | — |
| Gly-2 | | | | | | | |
| — | BB_t29 | Tyr tRNA | 0 | 6 | 8 | — | — |
| bdrN | BB_O27 | protein BdrN | 0 | 5 | 1 | — | — |
| — | BB_O30 | hypothetical protein | 0 | 5 | 2 | — | — |
| gatC | BB_0343 | glutamyl-tRNA(Gln) | 0 | 5 | 10 | — | — |
| | | amidotransferase subunit C | | | | | |
| — | BB_t23 | Lys tRNA | 0 | 5 | 0 | — | — |
| — | BB_0708 | hypothetical protein | 0 | 5 | 4 | — | — |
| — | BB_0549 | hypothetical protein | 0 | 4 | 0 | — | — |
| — | BB_0731 | hypothetical protein | 0 | 4 | 1 | — | — |
| — | BB_A33 | lipoprotein | 0 | 4 | 15 | — | — |
| — | BB_S03 | hypothetical protein | 0 | 3 | 10 | — | — |
| — | BB_M02 | hypothetical protein | 0 | 3 | 12 | — | — |
| bdrP | BB_L27 | protein BdrP | 0 | 3 | 4 | — | — |
| — | BB_L42 | hypothetical protein | 0 | 3 | 11 | — | — |
| — | BB_0287 | flagellar protein FlbA | 0 | 3 | 0 | — | — |
| — | BB_P33 | PF-49 protein | 0 | 2 | 0 | — | — |
| revA | BB_M27 | rev protein | 0 | 2 | 2 | — | — |
| bppA | BB_O36 | protein BppA | 0 | 2 | 4 | — | — |
| — | BB_L41 | hypothetical protein | 0 | 2 | 0 | — | — |
| — | BB_Q38 | hypothetical protein | 0 | 2 | 0 | — | — |
| — | BB_0044 | hypothetical protein | 0 | 2 | 0 | — | — |
| — | BB_0773 | hypothetical protein | 0 | 2 | 3 | — | — |
| — | BB_H40 | transposase-like protein | 0 | 2 | 3 | — | — |
| — | BB_J23 | hypothetical protein | 0 | 2 | 1 | — | — |
| — | BB_B13 | plasmid partition protein | 0 | 2 | 4 | — | — |
| — | BB_R34 | hypothetical protein | 0 | 1 | 2 | — | — |
| — | BB_M21 | hypothetical protein | 0 | 1 | 3 | — | — |
| — | BB_O02 | hypothetical protein | 0 | 1 | 0 | — | — |
| — | BB_O11 | hypothetical protein | 0 | 1 | 2 | — | — |
| — | BB_O12 | hypothetical protein | 0 | 1 | 5 | — | — |
| bdrM | BB_O34 | protein BdrM | 0 | 1 | 1 | — | — |
| — | BB_N12 | hypothetical protein | 0 | 1 | 17 | — | — |
| — | BB_Q06 | membrane protein | 0 | 1 | 9 | — | — |
| — | BB_Q85 | hypothetical protein | 0 | 1 | 8 | — | — |
| — | BB_t30 | Phe tRNA | 0 | 1 | 0 | — | — |
| — | BB_0308 | hypothetical protein | 0 | 1 | 0 | — | — |
| — | BB_D24 | hypothetical protein | 0 | 1 | 0 | — | — |
| — | BB_F03 | RepU | 0 | 1 | 0 | — | — |
| — | BB_H29 | plasmid partition protein | 0 | 1 | 0 | — | — |
| clpP | BB_{—}0757 | ATP-dependent Clp protease | 11 | 329 | 66 | 29.90909091 | 6 |
| | | proteolytic subunit | | | | | |
| — | BB_G32 | replicative DNA helicase | 2 | 49 | 22 | 24.5 | 11 |
| — | BB_0091 | V-type ATP synthase subunit I | 1 | 24 | 7 | 24 | 7 |
| — | BB_H25 | hypothetical protein | 4 | 81 | 144 | 20.25 | 36 |
| — | BB_0019 | hypothetical protein | 4 | 71 | 33 | 17.75 | 8.25 |
| grpE | BB_0519 | protein GrpE | 3 | 52 | 25 | 17.33333333 | 8.333333333 |
| — | BB_0578 | methyl-accepting chemotaxis | 1 | 17 | 12 | 17 | 12 |
| | | protein | | | | | |
| — | BB_0220 | alanine--tRNA ligase | 12 | 172 | 56 | 14.33333333 | 4.666666667 |
| — | BB_0158 | S2 lipoprotein | 3 | 40 | 22 | 13.33333333 | 7.333333333 |
| — | BB_0807 | integral membrane protein | 3 | 39 | 16 | 13 | 5.333333333 |
| — | BB_0172 | von Willebrand factor type A | 5 | 62 | 30 | 12.4 | 6 |
| | | domain-containing protein | | | | | |
| — | BB_A05 | S1 antigen | 5 | 58 | 65 | 11.6 | 13 |
| — | BB_0554 | hypothetical protein | 8 | 90 | 36 | 11.25 | 4.5 |
| — | BB_0563 | hypothetical protein | 15 | 167 | 64 | 11.13333333 | 4.266666667 |
| — | BB_0059 | CBS domain-containing protein | 2 | 22 | 23 | 11 | 11.5 |
| — | BB_0451 | chromate transport protein | 4 | 44 | 30 | 11 | 7.5 |
| — | BB_0818 | hypothetical protein | 2 | 22 | 6 | 11 | 3 |
| — | BB_0414 | chemotaxis protein | 7 | 76 | 42 | 10.85714286 | 6 |
| | | methyltransferase | | | | | |
| bmpD | BB_0385 | basic membrane protein D | 6 | 59 | 46 | 9.833333333 | 7.666666667 |
| — | BB_0171 | hypothetical protein | 7 | 66 | 20 | 9.428571429 | 2.857142857 |
| — | BB_0083 | hypothetical protein | 6 | 56 | 17 | 9.333333333 | 2.833333333 |
| rpmA | BB_0780 | 50S ribosomal protein L27 | 7 | 64 | 74 | 9.142857143 | 10.57142857 |
| — | BB_0661 | hypothetical protein | 25 | 227 | 91 | 9.08 | 3.64 |
| — | BB_S04 | hypothetical protein | 2 | 18 | 23 | 9 | 11.5 |
| argF | BB_0842 | ornithine carbamoyltransferase | 2 | 18 | 21 | 9 | 10.5 |
| ligA | BB_0552 | DNA ligase | 25 | 221 | 68 | 8.84 | 2.72 |
| — | BB_0088 | elongation factor EF-4 | 12 | 105 | 22 | 8.75 | 1.833333333 |
| — | BB_0830 | exonuclease SbcC | 7 | 60 | 23 | 8.571428571 | 3.285714286 |
| — | BB_A53 | Bbs27 protein | 16 | 137 | 90 | 8.5625 | 5.625 |
| — | BB_B25 | hypothetical protein | 4 | 34 | 8 | 8.5 | 2 |
| — | BB_0164 | K+-dependent Na+/Ca+ exchanger- | 15 | 126 | 63 | 8.4 | 4.2 |
| | | like protein | | | | | |
| — | BB_J16 | plasmid partition protein | 2 | 16 | 11 | 8 | 5.5 |
| — | BB_0543 | hypothetical protein | 13 | 103 | 13 | 7.923076923 | 1 |
| — | BB_0104 | periplasmic serine protease DO | 7 | 55 | 26 | 7.857142857 | 3.714285714 |
| — | BB_0819 | cytidylate kinase | 4 | 31 | 6 | 7.75 | 1.5 |
| — | BB_0016 | GlpE protein | 6 | 46 | 23 | 7.666666667 | 3.833333333 |
| — | BB_0200 | D-alanine--D-alanine ligase | 27 | 207 | 80 | 7.666666667 | 2.962962963 |
| — | BB_0116 | PTS system maltose and glucose- | 9 | 68 | 67 | 7.555555556 | 7.444444444 |
| | | specific transporter subunit IIABC | | | | | |
| ftsY | BB_0076 | signal recognition particle-docking | 2 | 15 | 16 | 7.5 | 8 |
| | | protein FtsY | | | | | |
| — | BB_0448 | phosphocarrier protein HPr | 8 | 59 | 35 | 7.375 | 4.375 |
| — | BB_0670 | purine-binding chemotaxis protein | 8 | 57 | 44 | 7.125 | 5.5 |
| rpoZ | BB_0820 | DNA-directed RNA polymerase | 10 | 71 | 26 | 7.1 | 2.6 |
| | | subunit omega | | | | | |
| — | BB_0201 | UDP-N-acetylmuramyl-tripeptide | 20 | 141 | 48 | 7.05 | 2.4 |
| | | synthetase | | | | | |
| bmpB | BB_0382 | basic membrane protein B | 6 | 42 | 18 | 7 | 3 |
| — | BB_A36 | lipoprotein | 7 | 49 | 59 | 7 | 8.428571429 |
| rpsE | BB_0495 | 30S ribosomal protein S5 | 6 | 40 | 22 | 6.666666667 | 3.666666667 |
| — | BB_0187 | hypothetical protein | 10 | 65 | 8 | 6.5 | 0.8 |
| erpG | BB_S41 | outer surface protein ErpG | 3 | 19 | 19 | 6.333333333 | 6.333333333 |
| — | BB_0186 | tRNA threonylcarbamoyladenosine | 21 | 133 | 28 | 6.333333333 | 1.333333333 |
| | | biosynthesis protein | | | | | |
| — | BB_0432 | hypothetical protein | 9 | 57 | 35 | 6.333333333 | 3.888888889 |
| map | BB_0105 | methionine aminopeptidase | 8 | 50 | 14 | 6.25 | 1.75 |
| — | BB_0748 | hypothetical protein | 9 | 56 | 25 | 6.222222222 | 2.777777778 |
| — | BB_0097 | hypothetical protein | 5 | 31 | 15 | 6.2 | 3 |
| — | BB_0340 | hypothetical protein | 19 | 114 | 47 | 6 | 2.473684211 |
| — | BB_0054 | protein-export membrane protein | 12 | 71 | 53 | 5.916666667 | 4.416666667 |
| | | SecG | | | | | |
| — | BB_0260 | hypothetical protein | 6 | 35 | 8 | 5.833333333 | 1.333333333 |
| — | BB_0644 | N-acetylmannosamine-6-phosphate | 12 | 70 | 28 | 5.833333333 | 2.333333333 |
| | | 2-epimerase | | | | | |
| — | BB_0758 | hypothetical protein | 10 | 58 | 12 | 5.8 | 1.2 |
| — | BB_0616 | integral membrane protein | 8 | 46 | 16 | 5.75 | 2 |
| — | BB_0637 | Na+/H+ antiporter family | 24 | 138 | 45 | 5.75 | 1.875 |
| — | BB_B10 | hypothetical protein | 4 | 23 | 6 | 5.75 | 1.5 |
| — | BB_0513 | phenylalanine--tRNA ligase | 11 | 63 | 24 | 5.727272727 | 2.181818182 |
| | | subunit alpha | | | | | |
| — | BB_0737 | tRNA-dihydrouridine synthase | 11 | 63 | 22 | 5.727272727 | 2 |
| valS | BB_0738 | valine--tRNA ligase | 27 | 153 | 80 | 5.666666667 | 2.962962963 |
| — | BB_J28 | hypothetical protein | 3 | 17 | 18 | 5.666666667 | 6 |
| — | BB_0671 | chemotaxis protein CheX | 24 | 134 | 95 | 5.583333333 | 3.958333333 |
| — | BB_0808 | hypothetical protein | 12 | 67 | 35 | 5.583333333 | 2.916666667 |
| — | BB_0227 | hypothetical protein | 7 | 39 | 23 | 5.571428571 | 3.285714286 |
| dnaJ | BB_0517 | chaperone protein DnaJ | 30 | 166 | 63 | 5.533333333 | 2.1 |
| tpiA | BB_0055 | triosephosphate isomerase | 17 | 94 | 60 | 5.529411765 | 3.529411765 |
| — | BB_J25 | hypothetical protein | 4 | 22 | 15 | 5.5 | 3.75 |
| — | BB_0035 | DNA topoisomerase IV subunit A | 8 | 43 | 21 | 5.375 | 2.625 |
| secD | BB_0652 | protein translocase subunit SecD | 20 | 107 | 50 | 5.35 | 2.5 |
| tyrS | BB_0370 | tyrosine--tRNA ligase | 9 | 48 | 22 | 5.333333333 | 2.444444444 |
| glyA | BB_0601 | serine hydroxymethyltransferase | 10 | 53 | 25 | 5.3 | 2.5 |
| acpP | BB_0704 | acyl carrier protein | 10 | 53 | 24 | 5.3 | 2.4 |
| — | BB_0114 | single-stranded DNA-binding | 4 | 21 | 9 | 5.25 | 2.25 |
| | | protein | | | | | |
| — | BB_J48 | hypothetical protein | 9 | 47 | 44 | 5.222222222 | 4.888888889 |
| — | BB_0090 | V-type ATP synthase subunit K | 5 | 26 | 1 | 5.2 | 0.2 |
| zwf | BB_0636 | glucose-6-phosphate 1- | 7 | 36 | 9 | 5.142857143 | 1.285714286 |
| | | dehydrogenase | | | | | |
| — | BB_0624 | hypothetical protein | 10 | 51 | 14 | 5.1 | 1.4 |
| blyA | BB_Q30 | holin protein | 2 | 10 | 10 | 5 | 5 |
| — | BB_0301 | cell division protein B | 3 | 15 | 6 | 5 | 2 |
| — | BB_0592 | hypothetical protein | 6 | 30 | 6 | 5 | 1 |
| — | BB_0405 | hypothetical protein | 36 | 179 | 79 | 4.972222222 | 2.194444444 |
| — | BB_0215 | phosphate ABC transporter | 10 | 49 | 12 | 4.9 | 1.2 |
| | | substrate-binding protein | | | | | |
| trxB | BB_0515 | thioredoxin reductase | 43 | 210 | 120 | 4.88372093 | 2.790697674 |
| — | BB_0267 | hypothetical protein | 7 | 34 | 17 | 4.857142857 | 2.428571429 |
| — | BB_0157 | hypothetical protein | 5 | 24 | 11 | 4.8 | 2.2 |
| — | BB_0729 | dicarboxylate/amino acid: cation | 54 | 258 | 179 | 4.777777778 | 3.314814815 |
| | | symporter | | | | | |
| — | BB_0463 | nucleoside diphosphate kinase | 4 | 19 | 23 | 4.75 | 5.75 |
| — | BB_0464 | hypothetical protein | 4 | 19 | 12 | 4.75 | 3 |
| — | BB_B24 | hypothetical protein | 8 | 38 | 8 | 4.75 | 1 |
| — | BB_0785 | septation protein SpoVG | 15 | 71 | 38 | 4.733333333 | 2.533333333 |
| — | BB_0036 | DNA topoisomerase IV subunit B | 6 | 28 | 17 | 4.666666667 | 2.833333333 |
| — | BB_0662 | hypothetical protein | 30 | 140 | 121 | 4.666666667 | 4.033333333 |
| — | BB_A51 | hypothetical protein | 16 | 74 | 59 | 4.625 | 3.6875 |
| — | BB_0266 | hypothetical protein | 7 | 32 | 20 | 4.571428571 | 2.857142857 |
| — | BB_0039 | hypothetical protein | 2 | 9 | 4 | 4.5 | 2 |
| — | BB_0354 | hypothetical protein | 14 | 63 | 13 | 4.5 | 0.928571429 |
| — | BB_0427 | rRNA small subunit | 2 | 9 | 8 | 4.5 | 4 |
| | | methyltransferase I | | | | | |
| — | BB_0442 | membrane protein insertase YidC | 6 | 27 | 10 | 4.5 | 1.666666667 |
| lysS | BB_0659 | lysine--tRNA ligase | 15 | 67 | 36 | 4.466666667 | 2.4 |
| — | BB_0539 | hypothetical protein | 9 | 40 | 21 | 4.444444444 | 2.333333333 |
| — | BB_0718 | penicillin-binding protein | 9 | 40 | 28 | 4.444444444 | 3.111111111 |
| — | BB_B29 | PTS system transporter subunit | 46 | 204 | 61 | 4.434782609 | 1.326086957 |
| | | IIBC | | | | | |
| — | BB_0197 | release factor glutamine | 7 | 31 | 17 | 4.428571429 | 2.428571429 |
| | | methyltransferase | | | | | |
| rplT | BB_0188 | 50S ribosomal protein L20 | 38 | 168 | 132 | 4.421052632 | 3.473684211 |
| — | BB_0761 | peptidoglycan-binding protein | 20 | 88 | 35 | 4.4 | 1.75 |
| — | BB_0712 | RNA polymerase sigma factor | 9 | 39 | 29 | 4.333333333 | 3.222222222 |
| | | RpoD | | | | | |
| trmD | BB_0698 | tRNA (guanine-N(1)-)- | 10 | 43 | 16 | 4.3 | 1.6 |
| | | methyltransferase | | | | | |
| — | BB_0535 | hypothetical protein | 7 | 30 | 47 | 4.285714286 | 6.714285714 |
| — | BB_0749 | hypothetical protein | 18 | 77 | 32 | 4.277777778 | 1.777777778 |
| — | BB_0246 | M23 peptidase domain-containing | 13 | 55 | 10 | 4.230769231 | 0.769230769 |
| | | protein | | | | | |
| — | BB_G17 | hypothetical protein | 5 | 21 | 45 | 4.2 | 9 |
| — | BB_J29 | hypothetical protein | 6 | 25 | 23 | 4.166666667 | 3.833333333 |
| lon | BB_0613 | ATP-dependent protease La | 53 | 220 | 126 | 4.150943396 | 2.377358491 |
| — | BB_0326 | hypothetical protein | 27 | 112 | 56 | 4.148148148 | 2.074074074 |
| lepB | BB_0030 | signal peptidase I | 14 | 57 | 61 | 4.071428571 | 4.357142857 |
| erpA | BB_P38 | protein ErpA | 8 | 32 | 9 | 4 | 1.125 |
| — | BB_0551 | protein CheY | 5 | 20 | 26 | 4 | 5.2 |
| — | BB_0852 | hypothetical protein | 1 | 4 | 3 | 4 | 3 |
| — | BB_0584 | integral membrane protein | 23 | 91 | 46 | 3.956521739 | 2 |
| — | BB_0173 | hypothetical protein | 20 | 79 | 56 | 3.95 | 2.8 |
| mvaD | BB_0686 | diphosphomevalonate | 24 | 94 | 101 | 3.916666667 | 4.208333333 |
| | | decarboxylase | | | | | |
| — | BB_0082 | hypothetical protein | 24 | 93 | 49 | 3.875 | 2.041666667 |
| nadD | BB_0782 | nicotinate-nucleotide | 8 | 31 | 53 | 3.875 | 6.625 |
| | | adenylyltransferase | | | | | |
| ptsP | BB_0558 | phosphoenolpyruvate-protein | 22 | 85 | 21 | 3.863636364 | 0.954545455 |
| | | phosphatase | | | | | |
| — | BB_B23 | guanine/xanthine permease | 52 | 200 | 73 | 3.846153846 | 1.403846154 |
| mrdB | BB_0719 | rod shape-determining protein | 19 | 73 | 39 | 3.842105263 | 2.052631579 |
| | | RodA | | | | | |
| — | BB_0341 | aspartyl/glutamyl-tRNA(Asn/Gln) | 22 | 84 | 43 | 3.818181818 | 1.954545455 |
| | | amidotransferase subunit B | | | | | |
| miaA | BB_0821 | tRNA dimethylallyltransferase | 11 | 42 | 11 | 3.818181818 | 1 |
| — | BB_A54 | hypothetical protein | 28 | 106 | 57 | 3.785714286 | 2.035714286 |
| — | BB_0468 | hypothetical protein | 12 | 45 | 30 | 3.75 | 2.5 |
| — | BB_0606 | chemoreceptor glutamine | 40 | 150 | 99 | 3.75 | 2.475 |
| | | deamidase CheD | | | | | |
| pth | BB_0787 | peptidyl-tRNA hydrolase | 4 | 15 | 4 | 3.75 | 1 |
| — | BB_J31 | hypothetical protein | 16 | 60 | 49 | 3.75 | 3.0625 |
| rpsN | BB_0491 | 30S ribosomal protein S14 | 10 | 37 | 16 | 3.7 | 1.6 |
| — | BB_0742 | ABC transporter ATP-binding | 10 | 37 | 36 | 3.7 | 3.6 |
| | | protein | | | | | |
| mfd | BB_0623 | transcription-repair coupling factor | 25 | 92 | 91 | 3.68 | 3.64 |
| — | BB_0126 | hypothetical protein | 3 | 11 | 11 | 3.666666667 | 3.666666667 |
| — | BB_0133 | hypothetical protein | 9 | 33 | 17 | 3.666666667 | 1.888888889 |
| — | BB_0277 | flagellar motor switch protein FliN | 12 | 44 | 28 | 3.666666667 | 2.333333333 |
| — | BB_H37 | lipoprotein | 9 | 33 | 31 | 3.666666667 | 3.444444444 |
| — | BB_0351 | hypothetical protein | 17 | 62 | 21 | 3.647058824 | 1.235294118 |
| — | BB_0369 | ATP-dependent Clp protease | 14 | 51 | 25 | 3.642857143 | 1.785714286 |
| | | subunit A | | | | | |
| fliI | BB_0288 | flagellum-specific ATP synthase | 8 | 29 | 35 | 3.625 | 4.375 |
| | | FliI | | | | | |
| — | BB_0668 | flagellar filament outer layer | 8 | 29 | 35 | 3.625 | 4.375 |
| | | protein | | | | | |
| — | BB_0231 | hypothetical protein | 5 | 18 | 7 | 3.6 | 1.4 |
| — | BB_0020 | diphosphate--fructose-6-phosphate | 17 | 61 | 29 | 3.588235294 | 1.705882353 |
| | | 1-phosphotransferase | | | | | |
| — | BB_0679 | ribose/galactose ABC transporter | 31 | 111 | 104 | 3.580645161 | 3.35483871 |
| | | permease | | | | | |
| — | BB_0564 | hypothetical protein | 7 | 25 | 17 | 3.571428571 | 2.428571429 |
| — | BB_0327 | glycerol-3-phosphate O- | 15 | 53 | 20 | 3.533333333 | 1.333333333 |
| | | acyltransferase | | | | | |
| — | predicted | antisense: grpE | 9716 | 34301 | 33514 | 3.530362289 | 3.449361877 |
| | RNA | | | | | | |
| — | BB_R31 | hypothetical protein | 2 | 7 | 0 | 3.5 | 0 |
| erpP | BB_N38 | protein ErpP | 4 | 14 | 3 | 3.5 | 0.75 |
| — | BB_0069 | aminopeptidase II | 10 | 35 | 26 | 3.5 | 2.6 |
| — | BB_0319 | hypothetical protein | 6 | 21 | 21 | 3.5 | 3.5 |
| — | BB_0363 | hypothetical protein | 12 | 42 | 53 | 3.5 | 4.416666667 |
| dnaK | BB_0518 | chaperone protein DnaK | 22 | 77 | 21 | 3.5 | 0.954545455 |
| — | predicted | antisense: hisS | 7531 | 26181 | 26631 | 3.476430753 | 3.536183774 |
| | RNA | | | | | | |
| coaE | BB_0547 | dephospho-CoA kinase | 16 | 55 | 46 | 3.4375 | 2.875 |
| — | BB_0165 | hypothetical protein | 35 | 120 | 87 | 3.428571429 | 2.485714286 |
| murG | BB_0767 | UDP-N-acetylglucosamine--N- | 21 | 72 | 90 | 3.428571429 | 4.285714286 |
| | | acetylmuramyl-(pentapeptide) | | | | | |
| | | pyrophosphoryl-undecaprenol N- | | | | | |
| | | acetylglucosamine transferase | | | | | |
| — | BB_0526 | hypothetical protein | 25 | 85 | 53 | 3.4 | 2.12 |
| — | BB_G25 | hypothetical protein | 5 | 17 | 39 | 3.4 | 7.8 |
| — | BB_0733 | hypothetical protein | 51 | 171 | 99 | 3.352941176 | 1.941176471 |
| trpS | BB_0005 | tryptophan--tRNA ligase | 12 | 40 | 44 | 3.333333333 | 3.666666667 |
| — | BB_0026 | bifunctional | 39 | 130 | 58 | 3.333333333 | 1.487179487 |
| | | methylenetetrahydrofolate | | | | | |
| | | dehydrogenase/methenyltetrahydro- | | | | | |
| | | folate cyclohydrolase | | | | | |
| — | BB_0579 | DNA polymerase III subunit alpha | 21 | 70 | 44 | 3.333333333 | 2.095238095 |
| — | BB_B03 | telomere resolvase ResT | 96 | 320 | 371 | 3.333333333 | 3.864583333 |
| — | BB_0206 | methyltransferase | 90 | 298 | 101 | 3.311111111 | 1.122222222 |
| — | BB_G30 | hypothetical protein | 10 | 33 | 15 | 3.3 | 1.5 |
| fbaA | BB_0445 | fructose-bisphosphate aldolase | 31 | 102 | 61 | 3.290322581 | 1.967741935 |
| — | BB_0651 | protein translocase subunit YajC | 14 | 46 | 17 | 3.285714286 | 1.214285714 |
| — | BB_0259 | transglycosylase SLT domain- | 27 | 88 | 51 | 3.259259259 | 1.888888889 |
| | | containing protein | | | | | |
| lepB | BB_0031 | signal peptidase I | 41 | 133 | 76 | 3.243902439 | 1.853658537 |
| — | BB_0323 | hypothetical protein | 31 | 100 | 98 | 3.225806452 | 3.161290323 |
| — | BB_0678 | sugar ABC transporter permease | 9 | 29 | 22 | 3.222222222 | 2.444444444 |
| — | BB_0569 | hypothetical protein | 14 | 45 | 74 | 3.214285714 | 5.285714286 |
| secF | BB_0653 | protein translocase subunit SecF | 44 | 141 | 46 | 3.204545455 | 1.045454545 |
| nusG | BB_0394 | transcription | 16 | 51 | 29 | 3.1875 | 1.8125 |
| | | termination/antitermination factor | | | | | |
| metK | BB_0376 | S-adenosylmethionine synthetase | 97 | 308 | 187 | 3.175257732 | 1.927835052 |
| — | BB_0080 | ABC transporter ATP-binding | 32 | 100 | 61 | 3.125 | 1.90625 |
| | | protein | | | | | |
| — | BB_0681 | methyl-accepting chemotaxis | 52 | 162 | 124 | 3.115384615 | 2.384615385 |
| | | protein | | | | | |
| gltX | BB_0372 | glutamate--tRNA ligase | 9 | 28 | 18 | 3.111111111 | 2 |
| efp | BB_0214 | elongation factor P | 372 | 1154 | 1154 | 3.102150538 | 3.102150538 |
| erpO | BB_L40 | ErpB1 protein | 101 | 311 | 114 | 3.079207921 | 1.128712871 |
| — | BB_0265 | hypothetical protein | 13 | 40 | 15 | 3.076923077 | 1.153846154 |
| — | BB_0567 | chemotaxis histidine kinase | 16 | 49 | 57 | 3.0625 | 3.5625 |
| — | BB_0754 | ABC transporter ATP-binding | 16 | 49 | 46 | 3.0625 | 2.875 |
| | | protein | | | | | |
| — | BB_0176 | ATPase | 32 | 97 | 88 | 3.03125 | 2.75 |
| pheT | BB_0514 | phenylalanine--tRNA sligase | 35 | 106 | 47 | 3.028571429 | 1.342857143 |
| | | subunit beta | | | | | |
| lon | BB_0253 | ATP-dependent protease La | 39 | 118 | 43 | 3.025641026 | 1.102564103 |
| rpsR | BB_0113 | 30S ribosomal protein S18 | 12 | 36 | 29 | 3 | 2.416666667 |
| — | BB_0146 | glycine/betaine ABC transporter | 10 | 30 | 37 | 3 | 3.7 |
| | | ATP-binding protein | | | | | |
| — | BB_0329 | ABC transporter substrate-binding | 18 | 54 | 40 | 3 | 2.222222222 |
| | | protein | | | | | |
| — | BB_0735 | rare lipoprotein A | 17 | 51 | 19 | 3 | 1.117647059 |
| — | BB_0783 | hypothetical protein | 3 | 9 | 17 | 3 | 5.666666667 |
| — | BB_0680 | methyl-accepting chemotaxis | 35 | 104 | 75 | 2.971428571 | 2.142857143 |
| | | protein | | | | | |
| guaA | BB_B18 | GMP synthase | 101 | 300 | 197 | 2.97029703 | 1.95049505 |
| — | BB_0408 | PTS system fructose-specific | 20 | 59 | 41 | 2.95 | 2.05 |
| | | transproter subunit IIABC | | | | | |
| — | BB_0183 | flagellar assembly protein FliW | 68 | 200 | 200 | 2.941176471 | 2.941176471 |
| malQ | BB_0166 | 4-alpha-glucanotransferase | 16 | 47 | 53 | 2.9375 | 3.3125 |
| — | BB_0296 | ATP-dependent protease subunit | 16 | 47 | 20 | 2.9375 | 1.25 |
| | | HslV | | | | | |
| xth | BB_0534 | exodeoxyribonuclease III | 68 | 199 | 180 | 2.926470588 | 2.647058824 |
| — | BB_0422 | 3-methyladenine DNA glycosylase | 12 | 35 | 79 | 2.916666667 | 6.583333333 |
| pyk | BB_0348 | pyruvate kinase | 72 | 207 | 150 | 2.875 | 2.083333333 |
| — | BB_0344 | DNA helicase | 22 | 63 | 77 | 2.863636364 | 3.5 |
| — | BB_0504 | ribonuclease Y | 13 | 37 | 31 | 2.846153846 | 2.384615385 |
| — | BB_0416 | pheromone shutdown protein | 81 | 230 | 155 | 2.839506173 | 1.913580247 |
| — | BB_0346 | export chaperone | 6 | 17 | 12 | 2.833333333 | 2 |
| erpB | BB_P39 | protein ErpB | 681 | 1926 | 837 | 2.828193833 | 1.22907489 |
| — | BB_0409 | hypothetical protein | 46 | 129 | 52 | 2.804347826 | 1.130434783 |
| — | BB_0093 | V-type ATP synthase subunit B | 10 | 28 | 27 | 2.8 | 2.7 |
| rpoS | BB_0771 | RNA polymerase sigma factor | 25 | 70 | 84 | 2.8 | 3.36 |
| — | BB_0245 | hypothetical protein | 42 | 117 | 96 | 2.785714286 | 2.285714286 |
| secA | BB_0154 | protein translocase subunit SecA | 23 | 64 | 33 | 2.782608696 | 1.434782609 |
| — | BB_0533 | protein PhnP | 50 | 139 | 107 | 2.78 | 2.14 |
| — | BB_0555 | hypothetical protein | 39 | 108 | 113 | 2.769230769 | 2.897435897 |
| hisS | BB_0135 | histidine--tRNA ligase | 20 | 55 | 29 | 2.75 | 1.45 |
| bmpC | BB_0384 | basic membrane protein C | 8 | 22 | 15 | 2.75 | 1.875 |
| — | BB_0400 | hypothetical protein | 20 | 55 | 42 | 2.75 | 2.1 |
| bppC | BB_S40 | protein BppC | 330 | 901 | 483 | 2.73030303 | 1.463636364 |
| — | BB_0222 | 6-phosphogluconolactonase | 36 | 98 | 74 | 2.722222222 | 2.055555556 |
| cysS | BB_0599 | cysteine--tRNA ligase | 7 | 19 | 7 | 2.714285714 | 1 |
| — | BB_B12 | PF-32 protein | 14 | 38 | 37 | 2.714285714 | 2.642857143 |
| — | BB_0806 | lipoprotein | 10 | 27 | 14 | 2.7 | 1.4 |
| — | BB_0379 | HIT family nucleosidase | 39 | 105 | 52 | 2.692307692 | 1.333333333 |
| — | BB_0645 | PTS system glucose-specific | 104 | 280 | 122 | 2.692307692 | 1.173076923 |
| | | transporter subunit IIBC | | | | | |
| — | BB_0724 | potassium transporter | 16 | 43 | 20 | 2.6875 | 1.25 |
| mgtE | BB_0380 | Mg2+ transport protein | 54 | 145 | 85 | 2.685185185 | 1.574074074 |
| — | BB_0752 | hypothetical protein | 50 | 134 | 109 | 2.68 | 2.18 |
| — | BB_0077 | hypothetical protein | 3 | 8 | 9 | 2.666666667 | 3 |
| ftsA | BB_0300 | cell division protein FtsA | 9 | 24 | 14 | 2.666666667 | 1.555555556 |
| — | BB_0358 | rRNA small subunit | 21 | 56 | 47 | 2.666666667 | 2.238095238 |
| | | methyltransferase E | | | | | |
| — | BB_0786 | 50S ribosomal protein L25/general | 15 | 40 | 14 | 2.666666667 | 0.933333333 |
| | | stress protein Ctc | | | | | |
| — | BB_B22 | guanine/xanthine permease | 54 | 144 | 47 | 2.666666667 | 0.87037037 |
| — | BB_0447 | Na+/H+ antiporter | 68 | 180 | 141 | 2.647058824 | 2.073529412 |
| — | BB_0415 | chemotaxis protein CheB | 65 | 172 | 106 | 2.646153846 | 1.630769231 |
| — | BB_B04 | chitibiose transporter protein ChbC | 394 | 1041 | 598 | 2.64213198 | 1.517766497 |
| cspA | BB_A68 | complement regulator-acquiring | 8 | 21 | 22 | 2.625 | 2.75 |
| | | surface protein 1 | | | | | |
| — | BB_0760 | protein Gp37 | 86 | 225 | 320 | 2.61627907 | 3.720930233 |
| — | BB_0844 | lipoprotein | 41 | 107 | 171 | 2.609756098 | 4.170731707 |
| rnz | BB_0755 | ribonuclease Z | 23 | 60 | 56 | 2.608695652 | 2.434782609 |
| — | BB_0631 | hypothetical protein | 28 | 73 | 40 | 2.607142857 | 1.428571429 |
| — | BB_0811 | hypothetical protein | 33 | 86 | 78 | 2.606060606 | 2.363636364 |
| — | BB_0195 | hypothetical protein | 45 | 111 | 132 | 2.6 | 2.933333333 |
| fni | BB_0684 | isopentenyl-diphosphate delta- | 40 | 104 | 90 | 2.6 | 2.25 |
| | | isomerase | | | | | |
| bppC | BB_R38 | BppC | 76 | 197 | 107 | 2.592105263 | 1.407894737 |
| — | BB_0779 | hypothetical protein | 22 | 57 | 28 | 2.590909091 | 1.272727273 |
| prfB | BB_0074 | peptide chain release factor 2 | 12 | 31 | 13 | 2.583333333 | 1.083333333 |
| rpsO | BB_0804 | 30S ribosomal protein S15 | 23 | 59 | 32 | 2.565217391 | 1.391304348 |
| — | BB_D0031 | hypothetical protein | 23 | 59 | 72 | 2.565217391 | 3.130434783 |
| — | BB_A48 | hypothetical protein | 9 | 23 | 31 | 2.555555556 | 3.444444444 |
| — | BB_0775 | flagellar hook-basal body complex | 13 | 33 | 31 | 2.538461538 | 2.384615385 |
| | | protein | | | | | |
| gyrA | BB_0435 | DNA gyrase subunit A | 42 | 106 | 97 | 2.523809524 | 2.30952381 |
| erpM | BB_O40 | protein ErpM | 2 | 5 | 0 | 2.5 | 0 |
| rsgA | BB_0099 | ribosome biogenesis GTPase RsgA | 4 | 10 | 12 | 2.5 | 3 |
| nusB | BB_0107 | transcription antitermination factor | 10 | 25 | 11 | 2.5 | 1.1 |
| | | NusB | | | | | |
| ychF | BB_0235 | ribosome-binding ATPase | 4 | 10 | 13 | 2.5 | 3.25 |
| — | BB_0528 | aldose reductase | 28 | 70 | 62 | 2.5 | 2.214285714 |
| — | BB_0725 | lectin | 8 | 20 | 23 | 2.5 | 2.875 |
| — | BB_J26 | ABC transporter ATP-binding | 12 | 30 | 42 | 2.5 | 3.5 |
| | | protein | | | | | |
| — | BB_A23 | hypothetical protein | 16 | 40 | 39 | 2.5 | 2.4375 |
| — | BB_0249 | phosphatidylcholine synthase | 142 | 349 | 488 | 2.457746479 | 3.436619718 |
| — | BB_0268 | hypothetical protein | 9 | 22 | 10 | 2.444444444 | 1.111111111 |
| — | BB_0175 | hypothetical protein | 148 | 361 | 347 | 2.439189189 | 2.344594595 |
| groS | BB_0741 | chaperonin GroS | 32 | 78 | 21 | 2.4375 | 0.65625 |
| rplU | BB_0778 | 50S ribosomal protein L21 | 14 | 34 | 15 | 2.428571429 | 1.071428571 |
| — | BB_0614 | hypothetical protein | 115 | 278 | 94 | 2.417391304 | 0.817391304 |
| trmE | BB_0179 | tRNA modification GTPase | 12 | 29 | 26 | 2.416666667 | 2.166666667 |
| def | BB_0065 | peptide deformylase | 17 | 41 | 56 | 2.411764706 | 3.294117647 |
| — | BB_0130 | hypothetical protein | 5 | 12 | 19 | 2.4 | 3.8 |
| — | BB_0458 | hypothetical protein | 19 | 45 | 19 | 2.368421053 | 1 |
| — | BB_0430 | hypothetical protein | 11 | 26 | 23 | 2.363636364 | 2.090909091 |
| — | BB_0455 | hypothetical protein | 11 | 26 | 23 | 2.363636364 | 2.090909091 |
| — | BB_0070 | hypothetical protein | 75 | 176 | 120 | 2.346666667 | 1.6 |
| — | BB_0002 | hypothetical protein | 15 | 35 | 37 | 2.333333333 | 2.466666667 |
| gidB | BB_0177 | rRNA small subunit | 3 | 7 | 9 | 2.333333333 | 3 |
| | | methyltransferase G | | | | | |
| glys | BB_0371 | glycine--tRNA ligase | 18 | 42 | 18 | 2.333333333 | 1 |
| rplK | BB_0393 | 50S ribosomal protein L11 | 9 | 21 | 11 | 2.333333333 | 1.222222222 |
| — | BB_0467 | laccase domain-containing protein | 6 | 14 | 11 | 2.333333333 | 1.833333333 |
| — | BB_0826 | hypothetical protein | 3 | 7 | 13 | 2.333333333 | 4.333333333 |
| — | predicted | antisense: rpsD | 17008 | 39303 | 27348 | 2.310853716 | 1.6079492 |
| | RNA | | | | | | |
| flhB | BB_0272 | flagellar biosynthesis protein FlhB | 26 | 60 | 59 | 2.307692308 | 2.269230769 |
| — | BB_0444 | nucleotide sugar epimerase | 27 | 62 | 37 | 2.296296296 | 1.37037037 |
| — | BB_0627 | aminopeptidase | 188 | 430 | 545 | 2.287234043 | 2.89893617 |
| — | BB_0058 | hypothetical protein | 7 | 16 | 22 | 2.285714286 | 3.142857143 |
| erpQ | BB_N39 | protein ErpQ | 130 | 295 | 134 | 2.269230769 | 1.030769231 |
| — | BB_0687 | phosphomevalonate kinase | 68 | 154 | 268 | 2.264705882 | 3.941176471 |
| — | BB_0527 | type III pantothenate kinase | 70 | 158 | 62 | 2.257142857 | 0.885714286 |
| — | BB_0604 | L-lactate permease | 111 | 250 | 256 | 2.252252252 | 2.306306306 |
| pgk | BB_0056 | phosphoglycerate kinase | 24 | 54 | 42 | 2.25 | 1.75 |
| — | BB_0322 | hypothetical protein | 4 | 9 | 1 | 2.25 | 0.25 |
| — | BB_0608 | aminoacyl-histidine dipeptidase | 12 | 27 | 33 | 2.25 | 2.75 |
| — | BB_0667 | hypothetical protein | 4 | 9 | 9 | 2.25 | 2.25 |
| — | BB_0784 | ribosomal silencing factor RsfS | 12 | 27 | 19 | 2.25 | 1.583333333 |
| — | BB_0010 | holo-acyl-carrier protein synthase | 9 | 20 | 11 | 2.222222222 | 1.222222222 |
| — | BB_0566 | hypothetical protein | 14 | 31 | 38 | 2.214285714 | 2.714285714 |
| lgt | BB_0362 | prolipoprotein diacylglyceryl | 61 | 135 | 139 | 2.213114754 | 2.278688525 |
| | | transferase | | | | | |
| — | BB_0769 | tRNA N6-adenosine | 54 | 119 | 169 | 2.203703704 | 3.12962963 |
| | | threonylcarbamoyltransferase | | | | | |
| polA | BB_0548 | DNA polymerase I | 20 | 44 | 49 | 2.2 | 2.45 |
| — | BB_0638 | Na+/H+ antiporter | 95 | 209 | 93 | 2.2 | 0.978947368 |
| uvrB | BB_0836 | excinuclease ABC subunit B | 5 | 11 | 12 | 2.2 | 2.4 |
| leuS | BB_0251 | leucine--tRNA ligase | 38 | 83 | 49 | 2.184210526 | 1.289473684 |
| cmk | BB_0128 | cytidylate kinase | 22 | 48 | 88 | 2.181818182 | 4 |
| p83/100 | BB_0744 | p83/100 antigen | 51 | 111 | 165 | 2.176470588 | 3.235294118 |
| fliE | BB_0292 | flagellar hook-basal body protein | 6 | 13 | 13 | 2.166666667 | 2.166666667 |
| | | FliE | | | | | |
| — | BB_0461 | DNA polymerase III subunits | 12 | 26 | 28 | 2.166666667 | 2.333333333 |
| | | gamma and tau | | | | | |
| — | BB_0544 | phosphoribosylpyrophosphate | 26 | 56 | 18 | 2.153846154 | 0.692307692 |
| | | synthetase | | | | | |
| — | BB_0417 | adenylate kinase | 249 | 536 | 419 | 2.152610442 | 1.682730924 |
| thrS | BB_0720 | threonine--tRNA ligase | 14 | 30 | 33 | 2.142857143 | 2.357142857 |
| — | BB_0137 | long-chain-fatty-acid CoA ligase | 22 | 47 | 20 | 2.136363636 | 0.909090909 |
| — | BB_A07 | ChpAI protein | 23 | 49 | 49 | 2.130434783 | 2.130434783 |
| — | BB_0037 | 1-acyl-sn-glycerol-3-phosphate | 8 | 17 | 11 | 2.125 | 1.375 |
| | | acyltransferase | | | | | |
| — | BB_0018 | ribosomal large subunit | 34 | 72 | 44 | 2.117647059 | 1.294117647 |
| | | pseudouridine synthase D | | | | | |
| — | BB_0693 | xylose operon regulatory protein | 9 | 19 | 25 | 2.111111111 | 2.777777778 |
| — | BB_0656 | oxygen-independent | 86 | 181 | 88 | 2.104651163 | 1.023255814 |
| | | coproporphyrinogen III oxidase | | | | | |
| — | predicted | antisense: BB_0578 | 21153 | 44470 | 42060 | 2.102302274 | 1.988370444 |
| | RNA | | | | | | |
| mlpI | BB_N28 | lipoprotein | 10 | 21 | 11 | 2.1 | 1.1 |
| gidA | BB_0178 | tRNA uridine 5- | 20 | 42 | 38 | 2.1 | 1.9 |
| | | carboxymethylaminomethyl | | | | | |
| | | modification protein GidA | | | | | |
| flhA | BB_0271 | flagellar biosynthesis protein FlhA | 10 | 21 | 21 | 2.1 | 2.1 |
| — | BB_0109 | acetyl-CoAC-acetyltransferase | 11 | 23 | 19 | 2.090909091 | 1.727272727 |
| — | predicted | antisense: BB_0219 | 15518 | 32402 | 29104 | 2.088026808 | 1.87549942 |
| | RNA | | | | | | |
| — | predicted | antisense: BB_0779 | 25448 | 52903 | 56940 | 2.078866709 | 2.23750393 |
| | RNA | | | | | | |
| uvrA | BB_0837 | excinuclease ABC subunit A | 13 | 27 | 35 | 2.076923077 | 2.692307692 |
| — | predicted | antisense: BB_0215 | 10979 | 22689 | 14137 | 2.066581656 | 1.28764004 |
| | RNA | | | | | | |
| — | BB_B16 | oligopeptide ABC transporter | 66 | 136 | 49 | 2.060606061 | 0.742424242 |
| | | OppAIV | | | | | |
| — | BB_0139 | hypothetical protein | 34 | 70 | 87 | 2.058823529 | 2.558823529 |
| tig | BB_0610 | trigger factor | 17 | 35 | 20 | 2.058823529 | 1.176470588 |
| — | predicted | antisense: tig | 13666 | 28118 | 30788 | 2.057515001 | 2.252890385 |
| | RNA | | | | | | |
| — | predicted | antisense: BB_0712 | 12262 | 25192 | 30600 | 2.054477247 | 2.495514598 |
| | RNA | | | | | | |
| — | BB_B14 | hypothetical protein | 233 | 477 | 681 | 2.0472103 | 2.922746781 |
| — | BB_0531 | hypothetical protein | 43 | 88 | 70 | 2.046511628 | 1.627906977 |
| pfs | BB_0375 | nucleosidase | 65 | 133 | 98 | 2.046153846 | 1.507692308 |
| — | BB_0174 | hypothetical protein | 23 | 47 | 31 | 2.043478261 | 1.347826087 |
| — | BB_0556 | hypothetical protein | 23 | 47 | 52 | 2.043478261 | 2.260869565 |
| pgsA | BB_0721 | CDP-diacylglycerol--glycerol-3- | 24 | 49 | 41 | 2.041666667 | 1.708333333 |
| | | phosphate 3- | | | | | |
| | | phosphatidyltransferase | | | | | |
| — | predicted | antisense: lysS | 15976 | 31965 | 26480 | 2.000813721 | 1.657486229 |
| | RNA | | | | | | |
| — | BB_Q13 | hypothetical protein | 2 | 4 | 14 | 2 | 7 |
| bppA | BB_Q43 | protein BppA | 3 | 6 | 13 | 2 | 4.333333333 |
| — | BB_0009 | hypothetical protein | 11 | 22 | 28 | 2 | 2.545454545 |
| — | BB_0066 | hypothetical protein | 4 | 8 | 14 | 2 | 3.5 |
| — | BB_0298 | hypothetical protein | 3 | 6 | 7 | 2 | 2.333333333 |
| — | BB_0345 | hypothetical protein | 7 | 14 | 12 | 2 | 1.714285714 |
| bmpA | BB_0383 | basic membrane protein A | 6 | 12 | 9 | 2 | 1.5 |
| — | BB_0505 | hypothetical protein | 17 | 34 | 32 | 2 | 1.882352941 |
| — | BB_0537 | hypothetical protein | 17 | 34 | 38 | 2 | 2.235294118 |
| — | BB_0605 | serine-type D-Ala-D-Ala | 40 | 80 | 62 | 2 | 1.55 |
| | | carboxypeptidase | | | | | |
| recD | BB_0632 | exodeoxyribonuclease V subunit | 7 | 14 | 14 | 2 | 2 |
| | | alpha | | | | | |
| — | BB_G20 | hypothetical protein | 6 | 12 | 19 | 2 | 3.166666667 |
| — | BB_H26 | hypothetical protein | 2 | 4 | 4 | 2 | 2 |
| — | BB_A38 | phage portal protein | 5 | 10 | 14 | 2 | 2.8 |
| bppC | BB_Q45 | protein BppC | 109 | 217 | 111 | 1.990825688 | 1.018348624 |
| — | BB_0536 | zinc protease | 242 | 479 | 809 | 1.979338843 | 3.342975207 |
| rpmH | BB_0440 | 50S ribosomal protein L34 | 40 | 79 | 35 | 1.975 | 0.875 |
| — | BB_0827 | ATP-dependent helicase | 39 | 77 | 101 | 1.974358974 | 2.58974359 |
| rpsL | BB_0387 | 30S ribosomal protein S12 | 35 | 69 | 27 | 1.971428571 | 0.771428571 |
| — | BB_0732 | penicillin-binding protein | 28 | 55 | 34 | 1.964285714 | 1.214285714 |
| — | BB_0683 | 3-hydroxy-3-methylglutaryl-CoA | 19 | 37 | 20 | 1.947368421 | 1.052631579 |
| | | synthase | | | | | |
| — | BB_M07 | hypothetical protein | 17 | 33 | 80 | 1.941176471 | 4.705882353 |
| — | BB_0640 | spermidine/putrescine ABC | 13 | 25 | 33 | 1.923076923 | 2.538461538 |
| | | transporter permease | | | | | |
| — | predicted | antisense: hisS | 11688 | 22464 | 32504 | 1.921971253 | 2.780971937 |
| | RNA | | | | | | |
| — | BB_0524 | inositol monophosphatase | 166 | 319 | 359 | 1.921686747 | 2.162650602 |
| — | predicted | antisense: BB_B10 | 20385 | 38903 | 60178 | 1.908413049 | 2.952072602 |
| | RNA | | | | | | |
| — | BB_0032 | hypothetical protein | 21 | 40 | 37 | 1.904761905 | 1.761904762 |
| — | BB_0781 | GTPase Obg | 20 | 38 | 36 | 1.9 | 1.8 |
| — | BB_0619 | DHH family protein | 48 | 91 | 74 | 1.895833333 | 1.541666667 |
| — | BB_0115 | 30S ribosomal protein S6 | 19 | 36 | 15 | 1.894736842 | 0.789473684 |
| — | predicted | antisense: BB_0381 | 11832 | 22391 | 22435 | 1.892410412 | 1.896129141 |
| | RNA | | | | | | |
| — | predicted | antisense: BB_P30 | 15254 | 28835 | 47546 | 1.890323849 | 3.11695293 |
| | RNA | | | | | | |
| — | BB_0192 | hypothetical protein | 9 | 17 | 13 | 1.888888889 | 1.444444444 |
| — | BB_A0078 | lipoprotein | 9 | 17 | 26 | 1.888888889 | 2.888888889 |
| — | predicted | antisense: BB_0786 | 46137 | 87099 | 97583 | 1.887834059 | 2.115070334 |
| | RNA | | | | | | |
| — | BB_0333 | peptide ABC transporter permease | 136 | 256 | 343 | 1.882352941 | 2.522058824 |
| — | BB_B19 | outer surface protein C | 346 | 650 | 982 | 1.878612717 | 2.838150289 |
| — | BB_0043 | hypothetical protein | 8 | 15 | 17 | 1.875 | 2.125 |
| murI | BB_0100 | glutamate racemase | 8 | 15 | 7 | 1.875 | 0.875 |
| rplM | BB_0339 | 50S ribosomal protein L13 | 48 | 90 | 21 | 1.875 | 0.4375 |
| infC | BB_0190 | translation initiation factor IF-3 | 52 | 97 | 78 | 1.865384615 | 1.5 |
| — | BB_0270 | flagellar biosynthesis regulator | 22 | 41 | 63 | 1.863636364 | 2.863636364 |
| | | FlhF | | | | | |
| — | BB_G15 | hypothetical protein | 22 | 41 | 95 | 1.863636364 | 4.318181818 |
| — | BB_0496 | 50S ribosomal protein L30 | 7 | 13 | 6 | 1.857142857 | 0.857142857 |
| — | BB_0795 | outer membrane protein | 14 | 26 | 24 | 1.857142857 | 1.714285714 |
| hbb | BB_0232 | DNA-binding protein HU | 20 | 37 | 15 | 1.85 | 0.75 |
| — | BB_0443 | spoIIIJ-associtated protein | 20 | 37 | 17 | 1.85 | 0.85 |
| — | BB_0743 | hypothetical protein | 20 | 37 | 54 | 1.85 | 2.7 |
| lspA | BB_0469 | lipoprotein signal peptidase | 13 | 24 | 19 | 1.846153846 | 1.461538462 |
| panF | BB_0814 | sodium/panthothenate symporter | 19 | 35 | 35 | 1.842105263 | 1.842105263 |
| — | predicted | antisense: rpmA | 12622 | 23222 | 23915 | 1.839803518 | 1.894707653 |
| | RNA | | | | | | |
| efbC | BB_0462 | nucleoid-associated protein EbfC | 24 | 44 | 41 | 1.833333333 | 1.708333333 |
| — | predicted | antisense: rplU | 15697 | 28773 | 27634 | 1.833025419 | 1.760463783 |
| | RNA | | | | | | |
| — | BB_0498 | protein translocase subunit SecY | 95 | 174 | 110 | 1.831578947 | 1.157894737 |
| — | predicted | antisense: dnaK | 13207 | 24137 | 27386 | 1.827591429 | 2.073597335 |
| | RNA | | | | | | |
| — | BB_0136 | penicillin-binding protein | 214 | 391 | 482 | 1.827102804 | 2.252336449 |
| — | BB_0685 | 3-hydroxy-3-methylglutaryl-CoA | 34 | 62 | 76 | 1.823529412 | 2.235294118 |
| | | reductase | | | | | |
| — | BB_0568 | chemotaxis response regulator | 51 | 92 | 86 | 1.803921569 | 1.68627451 |
| — | BB_0108 | basic membrane protein | 15 | 27 | 27 | 1.8 | 1.8 |
| — | BB_G26 | hypothetical protein | 5 | 9 | 22 | 1.8 | 4.4 |
| — | predicted | antisense: BB_S34 | 12380 | 22283 | 31741 | 1.799919225 | 2.563893376 |
| | RNA | | | | | | |
| — | BB_R03 | hypothetical protein | 19 | 34 | 64 | 1.789473684 | 3.368421053 |
| — | predicted | antisense: clpX | 11980 | 21399 | 24332 | 1.786227045 | 2.031051753 |
| | RNA | | | | | | |
| — | BB_0829 | exonuclease SbcD | 28 | 50 | 41 | 1.785714286 | 1.464285714 |
| — | BB_G14 | hypothetical protein | 14 | 25 | 44 | 1.785714286 | 3.142857143 |
| — | BB_0244 | hypothetical protein | 81 | 144 | 139 | 1.777777778 | 1.716049383 |
| — | BB_0261 | hypothetical protein | 27 | 48 | 30 | 1.777777778 | 1.111111111 |
| rplL | BB_0390 | 50S ribosomal protein L7/L12 | 9 | 16 | 9 | 1.777777778 | 1 |
| dbpB | BB_A25 | decorin-binding protein B | 175 | 311 | 811 | 1.777142857 | 4.634285714 |
| mlpA | BB_P28 | surface lipoprotein | 40 | 71 | 103 | 1.775 | 2.575 |
| fmt | BB_0064 | methionyl-tRNA formyltransferase | 53 | 94 | 129 | 1.773584906 | 2.433962264 |
| — | predicted | antisense: BB_0096 | 13035 | 23072 | 21584 | 1.770003836 | 1.655849636 |
| | RNA | | | | | | |
| — | BB_0145 | glycine/betaine ABC transporter | 39 | 69 | 53 | 1.769230769 | 1.358974359 |
| | | permease | | | | | |
| rpsD | BB_0615 | 30S ribosomal protein S4 | 78 | 138 | 55 | 1.769230769 | 0.705128205 |
| hflC | BB_0204 | protein HflC | 47 | 83 | 99 | 1.765957447 | 2.106382979 |
| — | predicted | — | 13193 | 23291 | 27045 | 1.765405897 | 2.049950731 |
| | RNA | | | | | | |
| — | BB_0715 | cell division protein FtsA | 25 | 44 | 15 | 1.76 | 0.6 |
| pgi | BB_0730 | glucose-6-phosphate isomerase | 86 | 151 | 148 | 1.755813953 | 1.720930233 |
| — | BB_0763 | acetoacetate metabolism regulatory | 347 | 609 | 1293 | 1.755043228 | 3.726224784 |
| | | protein AtoC | | | | | |
| — | BB_0347 | fibronectin/fibrinogen-binding | 12 | 21 | 22 | 1.75 | 1.833333333 |
| | | protein | | | | | |
| — | BB_0421 | haloacid dehalogenase-like | 8 | 14 | 24 | 1.75 | 3 |
| | | hydrolase | | | | | |
| — | BB_0426 | nucleoside 2- | 8 | 14 | 10 | 1.75 | 1.25 |
| | | deoxyribosyltransferase | | | | | |
| | | superfamily protein | | | | | |
| — | predicted | antisense: clpP | 13203 | 23082 | 25359 | 1.748239037 | 1.920699841 |
| | RNA | | | | | | |
| — | predicted | antisense: rho | 13001 | 22555 | 15380 | 1.734866549 | 1.182985924 |
| | RNA | | | | | | |
| — | BB_0330 | peptide ABC transporter substrate- | 79 | 137 | 142 | 1.734177215 | 1.797468354 |
| | | binding protein | | | | | |
| — | BB_0085 | hypothetical protein | 52 | 90 | 80 | 1.730769231 | 1.538461538 |
| — | BB_J45 | lipoprotein | 26 | 45 | 38 | 1.730769231 | 1.461538462 |
| flgB | BB_0294 | flagellar basal body rod protein | 11 | 19 | 5 | 1.727272727 | 0.454545455 |
| | | FlgB | | | | | |
| — | BB_0714 | hypothetical protein | 11 | 19 | 9 | 1.727272727 | 0.818181818 |
| glpK | BB_0241 | glycerol kinase | 107 | 184 | 208 | 1.719626168 | 1.943925234 |
| — | BB_0512 | hypothetical protein | 7 | 12 | 10 | 1.714285714 | 1.428571429 |
| flgC | BB_0293 | flagellar basal body rod protein | 17 | 29 | 12 | 1.705882353 | 0.705882353 |
| | | FlgC | | | | | |
| — | predicted | antisense: BB_0714 | 17359 | 29572 | 25147 | 1.703554352 | 1.448643355 |
| | RNA | | | | | | |
| — | BB_0304 | UDP-N-acetylmuramoyl-tripeptide-- | 20 | 34 | 45 | 1.7 | 2.25 |
| | | D-alanyl-D-alanine ligas | | | | | |
| — | BB_0325 | hypothetical protein | 169 | 287 | 240 | 1.698224852 | 1.420118343 |
| — | BB_0374 | hypothetical protein | 33 | 56 | 39 | 1.696969697 | 1.181818182 |
| bosR | BB_0647 | oxidative stress regulator BosR | 23 | 39 | 46 | 1.695652174 | 2 |
| — | BB_0765 | hypothetical protein | 36 | 61 | 109 | 1.694444444 | 3.027777778 |
| rplR | BB_0494 | 50S ribosomal protein L18 | 26 | 44 | 20 | 1.692307692 | 0.769230769 |
| mlpC | BB_S30 | lipoprotein | 35 | 59 | 76 | 1.685714286 | 2.171428571 |
| — | predicted | antisense: ftsA | 23827 | 39846 | 41790 | 1.672304528 | 1.753892643 |
| | RNA | | | | | | |
| — | predicted | antisense: BB_0621 | 13781 | 23013 | 25731 | 1.669907844 | 1.867135912 |
| | RNA | | | | | | |
| — | predicted | antisense: ftsA | 27455 | 45823 | 49049 | 1.669022036 | 1.786523402 |
| | RNA | | | | | | |
| — | BB_M05 | hypothetical protein | 3 | 5 | 23 | 1.666666667 | 7.666666667 |
| — | BB_0092 | V-type ATPase subunit D | 3 | 5 | 3 | 1.666666667 | 1 |
| — | BB_0096 | V-type ATP synthase subunit E | 9 | 15 | 8 | 1.666666667 | 0.888888889 |
| rrmJ | BB_0313 | rRNA large subunit | 3 | 5 | 6 | 1.666666667 | 2 |
| | | methyltransferase | | | | | |
| tRNA- | BB_t05 | Ala tRNA | 6 | 10 | 0 | 1.666666667 | 0 |
| Ala-1 | | | | | | | |
| — | BB_0473 | integral membrane protein | 18 | 30 | 34 | 1.666666667 | 1.888888889 |
| — | BB_0696 | hypothetical protein | 18 | 30 | 40 | 1.666666667 | 2.222222222 |
| — | BB_0570 | chemotaxis response regulator | 276 | 459 | 540 | 1.663043478 | 1.956521739 |
| — | predicted | antisense: BB_0715 | 24255 | 40327 | 45509 | 1.662626263 | 1.876272933 |
| | RNA | | | | | | |
| — | BB_0431 | CobQ/CobB/MinD/ParA nucleotide | 35 | 58 | 54 | 1.657142857 | 1.542857143 |
| | | binding domain-containing protein | | | | | |
| — | BB_0361 | ATP-binding protein | 99 | 164 | 114 | 1.656565657 | 1.151515152 |
| — | BB_0621 | 4-methyl-5(b-hydroxyethyl)- | 48 | 79 | 48 | 1.645833333 | 1 |
| | | thiazole monophosphate | | | | | |
| | | biosynthesis protein | | | | | |
| — | BB_Q20 | hypothetical protein | 14 | 23 | 91 | 1.642857143 | 6.5 |
| — | BB_0281 | motility protein A | 25 | 41 | 26 | 1.64 | 1.04 |
| — | BB_M04 | hypothetical protein | 11 | 18 | 37 | 1.636363636 | 3.363636364 |
| fliP | BB_0275 | flagellar biosynthesis protein FliP | 52 | 85 | 106 | 1.634615385 | 2.038461538 |
| — | predicted | antisense: groS | 18987 | 30852 | 18770 | 1.624901248 | 0.988571128 |
| | RNA | | | | | | |
| — | predicted | antisense: BB_0715 | 14453 | 23343 | 25596 | 1.615097212 | 1.770981803 |
| | RNA | | | | | | |
| — | BB_0764 | sensory transduction histidine | 57 | 92 | 157 | 1.614035088 | 2.754385965 |
| | | kinase | | | | | |
| — | predicted | antisense: BB_0724 | 21771 | 34870 | 30624 | 1.601671949 | 1.406641863 |
| | RNA | | | | | | |
| recJ | BB_0254 | single-stranded-DNA-specific | 30 | 48 | 34 | 1.6 | 1.133333333 |
| | | exonuclease | | | | | |
| murA | BB_0472 | UDP-N-acetylglucosamine 1- | 15 | 24 | 12 | 1.6 | 0.8 |
| | | carboxyvinyltransferase | | | | | |
| — | BB_0258 | undecaprenyl-diphosphatase | 118 | 188 | 158 | 1.593220339 | 1.338983051 |
| — | BB_0745 | endonuclease III | 86 | 137 | 138 | 1.593023256 | 1.604651163 |
| — | BB_0317 | integral membrane protein | 29 | 46 | 21 | 1.586206897 | 0.724137931 |
| — | predicted | antisense: ftsA | 25843 | 40980 | 36226 | 1.585729211 | 1.40177224 |
| | RNA | | | | | | |
| flgG | BB_0774 | flagellar basal body rod protein | 24 | 38 | 55 | 1.583333333 | 2.291666667 |
| | | FlgG | | | | | |
| — | BB_0796 | hypothetical protein | 12 | 19 | 34 | 1.583333333 | 2.833333333 |
| — | BB_0449 | hypothetical protein | 19 | 30 | 38 | 1.578947368 | 2 |
| — | predicted | antisense: BB_0176 | 17277 | 27257 | 25789 | 1.577646582 | 1.492678127 |
| | RNA | | | | | | |
| — | predicted | antisense: BB_0715 | 18485 | 29083 | 32592 | 1.573329727 | 1.763159318 |
| | RNA | | | | | | |
| trx | BB_0061 | thioredoxin | 21 | 33 | 12 | 1.571428571 | 0.571428571 |
| ftsZ | BB_0299 | cell division protein FtsZ | 7 | 11 | 16 | 1.571428571 | 2.285714286 |
| — | BB_G19 | hypothetical protein | 7 | 11 | 31 | 1.571428571 | 4.428571429 |
| — | BB_G29 | hypothetical protein | 7 | 11 | 11 | 1.571428571 | 1.571428571 |
| rbsA | BB_0677 | sugar ABC transporter ATP- | 16 | 25 | 19 | 1.5625 | 1.1875 |
| | | binding protein | | | | | |
| mvk | BB_0688 | mevalonate kinase | 1001 | 1560 | 2436 | 1.558441558 | 2.433566434 |
| bppA | BB_R36 | BppA | 9 | 14 | 27 | 1.555555556 | 3 |
| — | BB_t31 | Met tRNA | 18 | 28 | 21 | 1.555555556 | 1.166666667 |
| — | BB_G13 | hypothetical protein | 9 | 14 | 44 | 1.555555556 | 4.888888889 |
| — | BB_0163 | hypothetical protein | 167 | 259 | 238 | 1.550898204 | 1.425149701 |
| — | BB_0359 | peptidase S41 | 20 | 31 | 38 | 1.55 | 1.9 |
| — | predicted | antisense: BB_P31 | 23325 | 36053 | 46733 | 1.5456806 | 2.003558414 |
| | RNA | | | | | | |
| — | BB_0180 | flagellar protein FlbF | 13 | 20 | 12 | 1.538461538 | 0.923076923 |
| coaD | BB_0702 | phosphopantetheine | 13 | 20 | 8 | 1.538461538 | 0.615384615 |
| | | adenylyltransferase | | | | | |
| — | BB_0772 | flagellar basal body P-ring protein | 13 | 20 | 22 | 1.538461538 | 1.692307692 |
| — | BB_H41 | membrane protein | 15 | 23 | 19 | 1.533333333 | 1.266666667 |
| — | BB_0257 | DNA translocase FtsK | 562 | 861 | 646 | 1.53202847 | 1.149466192 |
| — | predicted | antisense: BB_0094 | 18820 | 28757 | 26181 | 1.528002125 | 1.391126461 |
| | RNA | | | | | | |
| flgK | BB_0181 | flagellar hook-associated protein | 150 | 229 | 191 | 1.526666667 | 1.273333333 |
| | | FlgK | | | | | |
| — | predicted | antisense: rpsD | 24606 | 37497 | 37801 | 1.523896611 | 1.536251321 |
| | RNA | | | | | | |
| — | predicted | antisense: BB_0666 | 18279 | 27562 | 15273 | 1.507850539 | 0.835548991 |
| | RNA | | | | | | |
| — | BB_R18 | hypothetical protein | 8 | 12 | 15 | 1.5 | 1.875 |
| — | BB_M20 | hypothetical protein | 2 | 3 | 3 | 1.5 | 1.5 |
| — | BB_N25 | hypothetical protein | 6 | 9 | 18 | 1.5 | 3 |
| — | BB_0242 | hypothetical protein | 6 | 9 | 2 | 1.5 | 0.333333333 |
| — | BB_0311 | NAD kinase | 10 | 15 | 13 | 1.5 | 1.3 |
| rplO | BB_0497 | 50S ribosomal protein L15 | 14 | 21 | 16 | 1.5 | 1.142857143 |
| — | BB_0770 | divergent polysaccharide | 22 | 33 | 33 | 1.5 | 1.5 |
| | | deacetylase superfamily protein | | | | | |
| — | BB_0840 | lipoprotein | 8 | 12 | 13 | 1.5 | 1.625 |
| — | predicted | antisense: BB_0231 hbb | 24725 | 36941 | 32185 | 1.494074823 | 1.301718908 |
| | RNA | | | | | | |
| — | predicted | antisense: rplS | 26105 | 38817 | 32580 | 1.486956522 | 1.248036775 |
| | RNA | | | | | | |
| pyrG | BB_0575 | CTP synthetase | 62 | 92 | 111 | 1.483870968 | 1.790322581 |
| — | BB_0149 | flagellar hook-associated protein | 23 | 34 | 40 | 1.47826087 | 1.739130435 |
| | | FliD | | | | | |
| cvpA | BB_0766 | colicin V production protein | 82 | 121 | 271 | 1.475609756 | 3.304878049 |
| — | BB_0596 | methyl-accepting chemotaxis | 59 | 87 | 87 | 1.474576271 | 1.474576271 |
| | | protein | | | | | |
| — | BB_0209 | hypothetical protein | 38 | 56 | 80 | 1.473684211 | 2.105263158 |
| — | BB_0625 | N-acetylmuramoyl-L-alanine | 55 | 81 | 25 | 1.472727273 | 0.454545455 |
| | | amidase | | | | | |
| — | BB_0565 | purine-binding chemotaxis protein | 36 | 53 | 39 | 1.472222222 | 1.083333333 |
| — | predicted | antisense: BB_0097 | 17800 | 26159 | 25474 | 1.469606742 | 1.431123596 |
| | RNA | | | | | | |
| — | BB_0047 | hypothetical protein | 47 | 69 | 34 | 1.468085106 | 0.723404255 |
| — | predicted | antisense: BB_0786 | 26757 | 39154 | 43090 | 1.46331801 | 1.610419703 |
| | RNA | | | | | | |
| — | BB_P04 | hypothetical protein | 22 | 32 | 77 | 1.454545455 | 3.5 |
| — | BB_R32 | hypothetical protein | 11 | 16 | 14 | 1.454545455 | 1.272727273 |
| — | BB_H32 | antigen P35 | 11 | 16 | 5 | 1.454545455 | 0.454545455 |
| — | BB_0052 | tRNA/rRNA methyltransferase | 218 | 316 | 374 | 1.449541284 | 1.71559633 |
| clpX | BB_0612 | ATP-dependent protease ATP- | 1221 | 1766 | 1220 | 1.446355446 | 0.999180999 |
| | | binding subunit ClpX | | | | | |
| gyrB | BB_0436 | DNA gyrase subunit B | 27 | 39 | 28 | 1.444444444 | 1.037037037 |
| fusA | BB_0540 | elongation factor G | 45 | 65 | 49 | 1.444444444 | 1.088888889 |
| — | BB_0089 | hypothetical protein | 16 | 23 | 11 | 1.4375 | 0.6875 |
| — | BB_0132 | transcription elongation factor | 30 | 43 | 42 | 1.433333333 | 1.4 |
| | | GreA | | | | | |
| — | BB_0334 | peptide ABC transporter ATP- | 538 | 768 | 1101 | 1.427509294 | 2.046468401 |
| | | binding protein | | | | | |
| — | BB_0110 | hypothetical protein | 108 | 154 | 117 | 1.425925926 | 1.083333333 |
| — | predicted | antisense: BB_0715 | 20895 | 29754 | 42674 | 1.423977028 | 2.042306772 |
| | RNA | | | | | | |
| — | predicted | antisense: BB_P31 | 15330 | 21758 | 30593 | 1.419308545 | 1.995629485 |
| | RNA | | | | | | |
| flgL | BB_0182 | flagellar hook-associated protein | 315 | 446 | 338 | 1.415873016 | 1.073015873 |
| | | FlgL | | | | | |
| — | predicted | antisense: pth BB_0786 | 23467 | 33206 | 37263 | 1.41500831 | 1.587889377 |
| | RNA | | | | | | |
| — | BB_0141 | membrane fusion protein | 29 | 41 | 81 | 1.413793103 | 2.793103448 |
| — | BB_0650 | hypothetical protein | 503 | 706 | 944 | 1.403578529 | 1.876739563 |
| bppA | BB_M35 | BppA | 5 | 7 | 11 | 1.4 | 2.2 |
| — | BB_0068 | haloacid dehalogenase-like | 10 | 14 | 12 | 1.4 | 1.2 |
| | | hydrolase | | | | | |
| — | BB_0156 | hypothetical protein | 5 | 7 | 7 | 1.4 | 1.4 |
| ftsW | BB_0302 | lipid II flippase FtsW | 45 | 63 | 50 | 1.4 | 1.111111111 |
| — | BB_0439 | hypothetical protein | 10 | 14 | 7 | 1.4 | 0.7 |
| ylqF | BB_0643 | ribosome biogenesis GTPase A | 30 | 42 | 56 | 1.4 | 1.866666667 |
| — | BB_0050 | hypothetical protein | 171 | 239 | 136 | 1.397660819 | 0.795321637 |
| — | BB_0072 | membrane protein | 48 | 67 | 75 | 1.395833333 | 1.5625 |
| rpsS | BB_0482 | 30S ribosomal protein S19 | 33 | 46 | 37 | 1.393939394 | 1.121212121 |
| tgt | BB_0809 | queuine tRNA-ribosyltransferase | 259 | 360 | 443 | 1.38996139 | 1.71042471 |
| phoU | BB_0042 | phosphate-specific transport system | 13 | 18 | 25 | 1.384615385 | 1.923076923 |
| | | accessory protein PhoU | | | | | |
| — | BB_0236 | hypothetical protein | 26 | 36 | 29 | 1.384615385 | 1.115384615 |
| metG | BB_0587 | methionine--tRNA ligase | 84 | 116 | 117 | 1.380952381 | 1.392857143 |
| — | BB_0800 | transcription | 21 | 29 | 51 | 1.380952381 | 2.428571429 |
| | | termination/antitermination protein NusA | | | | | |
| — | predicted | antisense: BB_0209 | 17088 | 23558 | 15447 | 1.378628277 | 0.903967697 |
| | RNA | | | | | | |
| bppA | BB_S38 | protein BppA | 16 | 22 | 46 | 1.375 | 2.875 |
| — | BB_O32 | PF-32 protein | 8 | 11 | 5 | 1.375 | 0.625 |
| — | BB_0838 | hypothetical protein | 24 | 33 | 63 | 1.375 | 2.625 |
| — | predicted | antisense: miaA | 24257 | 33333 | 27122 | 1.374160036 | 1.118110236 |
| | RNA | | | | | | |
| — | BB_Q14 | hypothetical protein | 19 | 26 | 44 | 1.368421053 | 2.315789474 |
| nagA | BB_0151 | N-acetylglucosamine-6-phosphate | 117 | 160 | 138 | 1.367521368 | 1.179487179 |
| | | deacetylase | | | | | |
| — | BB_0161 | hypothetical protein | 152 | 207 | 176 | 1.361842105 | 1.157894737 |
| rplS | BB_0699 | 50S ribosomal protein L19 | 36 | 49 | 18 | 1.361111111 | 0.5 |
| murD | BB_0585 | UDP-N-acetylmuramoylalanine-- | 114 | 155 | 158 | 1.359649123 | 1.385964912 |
| | | D-glutamate ligase | | | | | |
| — | BB_0314 | octaprenyl-diphosphate synthase | 51 | 69 | 80 | 1.352941176 | 1.568627451 |
| rplB | BB_0481 | 50S ribosomal protein L2 | 17 | 23 | 19 | 1.352941176 | 1.117647059 |
| — | BB_0600 | hypothetical protein | 17 | 23 | 15 | 1.352941176 | 0.882352941 |
| — | predicted | antisense: BB_0083 | 21449 | 29005 | 18734 | 1.352277495 | 0.873420672 |
| | RNA | | | | | | |
| — | BB_0490 | 50S ribosomal protein L5 | 32 | 43 | 18 | 1.34375 | 0.5625 |
| — | BB_S35 | PF-32 protein | 9 | 12 | 7 | 1.333333333 | 0.777777778 |
| — | BB_Q54 | hypothetical protein | 3 | 4 | 1 | 1.333333333 | 0.333333333 |
| arcA | BB_0841 | arginine deiminase | 9 | 12 | 13 | 1.333333333 | 1.444444444 |
| — | predicted | antisense: BB_0461 | 17112 | 22768 | 28852 | 1.330528284 | 1.686068256 |
| | RNA | | | | | | |
| gap | BB_0057 | glyceraldehyde 3-phosphate | 40 | 53 | 38 | 1.325 | 0.95 |
| | | dehydrogenase | | | | | |
| — | BB_0342 | glutamyl-tRNA(Gln) | 56 | 74 | 77 | 1.321428571 | 1.375 |
| | | amidotransferase subunit A | | | | | |
| — | predicted | antisense: BB_0775 | 18180 | 23873 | 27569 | 1.313146315 | 1.516446645 |
| | RNA | | | | | | |
| — | BB_0805 | polyribonucleotide | 16 | 21 | 16 | 1.3125 | 1 |
| | | nucleotidyltransferase | | | | | |
| — | BB_G16 | hypothetical protein | 16 | 21 | 73 | 1.3125 | 4.5625 |
| fliH | BB_0289 | flagellar assembly protein FliH | 29 | 38 | 89 | 1.310344828 | 3.068965517 |
| — | BB_A52 | outer membrane protein | 42 | 55 | 66 | 1.30952381 | 1.571428571 |
| bppC | BB_L38 | protein BppC | 112 | 146 | 98 | 1.303571429 | 0.875 |
| — | BB_0747 | oligopeptide ABC transporter | 86 | 112 | 101 | 1.302325581 | 1.174418605 |
| | | permease | | | | | |
| — | BB_0240 | glycerol uptake facilitator | 817 | 1062 | 1140 | 1.299877601 | 1.395348837 |
| — | predicted | antisense: nusG | 28038 | 36279 | 43188 | 1.293922534 | 1.540338113 |
| | RNA | | | | | | |
| ileS | BB_0833 | isoleucine--tRNA ligase | 41 | 53 | 77 | 1.292682927 | 1.87804878 |
| — | BB_D18 | hypothetical protein | 72 | 93 | 135 | 1.291666667 | 1.875 |
| — | predicted | antisense: tig | 24014 | 30953 | 47340 | 1.288956442 | 1.971350046 |
| | RNA | | | | | | |
| — | BB_0198 | guanosine-3′,5′-bis(diphosphate) 3′- | 104 | 134 | 127 | 1.288461538 | 1.221153846 |
| | | pyrophosphohydrolase | | | | | |
| — | BB_0418 | hypothetical protein | 1392 | 1791 | 1727 | 1.286637931 | 1.24066092 |
| — | predicted | antisense: dnaK | 16301 | 20898 | 40272 | 1.282007239 | 2.470523281 |
| | RNA | | | | | | |
| — | predicted | antisense: BB_0726 | 23070 | 29560 | 33732 | 1.281317729 | 1.462158648 |
| | RNA | | | | | | |
| rpsT | BB_0233 | 30S ribosomal protein S20 | 32 | 41 | 33 | 1.28125 | 1.03125 |
| — | BB_P15 | hypothetical protein | 139 | 178 | 459 | 1.28057554 | 3.302158273 |
| rpsP | BB_0695 | 30S ribosomal protein S16 | 25 | 32 | 26 | 1.28 | 1.04 |
| — | predicted | antisense: BB_0442 | 23386 | 29916 | 41128 | 1.279226888 | 1.758659027 |
| | RNA | | | | | | |
| — | predicted | antisense: rpmF | 20873 | 26583 | 24243 | 1.273559143 | 1.161452594 |
| | RNA | | | | | | |
| — | predicted | antisense: BB_0096 | 20600 | 26200 | 29681 | 1.27184466 | 1.440825243 |
| | RNA | | | | | | |
| — | predicted | — | 26791 | 34065 | 91735 | 1.271509089 | 3.424097645 |
| | RNA | | | | | | |
| — | BB_0713 | hypothetical protein | 52 | 66 | 163 | 1.269230769 | 3.134615385 |
| — | BB_S34 | hypothetical protein | 15 | 19 | 30 | 1.266666667 | 2 |
| — | BB_0094 | ATP synthase subunit A | 15 | 19 | 11 | 1.266666667 | 0.733333333 |
| — | BB_0243 | glycerol-3-phosphate | 166 | 209 | 163 | 1.259036145 | 0.981927711 |
| | | dehydrogenase | | | | | |
| — | BB_0006 | membrane protein | 58 | 73 | 85 | 1.25862069 | 1.465517241 |
| — | BB_0654 | hypothetical protein | 93 | 117 | 83 | 1.258064516 | 0.892473118 |
| bapA | BB_S42 | BapA protein | 4 | 5 | 10 | 1.25 | 2.5 |
| bppA | BB_N35 | protein BppA | 4 | 5 | 15 | 1.25 | 3.75 |
| — | BB_0285 | flagellar protein | 8 | 10 | 10 | 1.25 | 1.25 |
| apt | BB_0777 | adenine phosphoribosyltransferase | 16 | 20 | 36 | 1.25 | 2.25 |
| — | BB_J13 | hypothetical protein | 16 | 20 | 5 | 1.25 | 0.3125 |
| — | predicted | antisense: rplS trmD | 29045 | 36298 | 40945 | 1.249715958 | 1.409709072 |
| | RNA | | | | | | |
| pepF | BB_0248 | oligoendopeptidase F | 367 | 456 | 515 | 1.242506812 | 1.403269755 |
| — | BB_G18 | hypothetical protein | 29 | 36 | 70 | 1.24137931 | 2.413793103 |
| — | predicted | antisense: BB_0429 | 17868 | 22062 | 19047 | 1.234721289 | 1.065983882 |
| | RNA | | | | | | |
| — | BB_0381 | hypothetical protein | 47 | 58 | 44 | 1.234042553 | 0.936170213 |
| — | BB_0669 | chemotaxis protein CheA | 26 | 32 | 47 | 1.230769231 | 1.807692308 |
| groL | BB_0649 | chaperonin GroEL | 58 | 71 | 107 | 1.224137931 | 1.844827586 |
| — | BB_0264 | heat shock protein 70 | 94 | 115 | 81 | 1.223404255 | 0.861702128 |
| — | BB_R33 | plasmid partition protein, putative | 9 | 11 | 10 | 1.222222222 | 1.111111111 |
| erpY | BB_R42 | ErpY | 9 | 11 | 7 | 1.222222222 | 0.777777778 |
| — | predicted | antisense: BB_0715 | 22733 | 27750 | 37984 | 1.220692386 | 1.67087494 |
| | RNA | | | | | | |
| — | predicted | antisense: BB_0158 | 24644 | 30014 | 26749 | 1.217902938 | 1.085416329 |
| | RNA | | | | | | |
| — | predicted | antisense: BB_0785 | 45789 | 55681 | 49411 | 1.216034419 | 1.079101968 |
| | RNA | | | | | | |
| fliF | BB_0291 | flagellar MS-ring protein FliF | 14 | 17 | 23 | 1.214285714 | 1.642857143 |
| rpsC | BB_0484 | 30S ribosomal protein S3 | 14 | 17 | 19 | 1.214285714 | 1.357142857 |
| rimM | BB_0697 | ribosome maturation factor RimM | 127 | 154 | 137 | 1.212598425 | 1.078740157 |
| tmk | BB_0793 | thymidylate kinase | 80 | 97 | 103 | 1.2125 | 1.2875 |
| — | predicted | antisense: BB_0505 | 22116 | 26785 | 32397 | 1.211114126 | 1.464867065 |
| | RNA | | | | | | |
| — | BB_0142 | outer membrane efflux protein | 19 | 23 | 24 | 1.210526316 | 1.263157895 |
| — | BB_P18 | hypothetical protein | 48 | 58 | 86 | 1.208333333 | 1.791666667 |
| — | BB_0221 | flagellar motor switch protein | 53 | 64 | 123 | 1.20754717 | 2.320754717 |
| — | predicted | antisense: tig | 30584 | 36789 | 51004 | 1.202883861 | 1.66766937 |
| | RNA | | | | | | |
| rpmJ | BB_0499 | 50S ribosomal protein L36 | 60 | 72 | 14 | 1.2 | 0.233333333 |
| — | BB_0577 | hypothetical protein | 10 | 12 | 6 | 1.2 | 0.6 |
| — | BB_D15 | membrane protein | 5 | 6 | 12 | 1.2 | 2.4 |
| — | BB_0419 | response regulator | 72 | 86 | 34 | 1.194444444 | 0.472222222 |
| — | BB_0751 | hypothetical protein | 73 | 87 | 110 | 1.191780822 | 1.506849315 |
| — | BB_0629 | PTS system fructose-specific | 486 | 579 | 460 | 1.191358025 | 0.946502058 |
| | | transporter subunit IIABC | | | | | |
| — | BB_G08 | stage 0 sporulation protein SpoOJ | 21 | 25 | 87 | 1.19047619 | 4.142857143 |
| — | BB_A40 | hypothetical protein | 21 | 25 | 46 | 1.19047619 | 2.19047619 |
| — | predicted | antisense: nusG | 29641 | 35281 | 33268 | 1.190276981 | 1.122364293 |
| | RNA | | | | | | |
| — | BB_H33 | adenine deaminase | 16 | 19 | 2 | 1.1875 | 0.125 |
| — | BB_R41 | hypothetical protein | 11 | 13 | 20 | 1.181818182 | 1.818181818 |
| mreC | BB_0716 | rod shape-determining protein | 72 | 85 | 71 | 1.180555556 | 0.986111111 |
| | | MreC | | | | | |
| trmU | BB_0682 | tRNA-specific 2-thiouridylase | 89 | 105 | 108 | 1.179775281 | 1.213483146 |
| | | MnmA | | | | | |
| tRNA- | BB_t17 | Leu tRNA | 45 | 53 | 15 | 1.177777778 | 0.333333333 |
| Leu-4 | | | | | | | |
| coaBC | BB_0812 | bifunctional | 102 | 120 | 98 | 1.176470588 | 0.960784314 |
| | | phosphopantothenoylcysteine | | | | | |
| | | decarboxylase/phosphopantothenate-- | | | | | |
| | | cysteine ligase | | | | | |
| — | predicted | antisense: tilS | 26973 | 31653 | 34349 | 1.17350684 | 1.273458644 |
| | RNA | | | | | | |
| p66 | BB_0603 | integral outer membrane protein | 247 | 289 | 196 | 1.170040486 | 0.793522267 |
| | | p66 | | | | | |
| — | BB_N04 | hypothetical protein | 6 | 7 | 15 | 1.166666667 | 2.5 |
| — | BB_0843 | arginine-ornithine antiporter | 43 | 50 | 60 | 1.162790698 | 1.395348837 |
| prfA | BB_0196 | peptide chain release factor 1 | 56 | 65 | 86 | 1.160714286 | 1.535714286 |
| — | predicted | antisense: BB_0132 | 18235 | 21129 | 35397 | 1.158705786 | 1.941157115 |
| | RNA | | | | | | |
| — | predicted | antisense: flhB | 42457 | 49175 | 47245 | 1.15823068 | 1.112772923 |
| | RNA | | | | | | |
| — | BB_0466 | ABC transporter ATP-binding | 19 | 22 | 24 | 1.157894737 | 1.263157895 |
| | | protein | | | | | |
| — | BB_P26 | hypothetical protein | 351 | 406 | 355 | 1.156695157 | 1.011396011 |
| — | BB_Q22 | hypothetical protein | 13 | 15 | 35 | 1.153846154 | 2.692307692 |
| hslU | BB_0295 | ATP-dependent protease ATPase | 13 | 15 | 30 | 1.153846154 | 2.307692308 |
| | | subunit HslU | | | | | |
| — | BB_0717 | integral membrane protein | 46 | 53 | 58 | 1.152173913 | 1.260869565 |
| — | BB_0734 | Sua5/YciO/YrdC/YwlC family | 132 | 152 | 133 | 1.151515152 | 1.007575758 |
| | | protein | | | | | |
| — | BB_0794 | hypothetical protein | 33 | 38 | 75 | 1.151515152 | 2.272727273 |
| — | predicted | antisense: BB_0712 | 19448 | 22394 | 28668 | 1.151480872 | 1.474084739 |
| | RNA | | | | | | |
| — | predicted | antisense: tRNA-Trp-1 rpmG | 39226 | 44973 | 40055 | 1.146509968 | 1.021133942 |
| | RNA | | | | | | |
| — | BB_0297 | protein smf | 7 | 8 | 13 | 1.142857143 | 1.857142857 |
| — | predicted | antisense: prfB | 23877 | 27266 | 16597 | 1.141935754 | 0.695104075 |
| | RNA | | | | | | |
| — | predicted | antisense: infC | 23714 | 27036 | 19227 | 1.140086025 | 0.810786877 |
| | RNA | | | | | | |
| — | predicted | antisense: BB_0789 | 23189 | 26418 | 37528 | 1.139247057 | 1.61835353 |
| | RNA | | | | | | |
| mlpJ | BB_Q35 | protein MlpJ | 80 | 91 | 148 | 1.1375 | 1.85 |
| — | predicted | antisense: BB_S33 | 19846 | 22554 | 28208 | 1.13645067 | 1.421344352 |
| | RNA | | | | | | |
| rdgB | BB_0247 | non-canonical purine NTP | 22 | 25 | 39 | 1.136363636 | 1.772727273 |
| | | pyrophosphatase | | | | | |
| — | BB_P41 | hypothetical protein | 15 | 17 | 59 | 1.133333333 | 3.933333333 |
| rnmV | BB_0626 | ribonuclease M5 | 488 | 552 | 792 | 1.131147541 | 1.62295082 |
| — | predicted | antisense: rplL | 31854 | 35837 | 36895 | 1.125039242 | 1.158253281 |
| | RNA | | | | | | |
| thyX | BB_A76 | FAD-dependent thymidylate | 8 | 9 | 3 | 1.125 | 0.375 |
| | | synthase | | | | | |
| — | predicted | antisense: ftsA | 19555 | 21984 | 32302 | 1.124213756 | 1.651853746 |
| | RNA | | | | | | |
| — | BB_0355 | transcription factor | 57 | 64 | 33 | 1.122807018 | 0.578947368 |
| bppA | BB_P35 | protein BppA | 25 | 28 | 45 | 1.12 | 1.8 |
| — | BB_H0042 | hypothetical protein | 25 | 28 | 15 | 1.12 | 0.6 |
| — | BB_0276 | flagellar biosynthesis protein FliZ | 17 | 19 | 40 | 1.117647059 | 2.352941176 |
| — | BB_0318 | ABC transporter ATP-binding | 27 | 30 | 37 | 1.111111111 | 1.37037037 |
| | | protein | | | | | |
| — | BB_J43 | hypothetical protein | 18 | 20 | 18 | 1.111111111 | 1 |
| tuf | BB_0476 | elongation factor Tu | 37 | 41 | 33 | 1.108108108 | 0.891891892 |
| — | predicted | antisense: BB_0133 | 40842 | 45226 | 44493 | 1.107340483 | 1.089393272 |
| | RNA | | | | | | |
| — | predicted | antisense: rplA | 52924 | 58392 | 74816 | 1.103317965 | 1.413649762 |
| | RNA | | | | | | |
| — | predicted | antisense: clpX | 26799 | 29556 | 39941 | 1.102876973 | 1.490391433 |
| | RNA | | | | | | |
| uvrC | BB_0457 | excinuclease ABC subunit C | 78 | 86 | 127 | 1.102564103 | 1.628205128 |
| bppB | BB_S39 | protein BppB | 10 | 11 | 17 | 1.1 | 1.7 |
| rpmF | BB_0703 | 50S ribosomal protein L32 | 61 | 67 | 28 | 1.098360656 | 0.459016393 |
| manA | BB_0407 | mannose-6-phosphate isomerase | 112 | 123 | 139 | 1.098214286 | 1.241071429 |
| — | BB_J37 | hypothetical protein | 51 | 56 | 77 | 1.098039216 | 1.509803922 |
| — | BB_0098 | endonuclease MutS2 | 21 | 23 | 25 | 1.095238095 | 1.19047619 |
| — | BB_0075 | hypothetical protein | 11 | 12 | 11 | 1.090909091 | 1 |
| — | BB_0360 | hypothetical protein | 45 | 49 | 38 | 1.088888889 | 0.844444444 |
| — | predicted | antisense: infC | 24463 | 26460 | 23072 | 1.081633487 | 0.943138618 |
| | RNA | | | | | | |
| murC | BB_0817 | UDP-N-acetylmuramate--L-alanine | 25 | 27 | 23 | 1.08 | 0.92 |
| | | ligase | | | | | |
| rpsG | BB_0386 | 30S ribosomal protein S7 | 39 | 42 | 41 | 1.076923077 | 1.051282051 |
| rplV | BB_0483 | 50S ribosomal protein L22 | 13 | 14 | 17 | 1.076923077 | 1.307692308 |
| truA | BB_0012 | tRNA pseudouridine synthase A | 67 | 72 | 117 | 1.074626866 | 1.746268657 |
| — | predicted | antisense: BB_0158 | 25113 | 26987 | 19003 | 1.074622705 | 0.756699717 |
| | RNA | | | | | | |
| — | predicted | antisense: BB_0202 | 20023 | 21421 | 15133 | 1.069819707 | 0.755780852 |
| | RNA | | | | | | |
| — | BB_0205 | hypothetical protein | 29 | 31 | 25 | 1.068965517 | 0.862068966 |
| — | predicted | antisense: BB_0490 | 38173 | 40799 | 49529 | 1.068792078 | 1.297487753 |
| | RNA | | | | | | |
| — | BB_0328 | family 5 extracellular solute- | 44 | 47 | 56 | 1.068181818 | 1.272727273 |
| | | binding protein | | | | | |
| — | predicted | antisense: rpmA | 19393 | 20681 | 27719 | 1.066415717 | 1.429330171 |
| | RNA | | | | | | |
| — | predicted | antisense: BB_0096 | 22437 | 23890 | 30566 | 1.064759103 | 1.362303338 |
| | RNA | | | | | | |
| ackA | BB_0622 | acetate kinase | 1081 | 1149 | 1673 | 1.062904718 | 1.547641073 |
| — | BB_R12 | hypothetical protein | 16 | 17 | 34 | 1.0625 | 2.125 |
| rpmI | BB_0189 | 50S ribosomal protein L35 | 33 | 35 | 18 | 1.060606061 | 0.545454545 |
| ruvB | BB_0022 | Holliday junction DNA helicase | 17 | 18 | 24 | 1.058823529 | 1.411764706 |
| | | RuvB | | | | | |
| aspS | BB_0446 | aspartate--tRNA ligase | 366 | 387 | 452 | 1.057377049 | 1.234972678 |
| — | BB_G02 | hypothetical protein | 53 | 56 | 207 | 1.056603774 | 3.905660377 |
| — | predicted | antisense: rplO | 32675 | 34453 | 49266 | 1.05441469 | 1.507758225 |
| | RNA | | | | | | |
| — | predicted | antisense: BB_0712 | 18566 | 19564 | 38381 | 1.053754174 | 2.067273511 |
| | RNA | | | | | | |
| — | BB_0134 | hypothetical protein | 150 | 158 | 272 | 1.053333333 | 1.813333333 |
| — | BB_B17 | inosine 5-monophosphate | 3020 | 3175 | 3458 | 1.051324503 | 1.145033113 |
| | | dehydrogenase | | | | | |
| — | BB_0573 | ABC transporter ATP-binding | 44 | 46 | 52 | 1.045454545 | 1.181818182 |
| | | protein | | | | | |
| — | BB_0027 | hypothetical protein | 45 | 47 | 40 | 1.044444444 | 0.888888889 |
| — | BB_0045 | P115 protein | 23 | 24 | 36 | 1.043478261 | 1.565217391 |
| mraY | BB_0303 | phospho-N-acetylmuramoyl- | 116 | 121 | 129 | 1.043103448 | 1.112068966 |
| | | pentapeptide-transferase | | | | | |
| — | BB_0574 | integral membrane protein | 47 | 49 | 43 | 1.042553191 | 0.914893617 |
| — | BB_0509 | hypothetical protein | 26 | 27 | 24 | 1.038461538 | 0.923076923 |
| — | predicted | antisense: BB_0391 | 27040 | 27985 | 42739 | 1.034948225 | 1.58058432 |
| | RNA | | | | | | |
| — | predicted | antisense: pth | 24799 | 25415 | 38833 | 1.024839711 | 1.565909916 |
| | RNA | | | | | | |
| — | BB_0545 | xylulokinase | 338 | 346 | 237 | 1.023668639 | 0.701183432 |
| rnc | BB_0705 | ribonuclease III | 51 | 52 | 32 | 1.019607843 | 0.62745098 |
| — | predicted | antisense: eno | 31297 | 31783 | 25388 | 1.015528645 | 0.811195961 |
| | RNA | | | | | | |
| — | BB_0228 | hypothetical protein | 1348 | 1366 | 1555 | 1.013353116 | 1.153560831 |
| — | predicted | antisense: bmpA | 23009 | 23263 | 28336 | 1.011039159 | 1.231518102 |
| | RNA | | | | | | |
| clpP | BB_0611 | ATP-dependent Clp protease | 209 | 211 | 203 | 1.009569378 | 0.971291866 |
| | | proteolytic subunit | | | | | |
| — | BB_P12 | hypothetical protein | 82 | 82 | 254 | 1 | 3.097560976 |
| — | BB_M06 | hypothetical protein | 16 | 16 | 29 | 1 | 1.8125 |
| erpN | BB_L39 | ErpA8 protein | 4 | 4 | 4 | 1 | 1 |
| — | BB_N01 | hypothetical protein | 5 | 5 | 9 | 1 | 1.8 |
| — | BB_N30 | hypothetical protein | 7 | 7 | 1 | 1 | 0.142857143 |
| — | BB_Q39 | hypothetical protein | 3 | 3 | 0 | 1 | 0 |
| — | BB_0038 | hypothetical protein | 7 | 7 | 10 | 1 | 1.428571429 |
| — | BB_0280 | flagellar motor protein MotB | 30 | 30 | 19 | 1 | 0.633333333 |
| fliG | BB_0290 | flagellar motor switch protein FliG | 38 | 38 | 74 | 1 | 1.947368421 |
| rpoN | BB_0450 | RNA polymerase sigma-54 factor | 28 | 28 | 30 | 1 | 1.071428571 |
| — | BB_t21 | Arg tRNA | 3 | 3 | 0 | 1 | 0 |
| engA | BB_0508 | GTPase Der | 24 | 24 | 30 | 1 | 1.25 |
| — | BB_0553 | hypothetical protein | 17 | 17 | 31 | 1 | 1.823529412 |
| — | BB_0572 | glycosyl transferase | 10 | 10 | 17 | 1 | 1.7 |
| era | BB_0660 | GTPase Era | 25 | 25 | 46 | 1 | 1.84 |
| tilS | BB_0788 | tRNA(Ile)-lysidine synthase | 8 | 8 | 8 | 1 | 1 |
| rbfA | BB_0802 | ribosome-binding factor A | 24 | 24 | 12 | 1 | 0.5 |
| — | BB_J09 | outer surface protein D | 43 | 43 | 79 | 1 | 1.837209302 |
| — | predicted | antisense: BB_0715 | 26884 | 26882 | 43429 | 0.999925606 | 1.615421812 |
| | RNA | | | | | | |
| bppC | BB_P37 | site-specific recombinase | 176 | 174 | 172 | 0.988636364 | 0.977272727 |
| — | predicted | antisense: BB_0490 | 37127 | 36619 | 46171 | 0.986317235 | 1.243596305 |
| | RNA | | | | | | |
| — | BB_G09 | hypothetical protein | 59 | 58 | 161 | 0.983050847 | 2.728813559 |
| — | BB_0583 | integral membrane protein | 171 | 168 | 114 | 0.98245614 | 0.666666667 |
| — | predicted | antisense: rpsE | 30297 | 29714 | 43326 | 0.980757171 | 1.430042578 |
| | RNA | | | | | | |
| uppS | BB_0120 | isoprenyl transferase | 45 | 44 | 64 | 0.977777778 | 1.422222222 |
| rnhB | BB_0046 | ribonuclease HII | 1881 | 1832 | 2251 | 0.973950027 | 1.196703881 |
| rplC | BB_0478 | 50S ribosomal protein L3 | 38 | 37 | 35 | 0.973684211 | 0.921052632 |
| — | predicted | antisense: rpmE | 33530 | 32647 | 28685 | 0.973665374 | 0.855502535 |
| | RNA | | | | | | |
| — | predicted | antisense: eno | 29905 | 28992 | 24243 | 0.969469988 | 0.810667113 |
| | RNA | | | | | | |
| — | BB_0776 | hypothetical protein | 31 | 30 | 23 | 0.967741935 | 0.741935484 |
| — | BB_P31 | hypothetical protein | 30 | 29 | 22 | 0.966666667 | 0.733333333 |
| — | BB_N15 | hypothetical protein | 30 | 29 | 69 | 0.966666667 | 2.3 |
| — | predicted | antisense: BB_0789 | 31321 | 30220 | 45749 | 0.964847866 | 1.460649405 |
| | RNA | | | | | | |
| — | BB_P13 | hypothetical protein | 112 | 108 | 298 | 0.964285714 | 2.660714286 |
| — | predicted | antisense: BB_0504 | 30268 | 29158 | 36022 | 0.963327607 | 1.190101758 |
| | RNA | | | | | | |
| — | BB_0789 | ATP-dependent zinc | 26 | 25 | 19 | 0.961538462 | 0.730769231 |
| | | metalloprotease FtsH | | | | | |
| — | BB_R13 | hypothetical protein | 25 | 24 | 48 | 0.96 | 1.92 |
| — | predicted | antisense: gyrB | 86626 | 83151 | 90751 | 0.959885023 | 1.047618498 |
| | RNA | | | | | | |
| — | BB_0588 | MTA/SAH nucleosidase | 278 | 266 | 478 | 0.956834532 | 1.71942446 |
| — | BB_0185 | glycoprotease family protein | 183 | 175 | 50 | 0.956284153 | 0.273224044 |
| — | BB_0028 | hypothetical protein | 89 | 85 | 91 | 0.95505618 | 1.02247191 |
| pcrA | BB_0607 | ATP-dependent DNA helicase | 194 | 185 | 222 | 0.953608247 | 1.144329897 |
| recA | BB_0131 | recombinase A | 21 | 20 | 31 | 0.952380952 | 1.476190476 |
| — | BB_0420 | sensory transduction histidine | 20 | 19 | 22 | 0.95 | 1.1 |
| | | kinase | | | | | |
| — | BB_0801 | translation initiation factor IF-2 | 40 | 38 | 41 | 0.95 | 1.025 |
| — | BB_A34 | extracellular solute-binding protein, | 138 | 131 | 395 | 0.949275362 | 2.862318841 |
| | | family 5 | | | | | |
| — | predicted | antisense: flgD | 21907 | 20769 | 22567 | 0.948053134 | 1.030127357 |
| | RNA | | | | | | |
| — | BB_0709 | hypothetical protein | 19 | 18 | 20 | 0.947368421 | 1.052631579 |
| — | BB_0127 | 30S ribosomal protein S1 | 37 | 35 | 40 | 0.945945946 | 1.081081081 |
| recG | BB_0581 | ATP-dependent DNA helicase | 221 | 209 | 209 | 0.945701357 | 0.945701357 |
| | | RecG | | | | | |
| — | predicted | antisense: BB_0712 | 18479 | 17402 | 40972 | 0.941717625 | 2.217219547 |
| | RNA | | | | | | |
| — | predicted | antisense: BB_0132 | 27545 | 25605 | 33658 | 0.929569795 | 1.221927755 |
| | RNA | | | | | | |
| — | BB_N32 | PF-32 protein | 41 | 38 | 58 | 0.926829268 | 1.414634146 |
| — | predicted | antisense: BB_0094 | 22295 | 20640 | 25098 | 0.925768109 | 1.125723256 |
| | RNA | | | | | | |
| — | BB_0790 | hypothetical protein | 13 | 12 | 15 | 0.923076923 | 1.153846154 |
| — | BB_0834 | ATP-dependent Clp protease | 13 | 12 | 22 | 0.923076923 | 1.692307692 |
| | | subunit C | | | | | |
| — | predicted | antisense: rpsE | 38064 | 34929 | 61166 | 0.917638714 | 1.606925179 |
| | RNA | | | | | | |
| pfkB | BB_0630 | 1-phosphofructokinase | 734 | 673 | 855 | 0.916893733 | 1.164850136 |
| — | predicted | antisense: BB_0805 rpsO | 31123 | 28530 | 38333 | 0.91668541 | 1.231661472 |
| | RNA | | | | | | |
| — | BB_S15 | hypothetical protein | 58 | 53 | 126 | 0.913793103 | 2.172413793 |
| — | predicted | antisense: rplT | 24991 | 22754 | 18622 | 0.910487776 | 0.745148253 |
| | RNA | | | | | | |
| — | BB_0835 | phosphomannomutase | 11 | 10 | 32 | 0.909090909 | 2.909090909 |
| — | predicted | antisense: tuf | 54506 | 49517 | 69878 | 0.908468792 | 1.282023997 |
| | RNA | | | | | | |
| — | BB_0324 | hypothetical protein | 185 | 168 | 225 | 0.908108108 | 1.216216216 |
| — | predicted | antisense: rplA | 35725 | 32400 | 42483 | 0.906927922 | 1.18916725 |
| | RNA | | | | | | |
| dbpA | BB_A24 | decorin-binding protein A | 558 | 505 | 2780 | 0.905017921 | 4.982078853 |
| — | BB_A60 | surface lipoprotein P27 | 21 | 19 | 36 | 0.904761905 | 1.714285714 |
| — | BB_0219 | metal cation transporter permease | 355 | 321 | 156 | 0.904225352 | 0.43943662 |
| asnS | BB_0101 | asparagine--tRNA ligase | 193 | 174 | 100 | 0.901554404 | 0.518134715 |
| — | BB_0593 | long-chain-fatty-acid CoA ligase | 81 | 73 | 24 | 0.901234568 | 0.296296296 |
| — | BB_S44 | hypothetical protein | 10 | 9 | 15 | 0.9 | 1.5 |
| rplP | BB_0485 | 50S ribosomal protein L16 | 10 | 9 | 17 | 0.9 | 1.7 |
| — | BB_0084 | cysteine desulfurase | 98 | 88 | 146 | 0.897959184 | 1.489795918 |
| rho | BB_0230 | transcription termination factor Rho | 49 | 44 | 22 | 0.897959184 | 0.448979592 |
| — | predicted | antisense: rpoB | 24834 | 22295 | 29883 | 0.897761134 | 1.203309978 |
| | RNA | | | | | | |
| — | predicted | antisense: BB_A23 | 13946 | 12513 | 82442 | 0.897246522 | 5.911515847 |
| | RNA | | | | | | |
| — | predicted | antisense: dnaK | 25407 | 22781 | 64444 | 0.896642658 | 2.536466328 |
| | RNA | | | | | | |
| — | BB_0073 | hypothetical protein | 38 | 34 | 63 | 0.894736842 | 1.657894737 |
| rpsB | BB_0123 | 30S ribosomal protein S2 | 56 | 50 | 33 | 0.892857143 | 0.589285714 |
| rpoC | BB_0388 | DNA-directed RNA polymerase | 28 | 25 | 32 | 0.892857143 | 1.142857143 |
| | | subunit beta′ | | | | | |
| — | predicted | antisense: eno | 39845 | 35556 | 24373 | 0.892357887 | 0.611695319 |
| | RNA | | | | | | |
| — | predicted | antisense: recA | 28526 | 25416 | 34964 | 0.890976653 | 1.225688845 |
| | RNA | | | | | | |
| — | predicted | antisense: acpP | 33655 | 29974 | 32765 | 0.890625464 | 0.973555192 |
| | RNA | | | | | | |
| — | predicted | — | 24757 | 22016 | 29650 | 0.889283839 | 1.197641071 |
| | RNA | | | | | | |
| — | BB_D21 | hypothetical protein | 9 | 8 | 7 | 0.888888889 | 0.777777778 |
| — | predicted | antisense: ffh | 29125 | 25850 | 25303 | 0.887553648 | 0.868772532 |
| | RNA | | | | | | |
| — | BB_0103 | hypothetical protein | 80 | 71 | 62 | 0.8875 | 0.775 |
| — | predicted | — | 29480 | 26115 | 32571 | 0.885854817 | 1.104850746 |
| | RNA | | | | | | |
| ffh | BB_0694 | signal recognition particle protein | 26 | 23 | 17 | 0.884615385 | 0.653846154 |
| — | predicted | antisense: BB_0094 | 26986 | 23849 | 37688 | 0.883754539 | 1.396576002 |
| | RNA | | | | | | |
| — | BB_0452 | chromate transporter superfamily | 179 | 158 | 127 | 0.882681564 | 0.709497207 |
| | | protein | | | | | |
| — | BB_0194 | TatD family hydrolase | 332 | 293 | 408 | 0.88253012 | 1.228915663 |
| — | BB_P42 | phage terminase large subunit | 58 | 51 | 118 | 0.879310345 | 2.034482759 |
| — | BB_P29 | hypothetical protein | 375 | 328 | 377 | 0.874666667 | 1.005333333 |
| — | BB_0831 | xylose operon regulatory protein | 316 | 276 | 122 | 0.873417722 | 0.386075949 |
| — | predicted | antisense: tig | 21239 | 18405 | 40472 | 0.866566223 | 1.905551109 |
| | RNA | | | | | | |
| — | BB_0170 | hypothetical protein | 344 | 298 | 283 | 0.86627907 | 0.822674419 |
| — | BB_0665 | hypothetical protein | 67 | 58 | 112 | 0.865671642 | 1.671641791 |
| serS | BB_0226 | serine--tRNA ligase | 238 | 205 | 205 | 0.861344538 | 0.861344538 |
| — | BB_0560 | chaperone protein HtpG | 114 | 98 | 98 | 0.859649123 | 0.859649123 |
| — | predicted | antisense: BB_0496 | 22752 | 19550 | 35604 | 0.85926512 | 1.564873418 |
| | RNA | | | | | | |
| — | BB_S27 | hypothetical protein | 14 | 12 | 21 | 0.857142857 | 1.5 |
| — | BB_R44 | hypothetical protein | 7 | 6 | 7 | 0.857142857 | 1 |
| bppC | BB_M37 | BppC | 49 | 42 | 29 | 0.857142857 | 0.591836735 |
| — | BB_N03 | hypothetical protein | 7 | 6 | 7 | 0.857142857 | 1 |
| — | BB_0210 | hypothetical protein | 28 | 24 | 42 | 0.857142857 | 1.5 |
| — | BB_0756 | hypothetical protein | 27 | 23 | 29 | 0.851851852 | 1.074074074 |
| — | BB_0106 | hypothetical protein | 20 | 17 | 23 | 0.85 | 1.15 |
| — | BB_0306 | rRNA small subunit | 20 | 17 | 23 | 0.85 | 1.15 |
| | | methyltransferase H | | | | | |
| rpoB | BB_0389 | DNA-directed RNA polymerase | 20 | 17 | 17 | 0.85 | 0.85 |
| | | subunit beta | | | | | |
| — | BB_0454 | lipopolysaccharide biosynthesis- | 39 | 33 | 80 | 0.846153846 | 2.051282051 |
| | | like protein | | | | | |
| — | BB_R04 | hypothetical protein | 32 | 27 | 54 | 0.84375 | 1.6875 |
| — | BB_0642 | spermidine/putrescine ABC | 64 | 54 | 44 | 0.84375 | 0.6875 |
| | | transporter ATP-binding protein | | | | | |
| — | BB_B28 | hypothetical protein | 50 | 42 | 64 | 0.84 | 1.28 |
| — | BB_0286 | flagellar protein | 12 | 10 | 18 | 0.833333333 | 1.5 |
| — | BB_B05 | chitibiose transporter protein ChbA | 215 | 179 | 236 | 0.83255814 | 1.097674419 |
| — | BB_0507 | hypothetical protein | 212 | 176 | 161 | 0.830188679 | 0.759433962 |
| — | BB_0746 | oligopeptide ABC transporter | 234 | 193 | 244 | 0.824786325 | 1.042735043 |
| | | permease | | | | | |
| — | BB_P06 | hypothetical protein | 79 | 65 | 169 | 0.82278481 | 2.139240506 |
| — | BB_0674 | membrane protein | 701 | 575 | 521 | 0.820256776 | 0.743223966 |
| — | BB_P30 | hypothetical protein | 11 | 9 | 5 | 0.818181818 | 0.454545455 |
| — | BB_0239 | deoxyguanosine/deoxyadenosine | 11 | 9 | 14 | 0.818181818 | 1.272727273 |
| | | kinase | | | | | |
| dnaN | BB_0438 | DNA polymerase III subunit beta | 11 | 9 | 14 | 0.818181818 | 1.272727273 |
| — | predicted | antisense: BB_0283 | 27837 | 22723 | 27025 | 0.816287675 | 0.97083019 |
| | RNA | | | | | | |
| — | BB_P03 | hypothetical protein | 70 | 57 | 99 | 0.814285714 | 1.414285714 |
| — | BB_0404 | hypothetical protein | 16 | 13 | 10 | 0.8125 | 0.625 |
| rpmG | BB_0396 | 50S ribosomal protein L33 | 62 | 50 | 32 | 0.806451613 | 0.516129032 |
| — | BB_0459 | hypothetical protein | 51 | 41 | 40 | 0.803921569 | 0.784313725 |
| — | predicted | antisense: rplA | 31638 | 25388 | 45210 | 0.802452747 | 1.428977811 |
| | RNA | | | | | | |
| priA | BB_0014 | primosomal protein N | 232 | 186 | 439 | 0.801724138 | 1.892241379 |
| — | predicted | antisense: rpsT | 43570 | 34889 | 22068 | 0.800757402 | 0.506495295 |
| | RNA | | | | | | |
| — | BB_S01 | phage portal protein | 5 | 4 | 15 | 0.8 | 3 |
| — | BB_M29 | hypothetical protein | 25 | 20 | 28 | 0.8 | 1.12 |
| — | BB_0666 | N-acetylmuramoyl-L-alanine | 85 | 68 | 63 | 0.8 | 0.741176471 |
| | | amidase domain-containing protein | | | | | |
| — | BB_0839 | hypothetical protein | 15 | 12 | 26 | 0.8 | 1.733333333 |
| — | BB_0335 | peptide ABC transporter ATP- | 2201 | 1753 | 2879 | 0.796456156 | 1.308041799 |
| | | binding protein | | | | | |
| — | BB_P14 | hypothetical protein | 97 | 77 | 185 | 0.793814433 | 1.907216495 |
| — | BB_0366 | aminopeptidase | 130 | 103 | 69 | 0.792307692 | 0.530769231 |
| tsf | BB_0122 | elongation factor Ts | 24 | 19 | 20 | 0.791666667 | 0.833333333 |
| dnaA | BB_0437 | chromosomal replication initiation | 96 | 76 | 224 | 0.791666667 | 2.333333333 |
| | | protein | | | | | |
| — | predicted | antisense: rplR rpsE | 31912 | 25246 | 43718 | 0.791113061 | 1.369954876 |
| | RNA | | | | | | |
| — | BB_0824 | hypothetical protein | 278 | 219 | 98 | 0.787769784 | 0.352517986 |
| — | predicted | antisense: secA | 42660 | 33448 | 62849 | 0.784060009 | 1.473253633 |
| | RNA | | | | | | |
| — | predicted | antisense: rpsH | 40495 | 31668 | 48417 | 0.782022472 | 1.19562909 |
| | RNA | | | | | | |
| — | predicted | antisense: BB_0504 | 48236 | 37645 | 63875 | 0.780433701 | 1.324218426 |
| | RNA | | | | | | |
| — | BB_0159 | hypothetical protein | 122 | 95 | 80 | 0.778688525 | 0.655737705 |
| — | BB_0412 | membrane protein | 230 | 179 | 173 | 0.77826087 | 0.752173913 |
| — | predicted | antisense: BB_B16 | 44204 | 34349 | 57475 | 0.777056375 | 1.300221699 |
| | RNA | | | | | | |
| — | predicted | antisense: ffh | 32436 | 25203 | 36673 | 0.777007029 | 1.130626464 |
| | RNA | | | | | | |
| 16S | BB_r05 | 16S ribosomal RNA | 3488 | 2709 | 2243 | 0.776662844 | 0.643061927 |
| — | BB_P19 | hypothetical protein | 76 | 59 | 80 | 0.776315789 | 1.052631579 |
| — | BB_0168 | dnaK suppressor | 252 | 195 | 183 | 0.773809524 | 0.726190476 |
| — | predicted | antisense: BB_0789 | 63781 | 49134 | 77859 | 0.770354808 | 1.22072404 |
| | RNA | | | | | | |
| pstA | BB_0217 | phosphate ABC transporter | 378 | 290 | 275 | 0.767195767 | 0.727513228 |
| | | permease PstA | | | | | |
| — | predicted | antisense: BB_0093 | 33203 | 25451 | 38521 | 0.766527121 | 1.16016625 |
| | RNA | | | | | | |
| — | BB_0255 | M23 peptidase domain-containing | 400 | 306 | 342 | 0.765 | 0.855 |
| | | protein | | | | | |
| — | BB_0639 | spermidine/putrescine ABC | 34 | 26 | 17 | 0.764705882 | 0.5 |
| | | transporter substrate-binding | | | | | |
| | | protein | | | | | |
| — | BB_0792 | hypothetical protein | 68 | 52 | 57 | 0.764705882 | 0.838235294 |
| — | BB_0213 | lipoprotein | 2871 | 2183 | 3267 | 0.760362243 | 1.137931034 |
| — | BB_0071 | membrane protein | 75 | 57 | 87 | 0.76 | 1.16 |
| fliM | BB_0278 | flagellar motor switch protein FliM | 25 | 19 | 23 | 0.76 | 0.92 |
| — | BB_0506 | hypothetical protein | 141 | 107 | 133 | 0.758865248 | 0.943262411 |
| rplA | BB_0392 | 50S ribosomal protein L1 | 29 | 22 | 19 | 0.75862069 | 0.655172414 |
| — | BB_0269 | ATP-binding protein | 153 | 116 | 149 | 0.758169935 | 0.973856209 |
| — | BB_0493 | 50S ribosomal protein L6 | 70 | 53 | 54 | 0.757142857 | 0.771428571 |
| — | BB_G23 | hypothetical protein | 37 | 28 | 124 | 0.756756757 | 3.351351351 |
| — | predicted | antisense: BB_0149 | 37725 | 28431 | 31340 | 0.753638171 | 0.83074884 |
| | RNA | | | | | | |
| — | predicted | antisense: rpsO | 40369 | 30359 | 32962 | 0.752037454 | 0.816517625 |
| | RNA | | | | | | |
| — | BB_H09a | hypothetical protein | 197 | 148 | 238 | 0.751269036 | 1.208121827 |
| — | predicted | antisense: BB_0490 rpsN | 28914 | 21721 | 31674 | 0.751227779 | 1.095455489 |
| | RNA | | | | | | |
| — | BB_M08 | hypothetical protein | 8 | 6 | 19 | 0.75 | 2.375 |
| — | BB_M32 | PF-32 protein | 4 | 3 | 6 | 0.75 | 1.5 |
| — | BB_N42 | hypothetical protein | 4 | 3 | 10 | 0.75 | 2.5 |
| — | BB_G21 | hypothetical protein | 12 | 9 | 55 | 0.75 | 4.583333333 |
| — | BB_J19 | hypothetical protein | 8 | 6 | 3 | 0.75 | 0.375 |
| — | BB_0167 | outer membrane protein | 408 | 305 | 656 | 0.74754902 | 1.607843137 |
| — | predicted | antisense: ffh | 43499 | 32442 | 48517 | 0.745810249 | 1.115358974 |
| | RNA | | | | | | |
| rpsM | BB_0500 | 30S ribosomal protein S13 | 166 | 123 | 96 | 0.740963855 | 0.578313253 |
| — | BB_0771a | hypothetical protein | 27 | 20 | 46 | 0.740740741 | 1.703703704 |
| — | predicted | antisense: BB_B16 | 33243 | 24612 | 36826 | 0.740366393 | 1.107782089 |
| | RNA | | | | | | |
| — | predicted | antisense: flhB | 31829 | 23558 | 28490 | 0.740142637 | 0.895095667 |
| | RNA | | | | | | |
| — | BB_N02 | hypothetical protein | 19 | 14 | 25 | 0.736842105 | 1.315789474 |
| csrA | BB_0184 | carbon storage regulator | 345 | 254 | 247 | 0.736231884 | 0.715942029 |
| — | BB_0086 | Mg chelatase-like protein | 742 | 546 | 628 | 0.735849057 | 0.846361186 |
| — | predicted | antisense: BB_0442 | 31877 | 23416 | 50422 | 0.734573517 | 1.581767419 |
| | RNA | | | | | | |
| — | BB_0816 | hypothetical protein | 45 | 33 | 47 | 0.733333333 | 1.044444444 |
| — | BB_0413 | integral membrane protein | 127 | 93 | 98 | 0.732283465 | 0.771653543 |
| — | BB_A47 | hypothetical protein | 63 | 46 | 46 | 0.73015873 | 0.73015873 |
| — | predicted | antisense: BB_0504 | 33095 | 24143 | 23715 | 0.729505968 | 0.716573501 |
| | RNA | | | | | | |
| — | BB_B09 | lipoprotein | 151 | 110 | 218 | 0.728476821 | 1.443708609 |
| — | BB_A04 | S2 antigen | 320 | 233 | 532 | 0.728125 | 1.6625 |
| — | BB_0373 | hypothetical protein | 11 | 8 | 4 | 0.727272727 | 0.363636364 |
| rpmC | BB_0486 | 50S ribosomal protein L29 | 11 | 8 | 0 | 0.727272727 | 0 |
| — | BB_A73 | antigen P35 | 22 | 16 | 35 | 0.727272727 | 1.590909091 |
| — | predicted | antisense: trmE | 30104 | 21891 | 29646 | 0.727179112 | 0.984786075 |
| | RNA | | | | | | |
| — | BB_0602 | chaperonin | 72 | 52 | 61 | 0.722222222 | 0.847222222 |
| — | BB_A30 | hypothetical protein | 18 | 13 | 26 | 0.722222222 | 1.444444444 |
| — | predicted | antisense: ftsZ | 34527 | 24900 | 33269 | 0.721174733 | 0.963564746 |
| | RNA | | | | | | |
| — | BB_0595 | hypothetical protein | 110 | 79 | 135 | 0.718181818 | 1.227272727 |
| — | predicted | antisense: BB_0593 | 29996 | 21507 | 41099 | 0.716995599 | 1.370149353 |
| | RNA | | | | | | |
| — | predicted | antisense: BB_0236 | 32984 | 23615 | 30825 | 0.715953189 | 0.934544021 |
| | RNA | | | | | | |
| — | predicted | antisense: BB_0735 | 33000 | 23585 | 23944 | 0.71469697 | 0.725575758 |
| | RNA | | | | | | |
| bdrA | BB_P34 | protein BdrA | 21 | 15 | 12 | 0.714285714 | 0.571428571 |
| — | BB_0125 | hypothetical protein | 21 | 15 | 20 | 0.714285714 | 0.952380952 |
| — | BB_0117 | hemolysin III | 542 | 387 | 284 | 0.71402214 | 0.52398524 |
| — | BB_0155 | lipoprotein | 157 | 112 | 132 | 0.713375796 | 0.840764331 |
| — | BB_0740 | hypothetical protein | 59 | 42 | 66 | 0.711864407 | 1.118644068 |
| — | predicted | antisense: BB_A23 | 7231 | 5123 | 41911 | 0.708477389 | 5.796017148 |
| | RNA | | | | | | |
| — | predicted | antisense: rpoB | 32518 | 23037 | 40563 | 0.708438403 | 1.247401439 |
| | RNA | | | | | | |
| frr | BB_0121 | ribosome recycling factor | 24 | 17 | 15 | 0.708333333 | 0.625 |
| — | BB_0538 | hypothetical protein | 17 | 12 | 18 | 0.705882353 | 1.058823529 |
| — | BB_0542 | hypothetical protein | 34 | 24 | 29 | 0.705882353 | 0.852941176 |
| — | predicted | antisense: secA | 24868 | 17538 | 39042 | 0.705243687 | 1.569969439 |
| | RNA | | | | | | |
| — | predicted | antisense: BB_0805 | 30022 | 21154 | 42105 | 0.704616614 | 1.402471521 |
| | RNA | | | | | | |
| pncB | BB_0635 | nicotinate | 91 | 64 | 86 | 0.703296703 | 0.945054945 |
| | | phosphoribosyltransferase | | | | | |
| — | BB_M31 | hypothetical protein | 10 | 7 | 9 | 0.7 | 0.9 |
| fliS | BB_0550 | flagellar protein FliS | 10 | 7 | 5 | 0.7 | 0.5 |
| — | predicted | antisense: groL | 38526 | 26863 | 32316 | 0.697269377 | 0.838810154 |
| | RNA | | | | | | |
| — | predicted | antisense: rplM | 57061 | 39728 | 53232 | 0.69623736 | 0.932896374 |
| | RNA | | | | | | |
| — | predicted | antisense: rpoB | 32495 | 22616 | 39944 | 0.695983998 | 1.229235267 |
| | RNA | | | | | | |
| recB | BB_0633 | exodeoxyribonuclease V subunit | 13 | 9 | 14 | 0.692307692 | 1.076923077 |
| | | beta | | | | | |
| — | BB_A08 | hypothetical protein | 13 | 9 | 11 | 0.692307692 | 0.846153846 |
| — | predicted | antisense: BB_0281 | 31321 | 21635 | 30998 | 0.690750615 | 0.98968743 |
| | RNA | | | | | | |
| — | predicted | antisense: rnpA rpmH | 40235 | 27790 | 35636 | 0.690692183 | 0.885696533 |
| | RNA | | | | | | |
| — | BB_0406 | hypothetical protein | 339 | 234 | 293 | 0.690265487 | 0.864306785 |
| rpoA | BB_0502 | DNA-directed RNA polymerase | 29 | 20 | 23 | 0.689655172 | 0.793103448 |
| | | subunit alpha | | | | | |
| — | predicted | antisense: BB_0579 | 32417 | 22333 | 33565 | 0.688928649 | 1.035413518 |
| | RNA | | | | | | |
| — | predicted | antisense: BB_0498 | 32163 | 22147 | 49088 | 0.688586264 | 1.526225787 |
| | RNA | | | | | | |
| — | BB_0283 | flagellar hook protein FlgE | 16 | 11 | 15 | 0.6875 | 0.9375 |
| — | BB_A20 | PF-32 protein | 16 | 11 | 37 | 0.6875 | 2.3125 |
| — | predicted | antisense: BB_0805 | 32758 | 22416 | 41634 | 0.68429086 | 1.270956713 |
| | RNA | | | | | | |
| — | predicted | antisense: BB_0544 | 34812 | 23768 | 34599 | 0.682753074 | 0.99388142 |
| | RNA | | | | | | |
| — | BB_0434 | chromosome-partitioning protein | 188 | 128 | 271 | 0.680851064 | 1.441489362 |
| | | ParB | | | | | |
| — | BB_R17 | hypothetical protein | 259 | 176 | 357 | 0.67953668 | 1.378378378 |
| rpsH | BB_0492 | 30S ribosomal protein S8 | 31 | 21 | 14 | 0.677419355 | 0.451612903 |
| — | BB_J08 | surface protein | 70 | 47 | 51 | 0.671428571 | 0.728571429 |
| — | predicted | antisense: BB_B16 | 30221 | 20288 | 41303 | 0.671321267 | 1.366698653 |
| | RNA | | | | | | |
| rpmB | BB_0350 | 50S ribosomal protein L28 | 154 | 103 | 81 | 0.668831169 | 0.525974026 |
| — | BB_0250 | inner membrane protein | 997 | 666 | 1063 | 0.668004012 | 1.066198596 |
| — | BB_N43 | phage terminase large subunit | 6 | 4 | 7 | 0.666666667 | 1.166666667 |
| — | BB_J17 | PF-32 protein | 6 | 4 | 3 | 0.666666667 | 0.5 |
| — | BB_A46 | hypothetical protein | 33 | 22 | 34 | 0.666666667 | 1.03030303 |
| — | BB_0144 | glycine/betaine ABC transporter | 463 | 308 | 473 | 0.665226782 | 1.021598272 |
| | | substrate-binding protein | | | | | |
| — | predicted | antisense: dnaK | 16302 | 10814 | 36078 | 0.66335419 | 2.213102687 |
| | RNA | | | | | | |
| — | predicted | antisense: rnpA | 38978 | 25853 | 37140 | 0.663271589 | 0.952845195 |
| | RNA | | | | | | |
| — | BB_0202 | hemolysin | 323 | 214 | 202 | 0.6625387 | 0.625386997 |
| — | BB_G12 | hypothetical protein | 154 | 102 | 343 | 0.662337662 | 2.227272727 |
| — | BB_0676 | phosphoglycolate phosphatase | 428 | 283 | 267 | 0.661214953 | 0.623831776 |
| — | BB_J11 | hypothetical protein | 391 | 258 | 203 | 0.659846547 | 0.519181586 |
| — | predicted | antisense: BB_0283 | 37817 | 24792 | 40808 | 0.655578179 | 1.079091414 |
| | RNA | | | | | | |
| fusA | BB_0691 | elongation factor G | 55 | 36 | 52 | 0.654545455 | 0.945454545 |
| — | predicted | antisense: rpsC | 35844 | 23450 | 51378 | 0.654223859 | 1.433377971 |
| | RNA | | | | | | |
| — | predicted | antisense: BB_0355 | 53824 | 35102 | 44064 | 0.652162604 | 0.818668252 |
| | RNA | | | | | | |
| fliL | BB_0279 | flagellar basal body-associated | 20 | 13 | 6 | 0.65 | 0.3 |
| | | protein FliL | | | | | |
| eno | BB_0337 | enolase | 597 | 387 | 442 | 0.648241206 | 0.740368509 |
| — | predicted | antisense: flhA | 32742 | 21160 | 40526 | 0.646264736 | 1.237737463 |
| | RNA | | | | | | |
| — | BB_0315 | hypothetical protein | 62 | 40 | 61 | 0.64516129 | 0.983870968 |
| — | predicted | antisense: rplP | 30835 | 19833 | 41395 | 0.643197665 | 1.342467975 |
| | RNA | | | | | | |
| rseP | BB_0118 | RIP metalloprotease RseP | 42 | 27 | 54 | 0.642857143 | 1.285714286 |
| rpsJ | BB_0477 | 30S ribosomal protein S10 | 67 | 43 | 43 | 0.641791045 | 0.641791045 |
| — | predicted | antisense: BB_0059 | 32673 | 20918 | 26049 | 0.640222814 | 0.797263796 |
| | RNA | | | | | | |
| rplX | BB_0489 | 50S ribosomal protein L24 | 36 | 23 | 6 | 0.638888889 | 0.166666667 |
| — | predicted | antisense: BB_0114 | 49579 | 31623 | 33069 | 0.637830533 | 0.666996107 |
| | RNA | | | | | | |
| tRNA- | BB_t13 | Val tRNA | 80 | 51 | 52 | 0.6375 | 0.65 |
| Val-1 | | | | | | | |
| — | predicted | antisense: rpsL | 61764 | 39184 | 60570 | 0.63441487 | 0.98066835 |
| | RNA | | | | | | |
| — | predicted | antisense: ptsP | 50133 | 31799 | 58721 | 0.634292781 | 1.17130433 |
| | RNA | | | | | | |
| alr | BB_0160 | alanine racemase | 109 | 69 | 58 | 0.633027523 | 0.532110092 |
| — | BB_R45 | phage terminase large subunit | 19 | 12 | 28 | 0.631578947 | 1.473684211 |
| — | BB_N17 | hypothetical protein | 35 | 22 | 51 | 0.628571429 | 1.457142857 |
| — | predicted | antisense: BB_0442 | 28356 | 17791 | 38660 | 0.627415714 | 1.363379884 |
| | RNA | | | | | | |
| — | predicted | antisense: rpsH | 45679 | 28657 | 39764 | 0.627356116 | 0.870509424 |
| | RNA | | | | | | |
| — | predicted | antisense: BB_0557 | 44932 | 28179 | 40067 | 0.62714769 | 0.891725274 |
| | RNA | | | | | | |
| tRNA- | BB_t04 | Ile tRNA | 1691 | 1060 | 1489 | 0.626848019 | 0.880544057 |
| Ile-1 | | | | | | | |
| — | predicted | antisense: BB_0789 | 36901 | 23078 | 41830 | 0.625403106 | 1.133573616 |
| | RNA | | | | | | |
| bdrR | BB_N27 | BdrR protein | 8 | 5 | 0 | 0.625 | 0 |
| bdrV | BB_Q42 | protein BdrV | 8 | 5 | 1 | 0.625 | 0.125 |
| rnpA | BB_0441 | ribonuclease P protein component | 16 | 10 | 6 | 0.625 | 0.375 |
| — | BB_J18 | PF-32 protein | 8 | 5 | 5 | 0.625 | 0.625 |
| — | BB_0312 | purine-binding chemotaxis protein | 21 | 13 | 19 | 0.619047619 | 0.904761905 |
| — | predicted | antisense: ffh | 40282 | 24929 | 41914 | 0.618862023 | 1.040514374 |
| | RNA | | | | | | |
| — | BB_H04 | hypothetical protein | 76 | 47 | 52 | 0.618421053 | 0.684210526 |
| — | BB_Q18 | hypothetical protein | 89 | 55 | 150 | 0.617977528 | 1.685393258 |
| — | BB_A65 | lipoprotein | 636 | 393 | 1277 | 0.617924528 | 2.007861635 |
| — | BB_0460 | lipoprotein | 1122 | 692 | 1163 | 0.616755793 | 1.036541889 |
| — | predicted | antisense: trx | 44292 | 27201 | 35346 | 0.614128962 | 0.798022216 |
| | RNA | | | | | | |
| gnd | BB_0561 | 6-phosphogluconate dehydrogenase | 315 | 193 | 171 | 0.612698413 | 0.542857143 |
| — | BB_P02 | hypothetical protein | 113 | 69 | 145 | 0.610619469 | 1.283185841 |
| — | BB_R05 | hypothetical protein | 100 | 61 | 98 | 0.61 | 0.98 |
| — | predicted | antisense: rpsC | 35240 | 21447 | 41561 | 0.608598184 | 1.179370034 |
| | RNA | | | | | | |
| — | BB_0658 | 2,3-bisphosphoglycerate-dependent | 735 | 447 | 370 | 0.608163265 | 0.503401361 |
| | | phosphoglycerate mutase | | | | | |
| — | predicted | antisense: gyrB | 69422 | 42177 | 77544 | 0.607545159 | 1.116994613 |
| | RNA | | | | | | |
| — | BB_0007 | hypothetical protein | 89 | 54 | 144 | 0.606741573 | 1.617977528 |
| — | predicted | antisense: rplB | 52813 | 31972 | 75092 | 0.605381251 | 1.421846894 |
| | RNA | | | | | | |
| — | predicted | antisense: BB_0800 | 33687 | 20391 | 37337 | 0.605307685 | 1.108350402 |
| | RNA | | | | | | |
| 5S_rrlB | BB_r03 | 5S ribosomal RNA | 38 | 23 | 0 | 0.605263158 | 0 |
| — | BB_A31 | pbsx family phage terminase | 38 | 23 | 52 | 0.605263158 | 1.368421053 |
| — | BB_0225 | tRNA-dihydrouridine synthase A | 419 | 253 | 194 | 0.603818616 | 0.46300716 |
| — | predicted | antisense: BB_0805 | 36967 | 22313 | 39407 | 0.603592393 | 1.066004815 |
| | RNA | | | | | | |
| udk | BB_0015 | uridine kinase | 156 | 94 | 37 | 0.602564103 | 0.237179487 |
| — | BB_R01 | hypothetical protein | 5 | 3 | 6 | 0.6 | 1.2 |
| — | BB_0307 | hypothetical protein | 10 | 6 | 18 | 0.6 | 1.8 |
| — | BB_H09 | type II restriction enzyme | 5 | 3 | 7 | 0.6 | 1.4 |
| | | methylase subunit | | | | | |
| — | predicted | antisense: rpsR | 41655 | 24967 | 27740 | 0.599375825 | 0.665946465 |
| | RNA | | | | | | |
| — | predicted | antisense: rpsI | 69200 | 41409 | 50558 | 0.598395954 | 0.730606936 |
| | RNA | | | | | | |
| luxS | BB_0377 | S-ribosylhomocysteine lyase | 891 | 533 | 468 | 0.598204265 | 0.525252525 |
| — | predicted | antisense: BB_0346 | 36335 | 21722 | 30244 | 0.597825788 | 0.832365488 |
| | RNA | | | | | | |
| — | BB_Q15 | hypothetical protein | 94 | 56 | 118 | 0.595744681 | 1.255319149 |
| — | BB_R07 | hypothetical protein | 74 | 44 | 104 | 0.594594595 | 1.405405405 |
| bppB | BB_P36 | protein BppB | 27 | 16 | 37 | 0.592592593 | 1.37037037 |
| — | predicted | antisense: gyrB | 52360 | 31024 | 41019 | 0.592513369 | 0.783403361 |
| | RNA | | | | | | |
| — | BB_R06 | hypothetical protein | 49 | 29 | 80 | 0.591836735 | 1.632653061 |
| — | predicted | antisense: BB_0351 | 25091 | 14849 | 41532 | 0.591805827 | 1.655254872 |
| | RNA | | | | | | |
| topA | BB_0828 | DNA topoisomerase I | 120 | 71 | 120 | 0.591666667 | 1 |
| — | predicted | antisense: ptsP | 36729 | 21714 | 36898 | 0.591194969 | 1.004601269 |
| | RNA | | | | | | |
| — | BB_A43 | hypothetical protein | 17 | 10 | 14 | 0.588235294 | 0.823529412 |
| — | predicted | antisense: gyrB | 49961 | 29237 | 43295 | 0.585196453 | 0.866575929 |
| | RNA | | | | | | |
| — | predicted | antisense: BB_0505 | 28600 | 16707 | 35874 | 0.584160839 | 1.254335664 |
| | RNA | | | | | | |
| — | BB_0580 | integral membrane protein | 536 | 313 | 210 | 0.583955224 | 0.391791045 |
| — | BB_0586 | FemA protein | 141 | 82 | 74 | 0.581560284 | 0.524822695 |
| — | predicted | antisense: BB_0749 | 39504 | 22950 | 33701 | 0.580953827 | 0.853103483 |
| | RNA | | | | | | |
| mlpD | BB_R28 | lipoprotein | 31 | 18 | 40 | 0.580645161 | 1.290322581 |
| — | BB_0723 | adenylyl cyclase | 50 | 29 | 35 | 0.58 | 0.7 |
| — | BB_N09 | hypothetical protein | 19 | 11 | 23 | 0.578947368 | 1.210526316 |
| — | predicted | antisense: rplB | 28949 | 16752 | 46070 | 0.578672838 | 1.591419393 |
| | RNA | | | | | | |
| — | BB_O35 | hypothetical protein | 156 | 90 | 270 | 0.576923077 | 1.730769231 |
| — | BB_0238 | hypothetical protein | 59 | 34 | 26 | 0.576271186 | 0.440677966 |
| — | predicted | antisense: BB_0789 | 59217 | 33940 | 59002 | 0.573146225 | 0.996369286 |
| | RNA | | | | | | |
| — | predicted | antisense: rplK | 49219 | 28178 | 43797 | 0.572502489 | 0.88983929 |
| | RNA | | | | | | |
| — | BB_0208 | hypothetical protein | 7 | 4 | 14 | 0.571428571 | 2 |
| — | BB_D14 | hypothetical protein | 14 | 8 | 9 | 0.571428571 | 0.642857143 |
| — | predicted | antisense: ptsP | 47387 | 26968 | 36998 | 0.56910123 | 0.780762656 |
| | RNA | | | | | | |
| — | predicted | antisense: rpsB | 54387 | 30908 | 30425 | 0.568297571 | 0.559416772 |
| | RNA | | | | | | |
| — | predicted | antisense: BB_0094 | 29829 | 16925 | 39494 | 0.567400852 | 1.324013544 |
| | RNA | | | | | | |
| — | predicted | antisense: rpsM rpmJ | 60988 | 34592 | 40230 | 0.567193546 | 0.659637962 |
| | RNA | | | | | | |
| — | BB_0597 | methyl-accepting chemotaxis | 97 | 55 | 78 | 0.567010309 | 0.804123711 |
| | | protein | | | | | |
| — | predicted | antisense: rpoB | 29946 | 16932 | 43145 | 0.565417752 | 1.440760035 |
| | RNA | | | | | | |
| — | BB_R15 | hypothetical protein | 46 | 26 | 90 | 0.565217391 | 1.956521739 |
| — | BB_B06 | chitibiose transporter protein ChbB | 291 | 164 | 243 | 0.563573883 | 0.835051546 |
| — | BB_0008 | hypothetical protein | 32 | 18 | 27 | 0.5625 | 0.84375 |
| — | BB_0648 | hypothetical protein | 50 | 28 | 49 | 0.56 | 0.98 |
| — | predicted | antisense: BB_0147 | 73211 | 40956 | 51655 | 0.55942413 | 0.705563372 |
| | RNA | | | | | | |
| pstB | BB_0218 | phosphate ABC transporter ATP- | 1489 | 831 | 1255 | 0.55809268 | 0.842847549 |
| | | binding protein | | | | | |
| — | predicted | antisense: rplW | 34311 | 19073 | 45745 | 0.555885868 | 1.333245898 |
| | RNA | | | | | | |
| — | BB_L19 | hypothetical protein | 54 | 30 | 24 | 0.555555556 | 0.444444444 |
| — | predicted | antisense: tuf | 78847 | 43577 | 96508 | 0.552677971 | 1.223990767 |
| | RNA | | | | | | |
| — | predicted | antisense: BB_0789 | 35741 | 19721 | 37813 | 0.551775272 | 1.057972636 |
| | RNA | | | | | | |
| — | BB_0675 | hypothetical protein | 363 | 200 | 237 | 0.550964187 | 0.652892562 |
| — | BB_H02 | hypothetical protein | 91 | 50 | 208 | 0.549450549 | 2.285714286 |
| — | predicted | antisense: BB_0805 | 45473 | 24906 | 48324 | 0.54770963 | 1.062696545 |
| | RNA | | | | | | |
| — | BB_0753 | hypothetical protein | 503 | 275 | 320 | 0.546719682 | 0.636182903 |
| — | BB_0017 | integral membrane protein | 681 | 372 | 402 | 0.546255507 | 0.59030837 |
| — | predicted | antisense: rplN rplX | 33660 | 18385 | 31522 | 0.546197267 | 0.936482472 |
| | RNA | | | | | | |
| — | predicted | antisense: rpsS rplV | 45718 | 24911 | 64766 | 0.544883853 | 1.416641148 |
| | RNA | | | | | | |
| — | predicted | antisense: rpsB | 81822 | 44527 | 46482 | 0.544193493 | 0.568086823 |
| | RNA | | | | | | |
| — | predicted | antisense: guaA | 33531 | 18229 | 36441 | 0.543646178 | 1.086785363 |
| | RNA | | | | | | |
| — | predicted | antisense: gyrB | 42244 | 22926 | 43248 | 0.542704289 | 1.023766689 |
| | RNA | | | | | | |
| rpsI | BB_0338 | 30S ribosomal protein S9 | 321 | 174 | 66 | 0.542056075 | 0.205607477 |
| — | predicted | antisense: rplB | 54663 | 29581 | 74508 | 0.54115215 | 1.363042643 |
| | RNA | | | | | | |
| — | predicted | antisense: BB_0088 | 45623 | 24671 | 28339 | 0.540757951 | 0.621155996 |
| | RNA | | | | | | |
| — | predicted | antisense: secA | 38867 | 21017 | 39988 | 0.540741503 | 1.028841948 |
| | RNA | | | | | | |
| — | BB_0378 | hypothetical protein | 426 | 230 | 129 | 0.539906103 | 0.302816901 |
| — | predicted | antisense: BB_0088 | 40595 | 21823 | 28747 | 0.53757852 | 0.708141397 |
| | RNA | | | | | | |
| rplD | BB_0479 | 50S ribosomal protein L4 | 28 | 15 | 22 | 0.535714286 | 0.785714286 |
| — | BB_P32 | PF-32 protein | 43 | 23 | 15 | 0.534883721 | 0.348837209 |
| mutS | BB_0797 | DNA mismatch repair protein | 131 | 70 | 144 | 0.534351145 | 1.099236641 |
| | | MutS | | | | | |
| — | BB_0768 | pyridoxal kinase | 249 | 133 | 240 | 0.534136546 | 0.963855422 |
| — | BB_0722 | hypothetical protein | 190 | 101 | 111 | 0.531578947 | 0.584210526 |
| — | predicted | antisense: BB_0504 | 42929 | 22817 | 48071 | 0.531505509 | 1.11977917 |
| | RNA | | | | | | |
| — | BB_R19 | hypothetical protein | 17 | 9 | 48 | 0.529411765 | 2.823529412 |
| — | predicted | antisense: rplC rpsJ | 55472 | 29344 | 68136 | 0.528987597 | 1.228295356 |
| | RNA | | | | | | |
| — | BB_0620 | beta-glucosidase | 579 | 306 | 409 | 0.528497409 | 0.706390328 |
| — | BB_0471 | hypothetical protein | 106 | 56 | 98 | 0.528301887 | 0.924528302 |
| — | predicted | antisense: BB_0824 | 35891 | 18946 | 37684 | 0.527876069 | 1.049956814 |
| | RNA | | | | | | |
| rplQ | BB_0503 | 50S ribosomal protein L17 | 36 | 19 | 17 | 0.527777778 | 0.472222222 |
| — | BB_0087 | L-lactate dehydrogenase | 275 | 145 | 48 | 0.527272727 | 0.174545455 |
| — | BB_A37 | hypothetical protein | 114 | 60 | 267 | 0.526315789 | 2.342105263 |
| — | predicted | antisense: rpsI | 74195 | 39030 | 53828 | 0.52604623 | 0.725493632 |
| | RNA | | | | | | |
| — | BB_Q24 | hypothetical protein | 149 | 78 | 183 | 0.523489933 | 1.228187919 |
| — | predicted | antisense: BB_0238 | 51631 | 27025 | 33465 | 0.523425849 | 0.648157115 |
| | RNA | | | | | | |
| — | BB_S31 | hypothetical protein | 245 | 128 | 172 | 0.52244898 | 0.702040816 |
| — | predicted | antisense: guaA | 68920 | 35960 | 76873 | 0.521764364 | 1.11539466 |
| | RNA | | | | | | |
| — | predicted | antisense: ptsP | 57304 | 29897 | 61666 | 0.521726232 | 1.076120341 |
| | RNA | | | | | | |
| pta | BB_0589 | phosphate acetyltransferase | 246 | 128 | 269 | 0.520325203 | 1.093495935 |
| — | BB_t24 | Leu tRNA | 27 | 14 | 31 | 0.518518519 | 1.148148148 |
| — | BB_Q29 | hypothetical protein | 29 | 15 | 29 | 0.517241379 | 1 |
| — | predicted | antisense: guaA | 106958 | 55227 | 131875 | 0.516342864 | 1.232960601 |
| | RNA | | | | | | |
| — | predicted | antisense: gap | 83833 | 43247 | 85811 | 0.515870838 | 1.023594527 |
| | RNA | | | | | | |
| rpiA | BB_0657 | ribose 5-phosphate isomerase A | 2168 | 1116 | 1585 | 0.514760148 | 0.731088561 |
| — | predicted | antisense: rplB | 55617 | 28574 | 74016 | 0.513763777 | 1.330816117 |
| | RNA | | | | | | |
| — | BB_0399 | hypothetical protein | 37 | 19 | 26 | 0.513513514 | 0.702702703 |
| — | BB_0672 | chemotaxis protein CheY | 304 | 156 | 202 | 0.513157895 | 0.664473684 |
| — | BB_0001 | hypothetical protein | 129 | 66 | 120 | 0.511627907 | 0.930232558 |
| pstC | BB_0216 | phosphate ABC transporter | 259 | 132 | 128 | 0.50965251 | 0.494208494 |
| | | permease PstC | | | | | |
| — | predicted | antisense: fliM | 36905 | 18769 | 38932 | 0.508576074 | 1.054924807 |
| | RNA | | | | | | |
| — | predicted | antisense: rpoC | 36846 | 18738 | 39230 | 0.508549096 | 1.064701732 |
| | RNA | | | | | | |
| — | BB_0641 | spermidine/putrescine transport | 145 | 73 | 70 | 0.503448276 | 0.482758621 |
| | | system permease | | | | | |
| — | BB_0591 | competence locus E | 1753 | 881 | 1319 | 0.502567028 | 0.752424415 |
| — | BB_0825 | hypothetical protein | 3864 | 1941 | 2750 | 0.502329193 | 0.711697723 |
| mgsA | BB_0364 | methylglyoxal synthase | 1491 | 748 | 1658 | 0.501676727 | 1.112005366 |
| — | BB_0571 | uridylate kinase | 493 | 247 | 429 | 0.501014199 | 0.870182556 |
| — | predicted | antisense: rpsB | 51564 | 25801 | 32539 | 0.500368474 | 0.631041036 |
| | RNA | | | | | | |
| bdrE | BB_S37 | protein BdrE | 22 | 11 | 15 | 0.5 | 0.681818182 |
| — | BB_R09 | hypothetical protein | 2 | 1 | 3 | 0.5 | 1.5 |
| — | BB_M42 | phage terminase large subunit | 6 | 3 | 4 | 0.5 | 0.666666667 |
| — | BB_O33 | plasmid partition protein | 4 | 2 | 2 | 0.5 | 0.5 |
| — | BB_Q88 | hypothetical protein | 28 | 14 | 42 | 0.5 | 1.5 |
| — | BB_0011 | hypothetical protein | 518 | 259 | 458 | 0.5 | 0.884169884 |
| — | BB_0060 | endoribonuclease | 40 | 20 | 25 | 0.5 | 0.625 |
| — | BB_0081 | efflux ABC transporter permease | 42 | 21 | 28 | 0.5 | 0.666666667 |
| — | BB_0398 | lipoprotein | 10 | 5 | 12 | 0.5 | 1.2 |
| — | BB_G27 | hypothetical protein | 6 | 3 | 18 | 0.5 | 3 |
| — | BB_A03 | outer membrane protein | 26 | 13 | 14 | 0.5 | 0.538461538 |
| — | BB_A12 | BlyA family holin | 68 | 34 | 60 | 0.5 | 0.882352941 |
| osm28 | BB_A74 | outer membrane porin OMS28 | 34 | 17 | 18 | 0.5 | 0.529411765 |
| — | predicted | antisense: BB_0493 | 42471 | 20977 | 32166 | 0.493913494 | 0.757363848 |
| | RNA | | | | | | |
| — | predicted | antisense: flgC | 48385 | 23898 | 40991 | 0.493913403 | 0.847184045 |
| | RNA | | | | | | |
| — | predicted | antisense: BB_0132 | 40801 | 20135 | 47050 | 0.493492807 | 1.153158011 |
| | RNA | | | | | | |
| — | predicted | antisense: pgk | 70406 | 34715 | 75667 | 0.493068773 | 1.074723745 |
| | RNA | | | | | | |
| — | BB_R08 | hypothetical protein | 110 | 54 | 128 | 0.490909091 | 1.163636364 |
| — | predicted | antisense: tRNA-Gln-1 BB_0786 | 54021 | 26518 | 25214 | 0.490883175 | 0.466744414 |
| | RNA | | | | | | |
| — | BB_0706 | polynucleotide adenylyltransferase | 559 | 274 | 366 | 0.490161002 | 0.654740608 |
| — | predicted | antisense: BB_0087 | 48472 | 23647 | 32383 | 0.487848655 | 0.668076415 |
| | RNA | | | | | | |
| — | BB_0067 | peptidase | 234 | 114 | 136 | 0.487179487 | 0.581196581 |
| — | BB_R21 | hypothetical protein | 37 | 18 | 44 | 0.486486486 | 1.189189189 |
| — | BB_0707 | hypothetical protein | 72 | 35 | 35 | 0.486111111 | 0.486111111 |
| blyB | BB_M24 | holin protein | 103 | 50 | 89 | 0.485436893 | 0.86407767 |
| — | predicted | antisense: rpsC rplV | 40494 | 19628 | 41269 | 0.484713785 | 1.019138638 |
| | RNA | | | | | | |
| — | BB_S45 | phage terminase large subunit | 31 | 15 | 35 | 0.483870968 | 1.129032258 |
| — | BB_P08 | hypothetical protein | 899 | 429 | 1031 | 0.477196885 | 1.146829811 |
| — | BB_B01 | acylphosphatase | 543 | 257 | 392 | 0.473296501 | 0.721915285 |
| — | BB_0617 | hypothetical protein | 74 | 35 | 67 | 0.472972973 | 0.905405405 |
| bppB | BB_R37 | BppB | 36 | 17 | 35 | 0.472222222 | 0.972222222 |
| ospB | BB_A16 | outer surface protein B | 338 | 159 | 394 | 0.470414201 | 1.165680473 |
| — | BB_A11 | hypothetical protein | 83 | 39 | 84 | 0.469879518 | 1.012048193 |
| — | predicted | antisense: BB_0238 | 56873 | 26673 | 29197 | 0.468992316 | 0.513371899 |
| | RNA | | | | | | |
| — | predicted | antisense: BB_0067 | 37094 | 17396 | 35881 | 0.468970723 | 0.967299294 |
| | RNA | | | | | | |
| — | BB_0559 | PTS system glucose-specific | 595 | 279 | 177 | 0.468907563 | 0.297478992 |
| | | transporter subunit IIA | | | | | |
| — | BB_G0036 | hypothetical protein | 15 | 7 | 23 | 0.466666667 | 1.533333333 |
| — | predicted | antisense: flhA | 60393 | 28161 | 59685 | 0.466295763 | 0.988276787 |
| | RNA | | | | | | |
| cdr | BB_0728 | CoA-disulfide reductase | 1602 | 747 | 1032 | 0.466292135 | 0.644194757 |
| — | BB_0199 | hypothetical protein | 58 | 27 | 39 | 0.465517241 | 0.672413793 |
| — | BB_0051 | integral membrane protein | 492 | 229 | 255 | 0.465447154 | 0.518292683 |
| — | BB_R22 | hypothetical protein | 112 | 52 | 104 | 0.464285714 | 0.928571429 |
| — | BB_0262 | M23 peptidase domain-containing | 399 | 185 | 145 | 0.463659148 | 0.363408521 |
| | | protein | | | | | |
| — | BB_0368 | glycerol-3-phosphate | 371 | 172 | 95 | 0.46361186 | 0.25606469 |
| | | dehydrogenase | | | | | |
| — | predicted | antisense: pgk | 46633 | 21500 | 48490 | 0.461046898 | 1.039821586 |
| | RNA | | | | | | |
| — | BB_P17 | hypothetical protein | 1296 | 594 | 1476 | 0.458333333 | 1.138888889 |
| — | BB_0689 | hypothetical protein | 648 | 297 | 332 | 0.458333333 | 0.512345679 |
| — | BB_D10 | lipoprotein | 107 | 49 | 51 | 0.457943925 | 0.476635514 |
| revA | BB_P27 | surface protein | 439 | 201 | 289 | 0.45785877 | 0.658314351 |
| — | predicted | antisense: recA | 33340 | 15227 | 36224 | 0.456718656 | 1.086502699 |
| | RNA | | | | | | |
| — | predicted | antisense: rplC rplD | 40421 | 18440 | 46759 | 0.456198511 | 1.156799683 |
| | RNA | | | | | | |
| — | BB_0546 | hypothetical protein | 700 | 319 | 449 | 0.455714286 | 0.641428571 |
| — | BB_0153 | superoxide dismutase | 581 | 263 | 202 | 0.452667814 | 0.34767642 |
| — | BB_S16 | hypothetical protein | 137 | 62 | 185 | 0.452554745 | 1.350364964 |
| — | BB_P09 | hypothetical protein | 522 | 236 | 861 | 0.45210728 | 1.649425287 |
| — | BB_P07 | hypothetical protein | 374 | 168 | 365 | 0.449197861 | 0.975935829 |
| — | BB_Q50 | phage terminase large subunit | 9 | 4 | 12 | 0.444444444 | 1.333333333 |
| — | predicted | antisense: BB_0504 | 45882 | 20391 | 44271 | 0.444422649 | 0.964888191 |
| | RNA | | | | | | |
| — | predicted | antisense: groL | 59099 | 26247 | 46565 | 0.44411919 | 0.787915193 |
| | RNA | | | | | | |
| — | predicted | antisense: rplB | 30853 | 13678 | 37967 | 0.443328039 | 1.230577253 |
| | RNA | | | | | | |
| — | BB_0823 | lipoprotein | 8549 | 3787 | 6828 | 0.442975787 | 0.798689905 |
| rplW | BB_0480 | 50S ribosomal protein L23 | 34 | 15 | 23 | 0.441176471 | 0.676470588 |
| — | predicted | antisense: ffh | 63914 | 28122 | 56046 | 0.439997497 | 0.87689708 |
| | RNA | | | | | | |
| — | BB_0791 | thymidine kinase | 189 | 83 | 100 | 0.439153439 | 0.529100529 |
| — | predicted | antisense: rpsN rpsH | 38210 | 16772 | 31215 | 0.438942685 | 0.81693274 |
| | RNA | | | | | | |
| — | BB_0798 | competence protein F | 80 | 35 | 48 | 0.4375 | 0.6 |
| — | predicted | antisense: BB_0631 | 79845 | 34904 | 35398 | 0.437146972 | 0.44333396 |
| | RNA | | | | | | |
| mviN | BB_0810 | integral membrane protein MviN | 810 | 353 | 454 | 0.435802469 | 0.560493827 |
| — | BB_O29 | hypothetical protein | 106 | 46 | 46 | 0.433962264 | 0.433962264 |
| — | predicted | antisense: BB_A40 | 10427 | 4515 | 36844 | 0.433010454 | 3.533518749 |
| | RNA | | | | | | |
| — | predicted | antisense: gyrB | 86143 | 37243 | 79436 | 0.43233925 | 0.922141091 |
| | RNA | | | | | | |
| — | predicted | antisense: BB_0067 | 52774 | 22815 | 40078 | 0.432315155 | 0.759426991 |
| | RNA | | | | | | |
| — | predicted | antisense: pgk gap | 58885 | 25419 | 58511 | 0.431671903 | 0.993648637 |
| | RNA | | | | | | |
| rplI | BB_0112 | 50S ribosomal protein L9 | 51 | 22 | 23 | 0.431372549 | 0.450980392 |
| — | BB_0063 | hypothetical protein | 1012 | 436 | 895 | 0.43083004 | 0.884387352 |
| blyA | BB_P23 | holin protein | 140 | 60 | 64 | 0.428571429 | 0.457142857 |
| — | BB_L43 | phage terminase large subunit | 14 | 6 | 19 | 0.428571429 | 1.357142857 |
| — | predicted | antisense: pgk | 35559 | 15222 | 37841 | 0.42807728 | 1.064175033 |
| | RNA | | | | | | |
| argS | BB_0594 | arginine--tRNA ligase | 566 | 241 | 392 | 0.425795053 | 0.692579505 |
| — | predicted | antisense: BB_0355 | 55802 | 23743 | 34065 | 0.425486542 | 0.610461991 |
| | RNA | | | | | | |
| — | predicted | antisense: groL | 76651 | 32516 | 64273 | 0.424208425 | 0.838514827 |
| | RNA | | | | | | |
| — | predicted | antisense: rpoB | 30375 | 12851 | 36200 | 0.423078189 | 1.191769547 |
| | RNA | | | | | | |
| mlpG | BB_O28 | lipoprotein | 26 | 11 | 27 | 0.423076923 | 1.038461538 |
| — | BB_J27 | efflux ABC transporter permease | 102 | 43 | 77 | 0.421568627 | 0.754901961 |
| — | predicted | antisense: rpsJ | 69096 | 29059 | 72573 | 0.420559801 | 1.050321292 |
| | RNA | | | | | | |
| — | BB_R16 | hypothetical protein | 281 | 118 | 350 | 0.419928826 | 1.245551601 |
| — | BB_0655 | heat shock protein | 707 | 296 | 394 | 0.418670438 | 0.5572843 |
| — | BB_H17 | hypothetical protein | 43 | 18 | 89 | 0.418604651 | 2.069767442 |
| hflK | BB_0203 | protein HflK | 189 | 79 | 121 | 0.417989418 | 0.64021164 |
| — | BB_0799 | hypothetical protein | 12 | 5 | 2 | 0.416666667 | 0.166666667 |
| — | predicted | antisense: BB_0801 | 42670 | 17779 | 31432 | 0.416662761 | 0.736629951 |
| | RNA | | | | | | |
| — | BB_P16 | hypothetical protein | 793 | 330 | 1075 | 0.416141236 | 1.355611602 |
| — | BB_0349 | hypothetical protein | 368 | 153 | 223 | 0.41576087 | 0.605978261 |
| — | predicted | antisense: gyrA | 90201 | 37473 | 90726 | 0.415438853 | 1.005820335 |
| | RNA | | | | | | |
| — | predicted | antisense: rplD | 38995 | 16168 | 40902 | 0.414617259 | 1.048903706 |
| | RNA | | | | | | |
| 23S_rrlB | BB_r04 | 23S ribosomal RNA | 6339 | 2622 | 3039 | 0.41362991 | 0.479413157 |
| — | predicted | — | 220296 | 90190 | 107023 | 0.409403711 | 0.48581454 |
| | RNA | | | | | | |
| — | BB_0727 | phosphofructokinase | 203 | 83 | 48 | 0.408866995 | 0.236453202 |
| — | predicted | antisense: tpiA | 50329 | 20508 | 45323 | 0.40747879 | 0.900534483 |
| | RNA | | | | | | |
| — | BB_0062 | rRNA small subunit | 597 | 243 | 393 | 0.407035176 | 0.658291457 |
| | | methyltransferase E | | | | | |
| — | BB_G24 | hypothetical protein | 32 | 13 | 71 | 0.40625 | 2.21875 |
| — | predicted | antisense: BB_0458 | 45632 | 18449 | 35735 | 0.404299614 | 0.783112728 |
| | RNA | | | | | | |
| — | BB_0252 | hypothetical protein | 1087 | 439 | 497 | 0.403863845 | 0.457221711 |
| — | predicted | antisense: rpsC | 29640 | 11857 | 37691 | 0.400033738 | 1.271626181 |
| | RNA | | | | | | |
| — | BB_Q37 | hypothetical protein | 25 | 10 | 14 | 0.4 | 0.56 |
| rpsQ | BB_0487 | 30S ribosomal protein S17 | 5 | 2 | 2 | 0.4 | 0.4 |
| — | BB_R29 | hypothetical protein | 543 | 217 | 252 | 0.399631676 | 0.464088398 |
| 23S_rrlA | BB_r02 | 23S ribosomal RNA | 6312 | 2516 | 3005 | 0.39860583 | 0.476077313 |
| — | BB_S09 | hypothetical protein | 63 | 25 | 76 | 0.396825397 | 1.206349206 |
| — | BB_0456 | hypothetical protein | 58 | 23 | 35 | 0.396551724 | 0.603448276 |
| — | BB_0193 | lipoprotein | 48 | 19 | 15 | 0.395833333 | 0.3125 |
| ung | BB_0053 | uracil-DNA glycosylase | 3295 | 1299 | 1626 | 0.394233687 | 0.493474962 |
| — | predicted | antisense: gap | 68489 | 26996 | 59630 | 0.394165486 | 0.870650761 |
| | RNA | | | | | | |
| napA | BB_0690 | neutrophil activating protein A | 28 | 11 | 16 | 0.392857143 | 0.571428571 |
| | | (napA) | | | | | |
| — | BB_P11 | hypothetical protein | 467 | 181 | 439 | 0.3875803 | 0.940042827 |
| — | predicted | antisense: BB_0327 | 57402 | 22110 | 39518 | 0.385178217 | 0.688442911 |
| | RNA | | | | | | |
| — | predicted | antisense: BB_B16 | 37859 | 14574 | 37357 | 0.3849547 | 0.986740273 |
| | RNA | | | | | | |
| — | predicted | antisense: ptsP | 45602 | 17542 | 43002 | 0.384676111 | 0.942984957 |
| | RNA | | | | | | |
| — | BB_G07 | hypothetical protein | 26 | 10 | 41 | 0.384615385 | 1.576923077 |
| — | predicted | antisense: groL | 69367 | 26372 | 40993 | 0.380180778 | 0.590958237 |
| | RNA | | | | | | |
| — | BB_P10 | hypothetical protein | 501 | 190 | 635 | 0.379241517 | 1.26746507 |
| — | predicted | antisense: BB_A40 | 12605 | 4776 | 43757 | 0.378897263 | 3.471400238 |
| | RNA | | | | | | |
| — | BB_M10 | hypothetical protein | 37 | 14 | 42 | 0.378378378 | 1.135135135 |
| lepB | BB_0263 | signal peptidase I | 304 | 115 | 98 | 0.378289474 | 0.322368421 |
| — | predicted | antisense: BB_0355 | 117881 | 44515 | 91175 | 0.37762659 | 0.773449496 |
| | RNA | | | | | | |
| — | predicted | antisense: pgk | 49312 | 18583 | 40471 | 0.376845393 | 0.820713011 |
| | RNA | | | | | | |
| — | predicted | antisense: BB_0801 | 57366 | 21574 | 43192 | 0.376076422 | 0.752919848 |
| | RNA | | | | | | |
| — | predicted | antisense: BB_0789 | 38551 | 14469 | 37319 | 0.375321003 | 0.968042334 |
| | RNA | | | | | | |
| — | BB_Q53 | hypothetical protein | 8 | 3 | 6 | 0.375 | 0.75 |
| — | predicted | antisense: BB_0465 | 62532 | 23288 | 29768 | 0.372417322 | 0.476044265 |
| | RNA | | | | | | |
| murB | BB_0598 | UDP-N- | 513 | 191 | 249 | 0.372319688 | 0.485380117 |
| | | acetylenolpyruvoylglucosamine | | | | | |
| | | reductase | | | | | |
| — | BB_M03 | hypothetical protein | 19 | 7 | 14 | 0.368421053 | 0.736842105 |
| blyB | BB_P24 | holin protein | 243 | 89 | 165 | 0.366255144 | 0.679012346 |
| — | predicted | antisense: groL | 67821 | 24725 | 35872 | 0.364562599 | 0.52892172 |
| | RNA | | | | | | |
| — | BB_0525 | hypothetical protein | 184 | 67 | 73 | 0.364130435 | 0.39673913 |
| — | BB_0562 | hypothetical protein | 1296 | 471 | 842 | 0.363425926 | 0.649691358 |
| — | BB_0029 | hypothetical protein | 235 | 85 | 114 | 0.361702128 | 0.485106383 |
| rpsU | BB_0256 | 30S ribosomal protein S21 | 226 | 81 | 34 | 0.35840708 | 0.150442478 |
| — | predicted | antisense: BB_0545 | 52578 | 18799 | 38525 | 0.357544981 | 0.73272091 |
| | RNA | | | | | | |
| — | BB_0316 | integral membrane protein | 169 | 60 | 36 | 0.355029586 | 0.213017751 |
| — | BB_0211 | DNA mismatch repair protein | 195 | 69 | 113 | 0.353846154 | 0.579487179 |
| — | BB_0332 | peptide ABC transporter permease | 566 | 200 | 296 | 0.35335689 | 0.522968198 |
| rpsK | BB_0501 | 30S ribosomal protein S11 | 34 | 12 | 27 | 0.352941176 | 0.794117647 |
| — | BB_P01 | phage portal protein | 105 | 37 | 77 | 0.352380952 | 0.733333333 |
| — | predicted | antisense: rplI | 76228 | 26825 | 39982 | 0.351904812 | 0.524505431 |
| | RNA | | | | | | |
| — | BB_D01 | hypothetical protein | 165 | 58 | 221 | 0.351515152 | 1.339393939 |
| — | predicted | antisense: groL | 74218 | 26012 | 45467 | 0.350481015 | 0.612614191 |
| | RNA | | | | | | |
| — | BB_Q23 | hypothetical protein | 83 | 29 | 101 | 0.34939759 | 1.21686747 |
| ospA | BB_A15 | outer surface protein A | 318 | 111 | 213 | 0.349056604 | 0.669811321 |
| cheR | BB_0040 | chemotaxis protein | 560 | 194 | 233 | 0.346428571 | 0.416071429 |
| | | methyltransferase | | | | | |
| — | predicted | antisense: BB_0544 | 57847 | 20017 | 38154 | 0.346033502 | 0.65956748 |
| | RNA | | | | | | |
| — | predicted | antisense: rplQ | 73534 | 25184 | 42740 | 0.342481029 | 0.581227731 |
| | RNA | | | | | | |
| — | BB_0401 | glutamate transporter | 824 | 282 | 434 | 0.34223301 | 0.526699029 |
| — | BB_B02 | hypothetical protein | 2350 | 803 | 1461 | 0.341702128 | 0.621702128 |
| — | predicted | antisense: rpsB | 71740 | 24471 | 36869 | 0.341106774 | 0.513925286 |
| | RNA | | | | | | |
| — | BB_R43 | hypothetical protein | 250 | 85 | 111 | 0.34 | 0.444 |
| — | BB_0336 | hypothetical protein | 365 | 124 | 61 | 0.339726027 | 0.167123288 |
| — | predicted | antisense: tpiA | 52378 | 17675 | 43943 | 0.337450838 | 0.838959105 |
| | RNA | | | | | | |
| truB | BB_0803 | tRNA pseudouridine synthase B | 92 | 31 | 51 | 0.336956522 | 0.554347826 |
| — | BB_0646 | hypothetical protein | 939 | 316 | 444 | 0.336528222 | 0.47284345 |
| — | predicted | antisense: rplB rplW | 39815 | 13363 | 38530 | 0.335627276 | 0.967725732 |
| | RNA | | | | | | |
| proS | BB_0402 | proline--tRNA ligase | 257 | 86 | 87 | 0.33463035 | 0.338521401 |
| — | BB_O31 | hypothetical protein | 3 | 1 | 0 | 0.333333333 | 0 |
| bppB | BB_O37 | protein BppB | 15 | 5 | 8 | 0.333333333 | 0.533333333 |
| — | BB_L20 | hypothetical protein | 72 | 24 | 22 | 0.333333333 | 0.305555556 |
| — | BB_D04 | hypothetical protein | 72 | 24 | 66 | 0.333333333 | 0.916666667 |
| — | BB_A19 | hypothetical protein | 27 | 9 | 44 | 0.333333333 | 1.62962963 |
| — | predicted | antisense: gyrA | 90376 | 30117 | 61775 | 0.333241126 | 0.683533239 |
| | RNA | | | | | | |
| — | BB_Q17 | hypothetical protein | 127 | 42 | 131 | 0.330708661 | 1.031496063 |
| — | BB_J34 | lipoprotein | 103 | 34 | 54 | 0.330097087 | 0.524271845 |
| — | predicted | antisense: groL | 68330 | 22456 | 39402 | 0.328640421 | 0.576642763 |
| | RNA | | | | | | |
| — | BB_0664 | lipoprotein | 465 | 152 | 142 | 0.32688172 | 0.305376344 |
| — | predicted | antisense: BB_0795 | 48965 | 15928 | 35608 | 0.325293577 | 0.727213316 |
| | RNA | | | | | | |
| la7 | BB_0365 | lipoprotein LA7 | 326 | 106 | 112 | 0.325153374 | 0.343558282 |
| — | predicted | antisense: BB_0801 | 46073 | 14935 | 37641 | 0.324159486 | 0.816986087 |
| | RNA | | | | | | |
| blyA | BB_N23 | holin protein | 155 | 50 | 50 | 0.322580645 | 0.322580645 |
| — | BB_A69 | putative surface protein | 196 | 63 | 98 | 0.321428571 | 0.5 |
| — | predicted | antisense: BB_A61 | 43675 | 14034 | 63250 | 0.321327991 | 1.448196909 |
| | RNA | | | | | | |
| — | BB_0024 | hypothetical protein | 803 | 258 | 390 | 0.321295143 | 0.485678705 |
| — | BB_B26 | hypothetical protein | 611 | 196 | 660 | 0.320785597 | 1.080196399 |
| — | predicted | antisense: ptsP | 57572 | 18427 | 46666 | 0.320068783 | 0.810567637 |
| | RNA | | | | | | |
| — | predicted | antisense: tsf | 58070 | 18578 | 35706 | 0.319924229 | 0.614878595 |
| | RNA | | | | | | |
| — | BB_R14 | hypothetical protein | 47 | 15 | 61 | 0.319148936 | 1.29787234 |
| — | BB_H28 | PF-32 protein | 22 | 7 | 13 | 0.318181818 | 0.590909091 |
| — | BB_Q12 | hypothetical protein | 19 | 6 | 20 | 0.315789474 | 1.052631579 |
| — | BB_0673 | hypothetical protein | 38 | 12 | 15 | 0.315789474 | 0.394736842 |
| nagB | BB_0152 | glucosamine-6-phosphate | 1154 | 363 | 753 | 0.314558059 | 0.652512998 |
| | | deaminase | | | | | |
| — | BB_R20 | hypothetical protein | 175 | 55 | 73 | 0.314285714 | 0.417142857 |
| — | predicted | antisense: pgk | 50251 | 15705 | 36345 | 0.312531094 | 0.723269189 |
| | RNA | | | | | | |
| — | BB_A41 | hypothetical protein | 48 | 15 | 89 | 0.3125 | 1.854166667 |
| — | BB_0147 | flagellin | 221 | 69 | 73 | 0.312217195 | 0.330316742 |
| — | BB_O44 | phage terminase large subunit | 13 | 4 | 12 | 0.307692308 | 0.923076923 |
| — | BB_0397 | hypothetical protein | 26 | 8 | 27 | 0.307692308 | 1.038461538 |
| — | predicted | antisense: gyrA | 60806 | 18573 | 52076 | 0.305446831 | 0.856428642 |
| | RNA | | | | | | |
| — | BB_0352 | hypothetical protein | 132 | 40 | 66 | 0.303030303 | 0.5 |
| — | BB_0739 | hypothetical protein | 208 | 63 | 51 | 0.302884615 | 0.245192308 |
| — | predicted | antisense: BB_0789 | 55407 | 16638 | 36890 | 0.300286967 | 0.66580035 |
| | RNA | | | | | | |
| — | BB_P22 | hypothetical protein | 860 | 258 | 593 | 0.3 | 0.689534884 |
| — | BB_Q21 | hypothetical protein | 10 | 3 | 56 | 0.3 | 5.6 |
| — | BB_0331 | hypothetical protein | 10 | 3 | 5 | 0.3 | 0.5 |
| — | BB_0429 | hypothetical protein | 1770 | 531 | 1046 | 0.3 | 0.590960452 |
| — | BB_t20 | Ser tRNA | 30 | 9 | 7 | 0.3 | 0.233333333 |
| — | BB_0465 | hypothetical protein | 177 | 53 | 141 | 0.299435028 | 0.796610169 |
| — | BB_0129 | hypothetical protein | 137 | 41 | 86 | 0.299270073 | 0.627737226 |
| — | BB_D001 | lipoprotein | 67 | 20 | 77 | 0.298507463 | 1.149253731 |
| — | predicted | antisense: fusA | 107812 | 32086 | 45574 | 0.297610656 | 0.422717323 |
| | RNA | | | | | | |
| — | BB_0004 | phosphoglucomutase | 859 | 253 | 649 | 0.294528522 | 0.755529686 |
| — | BB_0663 | hypothetical protein | 1521 | 444 | 533 | 0.291913215 | 0.35042735 |
| — | predicted | antisense: rplQ | 80945 | 23399 | 50094 | 0.289072827 | 0.618864661 |
| | RNA | | | | | | |
| — | BB_J46 | hypothetical protein | 45 | 13 | 17 | 0.288888889 | 0.377777778 |
| — | predicted | antisense: fliE | 57125 | 16447 | 36435 | 0.287912473 | 0.637811816 |
| | RNA | | | | | | |
| — | predicted | antisense: rpsM | 81570 | 23376 | 33897 | 0.286575947 | 0.41555719 |
| | RNA | | | | | | |
| — | predicted | antisense: tuf | 52944 | 15127 | 51878 | 0.285716984 | 0.979865518 |
| | RNA | | | | | | |
| — | BB_P25 | hypothetical protein | 56 | 16 | 39 | 0.285714286 | 0.696428571 |
| — | BB_B07 | alpha3-beta1 integrin-binding | 1824 | 521 | 1012 | 0.285635965 | 0.554824561 |
| | | protein | | | | | |
| — | predicted | antisense: rpsG | 97339 | 27687 | 63802 | 0.28443892 | 0.65546184 |
| | RNA | | | | | | |
| — | BB_N19 | hypothetical protein | 39 | 11 | 21 | 0.282051282 | 0.538461538 |
| — | predicted | antisense: BB_0269 | 96916 | 27092 | 39823 | 0.279541046 | 0.410902225 |
| | RNA | | | | | | |
| — | predicted | antisense: guaA | 43695 | 12182 | 35261 | 0.278796201 | 0.806980204 |
| | RNA | | | | | | |
| ksgA | BB_0590 | rRNA small subunit | 512 | 142 | 177 | 0.27734375 | 0.345703125 |
| | | methyltransferase A | | | | | |
| — | predicted | antisense: tsf | 56929 | 15786 | 35464 | 0.277292768 | 0.622951396 |
| | RNA | | | | | | |
| — | BB_M26 | hypothetical protein | 379 | 104 | 222 | 0.274406332 | 0.585751979 |
| — | predicted | antisense: rpoA | 61632 | 16880 | 36602 | 0.273883697 | 0.593879803 |
| | RNA | | | | | | |
| — | BB_A59 | hypothetical protein | 910 | 242 | 688 | 0.265934066 | 0.756043956 |
| — | BB_0475 | lipoprotein | 34 | 9 | 29 | 0.264705882 | 0.852941176 |
| — | BB_R26 | hypothetical protein | 818 | 216 | 393 | 0.26405868 | 0.480440098 |
| — | BB_S26 | hypothetical protein | 1273 | 336 | 774 | 0.263943441 | 0.608012569 |
| — | predicted | antisense: BB_0147 | 122230 | 32260 | 73284 | 0.263928659 | 0.59955821 |
| | RNA | | | | | | |
| — | BB_S02 | hypothetical protein | 19 | 5 | 10 | 0.263157895 | 0.526315789 |
| — | predicted | antisense: cspA | 30317 | 7893 | 35044 | 0.260348979 | 1.155919121 |
| | RNA | | | | | | |
| — | BB_N31 | hypothetical protein | 27 | 7 | 21 | 0.259259259 | 0.777777778 |
| — | predicted | antisense: BB_B22 | 77813 | 19948 | 50036 | 0.256358192 | 0.6430288 |
| | RNA | | | | | | |
| — | predicted | — | 161854 | 41210 | 55448 | 0.254612181 | 0.34258035 |
| | RNA | | | | | | |
| — | BB_t33 | Thr tRNA | 59 | 15 | 11 | 0.254237288 | 0.186440678 |
| blyB | BB_Q31 | holin protein | 24 | 6 | 21 | 0.25 | 0.875 |
| — | BB_D22 | hypothetical protein | 16 | 4 | 5 | 0.25 | 0.3125 |
| — | BB_G01 | hypothetical protein | 12 | 3 | 19 | 0.25 | 1.583333333 |
| — | BB_G22 | hypothetical protein | 36 | 9 | 80 | 0.25 | 2.222222222 |
| — | BB_H27 | hypothetical protein | 8 | 2 | 4 | 0.25 | 0.5 |
| — | predicted | antisense: BB_A38 | 16406 | 4088 | 40494 | 0.24917713 | 2.468243326 |
| | RNA | | | | | | |
| — | BB_0759 | membrane protein | 2113 | 513 | 1041 | 0.242782773 | 0.492664458 |
| — | BB_S21 | hypothetical protein | 29 | 7 | 11 | 0.24137931 | 0.379310345 |
| — | predicted | antisense: BB_A61 | 31248 | 7521 | 42297 | 0.240687404 | 1.35359063 |
| | RNA | | | | | | |
| — | BB_N20 | hypothetical protein | 287 | 69 | 68 | 0.240418118 | 0.236933798 |
| bppB | BB_N36 | protein BppB | 25 | 6 | 22 | 0.24 | 0.88 |
| — | BB_R10 | hypothetical protein | 142 | 34 | 119 | 0.23943662 | 0.838028169 |
| — | BB_R11 | hypothetical protein | 38 | 9 | 45 | 0.236842105 | 1.184210526 |
| — | BB_A66 | outer surface protein | 917 | 217 | 892 | 0.236641221 | 0.972737186 |
| — | predicted | antisense: BB_A38 | 19570 | 4607 | 44617 | 0.235411344 | 2.279867144 |
| | RNA | | | | | | |
| — | predicted | antisense: BB_A38 | 13552 | 3189 | 41983 | 0.235315821 | 3.097919126 |
| | RNA | | | | | | |
| — | BB_0013 | hypothetical protein | 51 | 12 | 35 | 0.235294118 | 0.68627451 |
| — | BB_H06 | complement regulator-acquiring | 34 | 8 | 10 | 0.235294118 | 0.294117647 |
| | | surface protein | | | | | |
| — | predicted | antisense: BB_0034 | 148896 | 35007 | 64256 | 0.235110413 | 0.431549538 |
| | RNA | | | | | | |
| — | BB_H05 | hypothetical protein | 61 | 14 | 23 | 0.229508197 | 0.37704918 |
| — | predicted | antisense: ptsP | 56965 | 13026 | 39351 | 0.228666725 | 0.690792592 |
| | RNA | | | | | | |
| — | predicted | antisense: guaA | 72328 | 16144 | 46472 | 0.223205398 | 0.642517421 |
| | RNA | | | | | | |
| — | predicted | antisense: BB_B22 | 75811 | 16883 | 49510 | 0.222698553 | 0.653071454 |
| | RNA | | | | | | |
| — | BB_Q52 | hypothetical protein | 9 | 2 | 24 | 0.222222222 | 2.666666667 |
| — | predicted | antisense: BB_0137 | 50131 | 10862 | 41519 | 0.216672319 | 0.82821009 |
| | RNA | | | | | | |
| — | BB_N26 | hypothetical protein | 183 | 39 | 103 | 0.213114754 | 0.56284153 |
| — | BB_0102 | hypothetical protein | 170 | 36 | 53 | 0.211764706 | 0.311764706 |
| — | BB_P21 | hypothetical protein | 658 | 139 | 291 | 0.211246201 | 0.44224924 |
| lnt | BB_0237 | apolipoprotein N-acyltransferase | 5356 | 1129 | 2060 | 0.210791636 | 0.384615385 |
| — | predicted | antisense: rpsK | 81384 | 16444 | 41213 | 0.202054458 | 0.50640175 |
| | RNA | | | | | | |
| infA | BB_0169 | translation initiation factor IF-1 | 2415 | 486 | 522 | 0.201242236 | 0.216149068 |
| bdrF | BB_S29 | protein BdrF | 65 | 13 | 15 | 0.2 | 0.230769231 |
| — | BB_G28 | hypothetical protein | 5 | 1 | 67 | 0.2 | 13.4 |
| — | BB_G33 | hypothetical protein | 60 | 12 | 13 | 0.2 | 0.216666667 |
| cdd | BB_0618 | cytidine deaminase | 951 | 190 | 267 | 0.199789695 | 0.280757098 |
| — | BB_A10 | hypothetical protein | 106 | 21 | 61 | 0.198113208 | 0.575471698 |
| — | predicted | antisense: rpoA | 69943 | 13810 | 38584 | 0.197446492 | 0.5516492 |
| | RNA | | | | | | |
| — | BB_0353 | membrane protein | 233 | 45 | 31 | 0.193133047 | 0.13304721 |
| — | BB_0034 | outer membrane protein P13 | 285 | 55 | 41 | 0.192982456 | 0.143859649 |
| — | BB_N10 | hypothetical protein | 26 | 5 | 20 | 0.192307692 | 0.769230769 |
| — | BB_0628 | lipoprotein | 799 | 153 | 158 | 0.191489362 | 0.197747184 |
| — | predicted | antisense: BB_A43 | 32768 | 6191 | 36644 | 0.188934326 | 1.118286133 |
| | RNA | | | | | | |
| — | predicted | antisense: BB_B22 | 68518 | 12797 | 42142 | 0.18676844 | 0.61505006 |
| | RNA | | | | | | |
| — | BB_M11 | hypothetical protein | 43 | 8 | 30 | 0.186046512 | 0.697674419 |
| — | BB_0453 | small conductance | 1922 | 357 | 467 | 0.185744017 | 0.242976067 |
| | | mechanosensitive ion channel | | | | | |
| — | BB_0025 | transcriptional regulator | 1366 | 248 | 272 | 0.181551977 | 0.199121523 |
| lp6.6 | BB_A62 | 6.6 kDa lipoprotein | 478 | 86 | 115 | 0.179916318 | 0.240585774 |
| — | BB_A57 | P45-13 | 412 | 74 | 402 | 0.17961165 | 0.975728155 |
| dnaB | BB_0111 | replicative DNA helicase | 411 | 72 | 144 | 0.175182482 | 0.350364964 |
| — | BB_S33 | hypothetical protein | 87 | 15 | 29 | 0.172413793 | 0.333333333 |
| — | BB_N41 | hypothetical protein | 137 | 23 | 47 | 0.167883212 | 0.343065693 |
| — | BB_t25 | Gly tRNA | 3756 | 627 | 1187 | 0.166932907 | 0.316027689 |
| — | BB_S17 | hypothetical protein | 42 | 7 | 35 | 0.166666667 | 0.833333333 |
| — | BB_0838a | hypothetical protein | 12 | 2 | 4 | 0.166666667 | 0.333333333 |
| — | predicted | antisense: gyrA | 78093 | 13010 | 49816 | 0.166596238 | 0.637906086 |
| | RNA | | | | | | |
| — | BB_Q33 | hypothetical protein | 343 | 57 | 147 | 0.166180758 | 0.428571429 |
| — | predicted | antisense: BB_B22 | 93372 | 15380 | 47924 | 0.164717474 | 0.513258793 |
| | RNA | | | | | | |
| — | BB_Q28 | hypothetical protein | 63 | 10 | 22 | 0.158730159 | 0.349206349 |
| — | BB_0582 | carboxypeptidase | 442 | 69 | 96 | 0.156108597 | 0.21719457 |
| tRNA- | BB_t12 | Asp tRNA | 27 | 4 | 0 | 0.148148148 | 0 |
| Asp-1 | | | | | | | |
| — | BB_D09 | hypothetical protein | 54 | 8 | 42 | 0.148148148 | 0.777777778 |
| — | BB_D13 | hypothetical protein | 1530 | 226 | 934 | 0.147712418 | 0.610457516 |
| — | BB_A42 | hypothetical protein | 68 | 10 | 48 | 0.147058824 | 0.705882353 |
| — | BB_S07 | hypothetical protein | 14 | 2 | 13 | 0.142857143 | 0.928571429 |
| — | predicted | antisense: BB_A59 | 183085 | 25097 | 70553 | 0.137078406 | 0.385356528 |
| | RNA | | | | | | |
| — | BB_0832 | lipoprotein | 2040 | 278 | 753 | 0.13627451 | 0.369117647 |
| — | predicted | — | 273949 | 37199 | 63152 | 0.135788048 | 0.23052466 |
| | RNA | | | | | | |
| — | BB_B27 | lipoprotein | 267 | 36 | 42 | 0.134831461 | 0.157303371 |
| — | predicted | antisense: BB_0559 | 83624 | 10987 | 39487 | 0.131385727 | 0.472196977 |
| | RNA | | | | | | |
| — | predicted | — | 324978 | 41512 | 111706 | 0.127737878 | 0.343734037 |
| | RNA | | | | | | |
| — | BB_0367 | hypothetical protein | 500 | 63 | 176 | 0.126 | 0.352 |
| — | predicted | antisense: BB_B22 | 117230 | 13859 | 44966 | 0.118220592 | 0.383570758 |
| | RNA | | | | | | |
| — | BB_A09 | hypothetical protein | 102 | 12 | 50 | 0.117647059 | 0.490196078 |
| — | BB_L25 | hypothetical protein | 26 | 3 | 13 | 0.115384615 | 0.5 |
| — | BB_P20 | hypothetical protein | 1025 | 118 | 288 | 0.115121951 | 0.28097561 |
| bdrH | BB_R27 | BdrH | 79 | 9 | 15 | 0.113924051 | 0.189873418 |
| — | BB_0576 | hypothetical protein | 649 | 72 | 140 | 0.110939908 | 0.215716487 |
| — | predicted | antisense: BB_0147 | 223894 | 24690 | 71642 | 0.110275398 | 0.319981777 |
| | RNA | | | | | | |
| — | BB_J47 | hypothetical protein | 183 | 20 | 69 | 0.109289617 | 0.37704918 |
| — | BB_0143 | membrane protein insertion | 4042 | 440 | 1085 | 0.108857001 | 0.26843147 |
| | | efficiency factor | | | | | |
| — | BB_Q27 | hypothetical protein | 157 | 17 | 36 | 0.108280255 | 0.229299363 |
| — | BB_S25 | hypothetical protein | 389 | 42 | 120 | 0.107969152 | 0.30848329 |
| — | predicted | antisense: ospA ospB | 386319 | 40930 | 102443 | 0.105948711 | 0.265177224 |
| | RNA | | | | | | |
| — | BB_A64 | P35 antigen | 21094 | 2111 | 9453 | 0.100075851 | 0.448136911 |
| — | BB_0162 | hypothetical protein | 1622 | 158 | 401 | 0.097410604 | 0.247225647 |
| — | BB_A13 | hypothetical protein | 520 | 49 | 256 | 0.094230769 | 0.492307692 |
| smpB | BB_0033 | SsrA-binding protein | 448 | 41 | 176 | 0.091517857 | 0.392857143 |
| — | BB_G34 | hypothetical protein | 1371 | 122 | 439 | 0.088986142 | 0.32020423 |
| — | BB_Q32 | hypothetical protein | 71 | 6 | 14 | 0.084507042 | 0.197183099 |
| — | BB_J36 | lipoprotein | 334 | 28 | 90 | 0.083832335 | 0.269461078 |
| — | predicted | antisense: napA | 99610 | 8108 | 19231 | 0.08139745 | 0.193062945 |
| | RNA | | | | | | |
| — | BB_t22 | Xaa tRNA | 13 | 1 | 12 | 0.076923077 | 0.923076923 |
| tRNA- | BB_t11 | Asn tRNA | 159 | 12 | 26 | 0.075471698 | 0.163522013 |
| Asn-1 | | | | | | | |
| — | BB_A58 | hypothetical protein | 40093 | 2956 | 12536 | 0.073728581 | 0.312673035 |
| — | predicted | antisense: lp6.6 | 709503 | 52073 | 140114 | 0.073393629 | 0.197481899 |
| | RNA | | | | | | |
| — | BB_I39 | surface antigen | 43 | 3 | 8 | 0.069767442 | 0.186046512 |
| tRNA- | BB_t06 | Ser tRNA | 15 | 1 | 0 | 0.066666667 | 0 |
| Ser-3 | | | | | | | |
| — | BB_A61 | hypothetical protein | 681 | 45 | 133 | 0.066079295 | 0.195301028 |
| — | BB_H13 | protein RepU | 16 | 1 | 3 | 0.0625 | 0.1875 |
| — | BB_R25 | hypothetical protein | 115 | 7 | 43 | 0.060869565 | 0.373913043 |
| rpmE | BB_0229 | 50S ribosomal protein L31 | 365 | 22 | 52 | 0.060273973 | 0.142465753 |
| — | BB_t28 | Met tRNA | 42 | 2 | 0 | 0.047619048 | 0 |
| — | BB_0428 | hypothetical protein | 4727 | 195 | 627 | 0.04125238 | 0.132642268 |
| — | BB_S10 | hypothetical protein | 31 | 1 | 34 | 0.032258065 | 1.096774194 |
| — | BB_M18 | hypothetical protein | 2 | 0 | 4 | 0 | 2 |
| — | BB_L26 | hypothetical protein | 2 | 0 | 1 | 0 | 0.5 |
| — | BB_N14 | hypothetical protein | 2 | 0 | 4 | 0 | 2 |
| blyB | BB_N24 | holin protein | 3 | 0 | 0 | 0 | 0 |
| bppB | BB_Q44 | protein BppB | 3 | 0 | 6 | 0 | 2 |
| — | BB_Q62 | hypothetical protein | 6 | 0 | 1 | 0 | 0.166666667 |
| — | BB_Q0091 | hypothetical protein | 27 | 0 | 6 | 0 | 0.222222222 |
| — | BB_D0027 | hypothetical protein | 11 | 0 | 0 | 0 | 0 |
| — | BB_J0056 | hypothetical protein | 27 | 0 | 13 | 0 | 0.481481481 |
| — | BB_J41 | antigen P35 | 2 | 0 | 3 | 0 | 1.5 |
| — | BB_S06 | hypothetical protein | 0 | 0 | 0 | — | — |
| — | BB_S08 | hypothetical protein | 0 | 0 | 0 | — | — |
| — | BB_S11 | hypothetical protein | 0 | 0 | 0 | — | — |
| — | BB_S12 | hypothetical protein | 0 | 0 | 0 | — | — |
| — | BB_S13 | hypothetical protein | 0 | 0 | 0 | — | — |
| — | BB_S14 | hypothetical protein | 0 | 0 | 0 | — | — |
| — | BB_S18 | hypothetical protein | 0 | 0 | 0 | — | — |
| — | BB_S19 | hypothetical protein | 0 | 0 | 0 | — | — |
| — | BB_S20 | hypothetical protein | 0 | 0 | 0 | — | — |
| blyA | BB_S23 | holin protein | 0 | 0 | 0 | — | — |
| blyB | BB_S24 | holin protein | 0 | 0 | 0 | — | — |
| blyB | BB_R24 | hemolysin accessory protein | 0 | 0 | 0 | — | — |
| — | BB_M01 | phage portal protein | 0 | 0 | 0 | — | — |
| — | BB_M09 | hypothetical protein | 0 | 0 | 5 | — | — |
| — | BB_M12 | hypothetical protein | 0 | 0 | 0 | — | — |
| — | BB_M13 | hypothetical protein | 0 | 0 | 0 | — | — |
| — | BB_M14 | hypothetical protein | 0 | 0 | 0 | — | — |
| — | BB_M15 | hypothetical protein | 0 | 0 | 0 | — | — |
| — | BB_M16 | hypothetical protein | 0 | 0 | 5 | — | — |
| — | BB_M17 | hypothetical protein | 0 | 0 | 0 | — | — |
| — | BB_M19 | hypothetical protein | 0 | 0 | 2 | — | — |
| blyA | BB_M23 | holin protein | 0 | 0 | 0 | — | — |
| — | BB_M25 | hypothetical protein | 0 | 0 | 0 | — | — |
| mlpF | BB_M28 | lipoprotein | 0 | 0 | 0 | — | — |
| — | BB_M30 | hypothetical protein | 0 | 0 | 0 | — | — |
| — | BB_M33 | PF-49 protein | 0 | 0 | 0 | — | — |
| bdrK | BB_M34 | BdrK | 0 | 0 | 0 | — | — |
| erpK | BB_M38 | ErpK protein | 0 | 0 | 0 | — | — |
| — | BB_M39 | hypothetical protein | 0 | 0 | 0 | — | — |
| — | BB_M41 | hypothetical protein | 0 | 0 | 0 | — | — |
| — | BB_O01 | hypothetical protein | 0 | 0 | 4 | — | — |
| — | BB_O03 | hypothetical protein | 0 | 0 | 0 | — | — |
| — | BB_O04 | hypothetical protein | 0 | 0 | 0 | — | — |
| — | BB_O05 | hypothetical protein | 0 | 0 | 0 | — | — |
| — | BB_O06 | hypothetical protein | 0 | 0 | 0 | — | — |
| — | BB_O07 | hypothetical protein | 0 | 0 | 1 | — | — |
| — | BB_O08 | hypothetical protein | 0 | 0 | 0 | — | — |
| — | BB_O09 | hypothetical protein | 0 | 0 | 0 | — | — |
| — | BB_O10 | hypothetical protein | 0 | 0 | 0 | — | — |
| — | BB_O13 | hypothetical protein | 0 | 0 | 0 | — | — |
| — | BB_O14 | hypothetical protein | 0 | 0 | 0 | — | — |
| — | BB_O15 | hypothetical protein | 0 | 0 | 0 | — | — |
| — | BB_O16 | hypothetical protein | 0 | 0 | 0 | — | — |
| — | BB_O17 | hypothetical protein | 0 | 0 | 0 | — | — |
| — | BB_O18 | hypothetical protein | 0 | 0 | 0 | — | — |
| — | BB_O19 | hypothetical protein | 0 | 0 | 0 | — | — |
| — | BB_O20 | hypothetical protein | 0 | 0 | 0 | — | — |
| — | BB_O21 | hypothetical protein | 0 | 0 | 0 | — | — |
| — | BB_O22 | hypothetical protein | 0 | 0 | 0 | — | — |
| blyA | BB_O23 | holin protein | 0 | 0 | 0 | — | — |
| blyB | BB_O24 | holin protein | 0 | 0 | 0 | — | — |
| — | BB_O25 | hypothetical protein | 0 | 0 | 0 | — | — |
| — | BB_O26 | hypothetical protein | 0 | 0 | 0 | — | — |
| bppC | BB_O38 | protein BppC | 0 | 0 | 0 | — | — |
| erpL | BB_O39 | protein ErpL | 0 | 0 | 0 | — | — |
| — | BB_O41 | hypothetical protein | 0 | 0 | 0 | — | — |
| — | BB_O42 | hypothetical protein | 0 | 0 | 0 | — | — |
| — | BB_O43 | hypothetical protein | 0 | 0 | 5 | — | — |
| — | BB_L01 | phage portal protein | 0 | 0 | 0 | — | — |
| — | BB_L02 | hypothetical protein | 0 | 0 | 0 | — | — |
| — | BB_L03 | hypothetical protein | 0 | 0 | 0 | — | — |
| — | BB_L04 | hypothetical protein | 0 | 0 | 0 | — | — |
| — | BB_L06 | hypothetical protein | 0 | 0 | 0 | — | — |
| — | BB_L07 | hypothetical protein | 0 | 0 | 0 | — | — |
| — | BB_L08 | hypothetical protein | 0 | 0 | 0 | — | — |
| — | BB_L09 | hypothetical protein | 0 | 0 | 0 | — | — |
| — | BB_L10 | hypothetical protein | 0 | 0 | 0 | — | — |
| — | BB_L11 | hypothetical protein | 0 | 0 | 0 | — | — |
| — | BB_L12 | hypothetical protein | 0 | 0 | 0 | — | — |
| — | BB_L13 | hypothetical protein | 0 | 0 | 0 | — | — |
| — | BB_L14 | hypothetical protein | 0 | 0 | 0 | — | — |
| — | BB_L15 | hypothetical protein | 0 | 0 | 0 | — | — |
| — | BB_L16 | hypothetical protein | 0 | 0 | 0 | — | — |
| — | BB_L17 | hypothetical protein | 0 | 0 | 0 | — | — |
| — | BB_L18 | hypothetical protein | 0 | 0 | 0 | — | — |
| — | BB_L21 | hypothetical protein | 0 | 0 | 0 | — | — |
| — | BB_L22 | hypothetical protein | 0 | 0 | 0 | — | — |
| blyA | BB_L23 | holin protein | 0 | 0 | 0 | — | — |
| blyB | BB_L24 | holin protein | 0 | 0 | 0 | — | — |
| mlpH | BB_L28 | lipoprotein | 0 | 0 | 1 | — | — |
| — | BB_L29 | hypothetical protein | 0 | 0 | 0 | — | — |
| — | BB_L30 | hypothetical protein | 0 | 0 | 1 | — | — |
| — | BB_L31 | hypothetical protein | 0 | 0 | 0 | — | — |
| — | BB_L32 | PF-32 protein | 0 | 0 | 0 | — | — |
| bdrO | BB_L35 | protein BdrO | 0 | 0 | 0 | — | — |
| bppA | BB_L36 | protein BppA | 0 | 0 | 0 | — | — |
| bppB | BB_L37 | protein BppB | 0 | 0 | 1 | — | — |
| — | BB_N08 | hypothetical protein | 0 | 0 | 0 | — | — |
| — | BB_N33 | PF-49 protein | 0 | 0 | 1 | — | — |
| bdrQ | BB_N34 | BdrQ | 0 | 0 | 1 | — | — |
| — | BB_U01 | hypothetical protein | 0 | 0 | 0 | — | — |
| — | BB_U02 | hypothetical protein | 0 | 0 | 2 | — | — |
| — | BB_U04 | protein BBC01 | 0 | 0 | 0 | — | — |
| — | BB_U05 | PF-32 protein | 0 | 0 | 0 | — | — |
| — | BB_U06 | plasmid partition protein | 0 | 0 | 0 | — | — |
| — | BB_U07 | protein BBC01 | 0 | 0 | 0 | — | — |
| — | BB_U08 | SUA5 subfamily protein | 0 | 0 | 0 | — | — |
| — | BB_U09 | TM2 family protein | 0 | 0 | 0 | — | — |
| — | BB_U10 | protein BBC01 | 0 | 0 | 0 | — | — |
| — | BB_U11 | Sua5/YciO/YrdC/YwlC family | 0 | 0 | 0 | — | — |
| | | protein | | | | | |
| — | BB_Q03 | outer membrane protein | 0 | 0 | 0 | — | — |
| — | BB_Q05 | antigen, P35 | 0 | 0 | 0 | — | — |
| — | BB_Q07 | plasmid partition protein | 0 | 0 | 0 | — | — |
| — | BB_Q08 | PF-32 protein | 0 | 0 | 0 | — | — |
| — | BB_Q09 | hypothetical protein | 0 | 0 | 0 | — | — |
| bdrW | BB_Q34 | protein BdrW protein | 0 | 0 | 1 | — | — |
| — | BB_Q40 | PF-32 protein | 0 | 0 | 0 | — | — |
| erpX | BB_Q47 | protein ErpX | 0 | 0 | 0 | — | — |
| — | BB_Q48 | hypothetical protein | 0 | 0 | 0 | — | — |
| — | BB_Q49 | hypothetical protein | 0 | 0 | 0 | — | — |
| — | BB_Q67 | adenine specific DNA | 0 | 0 | 0 | — | — |
| | | methyltransferase | | | | | |
| — | BB_Q89 | lipoprotein | 0 | 0 | 5 | — | — |
| — | BB_T01 | hypothetical protein | 0 | 0 | 0 | — | — |
| — | BB_T02 | hypothetical protein | 0 | 0 | 0 | — | — |
| — | BB_T03 | hypothetical protein | 0 | 0 | 0 | — | — |
| — | BB_T04 | hypothetical protein | 0 | 0 | 0 | — | — |
| — | BB_T06 | Sua5/YciO/YrdC/YwlC family | 0 | 0 | 0 | — | — |
| | | protein | | | | | |
| — | BB_T07 | outer membrane protein | 0 | 0 | 0 | — | — |
| — | BB_0138 | hypothetical protein | 0 | 0 | 0 | — | — |
| — | BB_0305 | hypothetical protein | 0 | 0 | 0 | — | — |
| — | BB_0321 | hypothetical protein | 0 | 0 | 4 | — | — |
| tRNA- | BB_t03 | Trp tRNA | 0 | 0 | 1 | — | — |
| Trp-1 | | | | | | | |
| — | BB_t19 | Ser tRNA | 0 | 0 | 7 | — | — |
| tRNA- | BB_t07 | Lys tRNA | 0 | 0 | 0 | — | — |
| Lys-2 | | | | | | | |
| tRNA- | BB_t08 | His tRNA | 0 | 0 | 0 | — | — |
| His-1 | | | | | | | |
| — | BB_t26 | Arg tRNA | 0 | 0 | 0 | — | — |
| — | BB_t27 | Leu tRNA | 0 | 0 | 2 | — | — |
| tRNA- | BB_t14 | Thr tRNA | 0 | 0 | 0 | — | — |
| Thr-2 | | | | | | | |
| tRNA- | BB_t16 | Arg tRNA | 0 | 0 | 3 | — | — |
| Arg-3 | | | | | | | |
| — | BB_E09 | protein p23 | 0 | 0 | 0 | — | — |
| — | BB_E16 | hypothetical protein | 0 | 0 | 0 | — | — |
| — | BB_E17 | hypothetical protein | 0 | 0 | 0 | — | — |
| — | BB_E18 | hypothetical protein | 0 | 0 | 0 | — | — |
| — | BB_E19 | hypothetical protein | 0 | 0 | 0 | — | — |
| — | BB_E20 | hypothetical protein | 0 | 0 | 0 | — | — |
| — | BB_E21 | hypothetical protein | 0 | 0 | 0 | — | — |
| — | BB_E22 | pyrazinamidase/nicotinamidase | 0 | 0 | 0 | — | — |
| | | PncA | | | | | |
| — | BB_E31 | antigen, P35, putative | 0 | 0 | 0 | — | — |
| — | BB_F01 | hypothetical protein | 0 | 0 | 0 | — | — |
| — | BB_F02 | hypothetical protein | 0 | 0 | 0 | — | — |
| — | BB_F06 | hypothetical protein | 0 | 0 | 0 | — | — |
| — | BB_F08 | hypothetical protein | 0 | 0 | 0 | — | — |
| — | BB_F0034 | hypothetical protein | 0 | 0 | 0 | — | — |
| — | BB_F14 | hypothetical protein | 0 | 0 | 0 | — | — |
| — | BB_F17 | putative transmembrane protein | 0 | 0 | 0 | — | — |
| — | BB_F20 | BBF20 | 0 | 0 | 0 | — | — |
| — | BB_F23 | PF49 | 0 | 0 | 0 | — | — |
| — | BB_F24 | PF32 | 0 | 0 | 0 | — | — |
| — | BB_F25 | hypothetical protein | 0 | 0 | 0 | — | — |
| — | BB_F26 | hypothetical protein | 0 | 0 | 0 | — | — |
| — | BB_F0039 | hypothetical protein | 0 | 0 | 0 | — | — |
| — | BB_F0040 | hypothetical protein | 0 | 0 | 0 | — | — |
| — | BB_F0041 | outer surface protein VlsE1 | 0 | 0 | 0 | — | — |
| — | BB_I01 | hypothetical protein | 0 | 0 | 0 | — | — |
| — | BB_I02 | hypothetical protein | 0 | 0 | 0 | — | — |
| — | BB_I06 | MTA/SAH nucleosidase | 0 | 0 | 0 | — | — |
| — | BB_I12 | hypothetical protein | 0 | 0 | 0 | — | — |
| — | BB_I15 | virulence associated lipoprotein | 0 | 0 | 0 | — | — |
| vraA | BB_I16 | repetitive antigen A | 0 | 0 | 0 | — | — |
| — | BB_I18 | hypothetical protein | 0 | 0 | 0 | — | — |
| — | BB_I19 | hypothetical protein | 0 | 0 | 0 | — | — |
| — | BB_I20 | hypothetical protein | 0 | 0 | 0 | — | — |
| — | BB_I21 | PF-32 protein | 0 | 0 | 0 | — | — |
| — | BB_I22 | PF-49 protein | 0 | 0 | 0 | — | — |
| — | BB_I26 | multidrug-efflux transporter | 0 | 0 | 0 | — | — |
| — | BB_I29 | virulence associated lipoprotein | 0 | 0 | 0 | — | — |
| — | BB_I34 | virulence associated lipoprotein | 0 | 0 | 0 | — | — |
| — | BB_I36 | antigen P35 | 0 | 0 | 0 | — | — |
| — | BB_I38 | surface antigen | 0 | 0 | 0 | — | — |
| — | BB_I41 | hypothetical protein | 0 | 0 | 0 | — | — |
| — | BB_I42 | outer membrane protein | 0 | 0 | 0 | — | — |
| — | BB_K01 | lipoprotein | 0 | 0 | 0 | — | — |
| — | BB_K07 | lipoprotein | 0 | 0 | 0 | — | — |
| — | BB_K09 | hypothetical protein | 0 | 0 | 0 | — | — |
| — | BB_K13 | hypothetical protein | 0 | 0 | 0 | — | — |
| — | BB_K15 | antigen P35 | 0 | 0 | 0 | — | — |
| — | BB_K17 | adenine deaminase | 0 | 0 | 0 | — | — |
| — | BB_K19 | lipoprotein | 0 | 0 | 0 | — | — |
| — | BB_K21 | PF-32 protein | 0 | 0 | 0 | — | — |
| — | BB_K22 | hypothetical protein | 0 | 0 | 0 | — | — |
| — | BB_K23 | hypothetical protein | 0 | 0 | 0 | — | — |
| — | BB_K24 | PF-49 protein | 0 | 0 | 0 | — | — |
| — | BB_K54 | hypothetical protein | 0 | 0 | 0 | — | — |
| — | BB_K32 | fibronectin-binding protein | 0 | 0 | 0 | — | — |
| — | BB_K33 | hypothetical protein | 0 | 0 | 0 | — | — |
| — | BB_K34 | hypothetical protein | 0 | 0 | 0 | — | — |
| — | BB_K35 | hypothetical protein | 0 | 0 | 0 | — | — |
| — | BB_K0058 | hypothetical protein | 0 | 0 | 0 | — | — |
| — | BB_K40 | hypothetical protein | 0 | 0 | 0 | — | — |
| — | BB_K41 | hypothetical protein | 0 | 0 | 0 | — | — |
| — | BB_K42 | hypothetical protein | 0 | 0 | 0 | — | — |
| — | BB_K45 | immunogenic protein P37 | 0 | 0 | 0 | — | — |
| — | BB_K47 | hypothetical protein | 0 | 0 | 0 | — | — |
| — | BB_K48 | immunogenic protein P37 | 0 | 0 | 0 | — | — |
| — | BB_K49 | hypothetical protein | 0 | 0 | 0 | — | — |
| — | BB_K50 | immunogenic protein P37 | 0 | 0 | 0 | — | — |
| — | BB_K0059 | hypothetical protein | 0 | 0 | 0 | — | — |
| — | BB_K52 | lipoprotein | 0 | 0 | 0 | — | — |
| — | BB_K53 | outer membrane protein | 0 | 0 | 0 | — | — |
| — | BB_J0058 | hypothetical protein | 0 | 0 | 0 | — | — |
| — | BB_J42 | hypothetical protein | 0 | 0 | 0 | — | — |
| — | BB_A21 | PF-49 protein | 0 | 0 | 1 | — | — |

### Example 3: RT-PCR on Upregulated and Downregulated Candidate Genes of Amoxicillin Tolerant B. burgdorferi

For amoxicillin tolerant B. burgdorferi peristers, a total of 516 genes were differentially expressed compared with drug free control, with 342 genes being upregulated and 174 genes down-regulated (Table 2). A total of 41 up-regulated genes and 45 down-regulated genes were identified by more than two fold change (FIG. 1B). The up-regulated genes were classified into membrane protein (BB_A05, BB_A36, BB_0385, BB_0767, BB_0844), bacterial chemotaxis (BB_0414, BB_0670, BB_0567), DNA repair (BB_0422, BB_0623 or mfd, BB_0344), energy production (BB_0782 or nadD), transporter (BB_0164, BB_0729), terpenoid/steroid synthesis (BB_0686, BB_0687), virulence (BB_A24, BB_A25 and BB_0766), Clp protease (BB_0757), and P450 family dehydrogenase (BB_G17). On the other hand, the majority of the down-regulated genes belonged to outer membrane proteins (most are lipoproteins), ribosome proteins and glycolysis/gluconeogenesis.

TABLE 2
Genes upregulated or downregulated in order of fold change in amoxicillin tolerant B. burgdorferi persisters

| | | | Expression | | | | |
|---|---|---|---|---|---|---|---|
| Name | Synonym | Product | Ctrl | Dox | Amo | foldDox | foldAmo |
| — | BB_Q19 | hypothetical protein | 0 | 24 | 54 | — | — |
| fliQ | BB_0274 | flagellar biosynthesis protein | 0 | 42 | 50 | — | — |
| | | FliQ | | | | | |
| fliR | BB_0273 | flagellar biosynthetic protein | 0 | 18 | 28 | — | — |
| | | FliR | | | | | |
| — | BB_N07 | hypothetical protein | 0 | 10 | 27 | — | — |
| — | BB_A49 | hypothetical protein | 0 | 18 | 23 | — | — |
| blyA | BB_R23 | pore-forming hemolysin | 0 | 29 | 18 | — | — |
| — | BB_0282 | flagellar protein FlbD | 0 | 17 | 18 | — | — |
| secE | BB_0395 | protein translocase subunit SecE | 0 | 16 | 17 | — | — |
| bppB | BB_M36 | BppB | 0 | 11 | 17 | — | — |
| — | BB_N12 | hypothetical protein | 0 | 1 | 17 | — | — |
| — | BB_G31 | hypothetical protein | 0 | 27 | 15 | — | — |
| ruvA | BB_0023 | Holliday junction DNA helicase | 0 | 12 | 15 | — | — |
| | | RuvA | | | | | |
| tRNA- | BB_t15 | Pro tRNA | 0 | 7 | 15 | — | — |
| Pro-1 | | | | | | | |
| — | BB_A33 | lipoprotein | 0 | 4 | 15 | — | — |
| — | BB_A45 | hypothetical protein | 0 | 16 | 13 | — | — |
| rplN | BB_0488 | 50S ribosomal protein L14 | 0 | 12 | 12 | — | — |
| — | BB_M02 | hypothetical protein | 0 | 3 | 12 | — | — |
| — | BB_0403 | hypothetical protein | 0 | 16 | 11 | — | — |
| — | BB_L42 | hypothetical protein | 0 | 3 | 11 | — | — |
| — | BB_0726 | ATP-binding protein | 0 | 35 | 10 | — | — |
| — | BB_0391 | 50S ribosomal protein L10 | 0 | 9 | 10 | — | — |
| gatC | BB_0343 | glutamyl-tRNA(Gln) | 0 | 5 | 10 | — | — |
| | | amidotransferase subunit C | | | | | |
| — | BB_S03 | hypothetical protein | 0 | 3 | 10 | — | — |
| tRNA- | BB_t10 | Met tRNA | 0 | 11 | 9 | — | — |
| Met-3 | | | | | | | |
| — | BB_Q26 | hypothetical protein | 0 | 8 | 9 | — | — |
| — | BB_Q06 | membrane protein | 0 | 1 | 9 | — | — |
| — | BB_0557 | phosphocarrier protein HPr | 0 | 56 | 8 | — | — |
| flgD | BB_0284 | flagellar hook capping protein | 0 | 15 | 8 | — | — |
| tRNA- | BB_t01 | Cys tRNA | 0 | 14 | 8 | — | — |
| Cys-1 | | | | | | | |
| tRNA- | BB_t02 | Phe tRNA | 0 | 8 | 8 | — | — |
| Phe-2 | | | | | | | |
| — | BB_Q25 | hypothetical protein | 0 | 7 | 8 | — | — |
| — | BB_0309 | hypothetical protein | 0 | 6 | 8 | — | — |
| — | BB_t29 | Tyr tRNA | 0 | 6 | 8 | — | — |
| — | BB_Q85 | hypothetical protein | 0 | 1 | 8 | — | — |
| — | BB_t19 | Ser tRNA | 0 | 0 | 7 | — | — |
| — | BB_A32 | hypothetical protein | 0 | 7 | 6 | — | — |
| tRNA- | BB_t09 | Gly tRNA | 0 | 6 | 6 | — | — |
| Gly-2 | | | | | | | |
| — | BB_0124 | hypothetical protein | 0 | 8 | 5 | — | — |
| — | BB_O12 | hypothetical protein | 0 | 1 | 5 | — | — |
| — | BB_M09 | hypothetical protein | 0 | 0 | 5 | — | — |
| — | BB_M16 | hypothetical protein | 0 | 0 | 5 | — | — |
| — | BB_O43 | hypothetical protein | 0 | 0 | 5 | — | — |
| — | BB_Q89 | lipoprotein | 0 | 0 | 5 | — | — |
| — | BB_0708 | hypothetical protein | 0 | 5 | 4 | — | — |
| bdrP | BB_L27 | protein BdrP | 0 | 3 | 4 | — | — |
| bppA | BB_O36 | protein BppA | 0 | 2 | 4 | — | — |
| — | BB_B13 | plasmid partition protein | 0 | 2 | 4 | — | — |
| — | BB_O01 | hypothetical protein | 0 | 0 | 4 | — | — |
| — | BB_0321 | hypothetical protein | 0 | 0 | 4 | — | — |
| — | BB_r01 | 5S ribosomal RNA | 0 | 26 | 3 | — | — |
| — | BB_0773 | hypothetical protein | 0 | 2 | 3 | — | — |
| — | BB_H40 | transposase-like protein | 0 | 2 | 3 | — | — |
| — | BB_M21 | hypothetical protein | 0 | 1 | 3 | — | — |
| tRNA- | BB_t16 | Arg tRNA | 0 | 0 | 3 | — | — |
| Arg-3 | | | | | | | |
| — | BB_P40 | hypothetical protein | 0 | 10 | 2 | — | — |
| — | BB_0234 | integral membrane protein | 0 | 9 | 2 | — | — |
| — | BB_0212 | hypothetical protein | 0 | 8 | 2 | — | — |
| — | BB_0701 | hypothetical protein | 0 | 8 | 2 | — | — |
| — | BB_0095 | hypothetical protein | 0 | 7 | 2 | — | — |
| — | BB_O30 | hypothetical protein | 0 | 5 | 2 | — | — |
| revA | BB_M27 | rev protein | 0 | 2 | 2 | — | — |
| — | BB_R34 | hypothetical protein | 0 | 1 | 2 | — | — |
| — | BB_O11 | hypothetical protein | 0 | 1 | 2 | — | — |
| — | BB_M19 | hypothetical protein | 0 | 0 | 2 | — | — |
| — | BB_U02 | hypothetical protein | 0 | 0 | 2 | — | — |
| — | BB_t27 | Leu tRNA | 0 | 0 | 2 | — | — |
| bdrN | BB_O27 | protein BdrN | 0 | 5 | 1 | — | — |
| — | BB_0731 | hypothetical protein | 0 | 4 | 1 | — | — |
| — | BB_J23 | hypothetical protein | 0 | 2 | 1 | — | — |
| bdrM | BB_O34 | protein BdrM | 0 | 1 | 1 | — | — |
| — | BB_O07 | hypothetical protein | 0 | 0 | 1 | — | — |
| mlpH | BB_L28 | lipoprotein | 0 | 0 | 1 | — | — |
| — | BB_L30 | hypothetical protein | 0 | 0 | 1 | — | — |
| bppB | BB_L37 | protein BppB | 0 | 0 | 1 | — | — |
| — | BB_N33 | PF-49 protein | 0 | 0 | 1 | — | — |
| bdrQ | BB_N34 | BdrQ | 0 | 0 | 1 | — | — |
| bdrW | BB_Q34 | protein BdrW protein | 0 | 0 | 1 | — | — |
| tRNA- | BB_t03 | Trp tRNA | 0 | 0 | 1 | — | — |
| Trp-1 | | | | | | | |
| — | BB_A21 | PF-49 protein | 0 | 0 | 1 | — | — |
| — | BB_H25 | hypothetical protein | 4 | 81 | 144 | 20.25 | 36 |
| — | BB_G28 | hypothetical protein | 5 | 1 | 67 | 0.2 | 13.4 |
| — | BB_A05 | S1 antigen | 5 | 58 | 65 | 11.6 | 13 |
| — | BB_0578 | methyl-accepting chemotaxis | 1 | 17 | 12 | 17 | 12 |
| | | protein | | | | | |
| — | BB_0059 | CBS domain-containing protein | 2 | 22 | 23 | 11 | 11.5 |
| — | BB_S04 | hypothetical protein | 2 | 18 | 23 | 9 | 11.5 |
| — | BB_G32 | replicative DNA helicase | 2 | 49 | 22 | 24.5 | 11 |
| rpmA | BB_0780 | 50S ribosomal protein L27 | 7 | 64 | 74 | 9.142857143 | 10.57142857 |
| argF | BB_0842 | ornithine carbamoyltransferase | 2 | 18 | 21 | 9 | 10.5 |
| — | BB_G17 | hypothetical protein | 5 | 21 | 45 | 4.2 | 9 |
| — | BB_A36 | lipoprotein | 7 | 49 | 59 | 7 | 8.428571429 |
| grpE | BB_0519 | protein GrpE | 3 | 52 | 25 | 17.33333333 | 8.333333333 |
| — | BB_0019 | hypothetical protein | 4 | 71 | 33 | 17.75 | 8.25 |
| ftsY | BB_0076 | signal recognition particle- | 2 | 15 | 16 | 7.5 | 8 |
| | | docking protein FtsY | | | | | |
| — | BB_G25 | hypothetical protein | 5 | 17 | 39 | 3.4 | 7.8 |
| bmpD | BB_0385 | basic membrane protein D | 6 | 59 | 46 | 9.833333333 | 7.666666667 |
| — | BB_M05 | hypothetical protein | 3 | 5 | 23 | 1.666666667 | 7.666666667 |
| — | BB_0451 | chromate transport protein | 4 | 44 | 30 | 11 | 7.5 |
| — | BB_0116 | PTS system maltose and | 9 | 68 | 67 | 7.555555556 | 7.444444444 |
| | | glucose-specific transporter | | | | | |
| | | subunit IIABC | | | | | |
| — | BB_0158 | S2 lipoprotein | 3 | 40 | 22 | 13.33333333 | 7.333333333 |
| — | BB_0091 | V-type ATP synthase subunit I | 1 | 24 | 7 | 24 | 7 |
| — | BB_Q13 | hypothetical protein | 2 | 4 | 14 | 2 | 7 |
| — | BB_0535 | hypothetical protein | 7 | 30 | 47 | 4.285714286 | 6.714285714 |
| nadD | BB_0782 | nicotinate-nucleotide | 8 | 31 | 53 | 3.875 | 6.625 |
| | | adenylyltransferase | | | | | |
| — | BB_0422 | 3-methyladenine DNA | 12 | 35 | 79 | 2.916666667 | 6.583333333 |
| | | glycosylase | | | | | |
| — | BB_Q20 | hypothetical protein | 14 | 23 | 91 | 1.642857143 | 6.5 |
| erpG | BB_S41 | outer surface protein ErpG | 3 | 19 | 19 | 6.333333333 | 6.333333333 |
| clpP | BB_0757 | ATP-dependent Clp protease | 11 | 329 | 66 | 29.90909091 | 6 |
| | | proteolytic subunit | | | | | |
| — | BB_0172 | von Willebrand factor type A | 5 | 62 | 30 | 12.4 | 6 |
| | | domain-containing protein | | | | | |
| — | BB_0414 | chemotaxis protein | 7 | 76 | 42 | 10.85714286 | 6 |
| | | methyltransferase | | | | | |
| — | BB_J28 | hypothetical protein | 3 | 17 | 18 | 5.666666667 | 6 |
| — | predicted | antisense: BB_A23 | 13946 | 12513 | 82442 | 0.897246522 | 5.911515847 |
| | RNA | | | | | | |
| — | predicted | antisense: BB_A23 | 7231 | 5123 | 41911 | 0.708477389 | 5.796017148 |
| | RNA | | | | | | |
| — | BB_0463 | nucleoside diphosphate kinase | 4 | 19 | 23 | 4.75 | 5.75 |
| — | BB_0783 | hypothetical protein | 3 | 9 | 17 | 3 | 5.666666667 |
| — | BB_A53 | Bbs27 protein | 16 | 137 | 90 | 8.5625 | 5.625 |
| — | BB_Q21 | hypothetical protein | 10 | 3 | 56 | 0.3 | 5.6 |
| — | BB_J16 | plasmid partition protein | 2 | 16 | 11 | 8 | 5.5 |
| — | BB_0670 | purine-binding chemotaxis | 8 | 57 | 44 | 7.125 | 5.5 |
| | | protein | | | | | |
| — | BB_0807 | integral membrane protein | 3 | 39 | 16 | 13 | 5.333333333 |
| — | BB_0569 | hypothetical protein | 14 | 45 | 74 | 3.214285714 | 5.285714286 |
| — | BB_0551 | protein CheY | 5 | 20 | 26 | 4 | 5.2 |
| blyA | BB_Q30 | holin protein | 2 | 10 | 10 | 5 | 5 |
| dbpA | BB_A24 | decorin-binding protein A | 558 | 505 | 2780 | 0.905017921 | 4.982078853 |
| — | BB_J48 | hypothetical protein | 9 | 47 | 44 | 5.222222222 | 4.888888889 |
| — | BB_G13 | hypothetical protein | 9 | 14 | 44 | 1.555555556 | 4.888888889 |
| — | BB_M07 | hypothetical protein | 17 | 33 | 80 | 1.941176471 | 4.705882353 |
| — | BB_0220 | alanine--tRNA ligase | 12 | 172 | 56 | 14.33333333 | 4.666666667 |
| dbpB | BB_A25 | decorin-binding protein B | 175 | 311 | 811 | 1.777142857 | 4.634285714 |
| — | BB_G21 | hypothetical protein | 12 | 9 | 55 | 0.75 | 4.583333333 |
| — | BB_G16 | hypothetical protein | 16 | 21 | 73 | 1.3125 | 4.5625 |
| — | BB_0554 | hypothetical protein | 8 | 90 | 36 | 11.25 | 4.5 |
| — | BB_G19 | hypothetical protein | 7 | 11 | 31 | 1.571428571 | 4.428571429 |
| — | BB_0054 | protein-export membrane protein | 12 | 71 | 53 | 5.916666667 | 4.416666667 |
| | | SecG | | | | | |
| — | BB_0363 | hypothetical protein | 12 | 42 | 53 | 3.5 | 4.416666667 |
| — | BB_G26 | hypothetical protein | 5 | 9 | 22 | 1.8 | 4.4 |
| — | BB_0448 | phosphocarrier protein HPr | 8 | 59 | 35 | 7.375 | 4.375 |
| fliI | BB_0288 | flagellum- specific ATP synthase | 8 | 29 | 35 | 3.625 | 4.375 |
| | | FliI | | | | | |
| — | BB_0668 | flagellar filament outer layer | 8 | 29 | 35 | 3.625 | 4.375 |
| | | protein | | | | | |
| lepB | BB_0030 | signal peptidase I | 14 | 57 | 61 | 4.071428571 | 4.357142857 |
| — | BB_0826 | hypothetical protein | 3 | 7 | 13 | 2.333333333 | 4.333333333 |
| bppA | BB_Q43 | protein BppA | 3 | 6 | 13 | 2 | 4.333333333 |
| — | BB_G15 | hypothetical protein | 22 | 41 | 95 | 1.863636364 | 4.318181818 |
| murG | BB_0767 | UDP-N-acetylglucosamine--N- | 21 | 72 | 90 | 3.428571429 | 4.285714286 |
| | | acetylmuramyl-(pentapeptide) | | | | | |
| | | pyrophosphoryl-undecaprenol | | | | | |
| | | N-acetylglucosamine transferase | | | | | |
| — | BB_0563 | hypothetical protein | 15 | 167 | 64 | 11.13333333 | 4.266666667 |
| mvaD | BB_0686 | diphosphomevalonate | 24 | 94 | 101 | 3.916666667 | 4.208333333 |
| | | decarboxylase | | | | | |
| — | BB_0164 | K+-dependent Na+/Ca+ | 15 | 126 | 63 | 8.4 | 4.2 |
| | | exchanger-like protein | | | | | |
| — | BB_0844 | lipoprotein | 41 | 107 | 171 | 2.609756098 | 4.170731707 |
| — | BB_G08 | stage 0 sporulation protein | 21 | 25 | 87 | 1.19047619 | 4.142857143 |
| | | SpoOJ | | | | | |
| — | BB_0662 | hypothetical protein | 30 | 140 | 121 | 4.666666667 | 4.033333333 |
| — | BB_0427 | rRNA small subunit | 2 | 9 | 8 | 4.5 | 4 |
| | | methyltransferase I | | | | | |
| cmk | BB_0128 | cytidylate kinase | 22 | 48 | 88 | 2.181818182 | 4 |
| — | BB_0671 | chemotaxis protein CheX | 24 | 134 | 95 | 5.583333333 | 3.958333333 |
| — | BB_0687 | phosphomevalonate kinase | 68 | 154 | 268 | 2.264705882 | 3.941176471 |
| — | BB_P41 | hypothetical protein | 15 | 17 | 59 | 1.133333333 | 3.933333333 |
| — | BB_G02 | hypothetical protein | 53 | 56 | 207 | 1.056603774 | 3.905660377 |
| — | BB_0432 | hypothetical protein | 9 | 57 | 35 | 6.333333333 | 3.888888889 |
| — | BB_B03 | telomere resolvase ResT | 96 | 320 | 371 | 3.333333333 | 3.864583333 |
| — | BB_0016 | GlpE protein | 6 | 46 | 23 | 7.666666667 | 3.833333333 |
| — | BB_J29 | hypothetical protein | 6 | 25 | 23 | 4.166666667 | 3.833333333 |
| — | BB_0130 | hypothetical protein | 5 | 12 | 19 | 2.4 | 3.8 |
| — | BB_J25 | hypothetical protein | 4 | 22 | 15 | 5.5 | 3.75 |
| bppA | BB_N35 | protein BppA | 4 | 5 | 15 | 1.25 | 3.75 |
| — | BB_0763 | acetoacetate metabolism | 347 | 609 | 1293 | 1.755043228 | 3.726224784 |
| | | regulatory protein AtoC | | | | | |
| — | BB_0760 | protein Gp37 | 86 | 225 | 320 | 2.61627907 | 3.720930233 |
| — | BB_0104 | periplasmic serine protease DO | 7 | 55 | 26 | 7.857142857 | 3.714285714 |
| — | BB_0146 | glycine/betaine ABC transporter | 10 | 30 | 37 | 3 | 3.7 |
| | | ATP-binding protein | | | | | |
| — | BB_A51 | hypothetical protein | 16 | 74 | 59 | 4.625 | 3.6875 |
| rpsE | BB_0495 | 30S ribosomal protein S5 | 6 | 40 | 22 | 6.666666667 | 3.666666667 |
| — | BB_0126 | hypothetical protein | 3 | 11 | 11 | 3.666666667 | 3.666666667 |
| trpS | BB_0005 | tryptophan--tRNA ligase | 12 | 40 | 44 | 3.333333333 | 3.666666667 |
| — | BB_0661 | hypothetical protein | 25 | 227 | 91 | 9.08 | 3.64 |
| mfd | BB_0623 | transcription-repair coupling | 25 | 92 | 91 | 3.68 | 3.64 |
| | | factor | | | | | |
| — | BB_0742 | ABC transporter ATP-binding | 10 | 37 | 36 | 3.7 | 3.6 |
| | | protein | | | | | |
| — | BB_0567 | chemotaxis histidine kinase | 16 | 49 | 57 | 3.0625 | 3.5625 |
| — | predicted | antisense: hisS | 7531 | 26181 | 26631 | 3.476430753 | 3.536183774 |
| | RNA | | | | | | |
| — | predicted | antisense: BB_A40 | 10427 | 4515 | 36844 | 0.433010454 | 3.533518749 |
| | RNA | | | | | | |
| tpiA | BB_0055 | triosephosphate isomerase | 17 | 94 | 60 | 5.529411765 | 3.529411765 |
| — | BB_0319 | hypothetical protein | 6 | 21 | 21 | 3.5 | 3.5 |
| — | BB_0344 | DNA helicase | 22 | 63 | 77 | 2.863636364 | 3.5 |
| — | BB_J26 | ABC transporter ATP-binding | 12 | 30 | 42 | 2.5 | 3.5 |
| | | protein | | | | | |
| — | BB_0066 | hypothetical protein | 4 | 8 | 14 | 2 | 3.5 |
| — | BB_P04 | hypothetical protein | 22 | 32 | 77 | 1.454545455 | 3.5 |
| rplT | BB_0188 | 50S ribosomal protein L20 | 38 | 168 | 132 | 4.421052632 | 3.473684211 |
| — | predicted | antisense: BB_A40 | 12605 | 4776 | 43757 | 0.378897263 | 3.471400238 |
| | RNA | | | | | | |
| — | predicted | antisense: grpE | 9716 | 34301 | 33514 | 3.530362289 | 3.449361877 |
| | RNA | | | | | | |
| — | BB_H37 | lipoprotein | 9 | 33 | 31 | 3.666666667 | 3.444444444 |
| — | BB_A48 | hypothetical protein | 9 | 23 | 31 | 2.555555556 | 3.444444444 |
| — | BB_0249 | phosphatidylcholine synthase | 142 | 349 | 488 | 2.457746479 | 3.436619718 |
| — | predicted | — | 26791 | 34065 | 91735 | 1.271509089 | 3.424097645 |
| | RNA | | | | | | |
| — | BB_R03 | hypothetical protein | 19 | 34 | 64 | 1.789473684 | 3.368421053 |
| — | BB_M04 | hypothetical protein | 11 | 18 | 37 | 1.636363636 | 3.363636364 |
| rpoS | BB_0771 | RNA polymerase sigma factor | 25 | 70 | 84 | 2.8 | 3.36 |
| — | BB_0679 | ribose/galactose ABC | 31 | 111 | 104 | 3.580645161 | 3.35483871 |
| | | transporter permease | | | | | |
| — | BB_G23 | hypothetical protein | 37 | 28 | 124 | 0.756756757 | 3.351351351 |
| — | BB_0536 | zinc protease | 242 | 479 | 809 | 1.979338843 | 3.342975207 |
| — | BB_0729 | dicarboxylate/amino acid: cation | 54 | 258 | 179 | 4.777777778 | 3.314814815 |
| | | symporter | | | | | |
| malQ | BB_0166 | 4-alpha-glucanotransferase | 16 | 47 | 53 | 2.9375 | 3.3125 |
| cvpA | BB_0766 | colicin V production protein | 82 | 121 | 271 | 1.475609756 | 3.304878049 |
| — | BB_P15 | hypothetical protein | 139 | 178 | 459 | 1.28057554 | 3.302158273 |
| def | BB_0065 | peptide deformylase | 17 | 41 | 56 | 2.411764706 | 3.294117647 |
| — | BB_0830 | exonuclease SbcC | 7 | 60 | 23 | 8.571428571 | 3.285714286 |
| — | BB_0227 | hypothetical protein | 7 | 39 | 23 | 5.571428571 | 3.285714286 |
| ychF | BB_0235 | ribosome-binding ATPase | 4 | 10 | 13 | 2.5 | 3.25 |
| p83/100 | BB_0744 | p83/100 antigen | 51 | 111 | 165 | 2.176470588 | 3.235294118 |
| — | BB_0712 | RNA polymerase sigma factor | 9 | 39 | 29 | 4.333333333 | 3.222222222 |
| | | RpoD | | | | | |
| — | BB_G20 | hypothetical protein | 6 | 12 | 19 | 2 | 3.166666667 |
| — | BB_0323 | hypothetical protein | 31 | 100 | 98 | 3.225806452 | 3.161290323 |
| — | BB_0058 | hypothetical protein | 7 | 16 | 22 | 2.285714286 | 3.142857143 |
| — | BB_G14 | hypothetical protein | 14 | 25 | 44 | 1.785714286 | 3.142857143 |
| — | BB_0713 | hypothetical protein | 52 | 66 | 163 | 1.269230769 | 3.134615385 |
| — | BB_D0031 | hypothetical protein | 23 | 59 | 72 | 2.565217391 | 3.130434783 |
| — | BB_0769 | tRNA N6-adenosine | 54 | 119 | 169 | 2.203703704 | 3.12962963 |
| | | threonylcarbamoyltransferase | | | | | |
| — | predicted | antisense: BB_P30 | 15254 | 28835 | 47546 | 1.890323849 | 3.11695293 |
| | RNA | | | | | | |
| — | BB_0718 | penicillin-binding protein | 9 | 40 | 28 | 4.444444444 | 3.111111111 |
| efp | BB_0214 | elongation factor P | 372 | 1154 | 1154 | 3.102150538 | 3.102150538 |
| — | predicted | antisense: BB_A38 | 13552 | 3189 | 41983 | 0.235315821 | 3.097919126 |
| | RNA | | | | | | |
| — | BB_P12 | hypothetical protein | 82 | 82 | 254 | 1 | 3.097560976 |
| fliH | BB_0289 | flagellar assembly protein FliH | 29 | 38 | 89 | 1.310344828 | 3.068965517 |
| — | BB_J31 | hypothetical protein | 16 | 60 | 49 | 3.75 | 3.0625 |
| — | BB_0765 | hypothetical protein | 36 | 61 | 109 | 1.694444444 | 3.027777778 |
| — | BB_0818 | hypothetical protein | 2 | 22 | 6 | 11 | 3 |
| bmpB | BB_0382 | basic membrane protein B | 6 | 42 | 18 | 7 | 3 |
| — | BB_0097 | hypothetical protein | 5 | 31 | 15 | 6.2 | 3 |
| — | BB_0464 | hypothetical protein | 4 | 19 | 12 | 4.75 | 3 |
| — | BB_0852 | hypothetical protein | 1 | 4 | 3 | 4 | 3 |
| — | BB_0077 | hypothetical protein | 3 | 8 | 9 | 2.666666667 | 3 |
| rsgA | BB_0099 | ribosome biogenesis GTPase | 4 | 10 | 12 | 2.5 | 3 |
| | | RsgA | | | | | |
| gidB | BB_0177 | rRNA small subunit | 3 | 7 | 9 | 2.333333333 | 3 |
| | | methyltransferase G | | | | | |
| — | BB_0421 | haloacid dehalogenase-like | 8 | 14 | 24 | 1.75 | 3 |
| | | hydrolase | | | | | |
| bppA | BB_R36 | BppA | 9 | 14 | 27 | 1.555555556 | 3 |
| — | BB_N25 | hypothetical protein | 6 | 9 | 18 | 1.5 | 3 |
| — | BB_S01 | phage portal protein | 5 | 4 | 15 | 0.8 | 3 |
| — | BB_G27 | hypothetical protein | 6 | 3 | 18 | 0.5 | 3 |
| — | BB_0200 | D-alanine--D-alanine ligase | 27 | 207 | 80 | 7.666666667 | 2.962962963 |
| valS | BB_0738 | valine--tRNA ligase | 27 | 153 | 80 | 5.666666667 | 2.962962963 |
| — | predicted | antisense: BB_B10 | 20385 | 38903 | 60178 | 1.908413049 | 2.952072602 |
| | RNA | | | | | | |
| — | BB_0183 | flagellar assembly protein FliW | 68 | 200 | 200 | 2.941176471 | 2.941176471 |
| — | BB_0195 | hypothetical protein | 45 | 111 | 132 | 2.6 | 2.933333333 |
| — | BB_B14 | hypothetical protein | 233 | 477 | 681 | 2.0472103 | 2.922746781 |
| — | BB_0808 | hypothetical protein | 12 | 67 | 35 | 5.583333333 | 2.916666667 |
| — | BB_0835 | phosphomannomutase | 11 | 10 | 32 | 0.909090909 | 2.909090909 |
| — | BB_0627 | aminopeptidase | 188 | 430 | 545 | 2.287234043 | 2.89893617 |
| — | BB_0555 | hypothetical protein | 39 | 108 | 113 | 2.769230769 | 2.897435897 |
| — | BB_A0078 | lipoprotein | 9 | 17 | 26 | 1.888888889 | 2.888888889 |
| coaE | BB_0547 | dephospho-CoA kinase | 16 | 55 | 46 | 3.4375 | 2.875 |
| — | BB_0754 | ABC transporter ATP-binding | 16 | 49 | 46 | 3.0625 | 2.875 |
| | | protein | | | | | |
| — | BB_0725 | lectin | 8 | 20 | 23 | 2.5 | 2.875 |
| bppA | BB_S38 | protein BppA | 16 | 22 | 46 | 1.375 | 2.875 |
| — | BB_0270 | flagellar biosynthesis regulator | 22 | 41 | 63 | 1.863636364 | 2.863636364 |
| | | FlhF | | | | | |
| — | BB_A34 | extracellular solute-binding | 138 | 131 | 395 | 0.949275362 | 2.862318841 |
| | | protein, family 5 | | | | | |
| — | BB_0171 | hypothetical protein | 7 | 66 | 20 | 9.428571429 | 2.857142857 |
| — | BB_0266 | hypothetical protein | 7 | 32 | 20 | 4.571428571 | 2.857142857 |
| — | BB_B19 | outer surface protein C | 346 | 650 | 982 | 1.878612717 | 2.838150289 |
| — | BB_0083 | hypothetical protein | 6 | 56 | 17 | 9.333333333 | 2.833333333 |
| — | BB_0036 | DNA topoisomerase IV subunit | 6 | 28 | 17 | 4.666666667 | 2.833333333 |
| | | B | | | | | |
| — | BB_0796 | hypothetical protein | 12 | 19 | 34 | 1.583333333 | 2.833333333 |
| — | BB_R19 | hypothetical protein | 17 | 9 | 48 | 0.529411765 | 2.823529412 |
| — | BB_0173 | hypothetical protein | 20 | 79 | 56 | 3.95 | 2.8 |
| — | BB_A38 | phage portal protein | 5 | 10 | 14 | 2 | 2.8 |
| — | BB_0141 | membrane fusion protein | 29 | 41 | 81 | 1.413793103 | 2.793103448 |
| trxB | BB_0515 | thioredoxin reductase | 43 | 210 | 120 | 4.88372093 | 2.790697674 |
| — | predicted | antisense: hisS | 11688 | 22464 | 32504 | 1.921971253 | 2.780971937 |
| | RNA | | | | | | |
| — | BB_0748 | hypothetical protein | 9 | 56 | 25 | 6.222222222 | 2.777777778 |
| — | BB_0693 | xylose operon regulatory protein | 9 | 19 | 25 | 2.111111111 | 2.777777778 |
| — | BB_0764 | sensory transduction histidine | 57 | 92 | 157 | 1.614035088 | 2.754385965 |
| | | kinase | | | | | |
| — | BB_0176 | ATPase | 32 | 97 | 88 | 3.03125 | 2.75 |
| cspA | BB_A68 | complement regulator-acquiring | 8 | 21 | 22 | 2.625 | 2.75 |
| | | surface protein 1 | | | | | |
| — | BB_0608 | aminoacyl-histidine dipeptidase | 12 | 27 | 33 | 2.25 | 2.75 |
| — | BB_G09 | hypothetical protein | 59 | 58 | 161 | 0.983050847 | 2.728813559 |
| ligA | BB_0552 | DNA ligase | 25 | 221 | 68 | 8.84 | 2.72 |
| — | BB_0566 | hypothetical protein | 14 | 31 | 38 | 2.214285714 | 2.714285714 |
| — | BB_0093 | V-type ATP synthase subunit B | 10 | 28 | 27 | 2.8 | 2.7 |
| — | BB_0743 | hypothetical protein | 20 | 37 | 54 | 1.85 | 2.7 |
| uvrA | BB_0837 | excinuclease ABC subunit A | 13 | 27 | 35 | 2.076923077 | 2.692307692 |
| — | BB_Q22 | hypothetical protein | 13 | 15 | 35 | 1.153846154 | 2.692307692 |
| — | BB_Q52 | hypothetical protein | 9 | 2 | 24 | 0.222222222 | 2.666666667 |
| — | BB_P13 | hypothetical protein | 112 | 108 | 298 | 0.964285714 | 2.660714286 |
| xth | BB_0534 | exodeoxyribonuclease III | 68 | 199 | 180 | 2.926470588 | 2.647058824 |
| — | BB_B12 | PF-32 protein | 14 | 38 | 37 | 2.714285714 | 2.642857143 |
| — | BB_0035 | DNA topoisomerase IV subunit | 8 | 43 | 21 | 5.375 | 2.625 |
| | | A | | | | | |
| — | BB_0838 | hypothetical protein | 24 | 33 | 63 | 1.375 | 2.625 |
| rpoZ | BB_0820 | DNA-directed RNA polymerase | 10 | 71 | 26 | 7.1 | 2.6 |
| | | subunit omega | | | | | |
| — | BB_0069 | aminopeptidase II | 10 | 35 | 26 | 3.5 | 2.6 |
| — | BB_0827 | ATP-dependent helicase | 39 | 77 | 101 | 1.974358974 | 2.58974359 |
| mlpA | BB_P28 | surface lipoprotein | 40 | 71 | 103 | 1.775 | 2.575 |
| — | predicted | antisense: BB_S34 | 12380 | 22283 | 31741 | 1.799919225 | 2.563893376 |
| | RNA | | | | | | |
| — | BB_0139 | hypothetical protein | 34 | 70 | 87 | 2.058823529 | 2.558823529 |
| — | BB_0009 | hypothetical protein | 11 | 22 | 28 | 2 | 2.545454545 |
| — | BB_0640 | spermidine/putrescine ABC | 13 | 25 | 33 | 1.923076923 | 2.538461538 |
| | | transporter permease | | | | | |
| — | predicted | antisense: dnaK | 25407 | 22781 | 64444 | 0.896642658 | 2.536466328 |
| | RNA | | | | | | |
| — | BB_0785 | septation protein SpoVG | 15 | 71 | 38 | 4.733333333 | 2.533333333 |
| — | BB_0333 | peptide ABC transporter | 136 | 256 | 343 | 1.882352941 | 2.522058824 |
| | | permease | | | | | |
| secD | BB_0652 | protein translocase subunit SecD | 20 | 107 | 50 | 5.35 | 2.5 |
| glyA | BB_0601 | serine hydroxymethyltransferase | 10 | 53 | 25 | 5.3 | 2.5 |
| — | BB_0468 | hypothetical protein | 12 | 45 | 30 | 3.75 | 2.5 |
| bapA | BB_S42 | BapA protein | 4 | 5 | 10 | 1.25 | 2.5 |
| — | BB_N04 | hypothetical protein | 6 | 7 | 15 | 1.166666667 | 2.5 |
| — | BB_N42 | hypothetical protein | 4 | 3 | 10 | 0.75 | 2.5 |
| — | predicted | antisense: BB_0712 | 12262 | 25192 | 30600 | 2.054477247 | 2.495514598 |
| | RNA | | | | | | |
| — | BB_0165 | hypothetical protein | 35 | 120 | 87 | 3.428571429 | 2.485714286 |
| — | BB_0606 | chemoreceptor glutamine | 40 | 150 | 99 | 3.75 | 2.475 |
| | | deamidase CheD | | | | | |
| — | BB_0340 | hypothetical protein | 19 | 114 | 47 | 6 | 2.473684211 |
| — | predicted | antisense: dnaK | 16301 | 20898 | 40272 | 1.282007239 | 2.470523281 |
| | RNA | | | | | | |
| — | predicted | antisense: BB_A38 | 16406 | 4088 | 40494 | 0.24917713 | 2.468243326 |
| | RNA | | | | | | |
| — | BB_0002 | hypothetical protein | 15 | 35 | 37 | 2.333333333 | 2.466666667 |
| polA | BB_0548 | DNA polymerase I | 20 | 44 | 49 | 2.2 | 2.45 |
| tyrS | BB_0370 | tyrosine--tRNA ligase | 9 | 48 | 22 | 5.333333333 | 2.444444444 |
| — | BB_0678 | sugar ABC transporter permease | 9 | 29 | 22 | 3.222222222 | 2.444444444 |
| — | BB_A23 | hypothetical protein | 16 | 40 | 39 | 2.5 | 2.4375 |
| rnz | BB_0755 | ribonuclease Z | 23 | 60 | 56 | 2.608695652 | 2.434782609 |
| fmt | BB_0064 | methionyl-tRNA | 53 | 94 | 129 | 1.773584906 | 2.433962264 |
| | | formyltransferase | | | | | |
| mvk | BB_0688 | mevalonate kinase | 1001 | 1560 | 2436 | 1.558441558 | 2.433566434 |
| — | BB_0267 | hypothetical protein | 7 | 34 | 17 | 4.857142857 | 2.428571429 |
| — | BB_0197 | release factor glutamine | 7 | 31 | 17 | 4.428571429 | 2.428571429 |
| | | methyltransferase | | | | | |
| — | BB_0564 | hypothetical protein | 7 | 25 | 17 | 3.571428571 | 2.428571429 |
| — | BB_0800 | transcription | 21 | 29 | 51 | 1.380952381 | 2.428571429 |
| | | termination/antitermination | | | | | |
| | | protein NusA | | | | | |
| rpsR | BB_0113 | 30S ribosomal protein S18 | 12 | 36 | 29 | 3 | 2.416666667 |
| — | BB_G18 | hypothetical protein | 29 | 36 | 70 | 1.24137931 | 2.413793103 |
| — | BB_0201 | UDP-N-acetylmuramyl- | 20 | 141 | 48 | 7.05 | 2.4 |
| | | tripeptide synthetase | | | | | |
| acpP | BB_0704 | acyl carrier protein | 10 | 53 | 24 | 5.3 | 2.4 |
| lysS | BB_0659 | lysine--tRNA ligase | 15 | 67 | 36 | 4.466666667 | 2.4 |
| uvrB | BB_0836 | excinuclease ABC subunit B | 5 | 11 | 12 | 2.2 | 2.4 |
| — | BB_D15 | membrane protein | 5 | 6 | 12 | 1.2 | 2.4 |
| — | BB_0681 | methyl-accepting chemotaxis | 52 | 162 | 124 | 3.115384615 | 2.384615385 |
| | | protein | | | | | |
| — | BB_0504 | ribonuclease Y | 13 | 37 | 31 | 2.846153846 | 2.384615385 |
| — | BB_0775 | flagellar hook-basal body | 13 | 33 | 31 | 2.538461538 | 2.384615385 |
| | | complex protein | | | | | |
| lon | BB_0613 | ATP-dependent protease La | 53 | 220 | 126 | 4.150943396 | 2.377358491 |
| — | BB_M08 | hypothetical protein | 8 | 6 | 19 | 0.75 | 2.375 |
| — | BB_0811 | hypothetical protein | 33 | 86 | 78 | 2.606060606 | 2.363636364 |
| thrS | BB_0720 | threonine--tRNA ligase | 14 | 30 | 33 | 2.142857143 | 2.357142857 |
| — | BB_0276 | flagellar biosynthesis protein | 17 | 19 | 40 | 1.117647059 | 2.352941176 |
| | | FliZ | | | | | |
| — | BB_0175 | hypothetical protein | 148 | 361 | 347 | 2.439189189 | 2.344594595 |
| — | BB_A37 | hypothetical protein | 114 | 60 | 267 | 0.526315789 | 2.342105263 |
| — | BB_0644 | N-acetylmannosamine-6- | 12 | 70 | 28 | 5.833333333 | 2.333333333 |
| | | phosphate 2-epimerase | | | | | |
| — | BB_0539 | hypothetical protein | 9 | 40 | 21 | 4.444444444 | 2.333333333 |
| — | BB_0277 | flagellar motor switch protein | 12 | 44 | 28 | 3.666666667 | 2.333333333 |
| | | FliN | | | | | |
| — | BB_0461 | DNA polymerase III subunits | 12 | 26 | 28 | 2.166666667 | 2.333333333 |
| | | gamma and tau | | | | | |
| — | BB_0298 | hypothetical protein | 3 | 6 | 7 | 2 | 2.333333333 |
| dnaA | BB_0437 | chromosomal replication | 96 | 76 | 224 | 0.791666667 | 2.333333333 |
| | | initiation protein | | | | | |
| — | BB_0221 | flagellar motor switch protein | 53 | 64 | 123 | 1.20754717 | 2.320754717 |
| — | BB_Q14 | hypothetical protein | 19 | 26 | 44 | 1.368421053 | 2.315789474 |
| — | BB_A20 | PF-32 protein | 16 | 11 | 37 | 0.6875 | 2.3125 |
| gyrA | BB_0435 | DNA gyrase subunit A | 42 | 106 | 97 | 2.523809524 | 2.30952381 |
| hslU | BB_0295 | ATP-dependent protease | 13 | 15 | 30 | 1.153846154 | 2.307692308 |
| | | ATPase subunit HslU | | | | | |
| — | BB_0604 | L-lactate permease | 111 | 250 | 256 | 2.252252252 | 2.306306306 |
| — | BB_N15 | hypothetical protein | 30 | 29 | 69 | 0.966666667 | 2.3 |
| flgG | BB_0774 | flagellar basal body rod protein | 24 | 38 | 55 | 1.583333333 | 2.291666667 |
| | | FlgG | | | | | |
| — | BB_0245 | hypothetical protein | 42 | 117 | 96 | 2.785714286 | 2.285714286 |
| ftsZ | BB_0299 | cell division protein FtsZ | 7 | 11 | 16 | 1.571428571 | 2.285714286 |
| — | BB_H02 | hypothetical protein | 91 | 50 | 208 | 0.549450549 | 2.285714286 |
| — | predicted | antisense: BB_A38 | 19570 | 4607 | 44617 | 0.235411344 | 2.279867144 |
| | RNA | | | | | | |
| lgt | BB_0362 | prolipoprotein diacylglyceryl | 61 | 135 | 139 | 2.213114754 | 2.278688525 |
| | | transferase | | | | | |
| — | BB_0794 | hypothetical protein | 33 | 38 | 75 | 1.151515152 | 2.272727273 |
| flhB | BB_0272 | flagellar biosynthesis protein | 26 | 60 | 59 | 2.307692308 | 2.269230769 |
| | | FlhB | | | | | |
| — | BB_0556 | hypothetical protein | 23 | 47 | 52 | 2.043478261 | 2.260869565 |
| — | predicted | antisense: tig | 13666 | 28118 | 30788 | 2.057515001 | 2.252890385 |
| | RNA | | | | | | |
| — | BB_0136 | penicillin-binding protein | 214 | 391 | 482 | 1.827102804 | 2.252336449 |
| — | BB_0114 | single-stranded DNA-binding | 4 | 21 | 9 | 5.25 | 2.25 |
| | | protein | | | | | |
| fni | BB_0684 | isopentenyl-diphosphate delta- | 40 | 104 | 90 | 2.6 | 2.25 |
| | | isomerase | | | | | |
| — | BB_0667 | hypothetical protein | 4 | 9 | 9 | 2.25 | 2.25 |
| — | BB_0304 | UDP-N-acetylmuramoyl- | 20 | 34 | 45 | 1.7 | 2.25 |
| | | tripeptide--D-alanyl-D-alanine | | | | | |
| | | ligas | | | | | |
| apt | BB_0777 | adenine | 16 | 20 | 36 | 1.25 | 2.25 |
| | | phosphoribosyltransferase | | | | | |
| — | BB_0358 | rRNA small subunit | 21 | 56 | 47 | 2.666666667 | 2.238095238 |
| | | methyltransferase E | | | | | |
| — | predicted | antisense: BB_0779 | 25448 | 52903 | 56940 | 2.078866709 | 2.23750393 |
| | RNA | | | | | | |
| — | BB_0537 | hypothetical protein | 17 | 34 | 38 | 2 | 2.235294118 |
| — | BB_0685 | 3-hydroxy-3-methylglutaryl- | 34 | 62 | 76 | 1.823529412 | 2.235294118 |
| | | CoA reductase | | | | | |
| — | BB_G12 | hypothetical protein | 154 | 102 | 343 | 0.662337662 | 2.227272727 |
| — | BB_0329 | ABC transporter substrate- | 18 | 54 | 40 | 3 | 2.222222222 |
| | | binding protein | | | | | |
| — | BB_0696 | hypothetical protein | 18 | 30 | 40 | 1.666666667 | 2.222222222 |
| — | BB_G22 | hypothetical protein | 36 | 9 | 80 | 0.25 | 2.222222222 |
| — | BB_G24 | hypothetical protein | 32 | 13 | 71 | 0.40625 | 2.21875 |
| — | predicted | antisense: BB_0712 | 18479 | 17402 | 40972 | 0.941717625 | 2.217219547 |
| | RNA | | | | | | |
| — | BB_0528 | aldose reductase | 28 | 70 | 62 | 2.5 | 2.214285714 |
| — | predicted | antisense: dnaK | 16302 | 10814 | 36078 | 0.66335419 | 2.213102687 |
| | RNA | | | | | | |
| — | BB_0157 | hypothetical protein | 5 | 24 | 11 | 4.8 | 2.2 |
| bppA | BB_M35 | BppA | 5 | 7 | 11 | 1.4 | 2.2 |
| — | BB_0405 | hypothetical protein | 36 | 179 | 79 | 4.972222222 | 2.194444444 |
| — | BB_A40 | hypothetical protein | 21 | 25 | 46 | 1.19047619 | 2.19047619 |
| — | BB_0513 | phenylalanine--tRNA ligase | 11 | 63 | 24 | 5.727272727 | 2.181818182 |
| | | subunit alpha | | | | | |
| — | BB_0752 | hypothetical protein | 50 | 134 | 109 | 2.68 | 2.18 |
| — | BB_S15 | hypothetical protein | 58 | 53 | 126 | 0.913793103 | 2.172413793 |
| mlpC | BB_S30 | lipoprotein | 35 | 59 | 76 | 1.685714286 | 2.171428571 |
| trmE | BB_0179 | tRNA modification GTPase | 12 | 29 | 26 | 2.416666667 | 2.166666667 |
| fliE | BB_0292 | flagellar hook-basal body | 6 | 13 | 13 | 2.166666667 | 2.166666667 |
| | | protein FliE | | | | | |
| — | BB_0524 | inositol monophosphatase | 166 | 319 | 359 | 1.921686747 | 2.162650602 |
| — | BB_0680 | methyl-accepting chemotaxis | 35 | 104 | 75 | 2.971428571 | 2.142857143 |
| | | protein | | | | | |
| — | BB_0533 | protein PhnP | 50 | 139 | 107 | 2.78 | 2.14 |
| — | BB_P06 | hypothetical protein | 79 | 65 | 169 | 0.82278481 | 2.139240506 |
| — | BB_A07 | ChpAI protein | 23 | 49 | 49 | 2.130434783 | 2.130434783 |
| — | BB_0043 | hypothetical protein | 8 | 15 | 17 | 1.875 | 2.125 |
| — | BB_R12 | hypothetical protein | 16 | 17 | 34 | 1.0625 | 2.125 |
| — | BB_0526 | hypothetical protein | 25 | 85 | 53 | 3.4 | 2.12 |
| — | predicted | antisense: BB_0786 | 46137 | 87099 | 97583 | 1.887834059 | 2.115070334 |
| | RNA | | | | | | |
| hflC | BB_0204 | protein HflC | 47 | 83 | 99 | 1.765957447 | 2.106382979 |
| — | BB_0209 | hypothetical protein | 38 | 56 | 80 | 1.473684211 | 2.105263158 |
| dnaJ | BB_0517 | chaperone protein DnaJ | 30 | 166 | 63 | 5.533333333 | 2.1 |
| — | BB_0400 | hypothetical protein | 20 | 55 | 42 | 2.75 | 2.1 |
| flhA | BB_0271 | flagellar biosynthesis protein | 10 | 21 | 21 | 2.1 | 2.1 |
| | | FlhA | | | | | |
| — | BB_0579 | DNA polymerase III subunit | 21 | 70 | 44 | 3.333333333 | 2.095238095 |
| | | alpha | | | | | |
| — | BB_0430 | hypothetical protein | 11 | 26 | 23 | 2.363636364 | 2.090909091 |
| — | BB_0455 | hypothetical protein | 11 | 26 | 23 | 2.363636364 | 2.090909091 |
| pyk | BB_0348 | pyruvate kinase | 72 | 207 | 150 | 2.875 | 2.083333333 |
| — | BB_0326 | hypothetical protein | 27 | 112 | 56 | 4.148148148 | 2.074074074 |
| — | predicted | antisense: dnaK | 13207 | 24137 | 27386 | 1.827591429 | 2.073597335 |
| | RNA | | | | | | |
| — | BB_0447 | Na+/H+ antiporter | 68 | 180 | 141 | 2.647058824 | 2.073529412 |
| — | BB_H17 | hypothetical protein | 43 | 18 | 89 | 0.418604651 | 2.069767442 |
| — | predicted | antisense: BB_0712 | 18566 | 19564 | 38381 | 1.053754174 | 2.067273511 |
| | RNA | | | | | | |
| — | BB_0222 | 6-phosphogluconolactonase | 36 | 98 | 74 | 2.722222222 | 2.055555556 |
| mrdB | BB_0719 | rod shape-determining protein | 19 | 73 | 39 | 3.842105263 | 2.052631579 |
| | | RodA | | | | | |
| — | BB_0454 | lipopolysaccharide biosynthesis- | 39 | 33 | 80 | 0.846153846 | 2.051282051 |
| | | like protein | | | | | |
| — | BB_0408 | PTS system fructose-specific | 20 | 59 | 41 | 2.95 | 2.05 |
| | | transproter subunit IIABC | | | | | |
| — | predicted | — | 13193 | 23291 | 27045 | 1.765405897 | 2.049950731 |
| | RNA | | | | | | |
| — | BB_0334 | peptide ABC transporter ATP- | 538 | 768 | 1101 | 1.427509294 | 2.046468401 |
| | | binding protein | | | | | |
| — | predicted | antisense: BB_0715 | 20895 | 29754 | 42674 | 1.423977028 | 2.042306772 |
| | RNA | | | | | | |
| — | BB_0082 | hypothetical protein | 24 | 93 | 49 | 3.875 | 2.041666667 |
| fliP | BB_0275 | flagellar biosynthesis protein | 52 | 85 | 106 | 1.634615385 | 2.038461538 |
| | | FliP | | | | | |
| — | BB_A54 | hypothetical protein | 28 | 106 | 57 | 3.785714286 | 2.035714286 |
| — | BB_P42 | phage terminase large subunit | 58 | 51 | 118 | 0.879310345 | 2.034482759 |
| — | predicted | antisense: clpX | 11980 | 21399 | 24332 | 1.786227045 | 2.031051753 |
| | RNA | | | | | | |
| — | BB_A65 | lipoprotein | 636 | 393 | 1277 | 0.617924528 | 2.007861635 |
| — | predicted | antisense: BB_P31 | 23325 | 36053 | 46733 | 1.5456806 | 2.003558414 |
| | RNA | | | | | | |
| — | BB_0325 | hypothetical protein | 4 | 34 | 8 | 8.5 | 2 |
| — | BB_0616 | integral membrane protein | 8 | 46 | 16 | 5.75 | 2 |
| — | BB_0737 | tRNA-dihydrouridine synthase | 11 | 63 | 22 | 5.727272727 | 2 |
| — | BB_0301 | cell division protein B | 3 | 15 | 6 | 5 | 2 |
| — | BB_0039 | hypothetical protein | 2 | 9 | 4 | 4.5 | 2 |
| — | BB_0584 | integral membrane protein | 23 | 91 | 46 | 3.956521739 | 2 |
| gltX | BB_B72 | glutamate--tRNA ligase | 9 | 28 | 18 | 3.111111111 | 2 |
| — | BB_0346 | export chaperone | 6 | 17 | 12 | 2.833333333 | 2 |
| recD | BB_0632 | exodeoxyribonuclease V subunit | 7 | 14 | 14 | 2 | 2 |
| | | alpha | | | | | |
| — | BB_H26 | hypothetical protein | 2 | 4 | 4 | 2 | 2 |
| bosR | BB_0647 | oxidative stress regulator BosR | 23 | 39 | 46 | 1.695652174 | 2 |
| rrmj | BB_0313 | rRNA large subunit | 3 | 5 | 6 | 1.666666667 | 2 |
| | | methyltransferase | | | | | |
| — | BB_0449 | hypothetical protein | 19 | 30 | 38 | 1.578947368 | 2 |
| — | BB_S34 | hypothetical protein | 15 | 19 | 30 | 1.266666667 | 2 |
| — | BB_0208 | hypothetical protein | 7 | 4 | 14 | 0.571428571 | 2 |
| — | BB_M18 | hypothetical protein | 2 | 0 | 4 | 0 | 2 |
| — | BB_N14 | hypothetical protein | 2 | 0 | 4 | 0 | 2 |
| bppB | BB_Q44 | protein BppB | 3 | 0 | 6 | 0 | 2 |
| — | predicted | antisense: BB_P31 | 15330 | 21758 | 30593 | 1.419308545 | 1.995629485 |
| | RNA | | | | | | |
| — | predicted | antisense: BB_0578 | 21153 | 44470 | 42060 | 2.102302274 | 1.988370444 |
| | RNA | | | | | | |
| — | predicted | antisense: tig | 24014 | 30953 | 47340 | 1.288956442 | 1.971350046 |
| | RNA | | | | | | |
| fbaA | BB_0445 | fructose-bisphosphate aldolase | 31 | 102 | 61 | 3.290322581 | 1.967741935 |
| — | BB_0570 | chemotaxis response regulator | 276 | 459 | 540 | 1.663043478 | 1.956521739 |
| — | BB_R15 | hypothetical protein | 46 | 26 | 90 | 0.565217391 | 1.956521739 |
| — | BB_0341 | aspartyl/glutamyl- | 22 | 84 | 43 | 3.818181818 | 1.954545455 |
| | | tRNA(Asn/Gln) | | | | | |
| | | amidotransferase subunit B | | | | | |
| guaA | BB_B18 | GMP synthase | 101 | 300 | 197 | 2.97029703 | 1.95049505 |
| fliG | BB_0290 | flagellar motor switch protein | 38 | 38 | 74 | 1 | 1.947368421 |
| | | FliG | | | | | |
| glpK | BB_0241 | glycerol kinase | 107 | 184 | 208 | 1.719626168 | 1.943925234 |
| — | BB_0733 | hypothetical protein | 51 | 171 | 99 | 3.352941176 | 1.941176471 |
| — | predicted | antisense: BB_0132 | 18235 | 21129 | 35397 | 1.158705786 | 1.941157115 |
| | RNA | | | | | | |
| metK | BB_0376 | S-adenosylmethionine | 97 | 308 | 187 | 3.175257732 | 1.927835052 |
| | | synthetase | | | | | |
| phoU | BB_0042 | phosphate-specific transport | 13 | 18 | 25 | 1.384615385 | 1.923076923 |
| | | system accessory protein PhoU | | | | | |
| — | predicted | antisense: clpP | 13203 | 23082 | 25359 | 1.748239037 | 1.920699841 |
| | RNA | | | | | | |
| — | BB_R13 | hypothetical protein | 25 | 24 | 48 | 0.96 | 1.92 |
| — | BB_0416 | pheromone shutdown protein | 81 | 230 | 155 | 2.839506173 | 1.913580247 |
| — | BB_P14 | hypothetical protein | 97 | 77 | 185 | 0.793814433 | 1.907216495 |
| — | BB_0080 | ABC transporter ATP-binding | 32 | 100 | 61 | 3.125 | 1.90625 |
| | | protein | | | | | |
| — | predicted | antisense: tig | 21239 | 18405 | 40472 | 0.866566223 | 1.905551109 |
| | RNA | | | | | | |
| gidA | BB_0178 | tRNA uridine 5- | 20 | 42 | 38 | 2.1 | 1.9 |
| | | carboxymethylaminomethyl | | | | | |
| | | modification protein GidA | | | | | |
| — | BB_0359 | peptidase S41 | 20 | 31 | 38 | 1.55 | 1.9 |
| — | predicted | antisense: BB_0381 | 11832 | 22391 | 22435 | 1.892410412 | 1.896129141 |
| | RNA | | | | | | |
| — | predicted | antisense: rpmA | 12622 | 23222 | 23915 | 1.839803518 | 1.894707653 |
| | RNA | | | | | | |
| priA | BB_0014 | primosomal protein N | 232 | 186 | 439 | 0.801724138 | 1.892241379 |
| — | BB_0133 | hypothetical protein | 9 | 33 | 17 | 3.666666667 | 1.888888889 |
| — | BB_0259 | transglycosylase SLT domain- | 27 | 88 | 51 | 3.259259259 | 1.888888889 |
| | | containing protein | | | | | |
| — | BB_0473 | integral membrane protein | 18 | 30 | 34 | 1.666666667 | 1.888888889 |
| — | BB_0505 | hypothetical protein | 17 | 34 | 32 | 2 | 1.882352941 |
| ileS | BB_0833 | isoleucine--tRNA ligase | 41 | 53 | 77 | 1.292682927 | 1.87804878 |
| — | BB_0650 | hypothetical protein | 503 | 706 | 944 | 1.403578529 | 1.876739563 |
| — | predicted | antisense: BB_0715 | 24255 | 40327 | 45509 | 1.662626263 | 1.876272933 |
| | RNA | | | | | | |
| — | predicted | antisense: BB_0219 | 15518 | 32402 | 29104 | 2.088026808 | 1.87549942 |
| | RNA | | | | | | |
| — | BB_0637 | Na+/H+ antiporter family | 24 | 138 | 45 | 5.75 | 1.875 |
| bmpC | BB_0384 | basic membrane protein C | 8 | 22 | 15 | 2.75 | 1.875 |
| — | BB_R18 | hypothetical protein | 8 | 12 | 15 | 1.5 | 1.875 |
| — | BB_D18 | hypothetical protein | 72 | 93 | 135 | 1.291666667 | 1.875 |
| — | predicted | antisense: BB_0621 | 13781 | 23013 | 25731 | 1.669907844 | 1.867135912 |
| | RNA | | | | | | |
| ylqF | BB_0643 | ribosome biogenesis GTPase A | 30 | 42 | 56 | 1.4 | 1.866666667 |
| — | BB_0297 | protein smf | 7 | 8 | 13 | 1.142857143 | 1.857142857 |
| — | BB_A41 | hypothetical protein | 48 | 15 | 89 | 0.3125 | 1.854166667 |
| lepB | BB_0031 | signal peptidase I | 41 | 133 | 76 | 3.243902439 | 1.853658537 |
| mlpJ | BB_Q35 | protein MlpJ | 80 | 91 | 148 | 1.1375 | 1.85 |
| groL | BB_0649 | chaperonin GroEL | 58 | 71 | 107 | 1.224137931 | 1.844827586 |
| panF | BB_0814 | sodium/panthothenate symporter | 19 | 35 | 35 | 1.842105263 | 1.842105263 |
| era | BB_0660 | GTPase Era | 25 | 25 | 46 | 1 | 1.84 |
| — | BB_J09 | outer surface protein D | 43 | 43 | 79 | 1 | 1.837209302 |
| — | BB_0088 | elongation factor EF-4 | 12 | 105 | 22 | 8.75 | 1.833333333 |
| — | BB_0467 | laccase domain-containing | 6 | 14 | 11 | 2.333333333 | 1.833333333 |
| | | protein | | | | | |
| — | BB_0347 | fibronectin/fibrinogen-binding | 12 | 21 | 22 | 1.75 | 1.833333333 |
| | | protein | | | | | |
| — | BB_0553 | hypothetical protein | 17 | 17 | 31 | 1 | 1.823529412 |
| — | BB_R41 | hypothetical protein | 11 | 13 | 20 | 1.181818182 | 1.818181818 |
| — | BB_0134 | hypothetical protein | 150 | 158 | 272 | 1.053333333 | 1.813333333 |
| nusG | BB_0394 | transcription | 16 | 51 | 29 | 3.1875 | 1.8125 |
| | | termination/antitermination | | | | | |
| | | factor | | | | | |
| — | BB_M06 | hypothetical protein | 16 | 16 | 29 | 1 | 1.8125 |
| — | BB_0669 | chemotaxis protein CheA | 26 | 32 | 47 | 1.230769231 | 1.807692308 |
| — | BB_0781 | GTPase Obg | 20 | 38 | 36 | 1.9 | 1.8 |
| — | BB_0108 | basic membrane protein | 15 | 27 | 27 | 1.8 | 1.8 |
| bppA | BB_P35 | protein BppA | 25 | 28 | 45 | 1.12 | 1.8 |
| — | BB_N01 | hypothetical protein | 5 | 5 | 9 | 1 | 1.8 |
| — | BB_0307 | hypothetical protein | 10 | 6 | 18 | 0.6 | 1.8 |
| — | BB_0330 | peptide ABC transporter | 79 | 137 | 142 | 1.734177215 | 1.797468354 |
| | | substrate-binding protein | | | | | |
| — | BB_P18 | hypothetical protein | 48 | 58 | 86 | 1.208333333 | 1.791666667 |
| pyrG | BB_0575 | CTP synthetase | 62 | 92 | 111 | 1.483870968 | 1.790322581 |
| — | predicted | antisense: ftsA | 27455 | 45823 | 49049 | 1.669022036 | 1.786523402 |
| | RNA | | | | | | |
| — | BB_0369 | ATP-dependent Clp protease | 14 | 51 | 25 | 3.642857143 | 1.785714286 |
| | | subunit A | | | | | |
| — | BB_0749 | hypothetical protein | 18 | 77 | 32 | 4.277777778 | 1.777777778 |
| rdgB | BB_0247 | non-canonical purine NTP | 22 | 25 | 39 | 1.136363636 | 1.772727273 |
| | | pyrophosphatase | | | | | |
| — | predicted | antisense: BB_0715 | 14453 | 23343 | 25596 | 1.615097212 | 1.770981803 |
| | RNA | | | | | | |
| — | predicted | antisense: BB_0715 | 18485 | 29083 | 32592 | 1.573329727 | 1.763159318 |
| | RNA | | | | | | |
| — | BB_0032 | hypothetical protein | 21 | 40 | 37 | 1.904761905 | 1.761904762 |
| — | predicted | antisense: rplU | 15697 | 28773 | 27634 | 1.833025419 | 1.760463783 |
| | RNA | | | | | | |
| — | predicted | antisense: BB_0442 | 23386 | 29916 | 41128 | 1.279226888 | 1.758659027 |
| | RNA | | | | | | |
| — | predicted | antisense: ftsA | 23827 | 39846 | 41790 | 1.672304528 | 1.753892643 |
| | RNA | | | | | | |
| map | BB_0105 | methionine aminopeptidase | 8 | 50 | 14 | 6.25 | 1.75 |
| — | BB_0761 | peptidoglycan-binding protein | 20 | 88 | 35 | 4.4 | 1.75 |
| pgk | BB_0056 | phosphoglycerate kinase | 24 | 54 | 42 | 2.25 | 1.75 |
| truA | BB_0012 | tRNA pseudouridine synthase A | 67 | 72 | 117 | 1.074626866 | 1.746268657 |
| — | BB_0149 | flagellar hook-associated protein | 23 | 34 | 40 | 1.47826087 | 1.739130435 |
| | | FliD | | | | | |
| — | BB_0839 | hypothetical protein | 15 | 12 | 26 | 0.8 | 1.733333333 |
| — | BB_O35 | hypothetical protein | 156 | 90 | 270 | 0.576923077 | 1.730769231 |
| — | BB_0109 | acetyl-CoA C-acetyltransferase | 11 | 23 | 19 | 2.090909091 | 1.727272727 |
| pgi | BB_0730 | glucose-6-phosphate isomerase | 86 | 151 | 148 | 1.755813953 | 1.720930233 |
| — | BB_0588 | MTA/SAH nucleosidase | 278 | 266 | 478 | 0.956834532 | 1.71942446 |
| — | BB_0244 | hypothetical protein | 81 | 144 | 139 | 1.777777778 | 1.716049383 |
| — | BB_0052 | tRNA/rRNA methyltransferase | 218 | 316 | 374 | 1.449541284 | 1.71559633 |
| — | BB_0345 | hypothetical protein | 7 | 14 | 12 | 2 | 1.714285714 |
| — | BB_0795 | outer membrane protein | 14 | 26 | 24 | 1.857142857 | 1.714285714 |
| — | BB_A60 | surface lipoprotein P27 | 21 | 19 | 36 | 0.904761905 | 1.714285714 |
| tgt | BB_0809 | queuine tRNA-ribosyltransferase | 259 | 360 | 443 | 1.38996139 | 1.71042471 |
| pgsA | BB_0721 | CDP-diacylglycerol--glycerol-3- | 24 | 49 | 41 | 2.041666667 | 1.708333333 |
| | | phosphate 3- | | | | | |
| | | phosphatidyltransferase | | | | | |
| efbC | BB_0462 | nucleoid-associated protein | 24 | 44 | 41 | 1.833333333 | 1.708333333 |
| | | EbfC | | | | | |
| — | BB_0020 | diphosphate--fructose-6- | 17 | 61 | 29 | 3.588235294 | 1.705882353 |
| | | phosphate 1-phosphotransferase | | | | | |
| — | BB_0771a | hypothetical protein | 27 | 20 | 46 | 0.740740741 | 1.703703704 |
| bppB | BB_S39 | protein BppB | 10 | 11 | 17 | 1.1 | 1.7 |
| — | BB_0572 | glycosyl transferase | 10 | 10 | 17 | 1 | 1.7 |
| rplP | BB_0485 | 50S ribosomal protein L16 | 10 | 9 | 17 | 0.9 | 1.7 |
| — | BB_0772 | flagellar basal body P-ring | 13 | 20 | 22 | 1.538461538 | 1.692307692 |
| | | protein | | | | | |
| — | BB_0834 | ATP-dependent Clp protease | 13 | 12 | 22 | 0.923076923 | 1.692307692 |
| | | subunit C | | | | | |
| — | BB_R04 | hypothetical protein | 32 | 27 | 54 | 0.84375 | 1.6875 |
| — | BB_0568 | chemotaxis response regulator | 51 | 92 | 86 | 1.803921569 | 1.68627451 |
| — | predicted | antisense: BB_0461 | 17112 | 22768 | 28852 | 1.330528284 | 1.686068256 |
| | RNA | | | | | | |
| — | BB_Q18 | hypothetical protein | 89 | 55 | 150 | 0.617977528 | 1.685393258 |
| — | BB_0417 | adenylate kinase | 249 | 536 | 419 | 2.152610442 | 1.682730924 |
| — | BB_0665 | hypothetical protein | 67 | 58 | 112 | 0.865671642 | 1.671641791 |
| — | predicted | antisense: BB_0715 | 22733 | 27750 | 37984 | 1.220692386 | 1.67087494 |
| | RNA | | | | | | |
| — | predicted | antisense: tig | 30584 | 36789 | 51004 | 1.202883861 | 1.66766937 |
| | RNA | | | | | | |
| — | BB_0442 | membrane protein insertase | 6 | 27 | 10 | 4.5 | 1.666666667 |
| | | YidC | | | | | |
| — | BB_A04 | S2 antigen | 320 | 233 | 532 | 0.728125 | 1.6625 |
| — | BB_0073 | hypothetical protein | 38 | 34 | 63 | 0.894736842 | 1.657894737 |
| — | predicted | antisense: lysS | 15976 | 31965 | 26480 | 2.000813721 | 1.657486229 |
| | RNA | | | | | | |
| — | predicted | antisense: BB_0096 | 13035 | 23072 | 21584 | 1.770003836 | 1.655849636 |
| | RNA | | | | | | |
| — | predicted | antisense: BB_0351 | 25091 | 14849 | 41532 | 0.591805827 | 1.655254872 |
| | RNA | | | | | | |
| — | predicted | antisense: ftsA | 19555 | 21984 | 32302 | 1.124213756 | 1.651853746 |
| | RNA | | | | | | |
| — | BB_P09 | hypothetical protein | 522 | 236 | 861 | 0.45210728 | 1.649425287 |
| fliF | BB_0291 | flagellar MS-ring protein FliF | 14 | 17 | 23 | 1.214285714 | 1.642857143 |
| — | BB_R06 | hypothetical protein | 49 | 29 | 80 | 0.591836735 | 1.632653061 |
| — | BB_0415 | chemotaxis protein CheB | 65 | 172 | 106 | 2.646153846 | 1.630769231 |
| — | BB_A19 | hypothetical protein | 27 | 9 | 44 | 0.333333333 | 1.62962963 |
| uvrC | BB_0457 | excinuclease ABC subunit C | 78 | 86 | 127 | 1.102564103 | 1.628205128 |
| — | BB_0531 | hypothetical protein | 43 | 88 | 70 | 2.046511628 | 1.627906977 |
| — | BB_0840 | lipoprotein | 8 | 12 | 13 | 1.5 | 1.625 |
| rnmV | BB_0626 | ribonuclease M5 | 488 | 552 | 792 | 1.131147541 | 1.62295082 |
| — | predicted | antisense: BB_0789 | 23189 | 26418 | 37528 | 1.139247057 | 1.61835353 |
| | RNA | | | | | | |
| — | BB_0007 | hypothetical protein | 89 | 54 | 144 | 0.606741573 | 1.617977528 |
| — | predicted | antisense: BB_0715 | 26884 | 26882 | 43429 | 0.999925606 | 1.615421812 |
| | RNA | | | | | | |
| — | predicted | antisense: BB_0786 | 26757 | 39154 | 43090 | 1.46331801 | 1.610419703 |
| | RNA | | | | | | |
| — | predicted | antisense: rpsD | 17008 | 39303 | 27348 | 2.310853716 | 1.6079492 |
| | RNA | | | | | | |
| — | BB_0167 | outer membrane protein | 408 | 305 | 656 | 0.74754902 | 1.607843137 |
| — | predicted | antisense: rpsE | 38064 | 34929 | 61166 | 0.917638714 | 1.606925179 |
| | RNA | | | | | | |
| — | BB_0745 | endonuclease III | 86 | 137 | 138 | 1.593023256 | 1.604651163 |
| trmD | BB_0698 | tRNA (guanine-N(1)-)- | 10 | 43 | 16 | 4.3 | 1.6 |
| | | methyltransferase | | | | | |
| rpsN | BB_0491 | 30S ribosomal protein S14 | 10 | 37 | 16 | 3.7 | 1.6 |
| — | BB_0070 | hypothetical protein | 75 | 176 | 120 | 2.346666667 | 1.6 |
| — | predicted | antisense: rplB | 28949 | 16752 | 46070 | 0.578672838 | 1.591419393 |
| | RNA | | | | | | |
| — | BB_A73 | antigen P35 | 22 | 16 | 35 | 0.727272727 | 1.590909091 |
| — | predicted | antisense: pth BB_0786 | 23467 | 33206 | 37263 | 1.41500831 | 1.587889377 |
| | RNA | | | | | | |
| — | BB_0784 | ribosomal silencing factor RsfS | 12 | 27 | 19 | 2.25 | 1.583333333 |
| — | BB_G01 | hypothetical protein | 12 | 3 | 19 | 0.25 | 1.583333333 |
| — | predicted | antisense: BB_0442 | 31877 | 23416 | 50422 | 0.734573517 | 1.581767419 |
| | RNA | | | | | | |
| — | predicted | antisense: BB_0391 | 27040 | 27985 | 42739 | 1.034948225 | 1.58058432 |
| | RNA | | | | | | |
| — | BB_G07 | hypothetical protein | 26 | 10 | 41 | 0.384615385 | 1.576923077 |
| mgtE | BB_0380 | Mg2+ transport protein | 54 | 145 | 85 | 2.685185185 | 1.574074074 |
| — | BB_G29 | hypothetical protein | 7 | 11 | 11 | 1.571428571 | 1.571428571 |
| — | BB_A52 | outer membrane protein | 42 | 55 | 66 | 1.30952381 | 1.571428571 |
| — | predicted | antisense: secA | 24868 | 17538 | 39042 | 0.705243687 | 1.569969439 |
| | RNA | | | | | | |
| — | BB_0314 | octaprenyl-diphosphate synthase | 51 | 69 | 80 | 1.352941176 | 1.568627451 |
| — | predicted | antisense: pth | 24799 | 25415 | 38833 | 1.024839711 | 1.565909916 |
| | RNA | | | | | | |
| — | BB_0045 | P115 protein | 23 | 24 | 36 | 1.043478261 | 1.565217391 |
| — | predicted | antisense: BB_0496 | 22752 | 19550 | 35604 | 0.85926512 | 1.564873418 |
| | RNA | | | | | | |
| — | BB_0072 | membrane protein | 48 | 67 | 75 | 1.395833333 | 1.5625 |
| ftsA | BB_0300 | cell division protein FtsA | 9 | 24 | 14 | 2.666666667 | 1.555555556 |
| — | BB_0605 | serine-type D-Ala-D-Ala | 40 | 80 | 62 | 2 | 1.55 |
| | | carboxypeptidase | | | | | |
| ackA | BB_0622 | acetate kinase | 1081 | 1149 | 1673 | 1.062904718 | 1.547641073 |
| — | BB_0431 | CobQ/CobB/MinD/ParA | 35 | 58 | 54 | 1.657142857 | 1.542857143 |
| | | nucleotide binding domain- | | | | | |
| | | containing protein | | | | | |
| — | BB_0619 | DHH family protein | 48 | 91 | 74 | 1.895833333 | 1.541666667 |
| — | predicted | antisense: nusG | 28038 | 36279 | 43188 | 1.293922534 | 1.540338113 |
| | RNA | | | | | | |
| — | BB_0085 | hypothetical protein | 52 | 90 | 80 | 1.730769231 | 1.538461538 |
| — | predicted | antisense: rpsD | 24606 | 37497 | 37801 | 1.523896611 | 1.536251321 |
| | RNA | | | | | | |
| prfA | BB_0196 | peptide chain release factor 1 | 56 | 65 | 86 | 1.160714286 | 1.535714286 |
| — | BB_G0036 | hypothetical protein | 15 | 7 | 23 | 0.466666667 | 1.533333333 |
| — | predicted | antisense: BB_0498 | 32163 | 22147 | 49088 | 0.688586264 | 1.526225787 |
| | RNA | | | | | | |
| — | BB_B04 | chitibiose transporter protein | 394 | 1041 | 598 | 2.64213198 | 1.517766497 |
| | | ChbC | | | | | |
| — | predicted | antisense: BB_0775 | 18180 | 23873 | 27569 | 1.313146315 | 1.516446645 |
| | RNA | | | | | | |
| — | BB_J37 | hypothetical protein | 51 | 56 | 77 | 1.098039216 | 1.509803922 |
| — | predicted | antisense: rplO | 32675 | 34453 | 49266 | 1.05441469 | 1.507758225 |
| | RNA | | | | | | |
| pfs | BB_0375 | nucleosidase | 65 | 133 | 98 | 2.046153846 | 1.507692308 |
| — | BB_0751 | hypothetical protein | 73 | 87 | 110 | 1.191780822 | 1.506849315 |
| — | BB_0819 | cytidylate kinase | 4 | 31 | 6 | 7.75 | 1.5 |
| — | BB_B10 | hypothetical protein | 4 | 23 | 6 | 5.75 | 1.5 |
| — | BB_G30 | hypothetical protein | 10 | 33 | 15 | 3.3 | 1.5 |
| bmpA | BB_0383 | basic membrane protein A | 6 | 12 | 9 | 2 | 1.5 |
| infC | BB_0190 | translation initiation factor IF-3 | 52 | 97 | 78 | 1.865384615 | 1.5 |
| — | BB_M20 | hypothetical protein | 2 | 3 | 3 | 1.5 | 1.5 |
| — | BB_0770 | divergent polysaccharide | 22 | 33 | 33 | 1.5 | 1.5 |
| | | deacetylase superfamily protein | | | | | |
| — | BB_S44 | hypothetical protein | 10 | 9 | 15 | 0.9 | 1.5 |
| — | BB_S27 | hypothetical protein | 14 | 12 | 21 | 0.857142857 | 1.5 |
| — | BB_0210 | hypothetical protein | 28 | 24 | 42 | 0.857142857 | 1.5 |
| — | BB_0286 | flagellar protein | 12 | 10 | 18 | 0.833333333 | 1.5 |
| — | BB_M32 | PF-32 protein | 4 | 3 | 6 | 0.75 | 1.5 |
| — | BB_R09 | hypothetical protein | 2 | 1 | 3 | 0.5 | 1.5 |
| — | BB_Q88 | hypothetical protein | 28 | 14 | 42 | 0.5 | 1.5 |
| — | BB_J41 | antigen P35 | 2 | 0 | 3 | 0 | 1.5 |
| — | predicted | antisense: BB_0176 | 17277 | 27257 | 25789 | 1.577646582 | 1.492678127 |
| | RNA | | | | | | |
| — | predicted | antisense: clpX | 26799 | 29556 | 39941 | 1.102876973 | 1.490391433 |
| | RNA | | | | | | |
| — | BB_0084 | cysteine desulfurase | 98 | 88 | 146 | 0.897959184 | 1.489795918 |
| — | BB_0026 | bifunctional | 39 | 130 | 58 | 3.333333333 | 1.487179487 |
| | | methylenetetrahydrofolate | | | | | |
| | | dehydrogenase/methenyltetra- | | | | | |
| | | hydrofolate cyclohydrolase | | | | | |
| recA | BB_0131 | recombinase A | 21 | 20 | 31 | 0.952380952 | 1.476190476 |
| — | BB_0596 | methyl-accepting chemotaxis | 59 | 87 | 87 | 1.474576271 | 1.474576271 |
| | | protein | | | | | |
| — | predicted | antisense: BB_0712 | 19448 | 22394 | 28668 | 1.151480872 | 1.474084739 |
| | RNA | | | | | | |
| — | BB_R45 | phage terminase large subunit | 19 | 12 | 28 | 0.631578947 | 1.473684211 |
| — | predicted | antisense: secA | 42660 | 33448 | 62849 | 0.784060009 | 1.473253633 |
| | RNA | | | | | | |
| — | BB_0006 | membrane protein | 58 | 73 | 85 | 1.25862069 | 1.465517241 |
| — | predicted | antisense: BB_0505 | 22116 | 26785 | 32397 | 1.211114126 | 1.464867065 |
| | RNA | | | | | | |
| — | BB_0829 | exonuclease SbcD | 28 | 50 | 41 | 1.785714286 | 1.464285714 |
| bppC | BB_S40 | protein BppC | 330 | 901 | 483 | 2.73030303 | 1.463636364 |
| — | predicted | antisense: BB_0726 | 23070 | 29560 | 33732 | 1.281317729 | 1.462158648 |
| | RNA | | | | | | |
| lspA | BB_0469 | lipoprotein signal peptidase | 13 | 24 | 19 | 1.846153846 | 1.461538462 |
| — | BB_J45 | lipoprotein | 26 | 45 | 38 | 1.730769231 | 1.461538462 |
| — | predicted | antisense: BB_0789 | 31321 | 30220 | 45749 | 0.964847866 | 1.460649405 |
| | RNA | | | | | | |
| — | BB_N17 | hypothetical protein | 35 | 22 | 51 | 0.628571429 | 1.457142857 |
| hisS | BB_0135 | histidine--tRNA ligase | 20 | 55 | 29 | 2.75 | 1.45 |
| — | predicted | antisense: BB_0714 | 17359 | 29572 | 25147 | 1.703554352 | 1.448643355 |
| | RNA | | | | | | |
| — | predicted | antisense: BB_A61 | 43675 | 14034 | 63250 | 0.321327991 | 1.448196909 |
| | RNA | | | | | | |
| — | BB_0192 | hypothetical protein | 9 | 17 | 13 | 1.888888889 | 1.444444444 |
| arcA | BB_0841 | arginine deiminase | 9 | 12 | 13 | 1.333333333 | 1.444444444 |
| — | BB_A30 | hypothetical protein | 18 | 13 | 26 | 0.722222222 | 1.444444444 |
| — | BB_B09 | lipoprotein | 151 | 110 | 218 | 0.728476821 | 1.443708609 |
| — | BB_0434 | chromosome-partitioning protein | 188 | 128 | 271 | 0.680851064 | 1.441489362 |
| | | ParB | | | | | |
| — | predicted | antisense: BB_0096 | 20600 | 26200 | 29681 | 1.27184466 | 1.440825243 |
| | RNA | | | | | | |
| — | predicted | antisense: rpoB | 29946 | 16932 | 43145 | 0.565417752 | 1.440760035 |
| | RNA | | | | | | |
| secA | BB_0154 | protein translocase subunit SecA | 23 | 64 | 33 | 2.782608696 | 1.434782609 |
| — | predicted | antisense: rpsC | 35844 | 23450 | 51378 | 0.654223859 | 1.433377971 |
| | RNA | | | | | | |
| — | predicted | antisense: BB_0097 | 17800 | 26159 | 25474 | 1.469606742 | 1.431123596 |
| | RNA | | | | | | |
| — | predicted | antisense: rpsE | 30297 | 29714 | 43326 | 0.980757171 | 1.430042578 |
| | RNA | | | | | | |
| — | predicted | antisense: rpmA | 19393 | 20681 | 27719 | 1.066415717 | 1.429330171 |
| | RNA | | | | | | |
| — | predicted | antisense: rplA | 31638 | 25388 | 45210 | 0.802452747 | 1.428977811 |
| | RNA | | | | | | |
| — | BB_0631 | hypothetical protein | 28 | 73 | 40 | 2.607142857 | 1.428571429 |
| — | BB_0512 | hypothetical protein | 7 | 12 | 10 | 1.714285714 | 1.428571429 |
| — | BB_0038 | hypothetical protein | 7 | 7 | 10 | 1 | 1.428571429 |
| — | BB_0163 | hypothetical protein | 167 | 259 | 238 | 1.550898204 | 1.425149701 |
| uppS | BB_0120 | isoprenyl transferase | 45 | 44 | 64 | 0.977777778 | 1.422222222 |
| — | predicted | antisense: rplB | 52813 | 31972 | 75092 | 0.605381251 | 1.421846894 |
| | RNA | | | | | | |
| — | predicted | antisense: BB_S33 | 19846 | 22554 | 28208 | 1.13645067 | 1.421344352 |
| | RNA | | | | | | |
| — | BB_0325 | hypothetical protein | 169 | 287 | 240 | 1.698224852 | 1.420118343 |
| — | predicted | antisense: rpsS rplV | 45718 | 24911 | 64766 | 0.544883853 | 1.416641148 |
| | RNA | | | | | | |
| — | BB_N32 | PF-32 protein | 41 | 38 | 58 | 0.926829268 | 1.414634146 |
| — | BB_P03 | hypothetical protein | 70 | 57 | 99 | 0.814285714 | 1.414285714 |
| — | predicted | antisense: rplA | 52924 | 58392 | 74816 | 1.103317965 | 1.413649762 |
| | RNA | | | | | | |
| ruvB | BB_0022 | Holliday junction DNA helicase | 17 | 18 | 24 | 1.058823529 | 1.411764706 |
| | | RuvB | | | | | |
| — | predicted | antisense: rplS trmD | 29045 | 36298 | 40945 | 1.249715958 | 1.409709072 |
| | RNA | | | | | | |
| bppC | BB_R38 | BppC | 76 | 197 | 107 | 2.592105263 | 1.407894737 |
| — | predicted | antisense: BB_0724 | 21771 | 34870 | 30624 | 1.601671949 | 1.406641863 |
| | RNA | | | | | | |
| — | BB_R07 | hypothetical protein | 74 | 44 | 104 | 0.594594595 | 1.405405405 |
| — | BB_B23 | guanine/xanthine permease | 52 | 200 | 73 | 3.846153846 | 1.403846154 |
| pepF | BB_0248 | oligoendopeptidase F | 367 | 456 | 515 | 1.242506812 | 1.403269755 |
| — | predicted | antisense: BB_0805 | 30022 | 21154 | 42105 | 0.704616614 | 1.402471521 |
| | RNA | | | | | | |
| — | predicted | antisense: ftsA | 25843 | 40980 | 36226 | 1.585729211 | 1.40177224 |
| | RNA | | | | | | |
| — | BB_0624 | hypothetical protein | 10 | 51 | 14 | 5.1 | 1.4 |
| — | BB_0231 | hypothetical protein | 5 | 18 | 7 | 3.6 | 1.4 |
| — | BB_0806 | lipoprotein | 10 | 27 | 14 | 2.7 | 1.4 |
| — | BB_0132 | transcription elongation factor | 30 | 43 | 42 | 1.433333333 | 1.4 |
| | | GreA | | | | | |
| — | BB_0156 | hypothetical protein | 5 | 7 | 7 | 1.4 | 1.4 |
| — | BB_H09 | type II restriction enzyme | 5 | 3 | 7 | 0.6 | 1.4 |
| | | methylase subunit | | | | | |
| — | predicted | antisense: BB_0094 | 26986 | 23849 | 37688 | 0.883754539 | 1.396576002 |
| | RNA | | | | | | |
| — | BB_0240 | glycerol uptake facilitator | 817 | 1062 | 1140 | 1.299877601 | 1.395348837 |
| — | BB_0843 | arginine-ornithine antiporter | 43 | 50 | 60 | 1.162790698 | 1.395348837 |
| metG | BB_0587 | methionine--tRNA ligase | 84 | 116 | 117 | 1.380952381 | 1.392857143 |
| rpsO | BB_0804 | 30S ribosomal protein S15 | 23 | 59 | 32 | 2.565217391 | 1.391304348 |
| — | predicted | antisense: BB_0094 | 18820 | 28757 | 26181 | 1.528002125 | 1.391126461 |
| | RNA | | | | | | |
| murD | BB_0585 | UDP-N-acetylmuramoylalanine-- | 114 | 155 | 158 | 1.359649123 | 1.385964912 |
| | | D-glutamate ligase | | | | | |
| — | BB_R17 | hypothetical protein | 259 | 176 | 357 | 0.67953668 | 1.378378378 |
| — | BB_0037 | 1-acyl-sn-glycerol-3-phosphate | 8 | 17 | 11 | 2.125 | 1.375 |
| | | acyltransferase | | | | | |
| — | BB_0342 | glutamyl-tRNA(Gln) | 56 | 74 | 77 | 1.321428571 | 1.375 |
| | | amidotransferase subunit A | | | | | |
| — | BB_0444 | nucleotide sugar epimerase | 27 | 62 | 37 | 2.296296296 | 1.37037037 |
| — | BB_0318 | ABC transporter ATP-binding | 27 | 30 | 37 | 1.111111111 | 1.37037037 |
| | | protein | | | | | |
| bppB | BB_P36 | protein BppB | 27 | 16 | 37 | 0.592592593 | 1.37037037 |
| — | predicted | antisense: BB_0593 | 29996 | 21507 | 41099 | 0.716995599 | 1.370149353 |
| | RNA | | | | | | |
| — | predicted | antisense: rplR rpsE | 31912 | 25246 | 43718 | 0.791113061 | 1.369954876 |
| | RNA | | | | | | |
| — | BB_A31 | pbsx family phage terminase | 38 | 23 | 52 | 0.605263158 | 1.368421053 |
| — | predicted | antisense: BB_B16 | 30221 | 20288 | 41303 | 0.671321267 | 1.366698653 |
| | RNA | | | | | | |
| — | predicted | antisense: BB_0442 | 28356 | 17791 | 38660 | 0.627415714 | 1.363379884 |
| | RNA | | | | | | |
| — | predicted | antisense: rplB | 54663 | 29581 | 74508 | 0.54115215 | 1.363042643 |
| | RNA | | | | | | |
| — | predicted | antisense: BB_0096 | 22437 | 23890 | 30566 | 1.064759103 | 1.362303338 |
| | RNA | | | | | | |
| — | BB_0145 | glycine/betaine ABC transporter | 39 | 69 | 53 | 1.769230769 | 1.358974359 |
| | | permease | | | | | |
| rpsC | BB_0484 | 30S ribosomal protein S3 | 14 | 17 | 19 | 1.214285714 | 1.357142857 |
| — | BB_L43 | phage terminase large subunit | 14 | 6 | 19 | 0.428571429 | 1.357142857 |
| — | BB_P16 | hypothetical protein | 793 | 330 | 1075 | 0.416141236 | 1.355611602 |
| — | predicted | antisense: BB_A61 | 31248 | 7521 | 42297 | 0.240687404 | 1.35359063 |
| | RNA | | | | | | |
| — | BB_S16 | hypothetical protein | 137 | 62 | 185 | 0.452554745 | 1.350364964 |
| — | BB_0174 | hypothetical protein | 23 | 47 | 31 | 2.043478261 | 1.347826087 |
| pheT | BB_0514 | phenylalanine--tRNA sligase | 35 | 106 | 47 | 3.028571429 | 1.342857143 |
| | | subunit beta | | | | | |
| — | predicted | antisense: rplP | 30835 | 19833 | 41395 | 0.643197665 | 1.342467975 |
| | RNA | | | | | | |
| — | BB_D01 | hypothetical protein | 165 | 58 | 221 | 0.351515152 | 1.339393939 |
| — | BB_0258 | undecaprenyl-diphosphatase | 118 | 188 | 158 | 1.593220339 | 1.338983051 |
| — | BB_0186 | tRNA | 21 | 133 | 28 | 6.333333333 | 1.333333333 |
| | | threonylcarbamoyladenosine | | | | | |
| | | biosynthesis protein | | | | | |
| — | BB_0260 | hypothetical protein | 6 | 35 | 8 | 5.833333333 | 1.333333333 |
| — | BB_0327 | glycerol-3-phosphate O- | 15 | 53 | 20 | 3.533333333 | 1.333333333 |
| | | acyltransferase | | | | | |
| — | BB_0379 | HIT family nucleosidase | 39 | 105 | 52 | 2.692307692 | 1.333333333 |
| — | BB_Q50 | phage terminase large subunit | 9 | 4 | 12 | 0.444444444 | 1.333333333 |
| — | predicted | antisense: rplW | 34311 | 19073 | 45745 | 0.555885868 | 1.333245898 |
| | RNA | | | | | | |
| — | predicted | antisense: rplB | 55617 | 28574 | 74016 | 0.513763777 | 1.330816117 |
| | RNA | | | | | | |
| — | BB_B29 | PTS system transporter subunit | 46 | 204 | 61 | 4.434782609 | 1.326086957 |
| | | IIBC | | | | | |
| — | predicted | antisense: BB_0504 | 48236 | 37645 | 63875 | 0.780433701 | 1.324218426 |
| | RNA | | | | | | |
| — | predicted | antisense: BB_0094 | 29829 | 16925 | 39494 | 0.567400852 | 1.324013544 |
| | RNA | | | | | | |
| — | BB_N02 | hypothetical protein | 19 | 14 | 25 | 0.736842105 | 1.315789474 |
| — | BB_0335 | peptide ABC transporter ATP- | 2201 | 1753 | 2879 | 0.796456156 | 1.308041799 |
| | | binding protein | | | | | |
| rplV | BB_0483 | 50S ribosomal protein L22 | 13 | 14 | 17 | 1.076923077 | 1.307692308 |
| — | predicted | antisense: BB_0231 hbb | 24725 | 36941 | 32185 | 1.494074823 | 1.301718908 |
| | RNA | | | | | | |
| — | predicted | antisense: BB_B16 | 44204 | 34349 | 57475 | 0.777056375 | 1.300221699 |
| | RNA | | | | | | |
| — | BB_0311 | NAD kinase | 10 | 15 | 13 | 1.5 | 1.3 |
| — | BB_R14 | hypothetical protein | 47 | 15 | 61 | 0.319148936 | 1.29787234 |
| — | predicted | antisense: BB_0490 | 38173 | 40799 | 49529 | 1.068792078 | 1.297487753 |
| | RNA | | | | | | |
| — | BB_0018 | ribosomal large subunit | 34 | 72 | 44 | 2.117647059 | 1.294117647 |
| | | pseudouridine synthase D | | | | | |
| mlpD | BB_R28 | lipoprotein | 31 | 18 | 40 | 0.580645161 | 1.290322581 |
| leuS | BB_0251 | leucine--tRNA ligase | 38 | 83 | 49 | 2.184210526 | 1.289473684 |
| — | predicted | antisense: BB_0215 | 10979 | 22689 | 14137 | 2.066581656 | 1.28764004 |
| | RNA | | | | | | |
| tmk | BB_0793 | thymidylate kinase | 80 | 97 | 103 | 1.2125 | 1.2875 |
| zwf | BB_0636 | glucose-6-phosphate 1- | 7 | 36 | 9 | 5.142857143 | 1.285714286 |
| | | dehydrogenase | | | | | |
| rseP | BB_0118 | RIP metalloprotease RseP | 42 | 27 | 54 | 0.642857143 | 1.285714286 |
| — | BB_P02 | hypothetical protein | 113 | 69 | 145 | 0.610619469 | 1.283185841 |
| — | predicted | antisense: tuf | 54506 | 49517 | 69878 | 0.908468792 | 1.282023997 |
| | RNA | | | | | | |
| — | BB_B28 | hypothetical protein | 50 | 42 | 64 | 0.84 | 1.28 |
| — | predicted | antisense: tilS | 26973 | 31653 | 34349 | 1.17350684 | 1.273458644 |
| | RNA | | | | | | |
| flgK | BB_0181 | flagellar hook-associated protein | 150 | 229 | 191 | 1.526666667 | 1.273333333 |
| | | FlgK | | | | | |
| — | BB_0779 | hypothetical protein | 22 | 57 | 28 | 2.590909091 | 1.272727273 |
| — | BB_R32 | hypothetical protein | 11 | 16 | 14 | 1.454545455 | 1.272727273 |
| — | BB_0328 | family 5 extracellular solute- | 44 | 47 | 56 | 1.068181818 | 1.272727273 |
| | | binding protein | | | | | |
| — | BB_0239 | deoxyguanosine/deoxyadenosine | 11 | 9 | 14 | 0.818181818 | 1.272727273 |
| | | kinase | | | | | |
| dnaN | BB_0438 | DNA polymerase III subunit | 11 | 9 | 14 | 0.818181818 | 1.272727273 |
| | | beta | | | | | |
| — | predicted | antisense: rpsC | 29640 | 11857 | 37691 | 0.400033738 | 1.271626181 |
| | RNA | | | | | | |
| — | predicted | antisense: BB_0805 | 32758 | 22416 | 41634 | 0.68429086 | 1.270956713 |
| | RNA | | | | | | |
| — | BB_P10 | hypothetical protein | 501 | 190 | 635 | 0.379241517 | 1.26746507 |
| — | BB_H41 | membrane protein | 15 | 23 | 19 | 1.533333333 | 1.266666667 |
| — | BB_0142 | outer membrane efflux protein | 19 | 23 | 24 | 1.210526316 | 1.263157895 |
| — | BB_0466 | ABC transporter ATP-binding | 19 | 22 | 24 | 1.157894737 | 1.263157895 |
| | | protein | | | | | |
| — | BB_0717 | integral membrane protein | 46 | 53 | 58 | 1.152173913 | 1.260869565 |
| — | BB_Q15 | hypothetical protein | 94 | 56 | 118 | 0.595744681 | 1.255319149 |
| — | predicted | antisense: BB_0505 | 28600 | 16707 | 35874 | 0.584160839 | 1.254335664 |
| | RNA | | | | | | |
| — | BB_0296 | ATP-dependent protease subunit | 16 | 47 | 20 | 2.9375 | 1.25 |
| | | HslV | | | | | |
| — | BB_0724 | potassium transporter | 16 | 43 | 20 | 2.6875 | 1.25 |
| — | BB_0426 | nucleoside 2- | 8 | 14 | 10 | 1.75 | 1.25 |
| | | deoxyribosyltransferase | | | | | |
| | | superfamily protein | | | | | |
| — | BB_0285 | flagellar protein | 8 | 10 | 10 | 1.25 | 1.25 |
| engA | BB_0508 | GTPase Der | 24 | 24 | 30 | 1 | 1.25 |
| — | predicted | antisense: rplS | 26105 | 38817 | 32580 | 1.486956522 | 1.248036775 |
| | RNA | | | | | | |
| — | predicted | antisense: rpoB | 32518 | 23037 | 40563 | 0.708438403 | 1.247401439 |
| | RNA | | | | | | |
| — | BB_R16 | hypothetical protein | 281 | 118 | 350 | 0.419928826 | 1.245551601 |
| — | predicted | antisense: BB_0490 | 37127 | 36619 | 46171 | 0.986317235 | 1.243596305 |
| | RNA | | | | | | |
| manA | BB_0407 | mannose-6-phosphate isomerase | 112 | 123 | 139 | 1.098214286 | 1.241071429 |
| — | BB_0418 | hypothetical protein | 1392 | 1791 | 1727 | 1.286637931 | 1.24066092 |
| — | predicted | antisense: flhA | 32742 | 21160 | 40526 | 0.646264736 | 1.237737463 |
| | RNA | | | | | | |
| — | BB_0351 | hypothetical protein | 17 | 62 | 21 | 3.647058824 | 1.235294118 |
| aspS | BB_0446 | aspartate--tRNA ligase | 366 | 387 | 452 | 1.057377049 | 1.234972678 |
| — | predicted | antisense: guaA | 106958 | 55227 | 131875 | 0.516342864 | 1.232960601 |
| | RNA | | | | | | |
| — | predicted | antisense: BB_0805 rpsO | 31123 | 28530 | 38333 | 0.91668541 | 1.231661472 |
| | RNA | | | | | | |
| — | predicted | antisense: bmpA | 23009 | 23263 | 28336 | 1.011039159 | 1.231518102 |
| | RNA | | | | | | |
| — | predicted | antisense: rplB | 30853 | 13678 | 37967 | 0.443328039 | 1.230577253 |
| | RNA | | | | | | |
| — | predicted | antisense: rpoB | 32495 | 22616 | 39944 | 0.695983998 | 1.229235267 |
| | RNA | | | | | | |
| erpB | BB_P39 | protein ErpB | 681 | 1926 | 837 | 2.828193833 | 1.22907489 |
| — | BB_0194 | TatD family hydrolase | 332 | 293 | 408 | 0.88253012 | 1.228915663 |
| — | predicted | antisense: rplC rpsJ | 55472 | 29344 | 68136 | 0.528987597 | 1.228295356 |
| | RNA | | | | | | |
| — | BB_Q24 | hypothetical protein | 149 | 78 | 183 | 0.523489933 | 1.228187919 |
| — | BB_0595 | hypothetical protein | 110 | 79 | 135 | 0.718181818 | 1.227272727 |
| — | predicted | antisense: recA | 28526 | 25416 | 34964 | 0.890976653 | 1.225688845 |
| | RNA | | | | | | |
| — | predicted | antisense: tuf | 78847 | 43577 | 96508 | 0.552677971 | 1.223990767 |
| | RNA | | | | | | |
| rplK | BB_0393 | 50S ribosomal protein L11 | 9 | 21 | 11 | 2.333333333 | 1.222222222 |
| — | BB_0010 | holo-acyl-carrier protein | 9 | 20 | 11 | 2.222222222 | 1.222222222 |
| | | synthase | | | | | |
| — | predicted | antisense: BB_0132 | 27545 | 25605 | 33658 | 0.929569795 | 1.221927755 |
| | RNA | | | | | | |
| — | BB_0198 | guanosine-3′,5′-bis(diphosphate) | 104 | 134 | 127 | 1.288461538 | 1.221153846 |
| | | 3′-pyrophosphohydrolase | | | | | |
| — | predicted | antisense: BB_0789 | 63781 | 49134 | 77859 | 0.770354808 | 1.22072404 |
| | RNA | | | | | | |
| — | BB_Q23 | hypothetical protein | 83 | 29 | 101 | 0.34939759 | 1.21686747 |
| — | BB_0324 | hypothetical protein | 185 | 168 | 225 | 0.908108108 | 1.216216216 |
| — | BB_0651 | protein translocase subunit YajC | 14 | 46 | 17 | 3.285714286 | 1.214285714 |
| — | BB_0732 | penicillin-binding protein | 28 | 55 | 34 | 1.964285714 | 1.214285714 |
| trmU | BB_0682 | tRNA-specific 2-thiouridylase | 89 | 105 | 108 | 1.179775281 | 1.213483146 |
| | | MnmA | | | | | |
| — | BB_N09 | hypothetical protein | 19 | 11 | 23 | 0.578947368 | 1.210526316 |
| — | BB_H09a | hypothetical protein | 197 | 148 | 238 | 0.751269036 | 1.208121827 |
| — | BB_S09 | hypothetical protein | 63 | 25 | 76 | 0.396825397 | 1.206349206 |
| — | predicted | antisense: rpoB | 24834 | 22295 | 29883 | 0.897761134 | 1.203309978 |
| | RNA | | | | | | |
| — | BB_0758 | hypothetical protein | 10 | 58 | 12 | 5.8 | 1.2 |
| — | BB_0215 | phosphate ABC transporter | 10 | 49 | 12 | 4.9 | 1.2 |
| | | substrate-binding protein | | | | | |
| — | BB_0068 | haloacid dehalogenase-like | 10 | 14 | 12 | 1.4 | 1.2 |
| | | hydrolase | | | | | |
| — | BB_R01 | hypothetical protein | 5 | 3 | 6 | 0.6 | 1.2 |
| — | BB_0398 | lipoprotein | 10 | 5 | 12 | 0.5 | 1.2 |
| — | predicted | — | 24757 | 22016 | 29650 | 0.889283839 | 1.197641071 |
| | RNA | | | | | | |
| rnhB | BB_0046 | ribonuclease HII | 1881 | 1832 | 2251 | 0.973950027 | 1.196703881 |
| — | predicted | antisense: rpsH | 40495 | 31668 | 48417 | 0.782022472 | 1.19562909 |
| | RNA | | | | | | |
| — | predicted | antisense: rpoB | 30375 | 12851 | 36200 | 0.423078189 | 1.191769547 |
| | RNA | | | | | | |
| — | BB_0098 | endonuclease MutS2 | 21 | 23 | 25 | 1.095238095 | 1.19047619 |
| — | predicted | antisense: BB_0504 | 30268 | 29158 | 36022 | 0.963327607 | 1.190101758 |
| | RNA | | | | | | |
| — | BB_R21 | hypothetical protein | 37 | 18 | 44 | 0.486486486 | 1.189189189 |
| — | predicted | antisense: rplA | 35725 | 32400 | 42483 | 0.906927922 | 1.18916725 |
| | RNA | | | | | | |
| rbsA | BB_0677 | sugar ABC transporter ATP- | 16 | 25 | 19 | 1.5625 | 1.1875 |
| | | binding protein | | | | | |
| — | BB_R11 | hypothetical protein | 38 | 9 | 45 | 0.236842105 | 1.184210526 |
| — | predicted | antisense: rho | 13001 | 22555 | 15380 | 1.734866549 | 1.182985924 |
| | RNA | | | | | | |
| — | BB_0374 | hypothetical protein | 33 | 56 | 39 | 1.696969697 | 1.181818182 |
| — | BB_0573 | ABC transporter ATP-binding | 44 | 46 | 52 | 1.045454545 | 1.181818182 |
| | | protein | | | | | |
| nagA | BB_0151 | N-acetylglucosamine-6- | 117 | 160 | 138 | 1.367521368 | 1.179487179 |
| | | phosphate deacetylase | | | | | |
| — | predicted | antisense: rpsC | 35240 | 21447 | 41561 | 0.608598184 | 1.179370034 |
| | RNA | | | | | | |
| tig | BB_0610 | trigger factor | 17 | 35 | 20 | 2.058823529 | 1.176470588 |
| — | BB_0747 | oligopeptide ABC transporter | 86 | 112 | 101 | 1.302325581 | 1.174418605 |
| | | permease | | | | | |
| — | BB_0645 | PTS system glucose-specific | 104 | 280 | 122 | 2.692307692 | 1.173076923 |
| | | transporter subunit IIBC | | | | | |
| — | predicted | antisense: ptsP | 50133 | 31799 | 58721 | 0.634292781 | 1.17130433 |
| | RNA | | | | | | |
| — | BB_t31 | Met tRNA | 18 | 28 | 21 | 1.555555556 | 1.166666667 |
| — | BB_N43 | phage terminase large subunit | 6 | 4 | 7 | 0.666666667 | 1.166666667 |
| ospB | BB_A16 | outer surface protein B | 338 | 159 | 394 | 0.470414201 | 1.165680473 |
| pfkB | BB_0630 | 1-phosphofructokinase | 734 | 673 | 855 | 0.916893733 | 1.164850136 |
| — | BB_R08 | hypothetical protein | 110 | 54 | 128 | 0.490909091 | 1.163636364 |
| — | predicted | antisense: rpmF | 20873 | 26583 | 24243 | 1.273559143 | 1.161452594 |
| | RNA | | | | | | |
| — | predicted | antisense: BB_0093 | 33203 | 25451 | 38521 | 0.766527121 | 1.16016625 |
| | RNA | | | | | | |
| — | BB_0071 | membrane protein | 75 | 57 | 87 | 0.76 | 1.16 |
| — | predicted | antisense: rplL | 31854 | 35837 | 36895 | 1.125039242 | 1.158253281 |
| | RNA | | | | | | |
| — | BB_0498 | protein translocase subunit SecY | 95 | 174 | 110 | 1.831578947 | 1.157894737 |
| — | BB_0161 | hypothetical protein | 152 | 207 | 176 | 1.361842105 | 1.157894737 |
| — | predicted | antisense: rplC rplD | 40421 | 18440 | 46759 | 0.456198511 | 1.156799683 |
| | RNA | | | | | | |
| — | predicted | antisense: cspA | 30317 | 7893 | 35044 | 0.260348979 | 1.155919121 |
| | RNA | | | | | | |
| — | BB_0265 | hypothetical protein | 13 | 40 | 15 | 3.076923077 | 1.153846154 |
| — | BB_0790 | hypothetical protein | 13 | 12 | 15 | 0.923076923 | 1.153846154 |
| — | BB_0228 | hypothetical protein | 1348 | 1366 | 1555 | 1.013353116 | 1.153560831 |
| — | predicted | antisense: BB_0132 | 40801 | 20135 | 47050 | 0.493492807 | 1.153158011 |
| | RNA | | | | | | |
| — | BB_0361 | ATP-binding protein | 99 | 164 | 114 | 1.656565657 | 1.151515152 |
| — | BB_0106 | hypothetical protein | 20 | 17 | 23 | 0.85 | 1.15 |
| — | BB_0306 | rRNA small subunit | 20 | 17 | 23 | 0.85 | 1.15 |
| | | methyltransferase H | | | | | |
| — | BB_0257 | DNA translocase FtsK | 562 | 861 | 646 | 1.53202847 | 1.149466192 |
| — | BB_D001 | lipoprotein | 67 | 20 | 77 | 0.298507463 | 1.149253731 |
| — | BB_t24 | Leu tRNA | 27 | 14 | 31 | 0.518518519 | 1.148148148 |
| — | BB_P08 | hypothetical protein | 899 | 429 | 1031 | 0.477196885 | 1.146829811 |
| — | BB_B17 | inosine 5-monophosphate | 3020 | 3175 | 3458 | 1.051324503 | 1.145033113 |
| | | dehydrogenase | | | | | |
| pcrA | BB_0607 | ATP-dependent DNA helicase | 194 | 185 | 222 | 0.953608247 | 1.144329897 |
| rplO | BB_0497 | 50S ribosomal protein L15 | 14 | 21 | 16 | 1.5 | 1.142857143 |
| rpoC | BB_0388 | DNA-directed RNA polymerase | 28 | 25 | 32 | 0.892857143 | 1.142857143 |
| | | subunit beta′ | | | | | |
| — | BB_P17 | hypothetical protein | 1296 | 594 | 1476 | 0.458333333 | 1.138888889 |
| — | BB_0213 | lipoprotein | 2871 | 2183 | 3267 | 0.760362243 | 1.137931034 |
| — | BB_M10 | hypothetical protein | 37 | 14 | 42 | 0.378378378 | 1.135135135 |
| — | predicted | antisense: BB_0789 | 36901 | 23078 | 41830 | 0.625403106 | 1.133573616 |
| | RNA | | | | | | |
| recJ | BB_0254 | single-stranded-DNA-specific | 30 | 48 | 34 | 1.6 | 1.133333333 |
| | | exonuclease | | | | | |
| — | predicted | antisense: ffh | 32436 | 25203 | 36673 | 0.777007029 | 1.130626464 |
| | RNA | | | | | | |
| — | BB_0409 | hypothetical protein | 46 | 129 | 52 | 2.804347826 | 1.130434783 |
| — | BB_S45 | phage terminase large subunit | 31 | 15 | 35 | 0.483870968 | 1.129032258 |
| erpO | BB_L40 | ErpBl protein | 101 | 311 | 114 | 3.079207921 | 1.128712871 |
| — | predicted | antisense: BB_0094 | 22295 | 20640 | 25098 | 0.925768109 | 1.125723256 |
| | RNA | | | | | | |
| erpA | BB_P38 | protein ErpA | 8 | 32 | 9 | 4 | 1.125 |
| — | predicted | antisense: nusG | 29641 | 35281 | 33268 | 1.190276981 | 1.122364293 |
| | RNA | | | | | | |
| — | BB_0206 | methyltransferase | 90 | 298 | 101 | 3.311111111 | 1.122222222 |
| rpsS | BB_0482 | 30S ribosomal protein S19 | 33 | 46 | 37 | 1.393939394 | 1.121212121 |
| — | BB_M29 | hypothetical protein | 25 | 20 | 28 | 0.8 | 1.12 |
| — | predicted | antisense: BB_0504 | 42929 | 22817 | 48071 | 0.531505509 | 1.11977917 |
| | RNA | | | | | | |
| — | BB_0740 | hypothetical protein | 59 | 42 | 66 | 0.711864407 | 1.118644068 |
| — | predicted | antisense: BB_A43 | 32768 | 6191 | 36644 | 0.188934326 | 1.118286133 |
| | RNA | | | | | | |
| — | predicted | antisense: miaA | 24257 | 33333 | 27122 | 1.374160036 | 1.118110236 |
| | RNA | | | | | | |
| — | BB_0735 | rare lipoprotein A | 17 | 51 | 19 | 3 | 1.117647059 |
| rplB | BB_0481 | 50S ribosomal protein L2 | 17 | 23 | 19 | 1.352941176 | 1.117647059 |
| — | predicted | antisense: gyrB | 69422 | 42177 | 77544 | 0.607545159 | 1.116994613 |
| | RNA | | | | | | |
| — | predicted | antisense: guaA | 68920 | 35960 | 76873 | 0.521764364 | 1.11539466 |
| | RNA | | | | | | |
| — | BB_0236 | hypothetical protein | 26 | 36 | 29 | 1.384615385 | 1.115384615 |
| — | predicted | antisense: ffh | 43499 | 32442 | 48517 | 0.745810249 | 1.115358974 |
| | RNA | | | | | | |
| — | predicted | antisense: flhB | 42457 | 49175 | 47245 | 1.15823068 | 1.112772923 |
| | RNA | | | | | | |
| mraY | BB_0303 | phospho-N-acetylmuramoyl- | 116 | 121 | 129 | 1.043103448 | 1.112068966 |
| | | pentapeptide-transferase | | | | | |
| mgsA | BB_0364 | methylglyoxal synthase | 1491 | 748 | 1658 | 0.501676727 | 1.112005366 |
| — | BB_0268 | hypothetical protein | 9 | 22 | 10 | 2.444444444 | 1.111111111 |
| — | BB_0261 | hypothetical protein | 27 | 48 | 30 | 1.777777778 | 1.111111111 |
| ftsW | BB_0302 | lipid II flippase FtsW | 45 | 63 | 50 | 1.4 | 1.111111111 |
| — | BB_R33 | plasmid partition protein, | 9 | 11 | 10 | 1.222222222 | 1.111111111 |
| | | putative | | | | | |
| — | predicted | antisense: BB_0800 | 33687 | 20391 | 37337 | 0.605307685 | 1.108350402 |
| | RNA | | | | | | |
| — | predicted | antisense: BB_B16 | 33243 | 24612 | 36826 | 0.740366393 | 1.107782089 |
| | RNA | | | | | | |
| — | predicted | — | 29480 | 26115 | 32571 | 0.885854817 | 1.104850746 |
| | RNA | | | | | | |
| lon | BB_0253 | ATP-dependent protease La | 39 | 118 | 43 | 3.025641026 | 1.102564103 |
| nusB | BB_0107 | transcription antitermination | 10 | 25 | 11 | 2.5 | 1.1 |
| | | factor NusB | | | | | |
| mlpI | BB_N28 | lipoprotein | 10 | 21 | 11 | 2.1 | 1.1 |
| — | BB_0420 | sensory transduction histidine | 20 | 19 | 22 | 0.95 | 1.1 |
| | | kinase | | | | | |
| mutS | BB_0797 | DNA mismatch repair protein | 131 | 70 | 144 | 0.534351145 | 1.099236641 |
| | | MutS | | | | | |
| — | BB_B05 | chitibiose transporter protein | 215 | 179 | 236 | 0.83255814 | 1.097674419 |
| | | ChbA | | | | | |
| — | BB_S10 | hypothetical protein | 31 | 1 | 34 | 0.032258065 | 1.096774194 |
| — | predicted | antisense: BB_0490 rpsN | 28914 | 21721 | 31674 | 0.751227779 | 1.095455489 |
| | RNA | | | | | | |
| pta | BB_0589 | phosphate acetyltransferase | 246 | 128 | 269 | 0.520325203 | 1.093495935 |
| — | predicted | antisense: BB_0133 | 40842 | 45226 | 44493 | 1.107340483 | 1.089393272 |
| | RNA | | | | | | |
| fusA | BB_0540 | elongation factor G | 45 | 65 | 49 | 1.444444444 | 1.088888889 |
| — | predicted | antisense: guaA | 33531 | 18229 | 36441 | 0.543646178 | 1.086785363 |
| | RNA | | | | | | |
| — | predicted | antisense: recA | 33340 | 15227 | 36224 | 0.456718656 | 1.086502699 |
| | RNA | | | | | | |
| — | predicted | antisense: BB_0158 | 24644 | 30014 | 26749 | 1.217902938 | 1.085416329 |
| | RNA | | | | | | |
| prfB | BB_0074 | peptide chain release factor 2 | 12 | 31 | 13 | 2.583333333 | 1.083333333 |
| — | BB_0565 | purine-binding chemotaxis | 36 | 53 | 39 | 1.472222222 | 1.083333333 |
| | | protein | | | | | |
| — | BB_0110 | hypothetical protein | 108 | 154 | 117 | 1.425925926 | 1.083333333 |
| — | BB_0127 | 30S ribosomal protein S1 | 37 | 35 | 40 | 0.945945946 | 1.081081081 |
| — | BB_B26 | hypothetical protein | 611 | 196 | 660 | 0.320785597 | 1.080196399 |
| — | predicted | antisense: BB_0785 | 45789 | 55681 | 49411 | 1.216034419 | 1.079101968 |
| | RNA | | | | | | |
| — | predicted | antisense: BB_0283 | 37817 | 24792 | 40808 | 0.655578179 | 1.079091414 |
| | RNA | | | | | | |
| rimM | BB_0697 | ribosome maturation factor | 127 | 154 | 137 | 1.212598425 | 1.078740157 |
| | | RimM | | | | | |
| recB | BB_0633 | exodeoxyribonuclease V subunit | 13 | 9 | 14 | 0.692307692 | 1.076923077 |
| | | beta | | | | | |
| — | predicted | antisense: ptsP | 57304 | 29897 | 61666 | 0.521726232 | 1.076120341 |
| | RNA | | | | | | |
| — | predicted | antisense: pgk | 70406 | 34715 | 75667 | 0.493068773 | 1.074723745 |
| | RNA | | | | | | |
| — | BB_0756 | hypothetical protein | 27 | 23 | 29 | 0.851851852 | 1.074074074 |
| flgL | BB_0182 | flagellar hook-associated protein | 315 | 446 | 338 | 1.415873016 | 1.073015873 |
| | | FlgL | | | | | |
| rplU | BB_0778 | 50S ribosomal protein L21 | 14 | 34 | 15 | 2.428571429 | 1.071428571 |
| rpoN | BB_0450 | RNA polymerase sigma-54 | 28 | 28 | 30 | 1 | 1.071428571 |
| | | factor | | | | | |
| — | BB_0250 | inner membrane protein | 997 | 666 | 1063 | 0.668004012 | 1.066198596 |
| — | predicted | antisense: BB_0805 | 36967 | 22313 | 39407 | 0.603592393 | 1.066004815 |
| | RNA | | | | | | |
| — | predicted | antisense: BB_0429 | 17868 | 22062 | 19047 | 1.234721289 | 1.065983882 |
| | RNA | | | | | | |
| — | predicted | antisense: rpoC | 36846 | 18738 | 39230 | 0.508549096 | 1.064701732 |
| | RNA | | | | | | |
| — | predicted | antisense: pgk | 35559 | 15222 | 37841 | 0.42807728 | 1.064175033 |
| | RNA | | | | | | |
| — | predicted | antisense: BB_0805 | 45473 | 24906 | 48324 | 0.54770963 | 1.062696545 |
| | RNA | | | | | | |
| — | BB_0538 | hypothetical protein | 17 | 12 | 18 | 0.705882353 | 1.058823529 |
| — | predicted | antisense: BB_0789 | 35741 | 19721 | 37813 | 0.551775272 | 1.057972636 |
| | RNA | | | | | | |
| — | predicted | antisense: fliM | 36905 | 18769 | 38932 | 0.508576074 | 1.054924807 |
| | RNA | | | | | | |
| — | BB_0683 | 3-hydroxy-3-methylglutaryl- | 19 | 37 | 20 | 1.947368421 | 1.052631579 |
| | | CoA synthase | | | | | |
| — | BB_0709 | hypothetical protein | 19 | 18 | 20 | 0.947368421 | 1.052631579 |
| — | BB_P19 | hypothetical protein | 76 | 59 | 80 | 0.776315789 | 1.052631579 |
| — | BB_Q12 | hypothetical protein | 19 | 6 | 20 | 0.315789474 | 1.052631579 |
| rpsG | BB_0386 | 30S ribosomal protein S7 | 39 | 42 | 41 | 1.076923077 | 1.051282051 |
| — | predicted | antisense: rpsJ | 69096 | 29059 | 72573 | 0.420559801 | 1.050321292 |
| | RNA | | | | | | |
| — | predicted | antisense: BB_0824 | 35891 | 18946 | 37684 | 0.527876069 | 1.049956814 |
| | RNA | | | | | | |
| — | predicted | antisense: rplD | 38995 | 16168 | 40902 | 0.414617259 | 1.048903706 |
| | RNA | | | | | | |
| — | predicted | antisense: gyrB | 86626 | 83151 | 90751 | 0.959885023 | 1.047618498 |
| | RNA | | | | | | |
| secF | BB_0653 | protein translocase subunit SecF | 44 | 141 | 46 | 3.204545455 | 1.045454545 |
| — | BB_0816 | hypothetical protein | 45 | 33 | 47 | 0.733333333 | 1.044444444 |
| — | BB_0746 | oligopeptide ABC transporter | 234 | 193 | 244 | 0.824786325 | 1.042735043 |
| | | permease | | | | | |
| — | predicted | antisense: ffh | 40282 | 24929 | 41914 | 0.618862023 | 1.040514374 |
| | RNA | | | | | | |
| — | BB_0281 | motility protein A | 25 | 41 | 26 | 1.64 | 1.04 |
| rpsP | BB_0695 | 30S ribosomal protein S16 | 25 | 32 | 26 | 1.28 | 1.04 |
| — | predicted | antisense: pgk | 46633 | 21500 | 48490 | 0.461046898 | 1.039821586 |
| | RNA | | | | | | |
| mlpG | BB_O28 | lipoprotein | 26 | 11 | 27 | 0.423076923 | 1.038461538 |
| — | BB_0397 | hypothetical protein | 26 | 8 | 27 | 0.307692308 | 1.038461538 |
| gyrB | BB_0436 | DNA gyrase subunit B | 27 | 39 | 28 | 1.444444444 | 1.037037037 |
| — | BB_0460 | lipoprotein | 1122 | 692 | 1163 | 0.616755793 | 1.036541889 |
| — | predicted | antisense: BB_0579 | 32417 | 22333 | 33565 | 0.688928649 | 1.035413518 |
| | RNA | | | | | | |
| — | BB_Q17 | hypothetical protein | 127 | 42 | 131 | 0.330708661 | 1.031496063 |
| rpsT | BB_0233 | 30S ribosomal protein S20 | 32 | 41 | 33 | 1.28125 | 1.03125 |
| erpQ | BB_N39 | protein ErpQ | 130 | 295 | 134 | 2.269230769 | 1.030769231 |
| — | BB_A46 | hypothetical protein | 33 | 22 | 34 | 0.666666667 | 1.03030303 |
| — | predicted | antisense: flgD | 21907 | 20769 | 22567 | 0.948053134 | 1.030127357 |
| | RNA | | | | | | |
| — | predicted | antisense: secA | 38867 | 21017 | 39988 | 0.540741503 | 1.028841948 |
| | RNA | | | | | | |
| — | BB_0801 | translation initiation factor IF-2 | 40 | 38 | 41 | 0.95 | 1.025 |
| — | predicted | antisense: gyrB | 42244 | 22926 | 43248 | 0.542704289 | 1.023766689 |
| | RNA | | | | | | |
| — | predicted | antisense: gap | 83833 | 43247 | 85811 | 0.515870838 | 1.023594527 |
| | RNA | | | | | | |
| — | BB_0656 | oxygen-independent | 86 | 181 | 88 | 2.104651163 | 1.023255814 |
| | | coproporphyrinogen III oxidase | | | | | |
| — | BB_0028 | hypothetical protein | 89 | 85 | 91 | 0.95505618 | 1.02247191 |
| — | BB_0144 | glycine/betaine ABC transporter | 463 | 308 | 473 | 0.665226782 | 1.021598272 |
| | | substrate-binding protein | | | | | |
| — | predicted | antisense: tRNA-Trp-1 rpmG | 39226 | 44973 | 40055 | 1.146509968 | 1.021133942 |
| | RNA | | | | | | |
| — | predicted | antisense: rpsC rplV | 40494 | 19628 | 41269 | 0.484713785 | 1.019138638 |
| | RNA | | | | | | |
| bppC | BB_Q45 | protein BppC | 109 | 217 | 111 | 1.990825688 | 1.018348624 |
| — | BB_A11 | hypothetical protein | 83 | 39 | 84 | 0.469879518 | 1.012048193 |
| — | BB_P26 | hypothetical protein | 351 | 406 | 355 | 1.156695157 | 1.011396011 |
| — | BB_0734 | Sua5/YciO/YrdC/YwlC family | 132 | 152 | 133 | 1.151515152 | 1.007575758 |
| | | protein | | | | | |
| — | predicted | antisense: gyrA | 90201 | 37473 | 90726 | 0.415438853 | 1.005820335 |
| | RNA | | | | | | |
| — | BB_P29 | hypothetical protein | 375 | 328 | 377 | 0.874666667 | 1.005333333 |
| — | predicted | antisense: ptsP | 36729 | 21714 | 36898 | 0.591194969 | 1.004601269 |
| | RNA | | | | | | |
| — | BB_0543 | hypothetical protein | 13 | 103 | 13 | 7.923076923 | 1 |
| — | BB_0592 | hypothetical protein | 6 | 30 | 6 | 5 | 1 |
| — | BB_B24 | hypothetical protein | 8 | 38 | 8 | 4.75 | 1 |
| miaA | BB_0821 | tRNA dimethylallyltransferase | 11 | 42 | 11 | 3.818181818 | 1 |
| pth | BB_0787 | peptidyl-tRNA hydrolase | 4 | 15 | 4 | 3.75 | 1 |
| cysS | BB_0599 | cysteine--tRNA ligase | 7 | 19 | 7 | 2.714285714 | 1 |
| — | BB_0458 | hypothetical protein | 19 | 45 | 19 | 2.368421053 | 1 |
| glys | BB_0371 | glycine--tRNA ligase | 18 | 42 | 18 | 2.333333333 | 1 |
| rplL | BB_0390 | 50S ribosomal protein L7/L12 | 9 | 16 | 9 | 1.777777778 | 1 |
| — | BB_0092 | V-type ATPase subunit D | 3 | 5 | 3 | 1.666666667 | 1 |
| — | BB_0621 | 4-methyl-5(b-hydroxyethyl)- | 48 | 79 | 48 | 1.645833333 | 1 |
| | | thiazole monophosphate | | | | | |
| | | biosynthesis protein | | | | | |
| — | BB_0805 | polyribonucleotide | 16 | 21 | 16 | 1.3125 | 1 |
| | | nucleotidyltransferase | | | | | |
| — | BB_J43 | hypothetical protein | 18 | 20 | 18 | 1.111111111 | 1 |
| — | BB_0075 | hypothetical protein | 11 | 12 | 11 | 1.090909091 | 1 |
| erpN | BB_L39 | ErpA8 protein | 4 | 4 | 4 | 1 | 1 |
| tilS | BB_0788 | tRNA(Ile)-lysidine synthase | 8 | 8 | 8 | 1 | 1 |
| — | BB_R44 | hypothetical protein | 7 | 6 | 7 | 0.857142857 | 1 |
| — | BB_N03 | hypothetical protein | 7 | 6 | 7 | 0.857142857 | 1 |
| topA | BB_0828 | DNA topoisomerase I | 120 | 71 | 120 | 0.591666667 | 1 |
| — | BB_Q29 | hypothetical protein | 29 | 15 | 29 | 0.517241379 | 1 |
| clpX | BB_0612 | ATP-dependent protease ATP- | 1221 | 1766 | 1220 | 1.446355446 | 0.999180999 |
| | | binding subunit ClpX | | | | | |
| — | predicted | antisense: BB_0789 | 59217 | 33940 | 59002 | 0.573146225 | 0.996369286 |
| | RNA | | | | | | |
| — | predicted | antisense: BB_0544 | 34812 | 23768 | 34599 | 0.682753074 | 0.99388142 |
| | RNA | | | | | | |
| — | predicted | antisense: pgk gap | 58885 | 25419 | 58511 | 0.431671903 | 0.993648637 |
| | RNA | | | | | | |
| — | predicted | antisense: BB_0281 | 31321 | 21635 | 30998 | 0.690750615 | 0.98968743 |
| | RNA | | | | | | |
| — | predicted | antisense: groS | 18987 | 30852 | 18770 | 1.624901248 | 0.988571128 |
| | RNA | | | | | | |
| — | predicted | antisense: flhA | 60393 | 28161 | 59685 | 0.466295763 | 0.988276787 |
| | RNA | | | | | | |
| — | predicted | antisense: BB_B16 | 37859 | 14574 | 37357 | 0.3849547 | 0.986740273 |
| | RNA | | | | | | |
| mreC | BB_0716 | rod shape-determining protein | 72 | 85 | 71 | 1.180555556 | 0.986111111 |
| | | MreC | | | | | |
| — | predicted | antisense: trmE | 30104 | 21891 | 29646 | 0.727179112 | 0.984786075 |
| | RNA | | | | | | |
| — | BB_0315 | hypothetical protein | 62 | 40 | 61 | 0.64516129 | 0.983870968 |
| — | BB_0243 | glycerol-3-phosphate | 166 | 209 | 163 | 1.259036145 | 0.981927711 |
| | | dehydrogenase | | | | | |
| — | predicted | antisense: rpsL | 61764 | 39184 | 60570 | 0.63441487 | 0.98066835 |
| | RNA | | | | | | |
| — | BB_R05 | hypothetical protein | 100 | 61 | 98 | 0.61 | 0.98 |
| — | BB_0648 | hypothetical protein | 50 | 28 | 49 | 0.56 | 0.98 |
| — | predicted | antisense: tuf | 52944 | 15127 | 51878 | 0.285716984 | 0.979865518 |
| | RNA | | | | | | |
| — | BB_0638 | Na+/H+ antiporter | 95 | 209 | 93 | 2.2 | 0.978947368 |
| bppC | BB_P37 | site-specific recombinase | 176 | 174 | 172 | 0.988636364 | 0.977272727 |
| — | BB_P07 | hypothetical protein | 374 | 168 | 365 | 0.449197861 | 0.975935829 |
| — | BB_A57 | P45-13 | 412 | 74 | 402 | 0.17961165 | 0.975728155 |
| — | BB_0269 | ATP-binding protein | 153 | 116 | 149 | 0.758169935 | 0.973856209 |
| — | predicted | antisense: acpP | 33655 | 29974 | 32765 | 0.890625464 | 0.973555192 |
| | RNA | | | | | | |
| — | BB_A66 | outer surface protein | 917 | 217 | 892 | 0.236641221 | 0.972737186 |
| bppB | BB_R37 | BppB | 36 | 17 | 35 | 0.472222222 | 0.972222222 |
| clpP | BB_0611 | ATP-dependent Clp protease | 209 | 211 | 203 | 1.009569378 | 0.971291866 |
| | | proteolytic subunit | | | | | |
| — | predicted | antisense: BB_0283 | 27837 | 22723 | 27025 | 0.816287675 | 0.97083019 |
| | RNA | | | | | | |
| — | predicted | antisense: BB_0789 | 38551 | 14469 | 37319 | 0.375321003 | 0.968042334 |
| | RNA | | | | | | |
| — | predicted | antisense: rplB rplW | 39815 | 13363 | 38530 | 0.335627276 | 0.967725732 |
| | RNA | | | | | | |
| — | predicted | antisense: BB_0067 | 37094 | 17396 | 35881 | 0.468970723 | 0.967299294 |
| | RNA | | | | | | |
| — | predicted | antisense: BB_0504 | 45882 | 20391 | 44271 | 0.444422649 | 0.964888191 |
| | RNA | | | | | | |
| — | BB_0768 | pyridoxal kinase | 249 | 133 | 240 | 0.534136546 | 0.963855422 |
| — | predicted | antisense: ftsZ | 34527 | 24900 | 33269 | 0.721174733 | 0.963564746 |
| | RNA | | | | | | |
| coaBC | BB_0812 | bifunctional | 102 | 120 | 98 | 1.176470588 | 0.960784314 |
| | | phosphopantothenoylcysteine | | | | | |
| | | decarboxylase/phospho- | | | | | |
| | | pantothenate--cysteine ligase | | | | | |
| ptsP | BB_0558 | phosphoenolpyruvate-protein | 22 | 85 | 21 | 3.863636364 | 0.954545455 |
| | | phosphatase | | | | | |
| dnaK | BB_0518 | chaperone protein DnaK | 22 | 77 | 21 | 3.5 | 0.954545455 |
| — | predicted | antisense: rnpA | 38978 | 25853 | 37140 | 0.663271589 | 0.952845195 |
| | RNA | | | | | | |
| — | BB_0125 | hypothetical protein | 21 | 15 | 20 | 0.714285714 | 0.952380952 |
| gap | BB_0057 | glyceraldehyde 3-phosphate | 40 | 53 | 38 | 1.325 | 0.95 |
| | | dehydrogenase | | | | | |
| — | BB_0629 | PTS system fructose-specific | 486 | 579 | 460 | 1.191358025 | 0.946502058 |
| | | transporter subunit IIABC | | | | | |
| recG | BB_0581 | ATP-dependent DNA helicase | 221 | 209 | 209 | 0.945701357 | 0.945701357 |
| | | RecG | | | | | |
| fusA | BB_0691 | elongation factor G | 55 | 36 | 52 | 0.654545455 | 0.945454545 |
| pncB | BB_0635 | nicotinate | 91 | 64 | 86 | 0.703296703 | 0.945054945 |
| | | phosphoribosyltransferase | | | | | |
| — | BB_0506 | hypothetical protein | 141 | 107 | 133 | 0.758865248 | 0.943262411 |
| — | predicted | antisense: infC | 24463 | 26460 | 23072 | 1.081633487 | 0.943138618 |
| | RNA | | | | | | |
| — | predicted | antisense: ptsP | 45602 | 17542 | 43002 | 0.384676111 | 0.942984957 |
| | RNA | | | | | | |
| — | BB_P11 | hypothetical protein | 467 | 181 | 439 | 0.3875803 | 0.940042827 |
| — | BB_0283 | flagellar hook protein FlgE | 16 | 11 | 15 | 0.6875 | 0.9375 |
| — | predicted | antisense: rplN rplX | 33660 | 18385 | 31522 | 0.546197267 | 0.936482472 |
| | RNA | | | | | | |
| — | BB_0381 | hypothetical protein | 47 | 58 | 44 | 1.234042553 | 0.936170213 |
| — | predicted | antisense: BB_0236 | 32984 | 23615 | 30825 | 0.715953189 | 0.934544021 |
| | RNA | | | | | | |
| — | BB_0786 | 50S ribosomal protein | 15 | 40 | 14 | 2.666666667 | 0.933333333 |
| | | L25/general stress protein Ctc | | | | | |
| — | predicted | antisense: rplM | 57061 | 39728 | 53232 | 0.69623736 | 0.932896374 |
| | RNA | | | | | | |
| — | BB_0001 | hypothetical protein | 129 | 66 | 120 | 0.511627907 | 0.930232558 |
| — | BB_0354 | hypothetical protein | 14 | 63 | 13 | 4.5 | 0.928571429 |
| — | BB_R22 | hypothetical protein | 112 | 52 | 104 | 0.464285714 | 0.928571429 |
| — | BB_S07 | hypothetical protein | 14 | 2 | 13 | 0.142857143 | 0.928571429 |
| — | BB_0471 | hypothetical protein | 106 | 56 | 98 | 0.528301887 | 0.924528302 |
| — | BB_0180 | flagellar protein FlbF | 13 | 20 | 12 | 1.538461538 | 0.923076923 |
| — | BB_0509 | hypothetical protein | 26 | 27 | 24 | 1.038461538 | 0.923076923 |
| — | BB_O44 | phage terminase large subunit | 13 | 4 | 12 | 0.307692308 | 0.923076923 |
| — | BB_t22 | Xaa tRNA | 13 | 1 | 12 | 0.076923077 | 0.923076923 |
| — | predicted | antisense: gyrB | 86143 | 37243 | 79436 | 0.43233925 | 0.922141091 |
| | RNA | | | | | | |
| rplC | BB_0478 | 50S ribosomal protein L3 | 38 | 37 | 35 | 0.973684211 | 0.921052632 |
| murC | BB_0817 | UDP-N-acetylmuramate--L- | 25 | 27 | 23 | 1.08 | 0.92 |
| | | alanine ligase | | | | | |
| fliM | BB_0278 | flagellar motor switch protein | 25 | 19 | 23 | 0.76 | 0.92 |
| | | FliM | | | | | |
| — | BB_D04 | hypothetical protein | 72 | 24 | 66 | 0.333333333 | 0.916666667 |
| — | BB_0574 | integral membrane protein | 47 | 49 | 43 | 1.042553191 | 0.914893617 |
| — | BB_0137 | long-chain-fatty-acid CoA ligase | 22 | 47 | 20 | 2.136363636 | 0.909090909 |
| — | BB_0617 | hypothetical protein | 74 | 35 | 67 | 0.472972973 | 0.905405405 |
| — | BB_0312 | purine-binding chemotaxis | 21 | 13 | 19 | 0.619047619 | 0.904761905 |
| | | protein | | | | | |
| — | predicted | antisense: BB_0209 | 17088 | 23558 | 15447 | 1.378628277 | 0.903967697 |
| | RNA | | | | | | |
| — | predicted | antisense: tpiA | 50329 | 20508 | 45323 | 0.40747879 | 0.900534483 |
| | RNA | | | | | | |
| — | BB_M31 | hypothetical protein | 10 | 7 | 9 | 0.7 | 0.9 |
| — | predicted | antisense: flhB | 31829 | 23558 | 28490 | 0.740142637 | 0.895095667 |
| | RNA | | | | | | |
| — | BB_0654 | hypothetical protein | 93 | 117 | 83 | 1.258064516 | 0.892473118 |
| tuf | BB_0476 | elongation factor Tu | 37 | 41 | 33 | 1.108108108 | 0.891891892 |
| — | predicted | antisense: BB_0557 | 44932 | 28179 | 40067 | 0.62714769 | 0.891725274 |
| | RNA | | | | | | |
| — | predicted | antisense: rplK | 49219 | 28178 | 43797 | 0.572502489 | 0.88983929 |
| | RNA | | | | | | |
| — | BB_0096 | V-type ATP synthase subunit E | 9 | 15 | 8 | 1.666666667 | 0.888888889 |
| — | BB_0027 | hypothetical protein | 45 | 47 | 40 | 1.044444444 | 0.888888889 |
| — | BB_0527 | type III pantothenate kinase | 70 | 158 | 62 | 2.257142857 | 0.885714286 |
| — | predicted | antisense: rnpA rpmH | 40235 | 27790 | 35636 | 0.690692183 | 0.885696533 |
| | RNA | | | | | | |
| — | BB_0063 | hypothetical protein | 1012 | 436 | 895 | 0.43083004 | 0.884387352 |
| — | BB_0011 | hypothetical protein | 518 | 259 | 458 | 0.5 | 0.884169884 |
| — | BB_0600 | hypothetical protein | 17 | 23 | 15 | 1.352941176 | 0.882352941 |
| — | BB_A12 | BlyA family holin | 68 | 34 | 60 | 0.5 | 0.882352941 |
| tRNA- | BB_t04 | Ile tRNA | 1691 | 1060 | 1489 | 0.626848019 | 0.880544057 |
| Ile-1 | | | | | | | |
| bppB | BB_N36 | protein BppB | 25 | 6 | 22 | 0.24 | 0.88 |
| — | predicted | antisense: ffh | 63914 | 28122 | 56046 | 0.439997497 | 0.87689708 |
| | RNA | | | | | | |
| rpmH | BB_0440 | 50S ribosomal protein L34 | 40 | 79 | 35 | 1.975 | 0.875 |
| murI | BB_0100 | glutamate racemase | 8 | 15 | 7 | 1.875 | 0.875 |
| bppC | BB_L38 | protein BppC | 112 | 146 | 98 | 1.303571429 | 0.875 |
| blyB | BB_Q31 | holin protein | 24 | 6 | 21 | 0.25 | 0.875 |
| — | predicted | antisense: BB_0083 | 21449 | 29005 | 18734 | 1.352277495 | 0.873420672 |
| | RNA | | | | | | |
| — | predicted | antisense: gap | 68489 | 26996 | 59630 | 0.394165486 | 0.870650761 |
| | RNA | | | | | | |
| — | predicted | antisense: rpsH | 45679 | 28657 | 39764 | 0.627356116 | 0.870509424 |
| | RNA | | | | | | |
| — | BB_B22 | guanine/xanthine permease | 54 | 144 | 47 | 2.666666667 | 0.87037037 |
| — | BB_0571 | uridylate kinase | 493 | 247 | 429 | 0.501014199 | 0.870182556 |
| — | predicted | antisense: ffh | 29125 | 25850 | 25303 | 0.887553648 | 0.868772532 |
| | RNA | | | | | | |
| — | predicted | antisense: gyrB | 49961 | 29237 | 43295 | 0.585196453 | 0.866575929 |
| | RNA | | | | | | |
| — | BB_0406 | hypothetical protein | 339 | 234 | 293 | 0.690265487 | 0.864306785 |
| blyB | BB_M24 | holin protein | 103 | 50 | 89 | 0.485436893 | 0.86407767 |
| — | BB_0205 | hypothetical protein | 29 | 31 | 25 | 1.068965517 | 0.862068966 |
| — | BB_0264 | heat shock protein 70 | 94 | 115 | 81 | 1.223404255 | 0.861702128 |
| serS | BB_0226 | serine--tRNA ligase | 238 | 205 | 205 | 0.861344538 | 0.861344538 |
| — | BB_0560 | chaperone protein HtpG | 114 | 98 | 98 | 0.859649123 | 0.859649123 |
| — | BB_0496 | 50S ribosomal protein L30 | 7 | 13 | 6 | 1.857142857 | 0.857142857 |
| — | predicted | antisense: gyrA | 60806 | 18573 | 52076 | 0.305446831 | 0.856428642 |
| | RNA | | | | | | |
| — | predicted | antisense: rpmE | 33530 | 32647 | 28685 | 0.973665374 | 0.855502535 |
| | RNA | | | | | | |
| — | BB_0255 | M23 peptidase domain- | 400 | 306 | 342 | 0.765 | 0.855 |
| | | containing protein | | | | | |
| — | predicted | antisense: BB_0749 | 39504 | 22950 | 33701 | 0.580953827 | 0.853103483 |
| | RNA | | | | | | |
| — | BB_0542 | hypothetical protein | 34 | 24 | 29 | 0.705882353 | 0.852941176 |
| — | BB_0475 | lipoprotein | 34 | 9 | 29 | 0.264705882 | 0.852941176 |
| — | BB_0443 | spoIIIJ-associtated protein | 20 | 37 | 17 | 1.85 | 0.85 |
| rpoB | BB_0389 | DNA-directed RNA polymerase | 20 | 17 | 17 | 0.85 | 0.85 |
| | | subunit beta | | | | | |
| — | BB_0602 | chaperonin | 72 | 52 | 61 | 0.722222222 | 0.847222222 |
| — | predicted | antisense: flgC | 48385 | 23898 | 40991 | 0.493913403 | 0.847184045 |
| | RNA | | | | | | |
| — | BB_0086 | Mg chelatase-like protein | 742 | 546 | 628 | 0.735849057 | 0.846361186 |
| — | BB_A08 | hypothetical protein | 13 | 9 | 11 | 0.692307692 | 0.846153846 |
| — | BB_0360 | hypothetical protein | 45 | 49 | 38 | 1.088888889 | 0.844444444 |
| — | BB_0008 | hypothetical protein | 32 | 18 | 27 | 0.5625 | 0.84375 |
| pstB | BB_0218 | phosphate ABC transporter | 1489 | 831 | 1255 | 0.55809268 | 0.842847549 |
| | | ATP-binding protein | | | | | |
| — | BB_0155 | lipoprotein | 157 | 112 | 132 | 0.713375796 | 0.840764331 |
| — | predicted | antisense: tpiA | 52378 | 17675 | 43943 | 0.337450838 | 0.838959105 |
| | RNA | | | | | | |
| — | predicted | antisense: groL | 38526 | 26863 | 32316 | 0.697269377 | 0.838810154 |
| | RNA | | | | | | |
| — | predicted | antisense: groL | 76651 | 32516 | 64273 | 0.424208425 | 0.838514827 |
| | RNA | | | | | | |
| — | BB_0792 | hypothetical protein | 68 | 52 | 57 | 0.764705882 | 0.838235294 |
| — | BB_R10 | hypothetical protein | 142 | 34 | 119 | 0.23943662 | 0.838028169 |
| — | predicted | antisense: BB_0666 | 18279 | 27562 | 15273 | 1.507850539 | 0.835548991 |
| | RNA | | | | | | |
| — | BB_B06 | chitibiose transporter protein | 291 | 164 | 243 | 0.563573883 | 0.835051546 |
| | | ChbB | | | | | |
| tsf | BB_0122 | elongation factor Ts | 24 | 19 | 20 | 0.791666667 | 0.833333333 |
| — | BB_S17 | hypothetical protein | 42 | 7 | 35 | 0.166666667 | 0.833333333 |
| — | predicted | antisense: BB_0346 | 36335 | 21722 | 30244 | 0.597825788 | 0.832365488 |
| | RNA | | | | | | |
| — | predicted | antisense: BB_0149 | 37725 | 28431 | 31340 | 0.753638171 | 0.83074884 |
| | RNA | | | | | | |
| — | predicted | antisense: BB_0137 | 50131 | 10862 | 41519 | 0.216672319 | 0.82821009 |
| | RNA | | | | | | |
| — | BB_A43 | hypothetical protein | 17 | 10 | 14 | 0.588235294 | 0.823529412 |
| — | BB_0170 | hypothetical protein | 344 | 298 | 283 | 0.86627907 | 0.822674419 |
| — | predicted | antisense: pgk | 49312 | 18583 | 40471 | 0.376845393 | 0.820713011 |
| | RNA | | | | | | |
| — | predicted | antisense: BB_0355 | 53824 | 35102 | 44064 | 0.652162604 | 0.818668252 |
| | RNA | | | | | | |
| — | BB_0714 | hypothetical protein | 11 | 19 | 9 | 1.727272727 | 0.818181818 |
| — | BB_0614 | hypothetical protein | 115 | 278 | 94 | 2.417391304 | 0.817391304 |
| — | predicted | antisense: BB_0801 | 46073 | 14935 | 37641 | 0.324159486 | 0.816986087 |
| | RNA | | | | | | |
| — | predicted | antisense: rpsN rpsH | 38210 | 16772 | 31215 | 0.438942685 | 0.81693274 |
| | RNA | | | | | | |
| — | predicted | antisense: rpsO | 40369 | 30359 | 32962 | 0.752037454 | 0.816517625 |
| | RNA | | | | | | |
| — | predicted | antisense: eno | 31297 | 31783 | 25388 | 1.015528645 | 0.811195961 |
| | RNA | | | | | | |
| — | predicted | antisense: infC | 23714 | 27036 | 19227 | 1.140086025 | 0.810786877 |
| | RNA | | | | | | |
| — | predicted | antisense: eno | 29905 | 28992 | 24243 | 0.969469988 | 0.810667113 |
| | RNA | | | | | | |
| — | predicted | antisense: ptsP | 57572 | 18427 | 46666 | 0.320068783 | 0.810567637 |
| | RNA | | | | | | |
| — | predicted | antisense: guaA | 43695 | 12182 | 35261 | 0.278796201 | 0.806980204 |
| | RNA | | | | | | |
| — | BB_0597 | methyl-accepting chemotaxis | 97 | 55 | 78 | 0.567010309 | 0.804123711 |
| | | protein | | | | | |
| — | BB_0187 | hypothetical protein | 10 | 65 | 8 | 6.5 | 0.8 |
| murA | BB_0472 | UDP-N-acetylglucosamine 1- | 15 | 24 | 12 | 1.6 | 0.8 |
| | | carboxyvinyltransferase | | | | | |
| — | BB_0823 | lipoprotein | 8549 | 3787 | 6828 | 0.442975787 | 0.798689905 |
| — | predicted | antisense: trx | 44292 | 27201 | 35346 | 0.614128962 | 0.798022216 |
| | RNA | | | | | | |
| — | predicted | antisense: BB_0059 | 32673 | 20918 | 26049 | 0.640222814 | 0.797263796 |
| | RNA | | | | | | |
| — | BB_0465 | hypothetical protein | 177 | 53 | 141 | 0.299435028 | 0.796610169 |
| — | BB_0050 | hypothetical protein | 171 | 239 | 136 | 1.397660819 | 0.795321637 |
| rpsK | BB_0501 | 30S ribosomal protein S11 | 34 | 12 | 27 | 0.352941176 | 0.794117647 |
| p66 | BB_0603 | integral outer membrane protein | 247 | 289 | 196 | 1.170040486 | 0.793522267 |
| | | p66 | | | | | |
| rpoA | BB_0502 | DNA-directed RNA polymerase | 29 | 20 | 23 | 0.689655172 | 0.793103448 |
| | | subunit alpha | | | | | |
| — | BB_0115 | 30S ribosomal protein S6 | 19 | 36 | 15 | 1.894736842 | 0.789473684 |
| — | predicted | antisense: groL | 59099 | 26247 | 46565 | 0.44411919 | 0.787915193 |
| | RNA | | | | | | |
| rplD | BB_0479 | 50S ribosomal protein L4 | 28 | 15 | 22 | 0.535714286 | 0.785714286 |
| — | BB_0459 | hypothetical protein | 51 | 41 | 40 | 0.803921569 | 0.784313725 |
| — | predicted | antisense: gyrB | 52360 | 31024 | 41019 | 0.592513369 | 0.783403361 |
| | RNA | | | | | | |
| — | predicted | antisense: BB_0458 | 45632 | 18449 | 35735 | 0.404299614 | 0.783112728 |
| | RNA | | | | | | |
| — | predicted | antisense: ptsP | 47387 | 26968 | 36998 | 0.56910123 | 0.780762656 |
| | RNA | | | | | | |
| — | BB_S35 | PF-32 protein | 9 | 12 | 7 | 1.333333333 | 0.777777778 |
| erpY | BB_R42 | ErpY | 9 | 11 | 7 | 1.222222222 | 0.777777778 |
| — | BB_D21 | hypothetical protein | 9 | 8 | 7 | 0.888888889 | 0.777777778 |
| — | BB_N31 | hypothetical protein | 27 | 7 | 21 | 0.259259259 | 0.777777778 |
| — | BB_D09 | hypothetical protein | 54 | 8 | 42 | 0.148148148 | 0.777777778 |
| — | BB_0103 | hypothetical protein | 80 | 71 | 62 | 0.8875 | 0.775 |
| — | predicted | antisense: BB_0355 | 117881 | 44515 | 91175 | 0.37762659 | 0.773449496 |
| | RNA | | | | | | |
| — | BB_0413 | integral membrane protein | 127 | 93 | 98 | 0.732283465 | 0.771653543 |
| rpsL | BB_0387 | 30S ribosomal protein S12 | 35 | 69 | 27 | 1.971428571 | 0.771428571 |
| — | BB_0493 | 50S ribosomal protein L6 | 70 | 53 | 54 | 0.757142857 | 0.771428571 |
| — | BB_0246 | M23 peptidase domain- | 13 | 55 | 10 | 4.230769231 | 0.769230769 |
| | | containing protein | | | | | |
| rplR | BB_0494 | 50S ribosomal protein L18 | 26 | 44 | 20 | 1.692307692 | 0.769230769 |
| — | BB_N10 | hypothetical protein | 26 | 5 | 20 | 0.192307692 | 0.769230769 |
| — | BB_0507 | hypothetical protein | 212 | 176 | 161 | 0.830188679 | 0.759433962 |
| — | predicted | antisense: BB_0067 | 52774 | 22815 | 40078 | 0.432315155 | 0.759426991 |
| | RNA | | | | | | |
| — | predicted | antisense: BB_0493 | 42471 | 20977 | 32166 | 0.493913494 | 0.757363848 |
| | RNA | | | | | | |
| — | predicted | antisense: BB_0158 | 25113 | 26987 | 19003 | 1.074622705 | 0.756699717 |
| | RNA | | | | | | |
| — | BB_A59 | hypothetical protein | 910 | 242 | 688 | 0.265934066 | 0.756043956 |
| — | predicted | antisense: BB_0202 | 20023 | 21421 | 15133 | 1.069819707 | 0.755780852 |
| | RNA | | | | | | |
| — | BB_0004 | phosphoglucomutase | 859 | 253 | 649 | 0.294528522 | 0.755529686 |
| — | BB_J27 | efflux ABC transporter | 102 | 43 | 77 | 0.421568627 | 0.754901961 |
| | | permease | | | | | |
| — | predicted | antisense: BB_0801 | 57366 | 21574 | 43192 | 0.376076422 | 0.752919848 |
| | RNA | | | | | | |
| — | BB_0591 | competence locus E | 1753 | 881 | 1319 | 0.502567028 | 0.752424415 |
| — | BB_0412 | membrane protein | 230 | 179 | 173 | 0.77826087 | 0.752173913 |
| erpP | BB_N38 | protein ErpP | 4 | 14 | 3 | 3.5 | 0.75 |
| hbb | BB_0232 | DNA-binding protein HU | 20 | 37 | 15 | 1.85 | 0.75 |
| — | BB_Q53 | hypothetical protein | 8 | 3 | 6 | 0.375 | 0.75 |
| — | predicted | antisense: rplT | 24991 | 22754 | 18622 | 0.910487776 | 0.745148253 |
| | RNA | | | | | | |
| — | BB_0674 | membrane protein | 701 | 575 | 521 | 0.820256776 | 0.743223966 |
| — | BB_B16 | oligopeptide ABC transporter | 66 | 136 | 49 | 2.060606061 | 0.742424242 |
| | | OppAIV | | | | | |
| — | BB_0776 | hypothetical protein | 31 | 30 | 23 | 0.967741935 | 0.741935484 |
| — | BB_0666 | N-acetylmuramoyl-L-alanine | 85 | 68 | 63 | 0.8 | 0.741176471 |
| | | amidase domain-containing | | | | | |
| | | protein | | | | | |
| eno | BB_0337 | enolase | 597 | 387 | 442 | 0.648241206 | 0.740368509 |
| — | BB_M03 | hypothetical protein | 19 | 7 | 14 | 0.368421053 | 0.736842105 |
| — | predicted | antisense: BB_0801 | 42670 | 17779 | 31432 | 0.416662761 | 0.736629951 |
| | RNA | | | | | | |
| — | BB_0094 | ATP synthase subunit A | 15 | 19 | 11 | 1.266666667 | 0.733333333 |
| — | BB_P31 | hypothetical protein | 30 | 29 | 22 | 0.966666667 | 0.733333333 |
| — | BB_P01 | phage portal protein | 105 | 37 | 77 | 0.352380952 | 0.733333333 |
| — | predicted | antisense: BB_0545 | 52578 | 18799 | 38525 | 0.357544981 | 0.73272091 |
| | RNA | | | | | | |
| rpiA | BB_0657 | ribose 5-phosphate isomerase A | 2168 | 1116 | 1585 | 0.514760148 | 0.731088561 |
| — | BB_0789 | ATP-dependent zinc | 26 | 25 | 19 | 0.961538462 | 0.730769231 |
| | | metalloprotease FtsH | | | | | |
| — | predicted | antisense: rpsI | 69200 | 41409 | 50558 | 0.598395954 | 0.730606936 |
| | RNA | | | | | | |
| — | BB_A47 | hypothetical protein | 63 | 46 | 46 | 0.73015873 | 0.73015873 |
| — | BB_J08 | surface protein | 70 | 47 | 51 | 0.671428571 | 0.728571429 |
| pstA | BB_0217 | phosphate ABC transporter | 378 | 290 | 275 | 0.767195767 | 0.727513228 |
| | | permease PstA | | | | | |
| — | predicted | antisense: BB_0795 | 48965 | 15928 | 35608 | 0.325293577 | 0.727213316 |
| | RNA | | | | | | |
| — | BB_0168 | dnaK suppressor | 252 | 195 | 183 | 0.773809524 | 0.726190476 |
| — | predicted | antisense: BB_0735 | 33000 | 23585 | 23944 | 0.71469697 | 0.725575758 |
| | RNA | | | | | | |
| — | predicted | antisense: rpsI | 74195 | 39030 | 53828 | 0.52604623 | 0.725493632 |
| | RNA | | | | | | |
| — | BB_0317 | integral membrane protein | 29 | 46 | 21 | 1.586206897 | 0.724137931 |
| — | BB_0047 | hypothetical protein | 47 | 69 | 34 | 1.468085106 | 0.723404255 |
| — | predicted | antisense: pgk | 50251 | 15705 | 36345 | 0.312531094 | 0.723269189 |
| | RNA | | | | | | |
| — | BB_B01 | acylphosphatase | 543 | 257 | 392 | 0.473296501 | 0.721915285 |
| — | predicted | antisense: BB_0504 | 33095 | 24143 | 23715 | 0.729505968 | 0.716573501 |
| | RNA | | | | | | |
| csrA | BB_0184 | carbon storage regulator | 345 | 254 | 247 | 0.736231884 | 0.715942029 |
| — | BB_0825 | hypothetical protein | 3864 | 1941 | 2750 | 0.502329193 | 0.711697723 |
| — | BB_0452 | chromate transporter | 179 | 158 | 127 | 0.882681564 | 0.709497207 |
| | | superfamily protein | | | | | |
| — | predicted | antisense: BB_0088 | 40595 | 21823 | 28747 | 0.53757852 | 0.708141397 |
| | RNA | | | | | | |
| — | BB_0620 | beta-glucosidase | 579 | 306 | 409 | 0.528497409 | 0.706390328 |
| flgC | BB_0293 | flagellar basal body rod protein | 17 | 29 | 12 | 1.705882353 | 0.705882353 |
| | | FlgC | | | | | |
| — | BB_A42 | hypothetical protein | 68 | 10 | 48 | 0.147058824 | 0.705882353 |
| — | predicted | antisense: BB_0147 | 73211 | 40956 | 51655 | 0.55942413 | 0.705563372 |
| | RNA | | | | | | |
| rpsD | BB_0615 | 30S ribosomal protein S4 | 78 | 138 | 55 | 1.769230769 | 0.705128205 |
| — | BB_0399 | hypothetical protein | 37 | 19 | 26 | 0.513513514 | 0.702702703 |
| — | BB_S31 | hypothetical protein | 245 | 128 | 172 | 0.52244898 | 0.702040816 |
| — | BB_0545 | xylulokinase | 338 | 346 | 237 | 1.023668639 | 0.701183432 |
| — | BB_0439 | hypothetical protein | 10 | 14 | 7 | 1.4 | 0.7 |
| — | BB_0723 | adenylyl cyclase | 50 | 29 | 35 | 0.58 | 0.7 |
| — | BB_M11 | hypothetical protein | 43 | 8 | 30 | 0.186046512 | 0.697674419 |
| — | BB_P25 | hypothetical protein | 56 | 16 | 39 | 0.285714286 | 0.696428571 |
| — | predicted | antisense: prfB | 23877 | 27266 | 16597 | 1.141935754 | 0.695104075 |
| | RNA | | | | | | |
| argS | BB_0594 | arginine--tRNA ligase | 566 | 241 | 392 | 0.425795053 | 0.692579505 |
| — | BB_0544 | phosphoribosylpyrophosphate | 26 | 56 | 18 | 2.153846154 | 0.692307692 |
| | | synthetase | | | | | |
| — | predicted | antisense: ptsP | 56965 | 13026 | 39351 | 0.228666725 | 0.690792592 |
| | RNA | | | | | | |
| — | BB_P22 | hypothetical protein | 860 | 258 | 593 | 0.3 | 0.689534884 |
| — | predicted | antisense: BB_0327 | 57402 | 22110 | 39518 | 0.385178217 | 0.688442911 |
| | RNA | | | | | | |
| — | BB_0089 | hypothetical protein | 16 | 23 | 11 | 1.4375 | 0.6875 |
| — | BB_0642 | spermidine/putrescine ABC | 64 | 54 | 44 | 0.84375 | 0.6875 |
| | | transporter ATP-binding protein | | | | | |
| — | BB_0013 | hypothetical protein | 51 | 12 | 35 | 0.235294118 | 0.68627451 |
| — | BB_H04 | hypothetical protein | 76 | 47 | 52 | 0.618421053 | 0.684210526 |
| — | predicted | antisense: gyrA | 90376 | 30117 | 61775 | 0.333241126 | 0.683533239 |
| | RNA | | | | | | |
| bdrE | BB_S37 | protein BdrE | 22 | 11 | 15 | 0.5 | 0.681818182 |
| blyB | BB_P24 | holin protein | 243 | 89 | 165 | 0.366255144 | 0.679012346 |
| rplW | BB_0480 | 50S ribosomal protein L23 | 34 | 15 | 23 | 0.441176471 | 0.676470588 |
| — | BB_0199 | hypothetical protein | 58 | 27 | 39 | 0.465517241 | 0.672413793 |
| ospA | BB_A15 | outer surface protein A | 318 | 111 | 213 | 0.349056604 | 0.669811321 |
| — | predicted | antisense: BB_0087 | 48472 | 23647 | 32383 | 0.487848655 | 0.668076415 |
| | RNA | | | | | | |
| — | predicted | antisense: BB_0114 | 49579 | 31623 | 33069 | 0.637830533 | 0.666996107 |
| | RNA | | | | | | |
| — | BB_0583 | integral membrane protein | 171 | 168 | 114 | 0.98245614 | 0.666666667 |
| — | BB_M42 | phage terminase large subunit | 6 | 3 | 4 | 0.5 | 0.666666667 |
| — | BB_0081 | efflux ABC transporter | 42 | 21 | 28 | 0.5 | 0.666666667 |
| | | permease | | | | | |
| — | predicted | antisense: rpsR | 41655 | 24967 | 27740 | 0.599375825 | 0.665946465 |
| | RNA | | | | | | |
| — | predicted | antisense: BB_0789 | 55407 | 16638 | 36890 | 0.300286967 | 0.66580035 |
| | RNA | | | | | | |
| — | BB_0672 | chemotaxis protein CheY | 304 | 156 | 202 | 0.513157895 | 0.664473684 |
| — | predicted | antisense: rpsM rpmJ | 60988 | 34592 | 40230 | 0.567193546 | 0.659637962 |
| | RNA | | | | | | |
| — | predicted | antisense: BB_0544 | 57847 | 20017 | 38154 | 0.346033502 | 0.65956748 |
| | RNA | | | | | | |
| revA | BB_P27 | surface protein | 439 | 201 | 289 | 0.45785877 | 0.658314351 |
| — | BB_0062 | rRNA small subunit | 597 | 243 | 393 | 0.407035176 | 0.658291457 |
| | | methyltransferase E | | | | | |
| groS | BB_0741 | chaperonin GroS | 32 | 78 | 21 | 2.4375 | 0.65625 |
| — | BB_0159 | hypothetical protein | 122 | 95 | 80 | 0.778688525 | 0.655737705 |
| — | predicted | antisense: rpsG | 97339 | 27687 | 63802 | 0.28443892 | 0.65546184 |
| | RNA | | | | | | |
| rplA | BB_0392 | 50S ribosomal protein L1 | 29 | 22 | 19 | 0.75862069 | 0.655172414 |
| — | BB_0706 | polynucleotide | 559 | 274 | 366 | 0.490161002 | 0.654740608 |
| | | adenylyltransferase | | | | | |
| ffh | BB_0694 | signal recognition particle | 26 | 23 | 17 | 0.884615385 | 0.653846154 |
| | | protein | | | | | |
| — | predicted | antisense: BB_B22 | 75811 | 16883 | 49510 | 0.222698553 | 0.653071454 |
| | RNA | | | | | | |
| — | BB_0675 | hypothetical protein | 363 | 200 | 237 | 0.550964187 | 0.652892562 |
| nagB | BB_0152 | glucosamine-6-phosphate | 1154 | 363 | 753 | 0.314558059 | 0.652512998 |
| | | deaminase | | | | | |
| tRNA- | BB_t13 | Val tRNA | 80 | 51 | 52 | 0.6375 | 0.65 |
| Val-1 | | | | | | | |
| — | BB_0562 | hypothetical protein | 1296 | 471 | 842 | 0.363425926 | 0.649691358 |
| — | predicted | antisense: BB_0238 | 51631 | 27025 | 33465 | 0.523425849 | 0.648157115 |
| | RNA | | | | | | |
| cdr | BB_0728 | CoA-disulfide reductase | 1602 | 747 | 1032 | 0.466292135 | 0.644194757 |
| 16S | BB_r05 | 16S ribosomal RNA | 3488 | 2709 | 2243 | 0.776662844 | 0.643061927 |
| — | predicted | antisense: BB_B22 | 77813 | 19948 | 50036 | 0.256358192 | 0.6430288 |
| | RNA | | | | | | |
| — | BB_D14 | hypothetical protein | 14 | 8 | 9 | 0.571428571 | 0.642857143 |
| — | predicted | antisense: guaA | 72328 | 16144 | 46472 | 0.223205398 | 0.642517421 |
| | RNA | | | | | | |
| rpsJ | BB_0477 | 30S ribosomal protein S10 | 67 | 43 | 43 | 0.641791045 | 0.641791045 |
| — | BB_0546 | hypothetical protein | 700 | 319 | 449 | 0.455714286 | 0.641428571 |
| hflK | BB_0203 | protein HflK | 189 | 79 | 121 | 0.417989418 | 0.64021164 |
| — | predicted | antisense: gyrA | 78093 | 13010 | 49816 | 0.166596238 | 0.637906086 |
| | RNA | | | | | | |
| — | predicted | antisense: fliE | 57125 | 16447 | 36435 | 0.287912473 | 0.637811816 |
| | RNA | | | | | | |
| — | BB_0753 | hypothetical protein | 503 | 275 | 320 | 0.546719682 | 0.636182903 |
| — | BB_0280 | flagellar motor protein MotB | 30 | 30 | 19 | 1 | 0.633333333 |
| — | predicted | antisense: rpsB | 51564 | 25801 | 32539 | 0.500368474 | 0.631041036 |
| | RNA | | | | | | |
| — | BB_0129 | hypothetical protein | 137 | 41 | 86 | 0.299270073 | 0.627737226 |
| rnc | BB_0705 | ribonuclease III | 51 | 52 | 32 | 1.019607843 | 0.62745098 |
| — | BB_0202 | hemolysin | 323 | 214 | 202 | 0.6625387 | 0.625386997 |
| — | BB_O32 | PF-32 protein | 8 | 11 | 5 | 1.375 | 0.625 |
| — | BB_0404 | hypothetical protein | 16 | 13 | 10 | 0.8125 | 0.625 |
| frr | BB_0121 | ribosome recycling factor | 24 | 17 | 15 | 0.708333333 | 0.625 |
| — | BB_J18 | PF-32 protein | 8 | 5 | 5 | 0.625 | 0.625 |
| — | BB_0060 | endoribonuclease | 40 | 20 | 25 | 0.5 | 0.625 |
| — | BB_0676 | phosphoglycolate phosphatase | 428 | 283 | 267 | 0.661214953 | 0.623831776 |
| — | predicted | antisense: tsf | 56929 | 15786 | 35464 | 0.277292768 | 0.622951396 |
| | RNA | | | | | | |
| — | BB_B02 | hypothetical protein | 2350 | 803 | 1461 | 0.341702128 | 0.621702128 |
| — | predicted | antisense: BB_0088 | 45623 | 24671 | 28339 | 0.540757951 | 0.621155996 |
| | RNA | | | | | | |
| — | predicted | antisense: rplQ | 80945 | 23399 | 50094 | 0.289072827 | 0.618864661 |
| | RNA | | | | | | |
| coaD | BB_0702 | phosphopantetheine | 13 | 20 | 8 | 1.538461538 | 0.615384615 |
| | | adenylyltransferase | | | | | |
| — | predicted | antisense: BB_0322 | 68518 | 12797 | 42142 | 0.18676844 | 0.61505006 |
| | RNA | | | | | | |
| — | predicted | antisense: tsf | 58070 | 18578 | 35706 | 0.319924229 | 0.614878595 |
| | RNA | | | | | | |
| — | predicted | antisense: groL | 74218 | 26012 | 45467 | 0.350481015 | 0.612614191 |
| | RNA | | | | | | |
| — | predicted | antisense: eno | 39845 | 35556 | 24373 | 0.892357887 | 0.611695319 |
| | RNA | | | | | | |
| — | predicted | antisense: BB_0355 | 55802 | 23743 | 34065 | 0.425486542 | 0.610461991 |
| | RNA | | | | | | |
| — | BB_D13 | hypothetical protein | 1530 | 226 | 934 | 0.147712418 | 0.610457516 |
| — | BB_S26 | hypothetical protein | 1273 | 336 | 774 | 0.263943441 | 0.608012569 |
| — | BB_0349 | hypothetical protein | 368 | 153 | 223 | 0.41576087 | 0.605978261 |
| — | BB_0456 | hypothetical protein | 58 | 23 | 35 | 0.396551724 | 0.603448276 |
| — | BB_0715 | cell division protein FtsA | 25 | 44 | 15 | 1.76 | 0.6 |
| — | BB_0577 | hypothetical protein | 10 | 12 | 6 | 1.2 | 0.6 |
| — | BB_H0042 | hypothetical protein | 25 | 28 | 15 | 1.12 | 0.6 |
| — | BB_0798 | competence protein F | 80 | 35 | 48 | 0.4375 | 0.6 |
| — | predicted | antisense: BB_0147 | 122230 | 32260 | 73284 | 0.263928659 | 0.59955821 |
| | RNA | | | | | | |
| — | predicted | antisense: rpoA | 61632 | 16880 | 36602 | 0.273883697 | 0.593879803 |
| | RNA | | | | | | |
| bppC | BB_M37 | BppC | 49 | 42 | 29 | 0.857142857 | 0.591836735 |
| — | BB_0429 | hypothetical protein | 1770 | 531 | 1046 | 0.3 | 0.590960452 |
| — | predicted | antisense: groL | 69367 | 26372 | 40993 | 0.380180778 | 0.590958237 |
| | RNA | | | | | | |
| — | BB_H28 | PF-32 protein | 22 | 7 | 13 | 0.318181818 | 0.590909091 |
| — | BB_0017 | integral membrane protein | 681 | 372 | 402 | 0.546255507 | 0.59030837 |
| rpsB | BB_0123 | 30S ribosomal protein S2 | 56 | 50 | 33 | 0.892857143 | 0.589285714 |
| — | BB_M26 | hypothetical protein | 379 | 104 | 222 | 0.274406332 | 0.585751979 |
| — | BB_0722 | hypothetical protein | 190 | 101 | 111 | 0.531578947 | 0.584210526 |
| — | predicted | antisense: rplQ | 73534 | 25184 | 42740 | 0.342481029 | 0.581227731 |
| | RNA | | | | | | |
| — | BB_0067 | peptidase | 234 | 114 | 136 | 0.487179487 | 0.581196581 |
| — | BB_0211 | DNA mismatch repair protein | 195 | 69 | 113 | 0.353846154 | 0.579487179 |
| — | BB_0355 | transcription factor | 57 | 64 | 33 | 1.122807018 | 0.578947368 |
| rpsM | BB_0500 | 30S ribosomal protein S13 | 166 | 123 | 96 | 0.740963855 | 0.578313253 |
| — | predicted | antisense: groL | 68330 | 22456 | 39402 | 0.328640421 | 0.576642763 |
| | RNA | | | | | | |
| — | BB_A10 | hypothetical protein | 106 | 21 | 61 | 0.198113208 | 0.575471698 |
| trx | BB_0061 | thioredoxin | 21 | 33 | 12 | 1.571428571 | 0.571428571 |
| bdrA | BB_P34 | protein BdrA | 21 | 15 | 12 | 0.714285714 | 0.571428571 |
| napA | BB_0690 | neutrophil activating protein A | 28 | 11 | 16 | 0.392857143 | 0.571428571 |
| | | (napA) | | | | | |
| — | predicted | antisense: rpsB | 81822 | 44527 | 46482 | 0.544193493 | 0.568086823 |
| | RNA | | | | | | |
| — | BB_N26 | hypothetical protein | 183 | 39 | 103 | 0.213114754 | 0.56284153 |
| — | BB_0490 | 50S ribosomal protein L5 | 32 | 43 | 18 | 1.34375 | 0.5625 |
| mviN | BB_0810 | integral membrane protein MviN | 810 | 353 | 454 | 0.435802469 | 0.560493827 |
| — | BB_Q37 | hypothetical protein | 25 | 10 | 14 | 0.4 | 0.56 |
| — | predicted | antisense: rpsB | 54387 | 30908 | 30425 | 0.568297571 | 0.559416772 |
| | RNA | | | | | | |
| — | BB_0655 | heat shock protein | 707 | 296 | 394 | 0.418670438 | 0.5572843 |
| — | BB_B07 | alpha3-beta1 integrin-binding | 1824 | 521 | 1012 | 0.285635965 | 0.554824561 |
| | | protein | | | | | |
| truB | BB_0803 | tRNA pseudouridine synthase B | 92 | 31 | 51 | 0.336956522 | 0.554347826 |
| — | predicted | antisense: rpoA | 69943 | 13810 | 38584 | 0.197446492 | 0.5516492 |
| | RNA | | | | | | |
| rpmI | BB_0189 | 50S ribosomal protein L35 | 33 | 35 | 18 | 1.060606061 | 0.545454545 |
| gnd | BB_0561 | 6-phosphogluconate | 315 | 193 | 171 | 0.612698413 | 0.542857143 |
| | | dehydrogenase | | | | | |
| — | BB_A03 | outer membrane protein | 26 | 13 | 14 | 0.5 | 0.538461538 |
| — | BB_N19 | hypothetical protein | 39 | 11 | 21 | 0.282051282 | 0.538461538 |
| bppB | BB_O37 | protein BppB | 15 | 5 | 8 | 0.333333333 | 0.533333333 |
| alr | BB_0160 | alanine racemase | 109 | 69 | 58 | 0.633027523 | 0.532110092 |
| — | BB_0366 | aminopeptidase | 130 | 103 | 69 | 0.792307692 | 0.530769231 |
| osm28 | BB_A74 | outer membrane porin OMS28 | 34 | 17 | 18 | 0.5 | 0.529411765 |
| — | BB_0791 | thymidine kinase | 189 | 83 | 100 | 0.439153439 | 0.529100529 |
| — | predicted | antisense: groL | 67821 | 24725 | 35872 | 0.364562599 | 0.52892172 |
| | RNA | | | | | | |
| — | BB_0401 | glutamate transporter | 824 | 282 | 434 | 0.34223301 | 0.526699029 |
| — | BB_S02 | hypothetical protein | 19 | 5 | 10 | 0.263157895 | 0.526315789 |
| rpmB | BB_0350 | 50S ribosomal protein L28 | 154 | 103 | 81 | 0.668831169 | 0.525974026 |
| luxS | BB_0377 | S-ribosylhomocysteine lyase | 891 | 533 | 468 | 0.598204265 | 0.525252525 |
| — | BB_0586 | FemA protein | 141 | 82 | 74 | 0.581560284 | 0.524822695 |
| — | predicted | antisense: rplI | 76228 | 26825 | 39982 | 0.351904812 | 0.524505431 |
| | RNA | | | | | | |
| — | BB_J34 | lipoprotein | 103 | 34 | 54 | 0.330097087 | 0.524271845 |
| — | BB_0117 | hemolysin III | 542 | 387 | 284 | 0.71402214 | 0.52398524 |
| — | BB_0332 | peptide ABC transporter | 566 | 200 | 296 | 0.35335689 | 0.522968198 |
| | | permease | | | | | |
| — | BB_J11 | hypothetical protein | 391 | 258 | 203 | 0.659846547 | 0.519181586 |
| — | BB_0051 | integral membrane protein | 492 | 229 | 255 | 0.465447154 | 0.518292683 |
| asnS | BB_0101 | asparagine--tRNA ligase | 193 | 174 | 100 | 0.901554404 | 0.518134715 |
| rpmG | BB_0396 | 50S ribosomal protein L33 | 62 | 50 | 32 | 0.806451613 | 0.516129032 |
| — | predicted | antisense: rpsB | 71740 | 24471 | 36869 | 0.341106774 | 0.513925286 |
| | RNA | | | | | | |
| — | predicted | antisense: BB_0238 | 56873 | 26673 | 29197 | 0.468992316 | 0.513371899 |
| | RNA | | | | | | |
| — | predicted | antisense: BB_B22 | 93372 | 15380 | 47924 | 0.164717474 | 0.513258793 |
| | RNA | | | | | | |
| — | BB_0689 | hypothetical protein | 648 | 297 | 332 | 0.458333333 | 0.512345679 |
| — | predicted | antisense: rpsT | 43570 | 34889 | 22068 | 0.800757402 | 0.506495295 |
| | RNA | | | | | | |
| — | predicted | antisense: rpsK | 81384 | 16444 | 41213 | 0.202054458 | 0.50640175 |
| | RNA | | | | | | |
| — | BB_0658 | 2,3-bisphosphoglycerate- | 735 | 447 | 370 | 0.608163265 | 0.503401361 |
| | | dependent phosphoglycerate | | | | | |
| | | mutase | | | | | |
| rplS | BB_0699 | 50S ribosomal protein L19 | 36 | 49 | 18 | 1.361111111 | 0.5 |
| rbfA | BB_0802 | ribosome-binding factor A | 24 | 24 | 12 | 1 | 0.5 |
| — | BB_0639 | spermidine/putrescine ABC | 34 | 26 | 17 | 0.764705882 | 0.5 |
| | | transporter substrate-binding | | | | | |
| | | protein | | | | | |
| fliS | BB_0550 | flagellar protein FliS | 10 | 7 | 5 | 0.7 | 0.5 |
| — | BB_J17 | PF-32 protein | 6 | 4 | 3 | 0.666666667 | 0.5 |
| — | BB_O33 | plasmid partition protein | 4 | 2 | 2 | 0.5 | 0.5 |
| — | BB_A69 | putative surface protein | 196 | 63 | 98 | 0.321428571 | 0.5 |
| — | BB_0352 | hypothetical protein | 132 | 40 | 66 | 0.303030303 | 0.5 |
| — | BB_0331 | hypothetical protein | 10 | 3 | 5 | 0.3 | 0.5 |
| — | BB_H27 | hypothetical protein | 8 | 2 | 4 | 0.25 | 0.5 |
| — | BB_L25 | hypothetical protein | 26 | 3 | 13 | 0.115384615 | 0.5 |
| — | BB_L26 | hypothetical protein | 2 | 0 | 1 | 0 | 0.5 |
| pstC | BB_0216 | phosphate ABC transporter | 259 | 132 | 128 | 0.50965251 | 0.494208494 |
| | | permease PstC | | | | | |
| ung | BB_0053 | uracil-DNA glycosylase | 3295 | 1299 | 1626 | 0.394233687 | 0.493474962 |
| — | BB_0759 | membrane protein | 2113 | 513 | 1041 | 0.242782773 | 0.492664458 |
| — | BB_A13 | hypothetical protein | 520 | 49 | 256 | 0.094230769 | 0.492307692 |
| — | BB_A09 | hypothetical protein | 102 | 12 | 50 | 0.117647059 | 0.490196078 |
| — | BB_0707 | hypothetical protein | 72 | 35 | 35 | 0.486111111 | 0.486111111 |
| — | predicted | — | 220296 | 90190 | 107023 | 0.409403711 | 0.48581454 |
| | RNA | | | | | | |
| — | BB_0024 | hypothetical protein | 803 | 258 | 390 | 0.321295143 | 0.485678705 |
| murB | BB_0598 | UDP-N- | 513 | 191 | 249 | 0.372319688 | 0.485380117 |
| | | acetylenolpyruvoylglucosamine | | | | | |
| | | reductase | | | | | |
| — | BB_0029 | hypothetical protein | 235 | 85 | 114 | 0.361702128 | 0.485106383 |
| — | BB_0641 | spermidine/putrescine transport | 145 | 73 | 70 | 0.503448276 | 0.482758621 |
| | | system permease | | | | | |
| — | BB_J0056 | hypothetical protein | 27 | 0 | 13 | 0 | 0.481481481 |
| — | BB_R26 | hypothetical protein | 818 | 216 | 393 | 0.26405868 | 0.480440098 |
| 23S_rrlB | BB_r04 | 23S ribosomal RNA | 6339 | 2622 | 3039 | 0.41362991 | 0.479413157 |
| — | BB_D10 | lipoprotein | 107 | 49 | 51 | 0.457943925 | 0.476635514 |
| 23S_rrlA | BB_r02 | 23S ribosomal RNA | 6312 | 2516 | 3005 | 0.39860583 | 0.476077313 |
| — | predicted | antisense: BB_0465 | 62532 | 23288 | 29768 | 0.372417322 | 0.476044265 |
| | RNA | | | | | | |
| — | BB_0646 | hypothetical protein | 939 | 316 | 444 | 0.336528222 | 0.47284345 |
| — | BB_0419 | response regulator | 72 | 86 | 34 | 1.194444444 | 0.472222222 |
| rplQ | BB_0503 | 50S ribosomal protein L17 | 36 | 19 | 17 | 0.527777778 | 0.472222222 |
| — | predicted | antisense: BB_0559 | 83624 | 10987 | 39487 | 0.131385727 | 0.472196977 |
| | RNA | | | | | | |
| — | predicted | antisense: tRNA-Gln-1 | 54021 | 26518 | 25214 | 0.490883175 | 0.466744414 |
| | RNA | BB_0786 | | | | | |
| — | BB_R29 | hypothetical protein | 543 | 217 | 252 | 0.399631676 | 0.464088398 |
| — | BB_0225 | tRNA-dihydrouridine synthase | 419 | 253 | 194 | 0.603818616 | 0.46300716 |
| | | A | | | | | |
| rpmF | BB_0703 | 50S ribosomal protein L32 | 61 | 67 | 28 | 1.098360656 | 0.459016393 |
| — | BB_0252 | hypothetical protein | 1087 | 439 | 497 | 0.403863845 | 0.457221711 |
| blyA | BB_P23 | holin protein | 140 | 60 | 64 | 0.428571429 | 0.457142857 |
| flgB | BB_0294 | flagellar basal body rod protein | 11 | 19 | 5 | 1.727272727 | 0.454545455 |
| | | FlgB | | | | | |
| — | BB_0625 | N-acetylmuramoyl-L-alanine | 55 | 81 | 25 | 1.472727273 | 0.454545455 |
| | | amidase | | | | | |
| — | BB_H32 | antigen P35 | 11 | 16 | 5 | 1.454545455 | 0.454545455 |
| — | BB_P30 | hypothetical protein | 11 | 9 | 5 | 0.818181818 | 0.454545455 |
| rpsH | BB_0492 | 30S ribosomal protein S8 | 31 | 21 | 14 | 0.677419355 | 0.451612903 |
| rplI | BB_0112 | 50S ribosomal protein L9 | 51 | 22 | 23 | 0.431372549 | 0.450980392 |
| rho | BB_0230 | transcription termination factor | 49 | 44 | 22 | 0.897959184 | 0.448979592 |
| | | Rho | | | | | |
| — | BB_A64 | P35 antigen | 21094 | 2111 | 9453 | 0.100075851 | 0.448136911 |
| — | BB_L19 | hypothetical protein | 54 | 30 | 24 | 0.555555556 | 0.444444444 |
| — | BB_R43 | hypothetical protein | 250 | 85 | 111 | 0.34 | 0.444 |
| — | predicted | antisense: BB_0631 | 79845 | 34904 | 35398 | 0.437146972 | 0.44333396 |
| | RNA | | | | | | |
| — | BB_P21 | hypothetical protein | 658 | 139 | 291 | 0.211246201 | 0.44224924 |
| — | BB_0238 | hypothetical protein | 59 | 34 | 26 | 0.576271186 | 0.440677966 |
| — | BB_0219 | metal cation transporter | 355 | 321 | 156 | 0.904225352 | 0.43943662 |
| | | permease | | | | | |
| rplM | BB_0339 | 50S ribosomal protein L13 | 48 | 90 | 21 | 1.875 | 0.4375 |
| — | BB_O29 | hypothetical protein | 106 | 46 | 46 | 0.433962264 | 0.433962264 |
| — | predicted | antisense: BB_0034 | 148896 | 35007 | 64256 | 0.235110413 | 0.431549538 |
| | RNA | | | | | | |
| — | BB_Q33 | hypothetical protein | 343 | 57 | 147 | 0.166180758 | 0.428571429 |
| — | predicted | antisense: fusA | 107812 | 32086 | 45574 | 0.297610656 | 0.422717323 |
| | RNA | | | | | | |
| — | BB_R20 | hypothetical protein | 175 | 55 | 73 | 0.314285714 | 0.417142857 |
| cheR | BB_0040 | chemotaxis protein | 560 | 194 | 233 | 0.346428571 | 0.416071429 |
| | | methyltransferase | | | | | |
| — | predicted | antisense: rpsM | 81570 | 23376 | 33897 | 0.286575947 | 0.41555719 |
| | RNA | | | | | | |
| — | predicted | antisense: BB_0269 | 96916 | 27092 | 39823 | 0.279541046 | 0.410902225 |
| | RNA | | | | | | |
| rpsQ | BB_0487 | 30S ribosomal protein S17 | 5 | 2 | 2 | 0.4 | 0.4 |
| — | BB_0525 | hypothetical protein | 184 | 67 | 73 | 0.364130435 | 0.39673913 |
| — | BB_0673 | hypothetical protein | 38 | 12 | 15 | 0.315789474 | 0.394736842 |
| smpB | BB_0033 | SsrA-binding protein | 448 | 41 | 176 | 0.091517857 | 0.392857143 |
| — | BB_0580 | integral membrane protein | 536 | 313 | 210 | 0.583955224 | 0.391791045 |
| — | BB_0831 | xylose operon regulatory protein | 316 | 276 | 122 | 0.873417722 | 0.386075949 |
| — | predicted | antisense: BB_A59 | 183085 | 25097 | 70553 | 0.137078406 | 0.385356528 |
| | RNA | | | | | | |
| lnt | BB_0237 | apolipoprotein N-acyltransferase | 5356 | 1129 | 2060 | 0.210791636 | 0.384615385 |
| — | predicted | antisense: BB_B22 | 117230 | 13859 | 44966 | 0.118220592 | 0.383570758 |
| | RNA | | | | | | |
| — | BB_S21 | hypothetical protein | 29 | 7 | 11 | 0.24137931 | 0.379310345 |
| — | BB_J46 | hypothetical protein | 45 | 13 | 17 | 0.288888889 | 0.377777778 |
| — | BB_H05 | hypothetical protein | 61 | 14 | 23 | 0.229508197 | 0.37704918 |
| — | BB_J47 | hypothetical protein | 183 | 20 | 69 | 0.109289617 | 0.37704918 |
| thyX | BB_A76 | FAD-dependent thymidylate | 8 | 9 | 3 | 1.125 | 0.375 |
| | | synthase | | | | | |
| — | BB_J19 | hypothetical protein | 8 | 6 | 3 | 0.75 | 0.375 |
| rnpA | BB_0441 | ribonuclease P protein | 16 | 10 | 6 | 0.625 | 0.375 |
| | | component | | | | | |
| — | BB_R25 | hypothetical protein | 115 | 7 | 43 | 0.060869565 | 0.373913043 |
| — | BB_0832 | lipoprotein | 2040 | 278 | 753 | 0.13627451 | 0.369117647 |
| — | BB_0373 | hypothetical protein | 11 | 8 | 4 | 0.727272727 | 0.363636364 |
| — | BB_0262 | M23 peptidase domain- | 399 | 185 | 145 | 0.463659148 | 0.363408521 |
| | | containing protein | | | | | |
| — | BB_0824 | hypothetical protein | 278 | 219 | 98 | 0.787769784 | 0.352517986 |
| — | BB_0367 | hypothetical protein | 500 | 63 | 176 | 0.126 | 0.352 |
| — | BB_0663 | hypothetical protein | 1521 | 444 | 533 | 0.291913215 | 0.35042735 |
| dnaB | BB_0111 | replicative DNA helicase | 411 | 72 | 144 | 0.175182482 | 0.350364964 |
| — | BB_Q28 | hypothetical protein | 63 | 10 | 22 | 0.158730159 | 0.349206349 |
| — | BB_P32 | PF-32 protein | 43 | 23 | 15 | 0.534883721 | 0.348837209 |
| — | BB_0153 | superoxide dismutase | 581 | 263 | 202 | 0.452667814 | 0.34767642 |
| ksgA | BB_0590 | rRNA small subunit | 512 | 142 | 177 | 0.27734375 | 0.345703125 |
| | | methyltransferase A | | | | | |
| — | predicted | — | 324978 | 41512 | 111706 | 0.127737878 | 0.343734037 |
| | RNA | | | | | | |
| la7 | BB_0365 | lipoprotein LA7 | 326 | 106 | 112 | 0.325153374 | 0.343558282 |
| — | BB_N41 | hypothetical protein | 137 | 23 | 47 | 0.167883212 | 0.343065693 |
| — | predicted | — | 161854 | 41210 | 55448 | 0.254612181 | 0.34258035 |
| | RNA | | | | | | |
| proS | BB_0402 | proline--tRNA ligase | 257 | 86 | 87 | 0.33463035 | 0.338521401 |
| — | BB_0242 | hypothetical protein | 6 | 9 | 2 | 1.5 | 0.333333333 |
| — | BB_Q54 | hypothetical protein | 3 | 4 | 1 | 1.333333333 | 0.333333333 |
| tRNA- | BB_t17 | Leu tRNA | 45 | 53 | 15 | 1.177777778 | 0.333333333 |
| Leu-4 | | | | | | | |
| — | BB_S33 | hypothetical protein | 87 | 15 | 29 | 0.172413793 | 0.333333333 |
| — | BB_0838a | hypothetical protein | 12 | 2 | 4 | 0.166666667 | 0.333333333 |
| — | BB_0147 | flagellin | 221 | 69 | 73 | 0.312217195 | 0.330316742 |
| blyA | BB_N23 | holin protein | 155 | 50 | 50 | 0.322580645 | 0.322580645 |
| lepB | BB_0263 | signal peptidase I | 304 | 115 | 98 | 0.378289474 | 0.322368421 |
| — | BB_G34 | hypothetical protein | 1371 | 122 | 439 | 0.088986142 | 0.32020423 |
| — | predicted | antisense: BB_0147 | 223894 | 24690 | 71642 | 0.110275398 | 0.319981777 |
| | RNA | | | | | | |
| — | BB_t25 | Gly tRNA | 3756 | 627 | 1187 | 0.166932907 | 0.316027689 |
| — | BB_A58 | hypothetical protein | 40093 | 2956 | 12536 | 0.073728581 | 0.312673035 |
| — | BB_J13 | hypothetical protein | 16 | 20 | 5 | 1.25 | 0.3125 |
| — | BB_0193 | lipoprotein | 48 | 19 | 15 | 0.395833333 | 0.3125 |
| — | BB_D22 | hypothetical protein | 16 | 4 | 5 | 0.25 | 0.3125 |
| — | BB_0102 | hypothetical protein | 170 | 36 | 53 | 0.211764706 | 0.311764706 |
| — | BB_S25 | hypothetical protein | 389 | 42 | 120 | 0.107969152 | 0.30848329 |
| — | BB_L20 | hypothetical protein | 72 | 24 | 22 | 0.333333333 | 0.305555556 |
| — | BB_0664 | lipoprotein | 465 | 152 | 142 | 0.32688172 | 0.305376344 |
| — | BB_0378 | hypothetical protein | 426 | 230 | 129 | 0.539906103 | 0.302816901 |
| fliL | BB_0279 | flagellar basal body-associated | 20 | 13 | 6 | 0.65 | 0.3 |
| | | protein FliL | | | | | |
| — | BB_0559 | PTS system glucose-specific | 595 | 279 | 177 | 0.468907563 | 0.297478992 |
| | | transporter subunit IIA | | | | | |
| — | BB_0593 | long-chain-fatty-acid CoA ligase | 81 | 73 | 24 | 0.901234568 | 0.296296296 |
| — | BB_H06 | complement regulator-acquiring | 34 | 8 | 10 | 0.235294118 | 0.294117647 |
| | | surface protein | | | | | |
| — | BB_P20 | hypothetical protein | 1025 | 118 | 288 | 0.115121951 | 0.28097561 |
| cdd | BB_0618 | cytidine deaminase | 951 | 190 | 267 | 0.199789695 | 0.280757098 |
| — | BB_0185 | glycoprotease family protein | 183 | 175 | 50 | 0.956284153 | 0.273224044 |
| — | BB_J36 | lipoprotein | 334 | 28 | 90 | 0.083832335 | 0.269461078 |
| — | BB_0143 | membrane protein insertion | 4042 | 440 | 1085 | 0.108857001 | 0.26843147 |
| | | efficiency factor | | | | | |
| — | predicted | antisense: ospA ospB | 386319 | 40930 | 102443 | 0.105948711 | 0.265177224 |
| | RNA | | | | | | |
| — | BB_0368 | glycerol-3-phosphate | 371 | 172 | 95 | 0.46361186 | 0.25606469 |
| | | dehydrogenase | | | | | |
| — | BB_0322 | hypothetical protein | 4 | 9 | 1 | 2.25 | 0.25 |
| — | BB_0162 | hypothetical protein | 1622 | 158 | 401 | 0.097410604 | 0.247225647 |
| — | BB_0739 | hypothetical protein | 208 | 63 | 51 | 0.302884615 | 0.245192308 |
| — | BB_0453 | small conductance | 1922 | 357 | 467 | 0.185744017 | 0.242976067 |
| | | mechanosensitive ion channel | | | | | |
| lp6.6 | BB_A62 | 6.6 kDa lipoprotein | 478 | 86 | 115 | 0.179916318 | 0.240585774 |
| udk | BB_0015 | uridine kinase | 156 | 94 | 37 | 0.602564103 | 0.237179487 |
| — | BB_N20 | hypothetical protein | 287 | 69 | 68 | 0.240418118 | 0.236933798 |
| — | BB_0727 | phosphofructokinase | 203 | 83 | 48 | 0.408866995 | 0.236453202 |
| rpmJ | BB_0499 | 50S ribosomal protein L36 | 60 | 72 | 14 | 1.2 | 0.233333333 |
| — | BB_t20 | Ser tRNA | 30 | 9 | 7 | 0.3 | 0.233333333 |
| bdrF | BB_S29 | protein BdrF | 65 | 13 | 15 | 0.2 | 0.230769231 |
| — | predicted | — | 273949 | 37199 | 63152 | 0.135788048 | 0.23052466 |
| | RNA | | | | | | |
| — | BB_Q27 | hypothetical protein | 157 | 17 | 36 | 0.108280255 | 0.229299363 |
| — | BB_Q0091 | hypothetical protein | 27 | 0 | 6 | 0 | 0.222222222 |
| — | BB_0582 | carboxypeptidase | 442 | 69 | 96 | 0.156108597 | 0.21719457 |
| — | BB_G33 | hypothetical protein | 60 | 12 | 13 | 0.2 | 0.216666667 |
| infA | BB_0169 | translation initiation factor IF-1 | 2415 | 486 | 522 | 0.201242236 | 0.216149068 |
| — | BB_0576 | hypothetical protein | 649 | 72 | 140 | 0.110939908 | 0.215716487 |
| — | BB_0316 | integral membrane protein | 169 | 60 | 36 | 0.355029586 | 0.213017751 |
| rpsI | BB_0338 | 30S ribosomal protein S9 | 321 | 174 | 66 | 0.542056075 | 0.205607477 |
| — | BB_0090 | V-type ATP synthase subunit K | 5 | 26 | 1 | 5.2 | 0.2 |
| — | BB_0025 | transcriptional regulator | 1366 | 248 | 272 | 0.181551977 | 0.199121523 |
| — | BB_0628 | lipoprotein | 799 | 153 | 158 | 0.191489362 | 0.197747184 |
| — | predicted | antisense: lp6.6 | 709503 | 52073 | 140114 | 0.073393629 | 0.197481899 |
| | RNA | | | | | | |
| — | BB_Q32 | hypothetical protein | 71 | 6 | 14 | 0.084507042 | 0.197183099 |
| — | BB_A61 | hypothetical protein | 681 | 45 | 133 | 0.066079295 | 0.195301028 |
| — | predicted | antisense: napA | 99610 | 8108 | 19231 | 0.08139745 | 0.193062945 |
| | RNA | | | | | | |
| bdrH | BB_R27 | BdrH | 79 | 9 | 15 | 0.113924051 | 0.189873418 |
| — | BB_H13 | protein RepU | 16 | 1 | 3 | 0.0625 | 0.1875 |
| — | BB_t33 | Thr tRNA | 59 | 15 | 11 | 0.254237288 | 0.186440678 |
| — | BB_I39 | surface antigen | 43 | 3 | 8 | 0.069767442 | 0.186046512 |
| — | BB_0087 | L-lactate dehydrogenase | 275 | 145 | 48 | 0.527272727 | 0.174545455 |
| — | BB_0336 | hypothetical protein | 365 | 124 | 61 | 0.339726027 | 0.167123288 |
| rplX | BB_0489 | 50S ribosomal protein L24 | 36 | 23 | 6 | 0.638888889 | 0.166666667 |
| — | BB_0799 | hypothetical protein | 12 | 5 | 2 | 0.416666667 | 0.166666667 |
| — | BB_Q62 | hypothetical protein | 6 | 0 | 1 | 0 | 0.166666667 |
| tRNA- | BB_t11 | Asn tRNA | 159 | 12 | 26 | 0.075471698 | 0.163522013 |
| Asn-1 | | | | | | | |
| — | BB_B27 | lipoprotein | 267 | 36 | 42 | 0.134831461 | 0.157303371 |
| rpsU | BB_0256 | 30S ribosomal protein S21 | 226 | 81 | 34 | 0.35840708 | 0.150442478 |
| — | BB_0034 | outer membrane protein P13 | 285 | 55 | 41 | 0.192982456 | 0.143859649 |
| — | BB_N30 | hypothetical protein | 7 | 7 | 1 | 1 | 0.142857143 |
| rpmE | BB_0229 | 50S ribosomal protein L31 | 365 | 22 | 52 | 0.060273973 | 0.142465753 |
| — | BB_0353 | membrane protein | 233 | 45 | 31 | 0.193133047 | 0.13304721 |
| — | BB_0428 | hypothetical protein | 4727 | 195 | 627 | 0.04125238 | 0.132642268 |
| — | BB_H33 | adenine deaminase | 16 | 19 | 2 | 1.1875 | 0.125 |
| bdrV | BB_Q42 | protein BdrV | 8 | 5 | 1 | 0.625 | 0.125 |
| — | BB_R31 | hypothetical protein | 2 | 7 | 0 | 3.5 | 0 |
| erpM | BB_O40 | protein ErpM | 2 | 5 | 0 | 2.5 | 0 |
| tRNA- | BB_t05 | Ala tRNA | 6 | 10 | 0 | 1.666666667 | 0 |
| Ala-1 | | | | | | | |
| — | BB_Q39 | hypothetical protein | 3 | 3 | 0 | 1 | 0 |
| — | BB_t21 | Arg tRNA | 3 | 3 | 0 | 1 | 0 |
| rpmC | BB_0486 | 50S ribosomal protein L29 | 11 | 8 | 0 | 0.727272727 | 0 |
| bdrR | BB_N27 | BdrR protein | 8 | 5 | 0 | 0.625 | 0 |
| 5S_rrlB | BB_r03 | 5S ribosomal RNA | 38 | 23 | 0 | 0.605263158 | 0 |
| — | BB_O31 | hypothetical protein | 3 | 1 | 0 | 0.333333333 | 0 |
| tRNA- | BB_t12 | Asp tRNA | 27 | 4 | 0 | 0.148148148 | 0 |
| Asp-1 | | | | | | | |
| tRNA- | BB_t06 | Ser tRNA | 15 | 1 | 0 | 0.066666667 | 0 |
| Ser-3 | | | | | | | |
| — | BB_t28 | Met tRNA | 42 | 2 | 0 | 0.047619048 | 0 |
| blyB | BB_N24 | holin protein | 3 | 0 | 0 | 0 | 0 |
| — | BB_D0027 | hypothetical protein | 11 | 0 | 0 | 0 | 0 |
| tRNA- | BB_t18 | Gln tRNA | 0 | 16 | 0 | — | — |
| Gln-1 | | | | | | | |
| — | BB_J24 | hypothetical protein | 0 | 7 | 0 | — | — |
| — | BB_t23 | Lys tRNA | 0 | 5 | 0 | — | — |
| — | BB_0549 | hypothetical protein | 0 | 4 | 0 | — | — |
| — | BB_0287 | flagellar protein FlbA | 0 | 3 | 0 | — | — |
| — | BB_P33 | PF-49 protein | 0 | 2 | 0 | — | — |
| — | BB_L41 | hypothetical protein | 0 | 2 | 0 | — | — |
| — | BB_Q38 | hypothetical protein | 0 | 2 | 0 | — | — |
| — | BB_0044 | hypothetical protein | 0 | 2 | 0 | — | — |
| — | BB_O02 | hypothetical protein | 0 | 1 | 0 | — | — |
| — | BB_t30 | Phe tRNA | 0 | 1 | 0 | — | — |
| — | BB_0308 | hypothetical protein | 0 | 1 | 0 | — | — |
| — | BB_D24 | hypothetical protein | 0 | 1 | 0 | — | — |
| — | BB_F03 | RepU | 0 | 1 | 0 | — | — |
| — | BB_H29 | plasmid partition protein | 0 | 1 | 0 | — | — |
| — | BB_S06 | hypothetical protein | 0 | 0 | 0 | — | — |
| — | BB_S08 | hypothetical protein | 0 | 0 | 0 | — | — |
| — | BB_S11 | hypothetical protein | 0 | 0 | 0 | — | — |
| — | BB_S12 | hypothetical protein | 0 | 0 | 0 | — | — |
| — | BB_S13 | hypothetical protein | 0 | 0 | 0 | — | — |
| — | BB_S14 | hypothetical protein | 0 | 0 | 0 | — | — |
| — | BB_S18 | hypothetical protein | 0 | 0 | 0 | — | — |
| — | BB_S19 | hypothetical protein | 0 | 0 | 0 | — | — |
| — | BB_S20 | hypothetical protein | 0 | 0 | 0 | — | — |
| blyA | BB_S23 | holin protein | 0 | 0 | 0 | — | — |
| blyB | BB_S24 | holin protein | 0 | 0 | 0 | — | — |
| blyB | BB_R24 | hemolysin accessory protein | 0 | 0 | 0 | — | — |
| — | BB_M01 | phage portal protein | 0 | 0 | 0 | — | — |
| — | BB_M12 | hypothetical protein | 0 | 0 | 0 | — | — |
| — | BB_M13 | hypothetical protein | 0 | 0 | 0 | — | — |
| — | BB_M14 | hypothetical protein | 0 | 0 | 0 | — | — |
| — | BB_M15 | hypothetical protein | 0 | 0 | 0 | — | — |
| — | BB_M17 | hypothetical protein | 0 | 0 | 0 | — | — |
| blyA | BB_M23 | holin protein | 0 | 0 | 0 | — | — |
| — | BB_M25 | hypothetical protein | 0 | 0 | 0 | — | — |
| mlpF | BB_M28 | lipoprotein | 0 | 0 | 0 | — | — |
| — | BB_M30 | hypothetical protein | 0 | 0 | 0 | — | — |
| — | BB_M33 | PF-49 protein | 0 | 0 | 0 | — | — |
| bdrK | BB_M34 | BdrK | 0 | 0 | 0 | — | — |
| erpK | BB_M38 | ErpK protein | 0 | 0 | 0 | — | — |
| — | BB_M39 | hypothetical protein | 0 | 0 | 0 | — | — |
| — | BB_M41 | hypothetical protein | 0 | 0 | 0 | — | — |
| — | BB_O03 | hypothetical protein | 0 | 0 | 0 | — | — |
| — | BB_O04 | hypothetical protein | 0 | 0 | 0 | — | — |
| — | BB_O05 | hypothetical protein | 0 | 0 | 0 | — | — |
| — | BB_O06 | hypothetical protein | 0 | 0 | 0 | — | — |
| — | BB_O08 | hypothetical protein | 0 | 0 | 0 | — | — |
| — | BB_O09 | hypothetical protein | 0 | 0 | 0 | — | — |
| — | BB_O10 | hypothetical protein | 0 | 0 | 0 | — | — |
| — | BB_O13 | hypothetical protein | 0 | 0 | 0 | — | — |
| — | BB_O14 | hypothetical protein | 0 | 0 | 0 | — | — |
| — | BB_O15 | hypothetical protein | 0 | 0 | 0 | — | — |
| — | BB_O16 | hypothetical protein | 0 | 0 | 0 | — | — |
| — | BB_O17 | hypothetical protein | 0 | 0 | 0 | — | — |
| — | BB_O18 | hypothetical protein | 0 | 0 | 0 | — | — |
| — | BB_O19 | hypothetical protein | 0 | 0 | 0 | — | — |
| — | BB_O20 | hypothetical protein | 0 | 0 | 0 | — | — |
| — | BB_O21 | hypothetical protein | 0 | 0 | 0 | — | — |
| — | BB_O22 | hypothetical protein | 0 | 0 | 0 | — | — |
| blyA | BB_O23 | holin protein | 0 | 0 | 0 | — | — |
| blyB | BB_O24 | holin protein | 0 | 0 | 0 | — | — |
| — | BB_O25 | hypothetical protein | 0 | 0 | 0 | — | — |
| — | BB_O26 | hypothetical protein | 0 | 0 | 0 | — | — |
| bppC | BB_O38 | protein BppC | 0 | 0 | 0 | — | — |
| erpL | BB_O39 | protein ErpL | 0 | 0 | 0 | — | — |
| — | BB_O41 | hypothetical protein | 0 | 0 | 0 | — | — |
| — | BB_O42 | hypothetical protein | 0 | 0 | 0 | — | — |
| — | BB_L01 | phage portal protein | 0 | 0 | 0 | — | — |
| — | BB_L02 | hypothetical protein | 0 | 0 | 0 | — | — |
| — | BB_L03 | hypothetical protein | 0 | 0 | 0 | — | — |
| — | BB_L04 | hypothetical protein | 0 | 0 | 0 | — | — |
| — | BB_L06 | hypothetical protein | 0 | 0 | 0 | — | — |
| — | BB_L07 | hypothetical protein | 0 | 0 | 0 | — | — |
| — | BB_L08 | hypothetical protein | 0 | 0 | 0 | — | — |
| — | BB_L09 | hypothetical protein | 0 | 0 | 0 | — | — |
| — | BB_L10 | hypothetical protein | 0 | 0 | 0 | — | — |
| — | BB_L11 | hypothetical protein | 0 | 0 | 0 | — | — |
| — | BB_L12 | hypothetical protein | 0 | 0 | 0 | — | — |
| — | BB_L13 | hypothetical protein | 0 | 0 | 0 | — | — |
| — | BB_L14 | hypothetical protein | 0 | 0 | 0 | — | — |
| — | BB_L15 | hypothetical protein | 0 | 0 | 0 | — | — |
| — | BB_L16 | hypothetical protein | 0 | 0 | 0 | — | — |
| — | BB_L17 | hypothetical protein | 0 | 0 | 0 | — | — |
| — | BB_L18 | hypothetical protein | 0 | 0 | 0 | — | — |
| — | BB_L21 | hypothetical protein | 0 | 0 | 0 | — | — |
| — | BB_L22 | hypothetical protein | 0 | 0 | 0 | — | — |
| blyA | BB_L23 | holin protein | 0 | 0 | 0 | — | — |
| blyB | BB_L24 | holin protein | 0 | 0 | 0 | — | — |
| — | BB_L29 | hypothetical protein | 0 | 0 | 0 | — | — |
| — | BB_L31 | hypothetical protein | 0 | 0 | 0 | — | — |
| — | BB_L32 | PF-32 protein | 0 | 0 | 0 | — | — |
| bdrO | BB_L35 | protein BdrO | 0 | 0 | 0 | — | — |
| bppA | BB_L36 | protein BppA | 0 | 0 | 0 | — | — |
| — | BB_N08 | hypothetical protein | 0 | 0 | 0 | — | — |
| — | BB_U01 | hypothetical protein | 0 | 0 | 0 | — | — |
| — | BB_U04 | protein BBC01 | 0 | 0 | 0 | — | — |
| — | BB_U05 | PF-32 protein | 0 | 0 | 0 | — | — |
| — | BB_U06 | plasmid partition protein | 0 | 0 | 0 | — | — |
| — | BB_U07 | protein BBC01 | 0 | 0 | 0 | — | — |
| — | BB_U08 | SUA5 subfamily protein | 0 | 0 | 0 | — | — |
| — | BB_U09 | TM2 family protein | 0 | 0 | 0 | — | — |
| — | BB_U10 | protein BBC01 | 0 | 0 | 0 | — | — |
| — | BB_U11 | Sua5/YciO/YrdC/YwlC family | 0 | 0 | 0 | — | — |
| | | protein | | | | | |
| — | BB_Q03 | outer membrane protein | 0 | 0 | 0 | — | — |
| — | BB_Q05 | antigen, P35 | 0 | 0 | 0 | — | — |
| — | BB_Q07 | plasmid partition protein | 0 | 0 | 0 | — | — |
| — | BB_Q08 | PF-32 protein | 0 | 0 | 0 | — | — |
| — | BB_Q09 | hypothetical protein | 0 | 0 | 0 | — | — |
| — | BB_Q40 | PF-32 protein | 0 | 0 | 0 | — | — |
| erpX | BB_Q47 | protein ErpX | 0 | 0 | 0 | — | — |
| — | BB_Q48 | hypothetical protein | 0 | 0 | 0 | — | — |
| — | BB_Q49 | hypothetical protein | 0 | 0 | 0 | — | — |
| — | BB_Q67 | adenine specific DNA | 0 | 0 | 0 | — | — |
| | | methyltransferase | | | | | |
| — | BB_T01 | hypothetical protein | 0 | 0 | 0 | — | — |
| — | BB_T02 | hypothetical protein | 0 | 0 | 0 | — | — |
| — | BB_T03 | hypothetical protein | 0 | 0 | 0 | — | — |
| — | BB_T04 | hypothetical protein | 0 | 0 | 0 | — | — |
| — | BB_T06 | Sua5/YciO/YrdC/YwlC family | 0 | 0 | 0 | — | — |
| | | protein | | | | | |
| — | BB_T07 | outer membrane protein | 0 | 0 | 0 | — | — |
| — | BB_0138 | hypothetical protein | 0 | 0 | 0 | — | — |
| — | BB_0305 | hypothetical protein | 0 | 0 | 0 | — | — |
| tRNA- | BB_t07 | Lys tRNA | 0 | 0 | 0 | — | — |
| Lys-2 | | | | | | | |
| tRNA- | BB_t08 | His tRNA | 0 | 0 | 0 | — | — |
| His-1 | | | | | | | |
| — | BB_t26 | Arg tRNA | 0 | 0 | 0 | — | — |
| tRNA- | BB_t14 | Thr tRNA | 0 | 0 | 0 | — | — |
| Thr-2 | | | | | | | |
| — | BB_E09 | protein p23 | 0 | 0 | 0 | — | — |
| — | BB_E16 | hypothetical protein | 0 | 0 | 0 | — | — |
| — | BB_E17 | hypothetical protein | 0 | 0 | 0 | — | — |
| — | BB_E18 | hypothetical protein | 0 | 0 | 0 | — | — |
| — | BB_E19 | hypothetical protein | 0 | 0 | 0 | — | — |
| — | BB_E20 | hypothetical protein | 0 | 0 | 0 | — | — |
| — | BB_E21 | hypothetical protein | 0 | 0 | 0 | — | — |
| — | BB_E22 | pyrazinamidase/nicotinamidase | 0 | 0 | 0 | — | — |
| | | PncA | | | | | |
| — | BB_E31 | antigen, P35, putative | 0 | 0 | 0 | — | — |
| — | BB_F01 | hypothetical protein | 0 | 0 | 0 | — | — |
| — | BB_F02 | hypothetical protein | 0 | 0 | 0 | — | — |
| — | BB_F06 | hypothetical protein | 0 | 0 | 0 | — | — |
| — | BB_F08 | hypothetical protein | 0 | 0 | 0 | — | — |
| — | BB_F0034 | hypothetical protein | 0 | 0 | 0 | — | — |
| — | BB_F14 | hypothetical protein | 0 | 0 | 0 | — | — |
| — | BB_F17 | putative transmembrane protein | 0 | 0 | 0 | — | — |
| — | BB_F20 | BBF20 | 0 | 0 | 0 | — | — |
| — | BB_F23 | PF49 | 0 | 0 | 0 | — | — |
| — | BB_F24 | PF32 | 0 | 0 | 0 | — | — |
| — | BB_F25 | hypothetical protein | 0 | 0 | 0 | — | — |
| — | BB_F26 | hypothetical protein | 0 | 0 | 0 | — | — |
| — | BB_F0039 | hypothetical protein | 0 | 0 | 0 | — | — |
| — | BB_F0040 | hypothetical protein | 0 | 0 | 0 | — | — |
| — | BB_F0041 | outer surface protein VlsE1 | 0 | 0 | 0 | — | — |
| — | BB_I01 | hypothetical protein | 0 | 0 | 0 | — | — |
| — | BB_I02 | hypothetical protein | 0 | 0 | 0 | — | — |
| — | BB_I06 | MTA/SAH nucleosidase | 0 | 0 | 0 | — | — |
| — | BB_I12 | hypothetical protein | 0 | 0 | 0 | — | — |
| — | BB_I15 | virulence associated lipoprotein | 0 | 0 | 0 | — | — |
| vraA | BB_I16 | repetitive antigen A | 0 | 0 | 0 | — | — |
| — | BB_I18 | hypothetical protein | 0 | 0 | 0 | — | — |
| — | BB_I19 | hypothetical protein | 0 | 0 | 0 | — | — |
| — | BB_I20 | hypothetical protein | 0 | 0 | 0 | — | — |
| — | BB_I21 | PF-32 protein | 0 | 0 | 0 | — | — |
| — | BB_I22 | PF-49 protein | 0 | 0 | 0 | — | — |
| — | BB_I26 | multidrug-efflux transporter | 0 | 0 | 0 | — | — |
| — | BB_I29 | virulence associated lipoprotein | 0 | 0 | 0 | — | — |
| — | BB_I34 | virulence associated lipoprotein | 0 | 0 | 0 | — | — |
| — | BB_I36 | antigen P35 | 0 | 0 | 0 | — | — |
| — | BB_I38 | surface antigen | 0 | 0 | 0 | — | — |
| — | BB_I41 | hypothetical protein | 0 | 0 | 0 | — | — |
| — | BB_I42 | outer membrane protein | 0 | 0 | 0 | — | — |
| — | BB_K01 | lipoprotein | 0 | 0 | 0 | — | — |
| — | BB_K07 | lipoprotein | 0 | 0 | 0 | — | — |
| — | BB_K09 | hypothetical protein | 0 | 0 | 0 | — | — |
| — | BB_K13 | hypothetical protein | 0 | 0 | 0 | — | — |
| — | BB_K15 | antigen P35 | 0 | 0 | 0 | — | — |
| — | BB_K17 | adenine deaminase | 0 | 0 | 0 | — | — |
| — | BB_K19 | lipoprotein | 0 | 0 | 0 | — | — |
| — | BB_K21 | PF-32 protein | 0 | 0 | 0 | — | — |
| — | BB_K22 | hypothetical protein | 0 | 0 | 0 | — | — |
| — | BB_K23 | hypothetical protein | 0 | 0 | 0 | — | — |
| — | BB_K24 | PF-49 protein | 0 | 0 | 0 | — | — |
| — | BB_K54 | hypothetical protein | 0 | 0 | 0 | — | — |
| — | BB_K32 | fibronectin-binding protein | 0 | 0 | 0 | — | — |
| — | BB_K33 | hypothetical protein | 0 | 0 | 0 | — | — |
| — | BB_K34 | hypothetical protein | 0 | 0 | 0 | — | — |
| — | BB_K35 | hypothetical protein | 0 | 0 | 0 | — | — |
| — | BB_K0058 | hypothetical protein | 0 | 0 | 0 | — | — |
| — | BB_K40 | hypothetical protein | 0 | 0 | 0 | — | — |
| — | BB_K41 | hypothetical protein | 0 | 0 | 0 | — | — |
| — | BB_K42 | hypothetical protein | 0 | 0 | 0 | — | — |
| — | BB_K45 | immunogenic protein P37 | 0 | 0 | 0 | — | — |
| — | BB_K47 | hypothetical protein | 0 | 0 | 0 | — | — |
| — | BB_K48 | immunogenic protein P37 | 0 | 0 | 0 | — | — |
| — | BB_K49 | hypothetical protein | 0 | 0 | 0 | — | — |
| — | BB_K50 | immunogenic protein P37 | 0 | 0 | 0 | — | — |
| — | BB_K0059 | hypothetical protein | 0 | 0 | 0 | — | — |
| — | BB_K52 | lipoprotein | 0 | 0 | 0 | — | — |
| — | BB_K53 | outer membrane protein | 0 | 0 | 0 | — | — |
| — | BB_J0058 | hypothetical protein | 0 | 0 | 0 | — | — |
| — | BB_J42 | hypothetical protein | 0 | 0 | 0 | — | — |

### Example 4: Commonalties Among the Pathways of the Doxycycline Persisters and Amoxicillin Persisters

A comparison of the pathways of the doxycycline persisters and amoxicillin persisters revealed that they share several common features. For example, transporter (BB_0164, BB_0729), membrane protein (BB_A05, BB_0385), chemotaxis (BB_0414), ClpP protease (BB_0757), and DNA repair genes are up-regulated, while genes for outer membrane proteins/lipoproteins (BB_A61, BB_I39, BB_Q32) and ribosomal proteins (BB_0229) involved in protein synthesis were down-regulated. Besides these common pathways, genes for heat shock proteins and protein secretion protein were upregulated in doxycycline persisters, while amoxicillin persisters had virulence genes (dbpAB), terpenoid synthesis genes, and drug metabolism P450 family dehydrogenase gene uprgulated and glycolysis/gluconeogenesis genes down-regulated. These gene expression changes may play important roles in facilitating survival of B. burgdorferi persisters under antibiotic stress.

Amoxicillin inhibits the synthesis of bacterial cell wall and induces B. burgdorferi into round body form (Feng J, et al., PLoS One. 2015; 10(3):e0117207). In the amoxicillin tolerant persisters many down-regulated outer membrane lipoprotein genes were found, which may provide a persistence strategy for persisters. In addition, two terpenoid/steroid synthesis genes were found to be up-regulated in amoxicillin tolerant persisters. B. burgdorferi is an unusual prokaryote that possesses sterols in its membranes (LaRocca T J, et al. PLoS Pathog. 2013; 9(5):e1003353). Up-regulation of terpenoid/steroid synthesis genes in amoxicillin tolerant persisters may increase the content of sterols, and change the cell membrane component to allow survival under amoxicillin treatment. B. burgdorferi decorin-binding proteins (Dbp) A (BB_A24) and B (BB_A25) are mainly expressed during mammalian infection to mediate bacterial attachment to the proteoglycan decorin in decorin-expressing mammal cells (15). The elevated dbpA/B expression in B. burgdorferi persisters without mammalian infection by amoxicillin treatment may enhance interaction with host cell and promote pathogen persistence.

The findings not only shed new light on the mechanisms of persisters in B. burgdorferi but also have practical applications. For example, the upregulated genes identified in persisters may serve as targets for developing new drugs for more effective treatment, but also may serve as antigens for developing diagnostic tests for persistent Lyme disease, and for developing therapeutic vaccines for improved treatment.

### Example 5: Viability Assay Kit for Borrelia burgdorferi and Related Spirochete Species for Viability Assessment and Antibiotic Susceptibility Testing

The present invention also provides kits for use in treating and/or diagnosing Lyme disease. The kits of the present invention include one or more containers comprising B. burgdorferi therapeutics (siRNAs, antibodies, etc.), and in some embodiments, further comprise instructions for use in accordance with any of the methods described herein. The kit may further comprise a description of selecting an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

A patient can be diagnosed by adding a sample from the patient to a kit and detecting said biomarker conjugated with a biomarker antibody, specifically, by a method which comprises the steps of: (i) collecting sample from the patient; (ii) centrifuging sample; (iii) adding the sample from patient to a diagnostic kit; and, (iv) detecting said biomarker conjugated with an antibody. In this method, the antibodies are brought into contact with the patient's sample. If said biomarker is present in the sample, the antibodies will bind to the sample, or a portion thereof. In other kit and diagnostic embodiments, sample need not be collected from the patient (i.e., it is already collected).

The kit can also comprise a washing solution or instructions for making a washing solution, in which the combination of the capture agent and the washing solution allows capture of the biomarker or biomarkers on the solid support for subsequent detection by, e.g., an antibody or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected. In yet another embodiment, the kit can comprise one or more containers with biomarker samples, to be used as standard(s) for calibration.

### Example 6: Viability Assay Kit for Borrelia burgdorferi for Drug Screens in High Throughput Format

A high throughput screen is performed against B. burgdorferi to identify a family of structurally related compounds with inhibitory activity. The compounds are further optimized and verified in follow up screens and assays.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### REFERENCES

1. Lyme Disease. Center for Disease Control; 2015 [cited 2015 Jun. 5, 2015]; Available from: (www)cec.gov/lyme/.
2. Rizzoli A, Hauffe H, Carpi G, Vourc H G, Neteler M, Rosa R. Lyme borreliosis in Europe. Euro Surveill. 2011; 16(27).
3. Wormser G P, Dattwyler R J, Shapiro E D, Halperin J J, Steere A C, Klempner M S, et al. The clinical assessment, treatment, and prevention of lyme disease, human granulocytic anaplasmosis, and babesiosis: clinical practice guidelines by the Infectious Diseases Society of America. Clinical infectious diseases: an official publication of the Infectious Diseases Society of America. [Practice Guideline]. 2006 Nov. 1; 43(9):1089-134.
4. CDC. Post-Treatment Lyme Disease Syndrome. Available: (www)cdc.gov/lyme/postLDS/index.html. Accessed 2014 Sep. 13, 2014 [9/13/2014]; Available from: (www)cdc.gov/lyme/postLDS/index.html.
5. Bockenstedt L K, Gonzalez D G, Haberman A M, Belperron A A. Spirochete antigens persist near cartilage after murine Lyme borreliosis therapy. J Clin Invest. 2012 Jul. 2; 122(7):2652-60.
6. Steere A C, Gross D, Meyer A L, Huber B T. Autoimmune mechanisms in antibiotic treatment-resistant lyme arthritis. Journal of autoimmunity. 2001 May; 16(3):263-8.
7. Stricker R B. Counterpoint: long-term antibiotic therapy improves persistent symptoms associated with lyme disease. Clinical infectious diseases: an official publication of the Infectious Diseases Society of America. 2007 Jul. 15; 45(2):149-57.
8. Barthold S W, Hodzic E, Imai D M, Feng S, Yang X, Luft B J. Ineffectiveness of tigecycline against persistent Borrelia burgdorferi. Antimicrobial Agents and Chemotherapy. 2010 February; 54(2):643-51.
9. Embers M E, Barthold S W, Borda J T, Bowers L, Doyle L, Hodzic E, et al. Persistence of Borrelia burgdorferi in rhesus macaques following antibiotic treatment of disseminated infection. PLoS One. 2012; 7(1):e29914.
10. Straubinger R K, Summers B A, Chang Y F, Appel M J. Persistence of Borrelia burgdorferi in experimentally infected dogs after antibiotic treatment. J Clin Microbiol. 1997 January; 35(1):111-6.
11. Feng J, Wang T, Shi W, Zhang S, Sullivan D, Auwaerter P G, et al. Identification of Novel Activity against Borrelia burgdorferi Persisters Using an FDA Approved Drug Library. Emerg Microb Infect. 2014; Jul. 2, 2014:3, e49; doi:10.1038/emi.2014.53.
12. Zhang Y. Persisters, Persistent Infections and the Yin-Yang Model. Emerg Microb Infect (Nature Publishing Group). 2014; 3, e3; doi:10.1038/emi.2014.3.
13. Feng J, Auwaerter P G, Zhang Y. Drug Combinations against Borrelia burgdorferi Persisters In Vitro: Eradication Achieved by Using Daptomycin, Cefoperazone and Doxycycline. PLoS One. 2015; 10(3):e0117207.
14. LaRocca T J, Pathak P, Chiantia S, Toledo A, Silvius J R, Benach J L, et al. Proving lipid rafts exist: membrane domains in the prokaryote Borrelia burgdorferi have the same properties as eukaryotic lipid rafts. PLoS Pathog. 2013; 9(5):e1003353.
15. Salo J, Loimaranta V, Landenne P, Viljanen M K, Hytonen J. Decorin binding by DbpA and B of Borrelia garinii, Borrelia afzelii, and Borrelia burgdorferi sensu Stricto. J Infect Dis. 2011 Jul. 1; 204(1):65-73.

1. A method for diagnosing a Borrelia burgdorferi infection in a subject in need thereof comprising:
obtaining a sample from the subject;
incubating the sample with a binding agent to yield a binding agent complex;
performing an in vitro reaction on the binding agent complex to detect one or more Borrelia burgdorferi biomarkers; and
detecting the level of the one or more biomarkers and comparing the level to a reference.

2. The method of claim 1, further comprising computing a level of the one or more biomarkers.

3. The method of claim 1, wherein the Borrelia burgdorferi infection causes Lyme disease.

4. The method of claim 1, wherein the one or more biomarkers identify antibiotic tolerant persisters.

5. The method of claim 4, wherein the antibiotic is doxycline or amoxicillin.

6. The method of claim 1, wherein the binding agent comprises an antibody or a fragment thereof, a detectable protein, or a fragment thereof, or a nucleic acid molecule.

7. The method of claim 1, wherein the in vitro reaction comprises quantitative PCR or Northern Blot.

8. The method of claim 6, wherein the nucleic acid comprises cDNA.

9. The method of claim 1, wherein the binding agent comprises an antibody.

10. The method of claim 9, wherein the antibody is conjugated to a detectable moiety.

11. The method of claim 9, wherein the antibody comprises a polyclonal antibody or a monoclonal antibody.

12-14. (canceled)

15. The method of claim 1, wherein the subject is a human.

16. The method of claim 1, wherein the sample comprises plasma, whole blood, serum, saliva or urine.

17. The method of claim 1, wherein the sample comprises serum.

18. The method of claim 1, detecting further comprises an assay selected from the group consisting of an ELISA (enzyme-linked immunosorbent) assay, a Western blot, a mass spectrometry, a radioimmunoassay, and a fluoroimmunoassay.

19. A kit comprising a binding agent for detecting one or more biomarkers for diagnosing a Borrelia burgdorferi infection in a subject in need thereof,
wherein the binding agent binds to the one or more biomarkers, and yields a complex comprising the one or more biomarkers and the binding agent, thereby allowing detection of the level of the one or more biomarkers,
and instructions for using the binding agent for diagnosing a Borrelia burgdorferi infection.

20-22. (canceled)

23. A method of treating a Borrelia burgdorferi infection in a subject comprising,
measuring the level of one or more biomarkers in a subject, and
administering to said subject, an active agent for treating a Borrelia burgdorferi infection, based on the level of the one or more biomarkers.

24. The method of claim 23, wherein the active agent comprises doxycycline, amoxicillin, cefuroxime axetil, ceftriaxone, cefotaxime, penicillin G, or bismacine.

26. A method for detecting Post Treatment Lyme Disease Syndrome in a subject in need thereof comprising,
obtaining a sample from the subject;
incubating the sample with a binding agent to yield a binding agent complex;
performing an in vitro reaction on the binding agent complex to detect one or more Borrelia burgdorferi biomarkers; and
detecting the level of the one or more biomarkers and comparing the level to a reference to detect Post Treatment Lyme Disease Syndrome.
